(19) United States
(12) Patent Application Publication
(10) Pub. No.: US 2006/0069123 A1
(43) Pub. Date: Mar. 30, 2006

(54) Substituted dipiperidine CCR2 antagonists

(76) Inventors: Mingde Xia, Belle Mead, NJ (US); Michael P. Wachter, Bloomsbury, NJ (US); Meng Pan, Neshanic Station, NJ (US); Duane E. DeMong, Somerset, NJ (US); Scott R. Pollack, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/224,215

(22) Filed: Sep. 12, 2005

Related U.S. Application Data
(60) Provisional application No. 60/613,922, filed on Sep. 28, 2004.

(51) Int. Cl. A61K 31/4545; C07D 403/14

(52) U.S. Cl. 514/316; 546/187

(57) ABSTRACT

Substituted dipiperidine compounds of Formula (I) 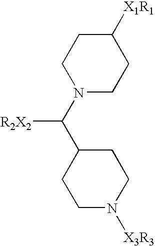 or a salt, isomer, prodrug, metabolite or polymorph thereof, which are CCR2 antagonists and are useful in preventing, treating or ameliorating CCR2 mediated inflammatory syndromes, disorders or diseases in a subject in need thereof.

### CROSS REFERENCE TO RELATED APPLICATIONS

This present application claims benefit of U.S. Provisional Patent Application Ser. No. 60/613,922, filed Sep. 28, 2004, which is incorporated herein by reference in its entirety and for all purposes.

### FIELD OF THE INVENTION

The invention is directed to substituted dipiperidine compounds, which are antagonists to the chemoattractant cytokine receptor 2 (CCR2), pharmaceutical compositions, and methods for use thereof. More particularly, the CCR2 antagonists are substituted dipiperidine carboxylic acid, alcohol and ester compounds useful for preventing, treating or ameliorating a CCR2 mediated inflammatory syndrome, disorder or disease.

### BACKGROUND OF THE INVENTION

CCR2 is a member of the GPCR family of receptors, as are all known chemokine receptors, and are expressed by monocytes and memory T-lymphocytes. The CCR2 signaling cascade involves activation of phospholipases (PLCβ2), protein kinases (PKC), and lipid kinases (PI-3 kinase).

Chemoattractant cytokines (i.e., chemokines) are relatively small proteins (8-10 kD), which stimulate the migration of cells. The chemokine family is divided into four subfamilies based on the number of amino acid residues between the first and second highly conserved cysteines.

Monocyte chemotactic protein-1 (MCP-1) is a member of the CC chemokine subfamily (wherein CC represents the subfamily having adjacent first and second cysteines) and binds to the cell-surface chemokine receptor 2 (CCR2). MCP-1 is a potent chemotactic factor, which, after binding to CCR2, mediates monocyte and lymphocyte migration (i.e., chemotaxis) toward a site of inflammation. MCP-1 is also expressed by cardiac muscle cells, blood vessel endothelial cells, fibroblasts, chondrocytes, smooth muscle cells, mesangial cells, alveolar cells, T-lymphocytes, marcophages, and the like.

After monocytes enter the inflammatory tissue and differentiate into macrophages, monocyte differentiation provides a secondary source of several proinflammatory modulators, including tumor necrosis factor-α (TNF-α), interleukin-1 (IL-1), IL-8 (a member of the CXC chemokine subfamily, wherein CXC represents one amino acid residue between the first and second cysteines), IL-12, arachidonic acid metabolites (e.g., PGE₂ and LTB₄), oxygen-derived free radicals, matrix metalloproteinases, and complement components.

Animal model studies of chronic inflammatory diseases have demonstrated that inhibition of binding between MCP-1 and CCR2 by an antagonist suppresses the inflammatory response. The interaction between MCP-1 and CCR2 has been implicated (see Rollins B J, Monocyte chemoattractant protein 1: a potential regulator of monocyte recruitment in inflammatory disease, Mol. Med. Today, 1996, 2:198; and Dawson J, et al., Targeting monocyte chemoattractant protein-1 signaling in disease, Expert Opin. Ther. Targets, 2003 Feb. 7 (1):35-48) in inflammatory disease pathologies such as psoriasis, uveitis, atherosclerosis, rheumatoid arthritis, multiple sclerosis, Crohn's Disease, nephritis, organ allograft rejection, fibroid lung, renal insufficiency, diabetes and diabetic complications, diabetic nephropathy, diabetic retinopathy, diabetic retinitis, diabetic microangiopathy, tuberculosis, sarcoidosis, invasive staphylococcia, inflammation after cataract surgery, allergic rhinitis, allergic conjunctivitis, chronic urticaria, Chronic Obstructive Pulmonary Disease (COPD), allergic asthma, periodontal diseases, periodonitis, gingivitis, gum disease, diastolic cardiomyopathies, cardiac infarction, myocarditis, chronic heart failure, angiostenosis, restenosis, reperfusion disorders, glomerulonephritis, solid tumors and cancers, chronic lymphocytic leukemia, chronic myelocytic leukemia, multiple myeloma, malignant myeloma, Hodgkin's disease, and carcinomas of the bladder, breast, cervix, colon, lung, prostate, and stomach.

Monocyte migration is inhibited by MCP-1 antagonists (either antibodies or soluble, inactive fragments of MCP-1), which have been shown to inhibit the development of arthritis, asthma, and uveitis. Both MCP-1 and CCR2 knockout (KO) mice have demonstrated that monocyte infiltration into inflammatory lesions is significantly decreased. In addition, such KO mice are resistant to the development of experimental allergic encephalomyelitis (EAE, a model of human MS), cockroach allergen-induced asthma, atherosclerosis, and uveitis. Rheumatoid arthritis and Crohn's Disease patients have improved during treatment with TNF-α antagonists (e.g., monoclonal antibodies and soluble receptors) at dose levels correlated with decreases in MCP-1 expression and the number of infiltrating macrophages.

MCP-1 has been implicated in the pathogenesis of seasonal and chronic allergic rhinitis, having been found in the nasal mucosa of most patients with dust mite allergies. MCP-1 has also been found to induce histamine release from basophils in vitro. During allergic conditions, both allergens and histamines have been shown to trigger (i.e., to up-regulate) the expression of MCP-1 and other chemokines in the nasal mucosa of people with allergic rhinitis, suggesting the presence of a positive feedback loop in such patients.

There remains a need for small molecule CCR2 antagonists for preventing, treating or ameliorating a CCR2 mediated inflammatory syndrome, disorder or disease resulting from MCP-1 induced monocyte and lymphocyte migration to a site of inflammation.

All documents cited herein are incorporated by reference.

### SUMMARY OF THE INVENTION

The invention provides substituted dipiperidine compounds of Formula (I) 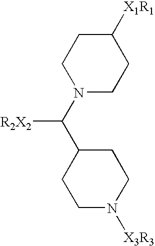
or a salt, isomer, prodrug, metabolite or polymorph thereof, which are CCR2 antagonists and are useful in preventing, treating or ameliorating CCR2 mediated inflammatory syndromes, disorders or diseases in a subject in need thereof.

The present invention also provides a method for preventing, treating or ameliorating a CCR2 mediated inflammatory syndrome, disorder or disease in a subject in need thereof comprising administering to the subject an effective amount of a compound of Formula (I) or a form, composition or medicament thereof.

### DETAILED DESCRIPTION OF THE INVENTION

The present invention is directed to a compound of Formula (I) 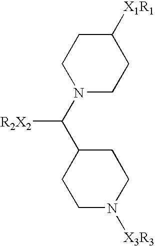
or a salt, isomer, prodrug, metabolite or polymorph thereof wherein
X₁ is absent, alkyl, carbonyl, alkylcarbamoyl or alkylcarbamoylalkyl,
R₁ is aryl or heterocyclyl, wherein heterocyclyl has an optionally present nitrogen atom and wherein the nitrogen atom is optionally oxidized, and wherein aryl and heterocyclyl are each optionally substituted with one or more of alkyl, alkoxy, cyano, halogen, hydroxy, hydroxyalkyl, nitro, amino (optionally substituted with one or more of alkyl, acyl, carbonylalkoxy, sulfonylalkyl, alkylcarboxy or alkylcarbonylalkoxy), alkylcarboxy, alkylcarbonylalkoxy, alkoxycarboxy, alkoxycarbonylalkoxy, alkylamino, alkylaminoalkyl, sulfonylamino, sulfonylaminoalkyl, alkylsulfonylamino, alkylsulfonylaminoalkyl, carboxy, acyl, carbonylalkoxy, carbamoyl or carbamoylalkyl,
X₂ is absent or alkyl,
R₂ is hydroxy, halogen, amino (optionally substituted with one or more of alkyl, formyl, acyl, sulfonylalkyl or carbonylalkoxy), cyano, nitro, alkoxy, carboxy, carbonylalkoxy, oxyacyl, oxyacylaryl, oxyacrylyl, oxyacrylylaryl (optionally substituted on aryl with one or more of alkyl, alkoxy, cyano, halogen, hydroxy, nitro, amino or aminoalkyl), oxycarbonylalkoxy, aminoacylamino, aminoacylaminoalkyl, carbamoyl, carbamoylalkyl, urea or ureaalkyl,
X₃ is carbonyl, carboxyl, acyl, acyloxy, acrylyl, carbonylalkynyl, carbonylalkoxy, carbamoyl, carbamoylalkyl, alkylcarbamoyl, thiocarbamyl or iminomethylaminocarbonyl, wherein when X₃ is carbonylalkoxy, then R₃ is optionally present, and R₃ is cycloalkyl, aryl or heterocyclyl each optionally substituted with one or more of alkyl, alkoxy, cyano, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl, carbamoylalkyl or aryl (optionally substituted on aryl with one or more of alkyl, alkoxy, halogen, hydroxy, nitro, amino or aminoalkyl).

An example of the invention is a compound of Formula (I) and a salt, isomer, prodrug, metabolite or polymorph thereof, wherein X₁ is absent, alkyl or alkylcarbamoylalkyl.

An example of the invention is a compound of Formula (I) and a salt, isomer, prodrug, metabolite or polymorph thereof, wherein X₁ is alkyl or alkylcarbamoylalkyl.

An example of the invention is a compound of Formula (I) and a salt, isomer, prodrug, metabolite or polymorph thereof, wherein X₁ is absent.

An example of the invention is a compound of Formula (I) and a salt, isomer, prodrug, metabolite or polymorph thereof, wherein R₁ is phenyl or heterocyclyl, wherein heterocyclyl has an optionally present nitrogen atom and wherein the nitrogen atom is optionally oxidized, and wherein phenyl and heterocyclyl are each optionally substituted with one or more of alkyl, alkoxy, cyano, halogen, hydroxy, alkylhydroxy, nitro, amino (optionally substituted with one or more of alkyl, acyl, carbonylalkoxy, sulfonylalkyl, alkylcarboxy or alkylcarbonylalkoxy), alkylcarboxy, alkylcarbonylalkoxy, alkoxycarboxy, alkoxycarbonylalkoxy, alkylamino, alkylaminoalkyl, sulfonylamino, sulfonylaminoalkyl, alkylsulfonylamino, alkylsulfonylaminoalkyl, carboxy, acyl, carbonylalkoxy, carbamoyl or carbamoylalkyl.

An example of the invention is a compound of Formula (I) and a salt, isomer, prodrug, metabolite or polymorph thereof, wherein R₁ is aryl or heterocyclyl, wherein heterocyclyl has an optionally present nitrogen atom and wherein the nitrogen atom is optionally oxidized, and wherein aryl and heterocyclyl are each optionally substituted with one or more of alkyl, alkoxy, cyano, halogen, hydroxy, alkylhydroxy, nitro, amino (optionally substituted with one or more of alkyl, acyl, carbonylalkoxy, sulfonylalkyl, alkylcarboxy or alkylcarbonylalkoxy), alkylcarboxy, alkylcarbonylalkoxy, alkoxycarboxy, alkoxycarbonylalkoxy, sulfonylamino, sulfonylaminoalkyl, alkylsulfonylamino, alkylsulfonylaminoalkyl, carboxy, acyl or carbonylalkoxy.

An example of the invention is a compound of Formula (I) and a salt, isomer, prodrug, metabolite or polymorph thereof, wherein R₁ is phenyl or heterocyclyl, wherein heterocyclyl has an optionally present nitrogen atom and wherein the nitrogen atom is optionally oxidized, and wherein phenyl and heterocyclyl are each optionally substituted with one or more of alkyl, alkoxy, cyano, halogen, hydroxy, alkylhydroxy, nitro, amino (optionally substituted with one or more of alkyl, acyl; carbonylalkoxy, sulfonylalkyl, alkylcarboxy or alkylcarbonylalkoxy), alkylcarboxy, alkylcarbonylalkoxy, alkoxycarboxy, alkoxycarbonylalkoxy, sulfonylamino, sulfonylaminoalkyl, alkylsulfonylamino, alkylsulfonylaminoalkyl, carboxy, acyl or carbonylalkoxy.

An example of the invention is a compound of Formula (I) and a salt, isomer, prodrug, metabolite or polymorph thereof, wherein R₁ is aryl or heterocyclyl, wherein heterocyclyl has an optionally present nitrogen atom and wherein the nitrogen atom is optionally oxidized, and wherein aryl and heterocyclyl are each optionally substituted with one or more of alkyl, alkoxy, halogen, hydroxy, amino (optionally substituted with one or more of alkyl, acyl, carbonylalkoxy or sulfonylalkyl), carboxy, acyl or carbonylalkoxy.

An example of the invention is a compound of Formula (I) and a salt, isomer, prodrug, metabolite or polymorph thereof, wherein R₁ is phenyl or heterocyclyl, wherein heterocyclyl has an optionally present nitrogen atom and wherein the nitrogen atom is optionally oxidized, and wherein phenyl and heterocyclyl are each optionally substituted with one or more of alkyl, alkoxy, halogen, hydroxy, amino (optionally substituted with one or more of alkyl, acyl, carbonylalkoxy or sulfonylalkyl), carboxy, acyl or carbonylalkoxy.

An example of the invention is a compound of Formula (I) and a salt, isomer, prodrug, metabolite or polymorph thereof, wherein X₂ is absent.

An example of the invention is a compound of Formula (I) and a salt, isomer, prodrug, metabolite or polymorph thereof, wherein X₂ is alkyl.

An example of the invention is a compound of Formula (I) and a salt, isomer, prodrug, metabolite or polymorph thereof, wherein R₂ is hydroxy, halogen, amino (optionally substituted with one or more of alkyl, formyl, acyl, sulfonylalkyl or carbonylalkoxy), cyano, nitro, alkoxy, carboxy, carbonylalkoxy, oxyacyl, oxyacylaryl, oxyacrylyl, oxyacrylylaryl (optionally substituted on aryl with one or more of alkyl, alkoxy, cyano, halogen, hydroxy, nitro, amino or aminoalkyl), oxycarbonylalkoxy, aminoacylamino, aminoacylaminoalkyl, carbamoyl, carbamoylalkyl, urea or ureaalkyl.

An example of the invention is a compound of Formula (I) and a salt, isomer, prodrug, metabolite or polymorph thereof, wherein R₂ is hydroxy, halogen, amino (optionally substituted with one or more of alkyl, formyl, acyl, sulfonylalkyl or carbonylalkoxy), alkoxy, carboxy, carbonylalkoxy, oxyacyl, oxyacrylylaryl (optionally substituted on aryl with one or more of halogen or nitro), oxycarbonylalkoxy, aminoacylamino, aminoacylaminoalkyl, carbamoyl or ureaalkyl.

An example of the invention is a compound of Formula (I) and a salt, isomer, prodrug, metabolite or polymorph thereof, wherein R₂ is hydroxy, halogen, amino (optionally substituted with one or more of alkyl, formyl, acyl, sulfonylalkyl or carbonylalkoxy), alkoxy, carboxy, carbonylalkoxy, oxyacyl, oxyacrylylphenyl (optionally substituted on phenyl with one or more of halogen or nitro), oxycarbonylalkoxy, aminoacylamino, aminoacylaminoalkyl, carbamoyl or ureaalkyl.

An example of the invention is a compound of Formula (I) and a salt, isomer, prodrug, metabolite or polymorph thereof, wherein X₃ is carbonyl, acyl, acyloxy, acrylyl, carbonylalkynyl, carbonylalkoxy, carbamoyl, carbamoylalkyl, thiocarbamyl or iminomethylaminocarbonyl, wherein when X₃ is carbonylalkoxy, then R₃ is optionally present.

An example of the invention is a compound of Formula (I) and a salt, isomer, prodrug, metabolite or polymorph thereof, wherein X₃ is carbonyl, acyl, acyloxy, acrylyl, carbonylalkynyl, carbonylalkoxy, carbamoyl, carbamoylalkyl, thiocarbamyl or iminomethylaminocarbonyl, wherein when X₃ is carbonylalkoxy, then R₃ is optionally present.

An example of the invention is a compound of Formula (I) and a salt, isomer, prodrug, metabolite or polymorph thereof, wherein R₃ is cycloalkyl, aryl or heterocyclyl each optionally substituted with one or more of alkyl, alkoxy, cyano, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl, carbamoylalkyl or phenyl (optionally substituted on phenyl with one or more of alkyl, alkoxy, halogen, hydroxy, nitro, amino or aminoalkyl).

An example of the invention is a compound of Formula (I) and a salt, isomer, prodrug, metabolite or polymorph thereof, wherein R₃ is cycloalkyl, phenyl or heterocyclyl each optionally substituted with one or more of alkyl, alkoxy, cyano, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl, carbamoylalkyl or aryl (optionally substituted on aryl with one or more of alkyl, alkoxy, halogen, hydroxy, nitro, amino or aminoalkyl).

An example of the invention is a compound of Formula (I) and a salt, isomer, prodrug, metabolite or polymorph thereof, wherein R₃ is cycloalkyl, phenyl or heterocyclyl each optionally substituted with one or more of alkyl, alkoxy, cyano, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl, carbamoylalkyl or phenyl (optionally substituted on phenyl with one or more of alkyl, alkoxy, halogen, hydroxy, nitro, amino or aminoalkyl).

An example of the invention is a compound of Formula (I) and a salt, isomer, prodrug, metabolite or polymorph thereof, wherein R₃ is cycloalkyl, aryl or heterocyclyl each optionally substituted with one or more of alkyl, alkoxy, halogen, alkyltrihalo, alkoxytrihalo, nitro, thioalkyl, thioalkyltrihalo, carbonylalkoxy or aryl (optionally substituted on aryl with one or more halogen).

An example of the invention is a compound of Formula (I) and a salt, isomer, prodrug, metabolite or polymorph thereof, wherein R₃ is cycloalkyl, phenyl or heterocyclyl each optionally substituted with one or more of alkyl, alkoxy, halogen, alkyltrihalo, alkoxytrihalo, nitro, thioalkyl, thioalkyltrihalo, carbonylalkoxy or aryl (optionally substituted on aryl with one or more halogen).

An example of the invention is a compound of Formula (I) and a salt, isomer, prodrug, metabolite or polymorph thereof, wherein R₃ is cycloalkyl optionally substituted with aryl, wherein aryl is optionally substituted with one or more of alkyl, alkoxy, halogen, hydroxy, nitro, amino or aminoalkyl.

An example of the invention is a compound of Formula (I) and a salt, isomer, prodrug, metabolite or polymorph thereof, wherein R₃ is cycloalkyl optionally substituted with aryl, wherein aryl is optionally substituted with one or more of halogen.

An example of the invention is a compound of Formula (I) and a salt, isomer, prodrug, metabolite or polymorph thereof, wherein R₃ is aryl optionally substituted with one or more of alkyl, alkoxy, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl or carbamoylalkyl.

An example of the invention is a compound of Formula (I) and a salt, isomer, prodrug, metabolite or polymorph thereof, wherein R₃ is phenyl optionally substituted with one or more of alkyl, alkoxy, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl or carbamoylalkyl.

An example of the invention is a compound of Formula (I) and a salt, isomer, prodrug, metabolite or polymorph thereof, wherein R₃ is aryl optionally substituted with one or more of alkyl, alkoxy, halogen, alkyltrihalo, alkoxytrihalo, nitro, thioalkyl, thioalkyltrihalo or carbonylalkoxy.

An example of the invention is a compound of Formula (I) and a salt, isomer, prodrug, metabolite or polymorph thereof, wherein R₃ is phenyl optionally substituted with one or more of alkyl, alkoxy, halogen, alkyltrihalo, alkoxytrihalo, nitro, thioalkyl, thioalkyltrihalo or carbonylalkoxy.

An example of the invention is a compound of Formula (I) and a salt, isomer, prodrug, metabolite or polymorph thereof, wherein R₃ is heterocyclyl optionally substituted with one or more of alkyl, alkoxy, halogen, hydroxy, amino or aminoalkyl.

An example of the invention is a compound of Formula (I) and a salt, isomer, prodrug, metabolite or polymorph thereof, wherein R₃ is heterocyclyl optionally substituted with one or more of halogen.

An example of the invention is a compound of Formula (I) and a salt, isomer, prodrug, metabolite or polymorph thereof, wherein
X₁ is absent, alkyl or alkylcarbamoylalkyl,
R₁ is aryl or heterocyclyl, wherein heterocyclyl has an optionally present nitrogen atom and wherein the nitrogen atom is optionally oxidized, and wherein aryl and heterocyclyl are each optionally substituted with one or more of alkyl, alkoxy, halogen, hydroxy, amino, (optionally substituted with one or more of alkyl, acyl, carbonylalkoxy or sulfonylalkyl), carboxy, acyl or carbonylalkoxy,
X₂ is absent or alkyl,
R₂ hydroxy, halogen, amino (optionally substituted with one or more of alkyl, formyl, acyl, sulfonylalkyl or carbonylalkoxy), alkoxy, carboxy, carbonylalkoxy, oxyacyl, oxyacrylylaryl (optionally substituted on aryl with one or more of halogen or nitro), oxycarbonylalkoxy, aminoacylamino, aminoacylaminoalkyl, carbamoyl or ureaalkyl,
X₃ is carbonyl, acyl, acyloxy, acrylyl, carbonylalkynyl, carbonylalkoxy, carbamoyl, carbamoylalkyl, thiocarbamyl or iminomethylaminocarbonyl, wherein when X₃ is carbonylalkoxy, then R₃ is optionally present, and
R₃ is cycloalkyl, aryl or heterocyclyl each optionally substituted with one or more of alkyl, alkoxy, halogen, alkyltrihalo, alkoxytrihalo, nitro, thioalkyl, thioalkyltrihalo, carbonylalkoxy or aryl (optionally substituted on aryl with one or more halogen).

An example of the invention is a compound of Formula (I) and a salt, isomer, prodrug, metabolite or polymorph thereof, wherein X₂R₂, X₁R₁, and X₃R₃ are dependently selected from

| Cpd | X₂R₂ | X₁R₁ | X₃R₃ |
|---|---|---|---|
| 1 | CO₂H | -4-Cl-phenyl | C(O)CH═CH-3,4-Cl₂-phenyl |
| 2 | CO₂H | -4-OCH₃-phenyl | C(O)CH═CH-3,5-F₂-phenyl |
| 3 | C(O)OCH₃ | -4-OCH₃-phenyl | C(O)CH═CH-3,5-F₂-phenyl |
| 4 | CO₂H | -4-Cl-phenyl | C(O)CH═CH-3,4,5-F₃-phenyl |
| 5 | CO₂H | -4-OCH₃-phenyl | C(O)CH═CH-3,4,5-F₃-phenyl |
| 6 | CO₂H | -indol-3-yl | C(O)CH═CH-3,4,5-F₃-phenyl |
| 7 | CO₂H | -indol-3-yl | C(O)CH═CH-3,5-F₂-phenyl |
| 8 | CO₂H | -5-F-indol-3-yl | C(O)CH═CH-3,5-F₂-phenyl |
| 9 | CO₂H | -5-F-indol-3-yl | C(O)CH═CH-3,4,5-F₃-phenyl |
| 10 | CO₂H | -indol-1-yl | C(O)CH═CH-3,4,5-F₃-phenyl |
| 11 | CO₂H | —CH₂-indol-3-yl | C(O)CH═CH-3,5-F₂-phenyl |
| 12 | CO₂H | —CH₂-indol-3-yl | C(O)CH═CH-3,4,5-F₃-phenyl |
| 13 | (S)-CO₂H | -indol-3-yl | C(O)CH═CH-3,4,5-F₃-phenyl |
| 14 | (R)-CO₂H | -indol-3-yl | C(O)CH═CH-3,4,5-F₃-phenyl |
| 15 | CO₂H | -5-OH-indol-3-yl | C(O)CH═CH-3,4,5-F₃-phenyl |
| 16 | CO₂H | -5-OH-indol-3-yl | C(O)CH═CH-3,5-F₂-phenyl |
| 17 | CO₂H | -5-NHC(O)CH₃-indol-3-yl | C(O)CH═CH-3,4,5-F₃-phenyl |
| 18 | CO₂H | -indol-3-yl | C(O)CH═CH-3,4-Cl₂-phenyl |
| 19 | CO₂H | -5-F-indol-3-yl | C(O)CH═CH-3,4-Cl₂-phenyl |
| 20 | CO₂H | -indol-3-yl | C(O)NH-3,4-Cl₂-phenyl |
| 21 | CO₂H | -1-C(O)CH₃-indol-3-yl | C(O)CH═CH-3,5-F₂-phenyl |
| 22 | CO₂H | -indol-3-yl | C(O)CH═CH-3,4-F₂-phenyl |
| 23 | CO₂H | -indol-3-yl | C(O)CH═CH-4-CF₃-phenyl |
| 24 | CO₂H | -6-F-indol-3-yl | C(O)CH═CH-3,5-F₂-phenyl |
| 25 | CO₂H | -6-Cl-indol-3-yl | C(O)CH═CH-3,5-F₂-phenyl |
| 26 | CO₂H | -5-OCH₃-indol-3-yl | C(O)CH═CH-3,5-F₂-phenyl |
| 27 | CO₂H | -indol-3-yl | C(O)CH═CH-phenyl |
| 28 | CO₂H | -indol-3-yl | C(O)NH-3,5-F₂-phenyl |
| 29 | CO₂H | -5-NHSO₂CH₃-indol-3-yl | C(O)CH═CH-3,5-F₂-phenyl |
| 30 | CO₂H | -5-OCH₃-indol-3-yl | C(O)CH═CH-3,4,5-F₃-phenyl |
| 31 | CO₂H | -6-Cl-indol-3-yl | C(O)CH═CH-3,4,5-F₃-phenyl |
| 32 | CO₂H | -indol-3-yl | C(O)NH-phenyl |
| 33 | CO₂H | -indol-3-yl | C(O)NH-3,5-Cl₂-phenyl |
| 34 | CO₂H | -indol-3-yl | C(O)CH═CH-4-Cl-phenyl |
| 35 | CO₂H | -indol-3-yl | C(O)CH═CH-3-CF₃-phenyl |
| 36 | CO₂H | -indol-3-yl | C(O)CH═CH-3-Br-4-F-phenyl |
| 37 | CO₂H | -indol-3-yl | C(O)CH═CH-4-OCH₃-phenyl |
| 38 | CO₂H | -6-OCH₃-indol-3-yl | C(O)CH═CH-3,5-F₂-phenyl |
| 39 | CO₂H | -6-F-indol-3-yl | C(O)CH═CH-3,4-Cl₂-phenyl |
| 40 | CO₂H | -indol-3-yl | C(O)NH-3,4-F₂-phenyl |
| 41 | CO₂H | -4-OCH₃-indol-3-yl | C(O)CH═CH-3,5-F₂-phenyl |
| 42 | CO₂H | -7-OCH₃-indol-3-yl | C(O)CH═CH-3,5-F₂-phenyl |
| 43 | CO₂H | -indol-3-yl | C(═S)NH-phenyl |
| 44 | CO₂H | -indol-3-yl | C(═S)NH-2,4-F₂-phenyl |
| 45 | CO₂H | -indol-3-yl | C(═S)NH-3,5-Cl₂-phenyl |
| 46 | CO₂H | -6-Cl-indol-3-yl | C(O)CH═CH-3,4-Cl₂-phenyl |
| 47 | CO₂H | -5-OCH₃-indol-3-yl | C(O)CH═CH-3,4-Cl₂-phenyl |
| 48 | CO₂H | -indol-3-yl | C(O)NH-3-Cl-4-F-phenyl |
| 49 | CO₂H | -indol-3-yl | C(O)NH-3-Cl-4-CH₃-phenyl |
| 50 | CO₂H | -indol-3-yl | C(═NH)NHC(O)-3,4-Cl₂-phenyl |
| 51 | CO₂H | -indol-3-yl | C(═NH)NHC(O)-3,5-F₂-phenyl |
| 52 | CO₂H | -indol-3-yl | C(═NH)NHC(O)-3,4,5-F₃-phenyl |
| 53 | CO₂H | -5-NHSO₂CH₃-indol-3-yl | C(O)CH═CH-3,4,5-F₃-phenyl |
| 54 | CO₂H | -indol-3-yl | C(═NH)NHC(O)-3-F-phenyl |
| 55 | CO₂H | -indol-3-yl | C(═S)NH-3,5-F₂-phenyl |
| 56 | CO₂H | -indol-3-yl | C(═S)NH-3-Br-phenyl |
| 57 | CO₂H | -indol-3-yl | C(O)NH-3-CF₃-4-Cl-phenyl |
| 58 | CO₂H | -indol-3-yl | C(O)NH-3-CF₃-4-F-phenyl |
| 59 | CO₂H | -indol-3-yl | C(O)CH═CH-4-NO₂-phenyl |
| 60 | CO₂H | -indol-3-yl | C(O)CH═CH-4-Br-phenyl |
| 61 | CO₂H | -indol-3-yl | C(O)CH═CH-4-CH₃-phenyl |
| 62 | CO₂H | -indol-3-yl | C(O)CH═CH-3-F-phenyl |
| 63 | CO₂H | -indol-3-yl | C(O)CH═CH-3,4-(OCH₃)₂-phenyl |
| 64 | CO₂H | -indol-3-yl | C(═S)NH-3,4-Cl₂-phenyl |
| 65 | CO₂H | -indol-3-yl | C(O)NH-3-CF₃-5-F-phenyl |
| 66 | CO₂H | -indol-3-yl | C(O)NH-3,4-(OCH₃)₂-phenyl |
| 67 | CO₂H | -indol-3-yl | C(O)NH-3-Cl-4-OCH₃-phenyl |
| 68 | CO₂H | -indol-3-yl | C(O)NH-4-C(O)OCH₃-phenyl |
| 69 | CO₂H | -indol-3-yl | C(O)NH-4-OCH₃-phenyl |
| 70 | CO₂H | -indol-3-yl | C(O)CH═CH-3-CH₃-phenyl |
| 71 | CO₂H | -indol-3-yl | C(O)CH═CH-3-Br-phenyl |
| 72 | CO₂H | -indol-3-yl | C(O)CH═CH-3-OCH₃-phenyl |
| 73 | CO₂H | -indol-3-yl | C(═NH)NHC(O)-3-CF₃-phenyl |
| 74 | CO₂H | -indol-3-yl | C(O)CH═CH-3-F-4-CH₃-phenyl |
| 75 | CO₂H | -indol-3-yl | C(O)CH═CH-3-F-4-CF₃-phenyl |
| 76 | CO₂H | -indol-3-yl | C(O)CH═CH-3-Cl-4-F-phenyl |
| 77 | CO₂H | -indol-3-yl | C(O)CH═CH-4-F-phenyl |
| 78 | CO₂H | -indol-3-yl | C(═S)NH-4-CH₃-phenyl |
| 79 | CO₂H | -indol-3-yl | C(═S)NH-3-CF₃-phenyl |
| 80 | CO₂H | -indol-3-yl | C(═S)NH-4-CF₃-phenyl |
| 81 | CO₂H | -5-NHC(O)O—C(CH₃)₃- | C(O)CH═CH-3,4,5-F₃-phenyl |
| | | indol-3-yl | |
| 82 | CO₂H | -6-NHSO₂CH₃-indol-3-yl | C(O)CH═CH-3,4,5-F₃-phenyl |
| 83 | CO₂H | -5-NH₂-indol-3-yl | C(O)CH═CH-3,4,5-F₃-phenyl |
| 84 | CO₂H | -indol-3-yl | C(O)NHCH₂-3,4-Cl₂-phenyl |
| 85 | CO₂H | -indol-3-yl | C(O)NH-3-Br-phenyl |
| 86 | CO₂H | -indol-3-yl | C(O)NH-3-Cl-phenyl |
| 87 | C(O)OCH₃ | -indol-3-yl | C(O)CH═CH-3,4,5-F₃-phenyl |
| 88 | CO₂H | -indol-3-yl | C(O)NH-4-Cl-phenyl |
| 89 | CO₂H | -indol-3-yl | C(O)NH-4-Br-phenyl |
| 90 | CO₂H | -indol-3-yl | C(O)NH-4-F-phenyl |
| 91 | CO₂H | -indol-3-yl | C(O)NH-3-F-phenyl |
| 92 | CO₂H | -indol-3-yl | C(O)CH═CH-3-NO₂-phenyl |
| 93 | CO₂H | -indol-3-yl | C(O)CH═CH-3-Cl-phenyl |
| 94 | CO₂H | -5-OCH₃-indol-3-yl | C(O)NH-3,4-Cl₂-phenyl |
| 95 | CO₂H | -6-OCH₃-indol-3-yl | C(O)NH-3,4-Cl₂-phenyl |
| 96 | CO₂H | -indol-3-yl | C(O)NH-4-CF₃-phenyl |
| 97 | CO₂H | -indol-3-yl | C(O)NH-3-CF₃-phenyl |
| 98 | CO₂H | -indol-3-yl | C(O)NH-3-CH₃-phenyl |
| 99 | CO₂H | -indol-3-yl | C(O)NH-4-CH₃-phenyl |
| 100 | CO₂H | -indol-3-yl | C(O)NH-3,4-(CH₃)₂-phenyl |
| 101 | CO₂H | -indol-3-yl | C(O)NH-3-CH₃-4-Br-phenyl |
| 102 | CO₂H | -indol-3-yl | C(O)NH-3-CH₃-4-F-phenyl |
| 103 | CO₂H | -indol-3-yl | C(O)CH═CH-thien-2-yl |
| 104 | CO₂H | -indol-3-yl | C(O)CH═CH-thien-3-yl |
| 105 | CO₂H | -indol-3-yl | C(O)NH-3-F-4-CH₃-phenyl |
| 106 | CO₂H | -indol-3-yl | C(O)NH-3-CF₃-4-CH₃-phenyl |
| 107 | C(O)NH₂ | -indol-3-yl | C(O)CH═CH-3,4,5-F₃-phenyl |
| 108 | CO₂H | -7-OCH₃-indol-3-yl | C(O)CH═CH-3,4,5-F₃-phenyl |
| 109 | CO₂H | -5-NHSO₂CH₃-indol-3-yl | C(O)NH-3,4-Cl₂-phenyl |
| 110 | CO₂H | -indol-3-yl | C(O)NH-2,3-Cl₂-phenyl |
| 111 | CO₂H | -indol-3-yl | C(O)NH-2,4-Cl₂-phenyl |
| 112 | CH₂OH | -indol-3-yl | C(O)CH═CH-3,4,5-F₃-phenyl |
| 113 | CH₂OH | -indol-3-yl | C(O)CH═CH-3,4-F₂-phenyl |
| 114 | CO₂H | -indol-3-yl | C(O)CH₂O-3,4-Cl₂-phenyl |
| 115 | CO₂H | -indol-3-yl | C(O)(CH₂)₂-3,4-Cl₂-phenyl |
| 116 | CH₂OH | -indol-3-yl | C(O)CH═CH-3,5-F₂-phenyl |
| 117 | CO₂H | -indol-3-yl | C(O)NH-2-F-4-Cl-phenyl |
| 118 | C(O)OCH₃ | -7-OCH₃-indol-3-yl | C(O)CH═CH-3,4,5-F₃-phenyl |
| 119 | CH₂OH | -indol-3-yl | C(O)CH═CH-3-CF₃-phenyl |
| 120 | CH₂OH | -indol-3-yl | C(═S)NH-3-CF₃-phenyl |
| 121 | CH₂OH | -indol-3-yl | C(O)CH═CH-3,4-Cl₂-phenyl |
| 122 | CH₂OH | -indol-3-yl | C(═S)NH-3,4-Cl₂-phenyl |
| 123 | CH₂OH | -indol-3-yl | C(O)NH-3,4-Cl₂-phenyl |
| 124 | CH₂OH | -indol-3-yl | C(═S)NH-3,5-F₂-phenyl |
| 125 | CO₂H | -indol-3-yl | C(O)NH-2,3,4-F₃-phenyl |
| 126 | CO₂H | -indol-3-yl | C(O)NH-2,4,5-Cl₃-phenyl |
| 127 | CO₂H | -indol-3-yl | C(O)NH-4-SCH₃-phenyl |
| 128 | CH₂OH | -indol-3-yl | C(═NH)NHC(O)-3,4-Cl₂-phenyl |
| 129 | CH₂OH | -indol-3-yl | C(O)NH-3,5-F₂-phenyl |
| 130 | CH₂N(CH₃)₂ | -indol-3-yl | C(O)CH═CH-3,5-F₂-phenyl |
| 131 | CH₂OH | -7-OCH₃-indol-3-yl | C(O)OC(CH₃)₃ |
| 132 | CH₂OH | -6-OCH₃-indol-3-yl | C(O)CH═CH-3,4,5-F₃-phenyl |
| 133 | CH₂OH | -7-OCH₃-indol-3-yl | C(O)CH═CH-3,4,5-F₃-phenyl |
| 134 | CH₂N— | -indol-3-yl | C(O)CH═CH-3,5-F₂-phenyl |
| | (SO₂CH₃)₂ | | |
| 135 | CO₂H | -indol-3-yl | C(O)NH-3,5-(CH₃)₂-phenyl |
| 136 | CO₂H | -indol-3-yl | C(O)NH-3,5-(CF₃)₂-phenyl |
| 137 | CH₂OH | -4-OCH₃-phenyl | C(O)CH═CH-3,5-F₂-phenyl |
| 138 | CH₂OH | -4-OCH₃-phenyl | C(O)CH═CH-3,4-F₂-phenyl |
| 139 | CH₂OH | -4-OCH₃-phenyl | C(O)CH═CH-3,4-Cl₂-phenyl |
| 140 | CH₂OH | -4-OCH₃-phenyl | C(O)CH═CH-2,4,5-F₃-phenyl |
| 141 | CH₂OH | -4-OCH₃-phenyl | C(O)NH-3,4-F₂-phenyl |
| 142 | CO₂H | -indol-3-yl | C(O)NH-4-SCF₃-phenyl |
| 143 | CO₂H | -indol-3-yl | C(O)NH-4-OCF₃-phenyl |
| 144 | CO₂H | -indol-3-yl | C(O)NH-3-SCH₃-phenyl |
| 145 | CO₂H | -4-C(O)OCH₃-phenyl | C(O)CH═CH-3,5-F₂-phenyl |
| 146 | CO₂H | -5-C(O)OCH₃-indol-3-yl | C(O)CH═CH-3,4,5-F₃-phenyl |
| 147 | CO₂H | -5-CO₂H-indol-3-yl | C(O)CH═CH-3,4,5-F₃-phenyl |
| 148 | CO₂H | -CH₂C(O)NH-benzyl | C(O)CH═CH-3,5-F₂-phenyl |
| 149 | CO₂H | -CH₂C(O)NH-benzyl | C(O)CH═CH-3,4,5-F₃-phenyl |
| 150 | CO₂H | -pyrrol-3-yl | C(O)CH═CH-3,4,5-F₃-phenyl |
| 151 | CO₂H | -1H-pyrrolo[2,3-b]pyridin- | C(O)CH═CH-3,4,5-F₃-phenyl |
| | | 3-yl | |
| 152 | C(O)O— | -1H-pyrrolo[2,3-b]pyridin- | C(O)CH═CH-3,4,5-F₃-phenyl |
| | CH₂CH₃ | 3-yl | |
| 153 | CH₂OH | -1H-pyrrolo[2,3-b]pyridin- | C(O)CH═CH-3,4,5-F₃-phenyl |
| | | 3-yl | |
| 154 | CH₂OH | -indol-3-yl | C(O)-benzo[b]furan-2-yl |
| 155 | CH₂OH | -pyrazol-3-yl | C(O)CH═CH-3,4-Cl₂-phenyl |
| 156 | CH₂OH | -pyrazol-3-yl | C(O)CH═CH-3,4,5-F₃-phenyl |
| 157 | CH₂OH | -indol-3-yl | C(O)-5-Cl-benzo[b]furan-2-yl |
| 158 | CH₂OH | -4-OCH₃-phenyl | C(O)CH═CH-3,4,5-F₃-phenyl |
| 159 | CH₂OH | -4-OCH₃-phenyl | C(O)CH═CH-phenyl |
| 160 | CH₂OH | -4-OCH₃-phenyl | C(O)-5-Cl-benzo[b]furan-2-yl |
| 161 | CH₂OH | -4-OCH₃-phenyl | C(O)CH═CH-3-Br-4-F-phenyl |
| 162 | CH₂OH | -5-OCH₃-indol-3-yl | C(O)CH═CH-3,4-Cl₂-phenyl |
| 163 | CH₂OH | -6-OCH₃-indol-3-yl | C(O)CH═CH-3,4-Cl₂-phenyl |
| 164 | CH₂OH | -5-OCH₃-indol-3-yl | C(O)CH═CH-3,5-F₂-phenyl |
| 165 | CH₂OH | -6-OCH₃-indol-3-yl | C(O)CH═CH-3,5-F₂-phenyl |
| 166 | CH₂OH | -5-OCH₃-indol-3-yl | C(O)CH═CH-3,4,5-F₃-phenyl |
| 167 | CH₂OH | -6-OCH₃-indol-3-yl | C(O)CH═CH-3,4,5-F₃-phenyl |
| 168 | CH₂OH | -5-F-indol-3-yl | C(O)CH═CH-3,4-Cl₂-phenyl |
| 169 | CH₂OH | -5-F-indol-3-yl | C(O)CH═CH-4-F-phenyl |
| 170 | CH₂OH | -5-F-indol-3-yl | C(O)CH═CH-3,4,5-F₃-phenyl |
| 171 | CH₂OH | -5-F-indol-3-yl | C(O)CH═CH-3,5-F₂-phenyl |
| 172 | CH₂OH | -5-F-indol-3-yl | C(O)CH═CH-3-Br-4-F-phenyl |
| 173 | CH₂OH | -indazol-3-yl | C(O)CH═CH-3,5-F₂-phenyl |
| 174 | CH₂OH | -benzoimidazol-2-yl | C(O)CH═CH-3,5-F₂-phenyl |
| 175 | CH₂OH | -benzoimidazol-2-yl | C(O)CH═CH-3,4,5-F₃-phenyl |
| 176 | CH₂OH | -benzoimidazol-2-yl | C(O)CH═CH-3,4-Cl₂-phenyl |
| 177 | CO₂H | -indazol-3-yl | C(O)CH═CH-3,5-F₂-phenyl |
| 178 | CO₂H | -5-NH₂-1H-pyrrolo[3,2- | C(O)CH═CH-3,4,5-F₃-phenyl |
| | | b]pyridin-3-yl | |
| 179 | CO₂H | -5-NH₂-1H-pyrrolo[2,3- | C(O)CH═CH-3,4,5-F₃-phenyl |
| | | c]pyridin-3-yl | |
| 180 | (S)-CH₂OH | -4-OCH₃-phenyl | C(O)CH═CH-3,5-F₂-phenyl |
| 181 | (R)-CH₂OH | -4-OCH₃-phenyl | C(O)CH═CH-3,5-F₂-phenyl |
| 182 | CH₂OH | -pyridin-4-yl | C(O)CH═CH-3,5-F₂-phenyl |
| 183 | CH₂OH | -pyridin-4-yl | C(O)CH═CH-3,4,5-F₃-phenyl |
| 184 | CH₂OH | -pyridin-4-yl | C(O)CH═CH-3-CF₃-phenyl |
| 185 | CH₂OH | -pyridin-4-yl | C(O)CH═CH-3,4-Cl₂-phenyl |
| 186 | CH₂OH | -pyridin-4-yl | C(O)CH═CH-3-Br-4-F-phenyl |
| 187 | (S)-CH₂OH | -indol-3-yl | C(O)CH═CH-3,4,5-F₃-phenyl |
| 188 | (R)-CH₂OH | -indol-3-yl | C(O)CH═CH-3,4,5-F₃-phenyl |
| 189 | CH₂OH | -benzo[1,3]dioxol-5-yl | C(O)CH═CH-3,4,5-F₃-phenyl |
| 190 | CH₂OH | -benzo[1,3]dioxol-5-yl | C(O)CH═CH-3,5-F₂-phenyl |
| 191 | CH₂OH | -5-NH₂-1H-pyrrolo[3,2- | C(O)CH═CH-3,4,5-F₃-phenyl |
| | | b]pyridin-3-yl | |
| 192 | CH₂OH | -4-F-phenyl | C(O)CH═CH-3,5-F₂-phenyl |
| 193 | CH₂OH | -4-F-phenyl | C(O)CH═CH-3,4,5-F₃-phenyl |
| 194 | CH₂OH | -thiazol-2-yl | C(O)CH═CH-3,5-F₂-phenyl |
| 195 | CH₂OH | -thiazol-2-yl | C(O)CH═CH-3,4,5-F₃-phenyl |
| 196 | CH₂OH | -thiazol-2-yl | C(O)CH═CH-3,4-Cl₂-phenyl |
| 197 | CH₂OH | -3-OCH₃-phenyl | C(O)CH═CH-3,5-F₂-phenyl |
| 198 | CH₂OH | -5-NHSO₂CH₃-indol-3-yl | C(O)CH═CH-3,4-Cl₂-phenyl |
| 199 | CH₂OC(O)— | -5-NHSO₂CH₃-indol-3-yl | C(O)CH═CH-3,5-F₂-phenyl |
| | CH═CH-3,5- | | |
| | F₂-phenyl | | |
| 200 | CH₂OH | -pyridin-2-yl | C(O)CH═CH-3,5-F₂-phenyl |
| 201 | CH₂OH | -5-NHSO₂CH₃-indol-3-yl | C(O)CH═CH-3,4,5-F₃-phenyl |
| 202 | CH₂OH | -1H-pyrrolo[2,3-b]pyridin- | C(O)CH═CH-3,5-F₂-phenyl |
| | | 3-yl | |
| 203 | CH₂OH | -2-OCH₃-phenyl | C(O)CH═CH-3,5-F₂-phenyl |
| 204 | CO₂H | -2-CH₃-indol-3-yl | C(O)CH═CH-3,4,5-F₃-phenyl |
| 205 | CH₂OH | -7-oxy-1H-pyrrolo[2,3- | C(O)CH═CH-3,5-F₂-phenyl |
| | | b]pyridin-3-yl | |
| 206 | CO₂H | -4-NHSO₂CH₃-phenyl | C(O)CH═CH-3,4,5-F₃-phenyl |
| 207 | CO₂H | -1H-pyrrolo[3,2-b]pyridin- | C(O)CH═CH-3,4,5-F₃-phenyl |
| | | 3-yl | |
| 208 | CH₂OH | -1H-pyrrolo[2,3-b]pyridin- | C(O)CH═CH-3,4-Cl₂-phenyl |
| | | 3-yl | |
| 209 | CH₂OH | -4-NHSO₂CH₃-phenyl | C(O)CH═CH-3,4,5-F₃-phenyl |
| 210 | CH₂OH | -4-NHSO₂CH₃-phenyl | C(O)CH═CH-3,4-Cl₂-phenyl |
| 211 | CO₂H | -6-F-indol-3-yl | C(O)CH═CH-3,4,5-F₃-phenyl |
| 212 | CH₂OH | -indol-3-yl | C(O)-2-(3,4-Cl₂-phenyl)- |
| | | | cyclopropyl |
| 213 | CH₂NH— | -indol-3-yl | C(O)CH═CH-3,4,5-F₃-phenyl |
| | C(O)CH₃ | | |
| 214 | CH₂NH— | -indol-3-yl | C(O)CH═CH-3,5-F₂-phenyl |
| | C(O)CH₃ | | |
| 215 | CH₂NH— | -indol-3-yl | C(O)CH═CH-3,4-Cl₂-phenyl |
| | C(O)CH₃ | | |
| 216 | CH₂NH— | -indol-3-yl | C(O)CH═CH-3-CH₃-phenyl |
| | C(O)CH₃ | | |
| 217 | CH₂NH— | -indol-3-yl | C(O)NH-3,4-Cl₂-phenyl |
| | C(O)CH₃ | | |
| 218 | CH₂NH— | -indol-3-yl | C(O)CH═CH-3-CF₃-phenyl |
| | C(O)CH₃ | | |
| 219 | CH₂NH— | -indol-3-yl | C(O)CH═CH-thien-3-yl |
| | C(O)CH₃ | | |
| 220 | CH₂NH— | -indol-3-yl | C(O)CH═CH-3,4,5-F₃-phenyl |
| | C(O)H | | |
| 221 | CH₂NH— | -indol-3-yl | C(O)CH═CH-3,5-F₂-phenyl |
| | C(O)H | | |
| 222 | CH₂NH— | -indol-3-yl | C(O)CH═CH-3,4-Cl₂-phenyl |
| | C(O)H | | |
| 223 | CH₂NH— | -indol-3-yl | C(O)CH═CH-3-CH₃-phenyl |
| | C(O)H | | |
| 224 | CH₂NH— | -indol-3-yl | C(O)CH═CH-3-CF₃-phenyl |
| | C(O)H | | |
| 225 | CH₂NH— | -indol-3-yl | C(O)CH═CH-thien-3-yl |
| | C(O)H | | |
| 226 | CH₂NH— | -indol-3-yl | C(O)NH-3,4-Cl₂-phenyl |
| | C(O)H | | |
| 227 | C(O)NH₂ | -1H-pyrrolo[2,3-b]pyridin- | C(O)CH═CH-3,4,5-F₃-phenyl |
| | | 3-yl | |
| 228 | CH₂NH— | -indol-3-yl | C(O)CH═CH-3,4,5-F₃-phenyl |
| | C(O)NH— | | |
| | CH₂CH₃ | | |
| 229 | CH₂NH— | -indol-3-yl | C(O)CH═CH-3,5-F₂-phenyl |
| | C(O)NH— | | |
| | CH₂CH₃ | | |
| 230 | CH₂NH— | -indol-3-yl | C(O)CH═CH-3,4-Cl₂-phenyl |
| | C(O)NH— | | |
| | CH₂CH₃ | | |
| 231 | CH₂NH— | -indol-3-yl | C(O)CH═CH-3-CH₃-phenyl |
| | C(O)NH— | | |
| | CH₂CH₃ | | |
| 232 | CH₂NH— | -indol-3-yl | C(O)CH═CH-3-CF₃-phenyl |
| | C(O)NH— | | |
| | CH₂CH₃ | | |
| 233 | CH₂NH— | -indol-3-yl | C(O)CH═CH-3-Br-4-F-phenyl |
| | C(O)NH— | | |
| | CH₂CH₃ | | |
| 234 | CH₂O— | -indol-3-yl | C(O)OC(CH₃)₃ |
| | C(O)CH₃ | | |
| 235 | CH₂O— | -indol-3-yl | C(O)CH═CH-3,5-F₂-phenyl |
| | C(O)CH₃ | | |
| 236 | CH₂O— | -indol-3-yl | C(O)CH═CH-3,4,5-F₃-phenyl |
| | C(O)CH₃ | | |
| 237 | CH₂O— | -indol-3-yl | C(O)CH═CH-3,4-Cl₂-phenyl |
| | C(O)CH₃ | | |
| 238 | CH₂NH— | -indol-3-yl | C(O)CH═CH-3,4,5-F₃-phenyl |
| | C(O)OCH₃ | | |
| 239 | CH₂NH— | -indol-3-yl | C(O)CH═CH-3,5-F₂-phenyl |
| | C(O)OCH₃ | | |
| 240 | CH₂NH— | -indol-3-yl | C(O)CH═CH-3,4-Cl₂-phenyl |
| | C(O)OCH₃ | | |
| 241 | CH₂NH— | -indol-3-yl | C(O)CH═CH-3-CH₃-phenyl |
| | C(O)OCH₃ | | |
| 242 | CH₂O— | -indol-3-yl | C(O)NH-3,4-Cl₂-phenyl |
| | C(O)CH₃ | | |
| 243 | CH₂O— | -{5-N[C(O)CH₃—SO₂CH₃]}- | C(O)CH═CH-3,5-F₂-phenyl |
| | C(O)CH₃ | indol-3-yl | |
| 244 | CH₂OH | -4-Cl-phenyl | C(O)CH═CH-3,4-Cl₂-phenyl |
| 245 | CH₂Cl | -4-Cl-phenyl | C(O)CH═CH-3,4-Cl₂-phenyl |
| 246 | CH₂OH | -4-Cl-phenyl | C(O)CH═CH-3,4,5-F₃-phenyl |
| 247 | CH₂Cl | -4-Cl-phenyl | C(O)CH═CH-4-CF₃-phenyl |
| 248 | CH₂OH | -furo[2,3-b]pyridin-3-yl | C(O)CH═CH-3,4,5-F₃-phenyl |
| 249 | CH₂OH | -4-Cl-phenyl | C(O)CH═CH-3,5-F₂-phenyl |
| 250 | CH₂O— | -4-Cl-phenyl | C(O)CH═CH-3,5-F₂-phenyl |
| | C(O)OCH₃ | | |
| 251 | CH₂OC(O)— | -indol-3-yl | C(O)CH═CH-4-NO₂-phenyl |
| | CH═CH-4- | | |
| | NO₂-phenyl | | |
| 252 | CH₂NH— | -indol-3-yl | C(O)CH═CH-3,5-F₂-phenyl |
| | C(O)CH₂— | | |
| | N(CH₃)₂ | | |
| 253 | CH₂OH | -4-OCH₃-phenyl | C(O)CH≡CH-3,4,5-F₃-phenyl |
| 254 | CH₂OH | -5-F-indol-3-yl | C(O)CH═CH-3,4-F₂-phenyl |
| 255 | CH₂OCH₃ | -4-OCH₃-phenyl | C(O)CH═CH-3,5-F₂-phenyl |
| 256 | CH₂OH | -5,6-Cl₂-1H-benzoimidazol- | C(O)CH═CH-3,5-F₂-phenyl |
| | | 2-yl | |
| 257 | CH₂OH | -5,6-Cl₂-1H-benzoimidazol- | C(O)CH═CH-3,4,5-F₃-phenyl |
| | | 2-yl | |
| 258 | CH₂OH | -4-Cl-phenyl | C(O)CH═CH-4-Cl-phenyl |
| 259 | CH₂OH | -5-OH-indol-3-yl | C(O)CH═CH-3,5-F₂-phenyl |

An example of the invention is a compound of Formula (I) and a salt, isomer, prodrug, metabolite or polymorph thereof represented as follows: 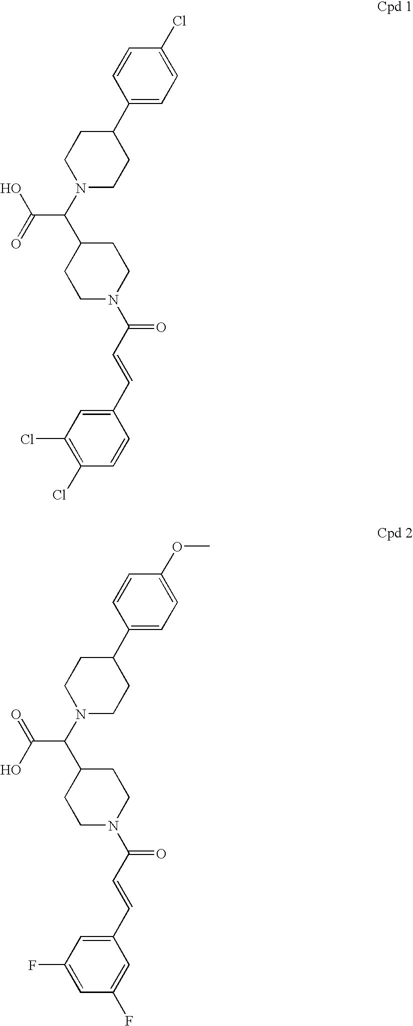 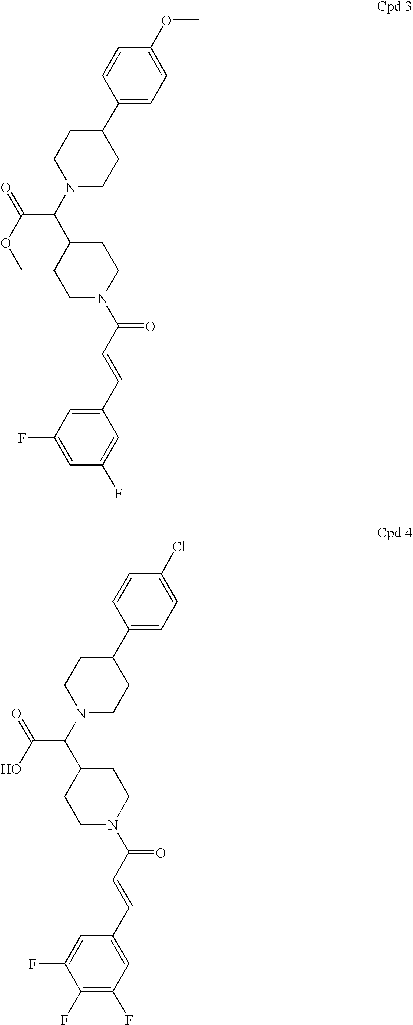 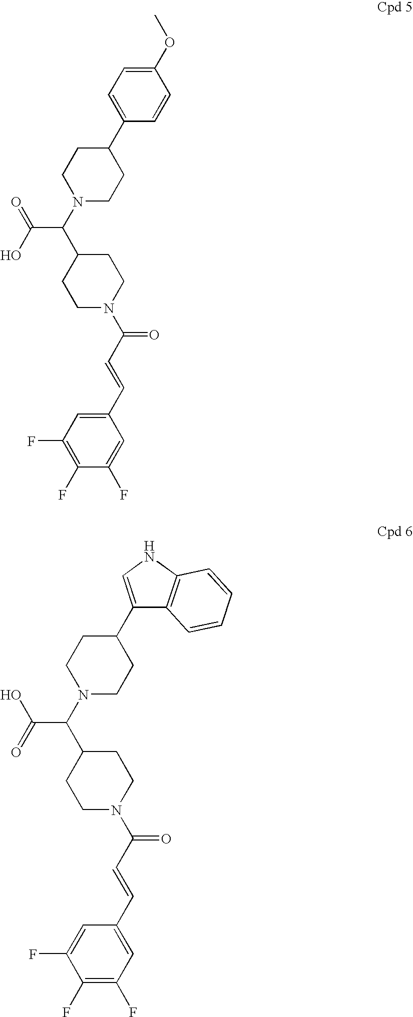 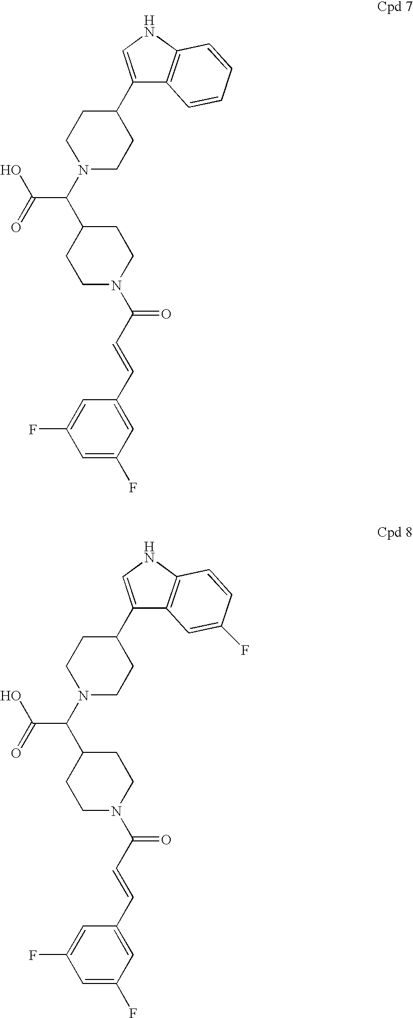 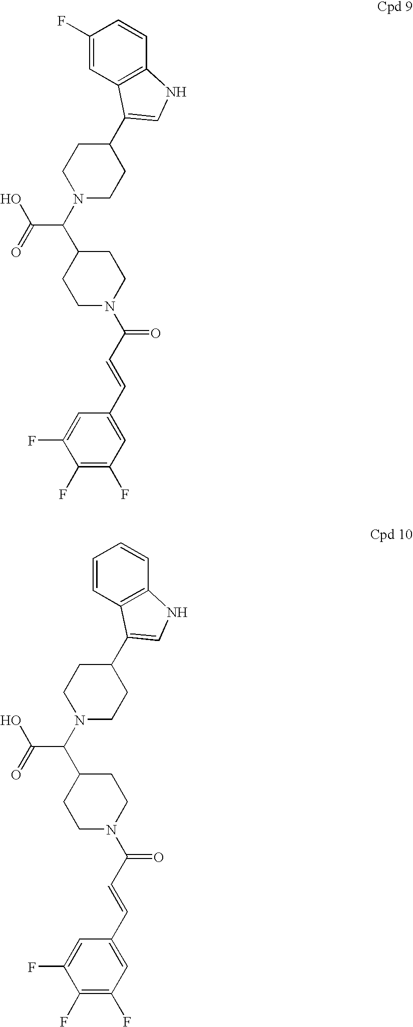 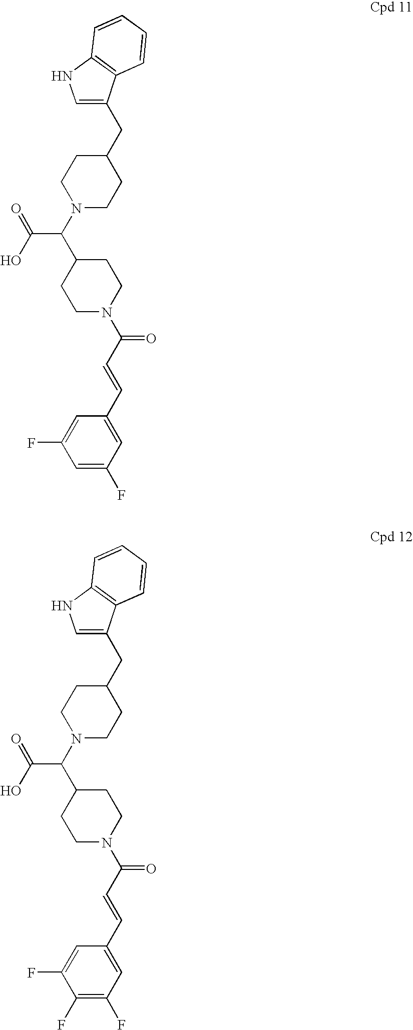 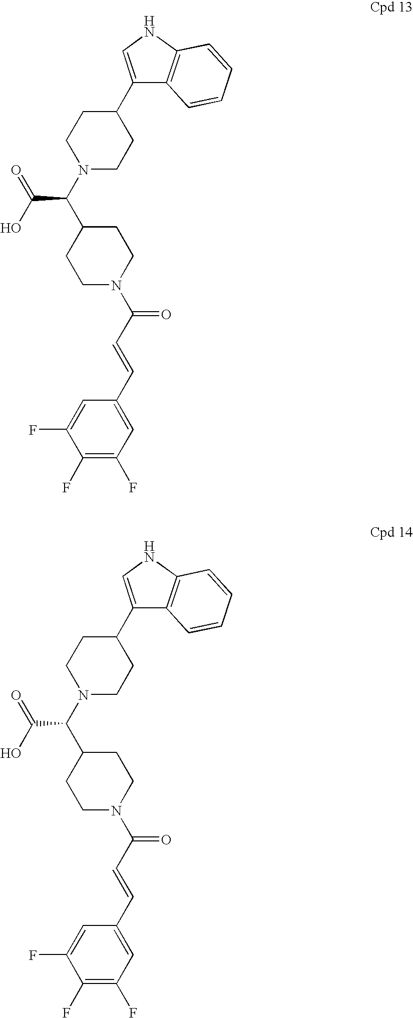 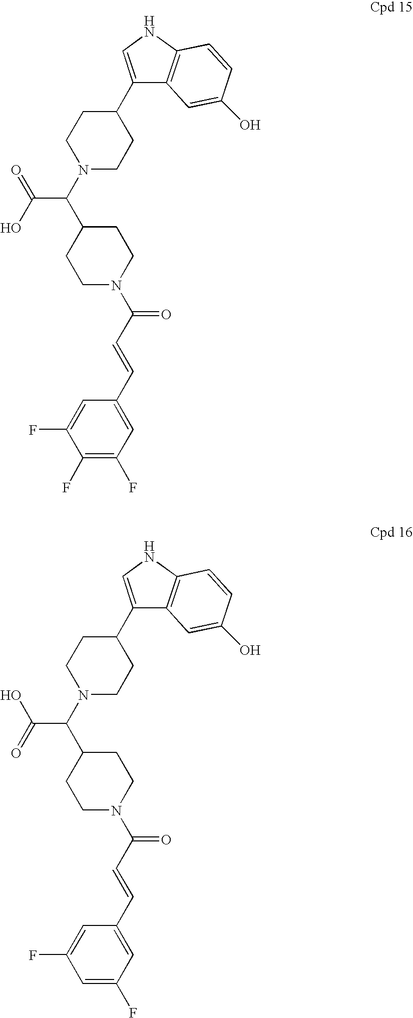 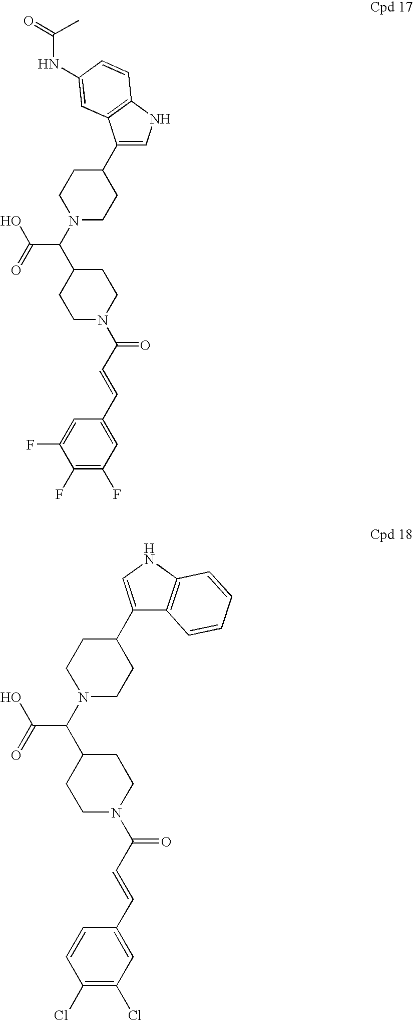 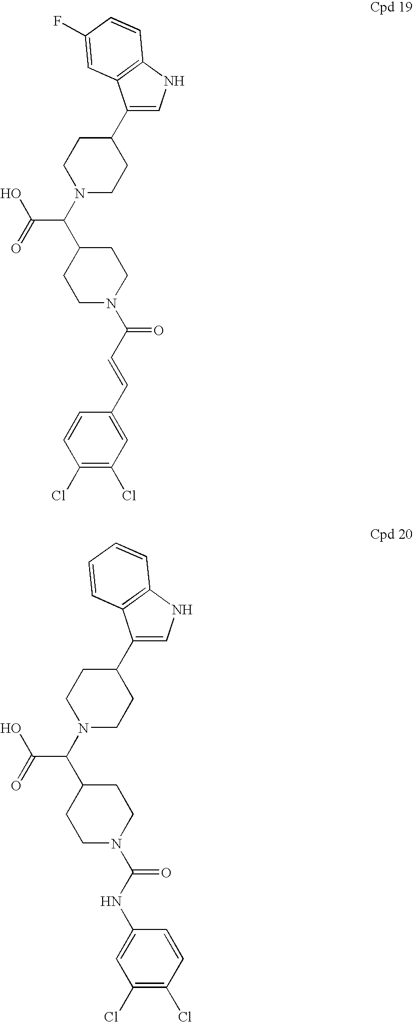 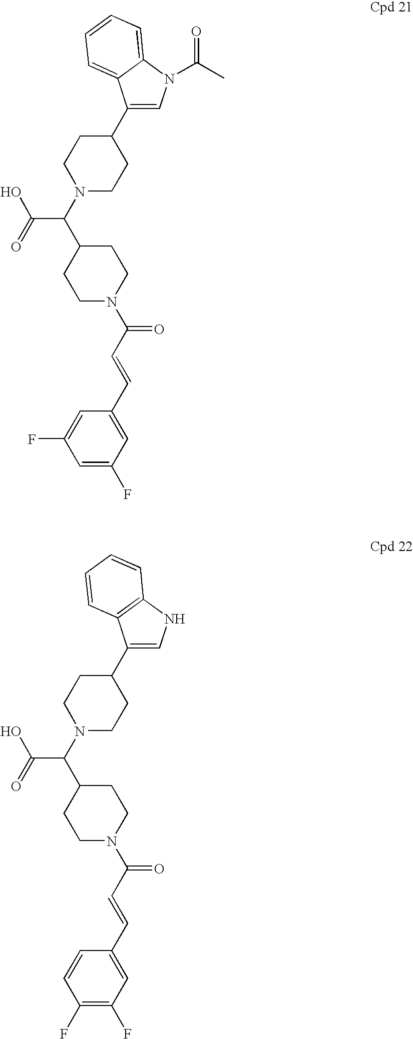 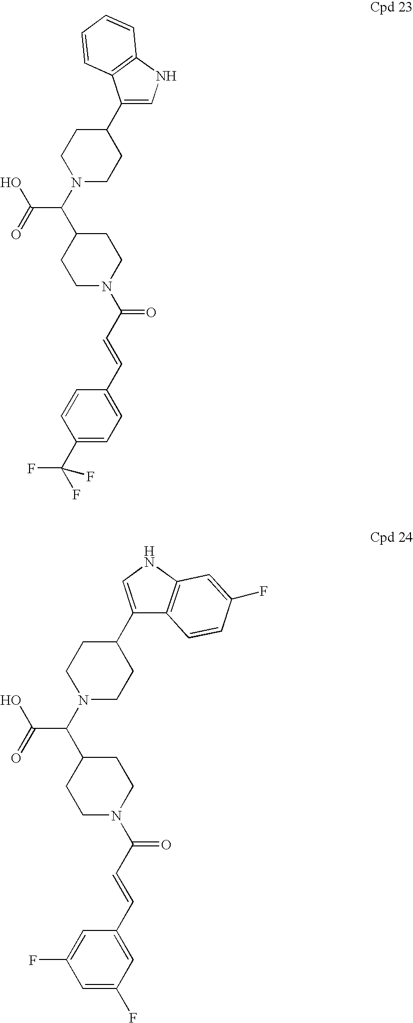 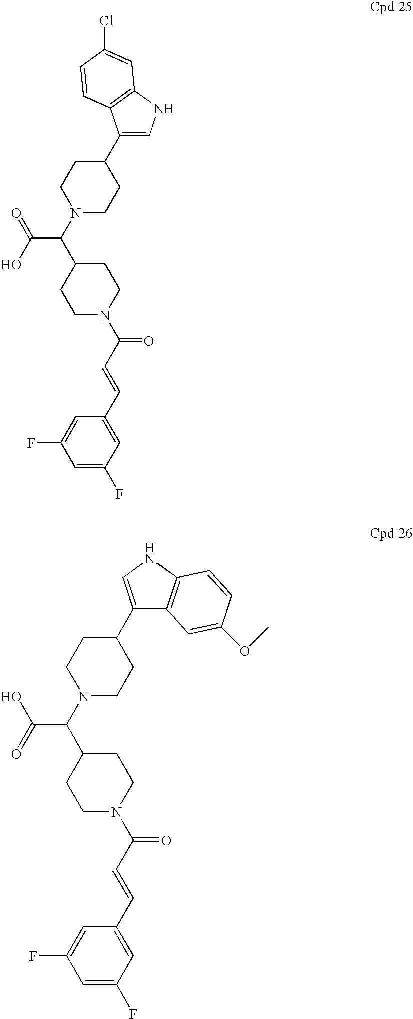 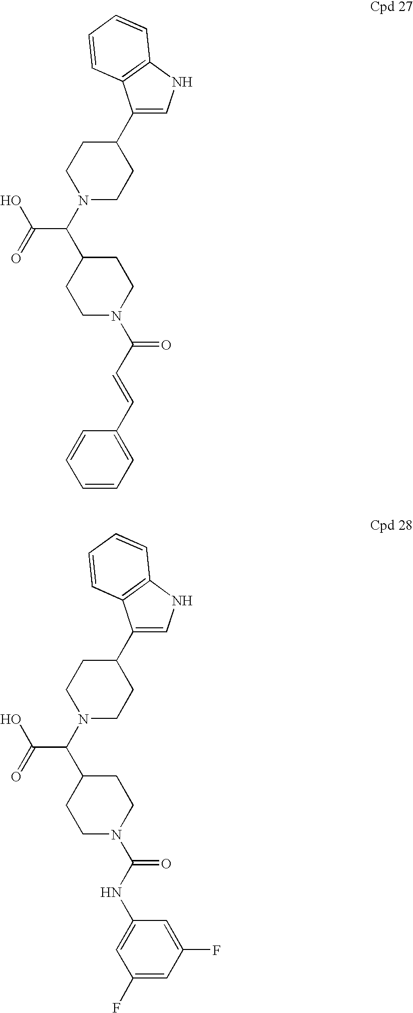 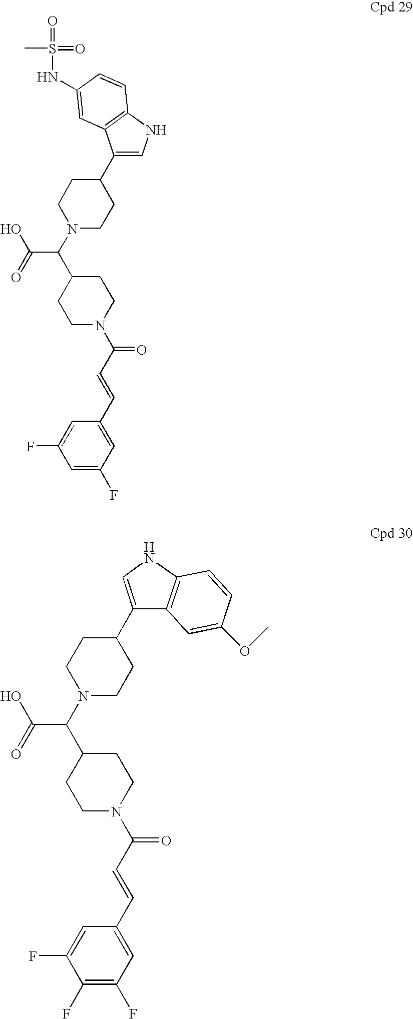 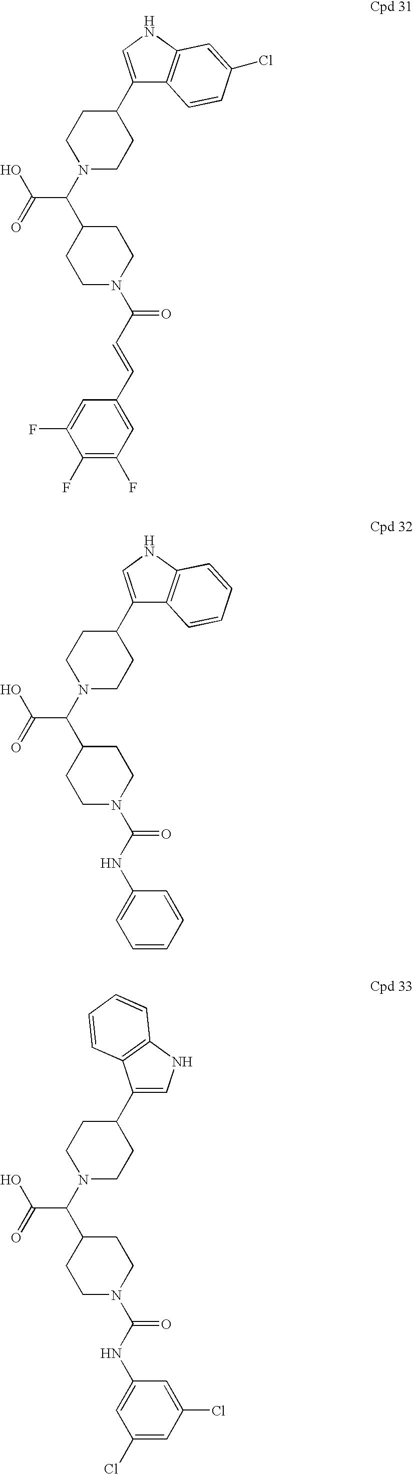 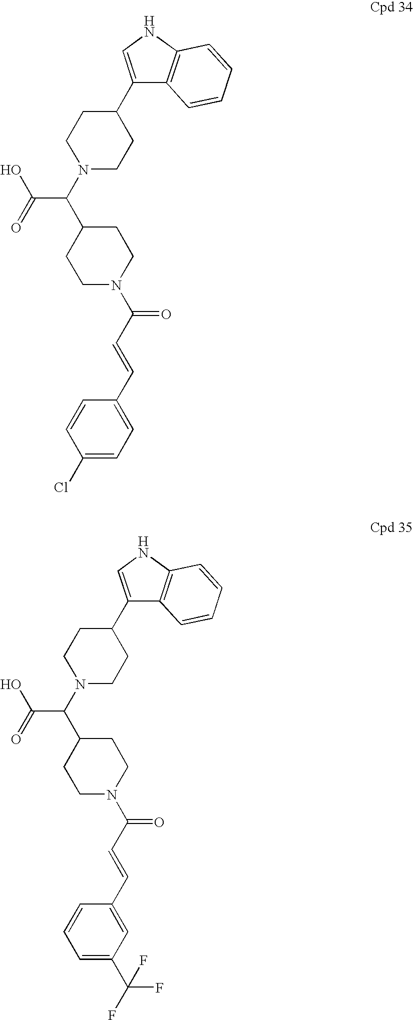 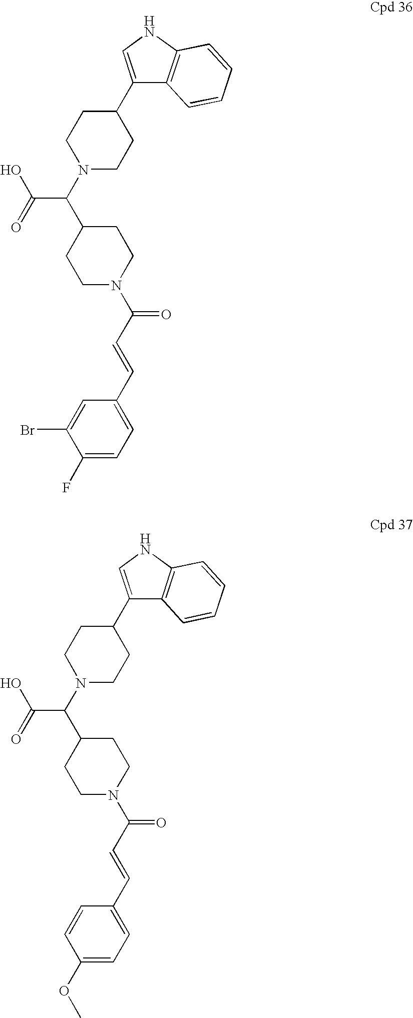 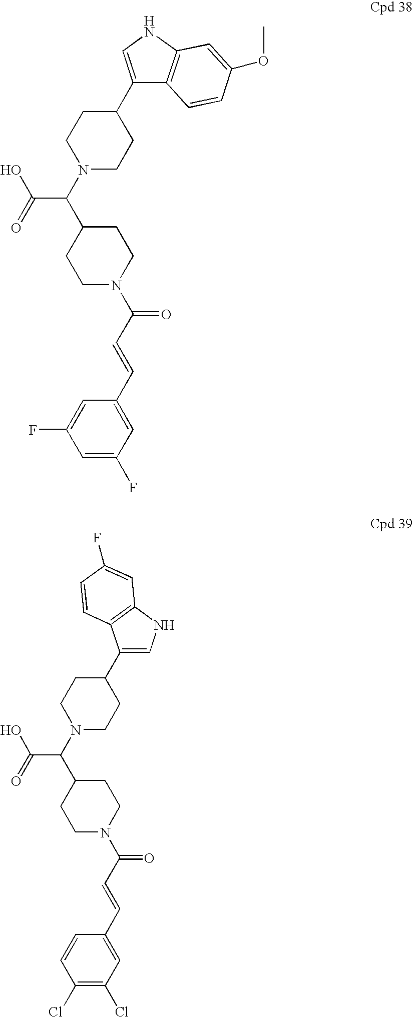 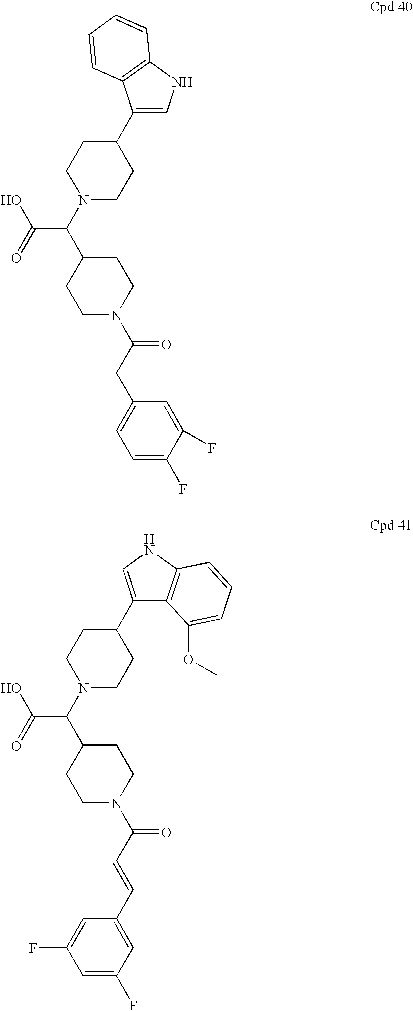 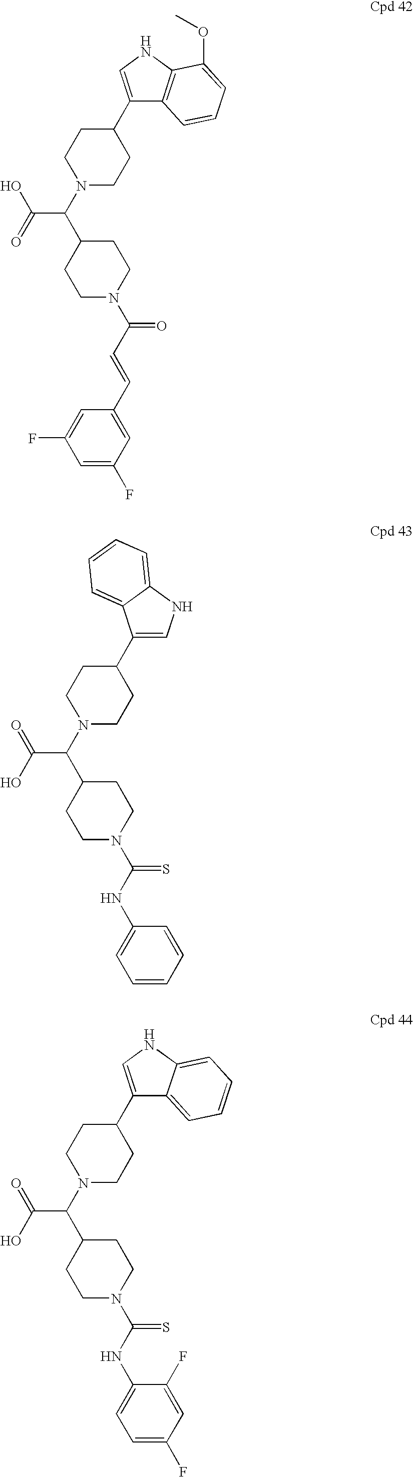 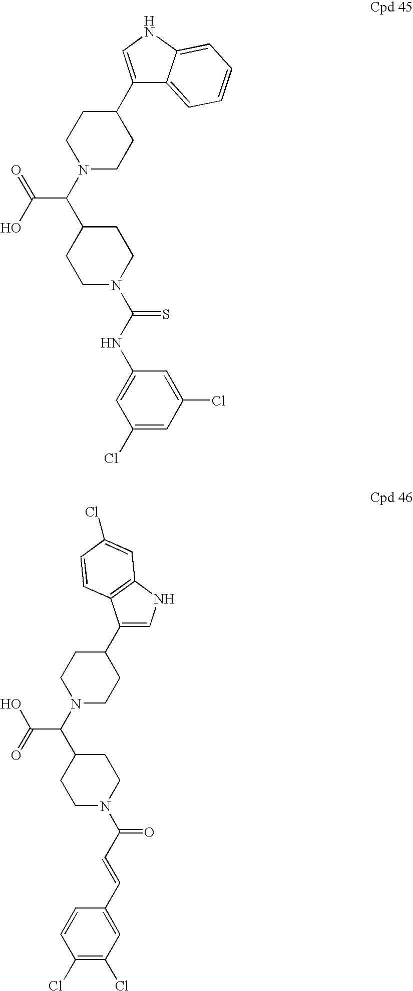 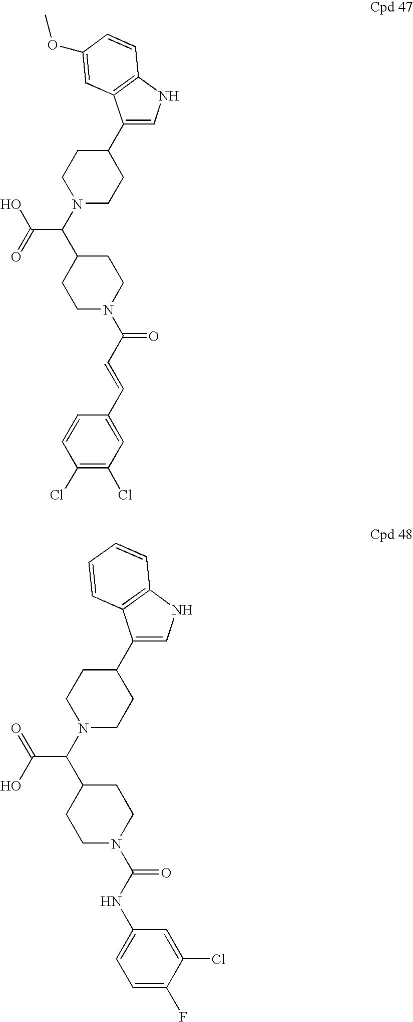 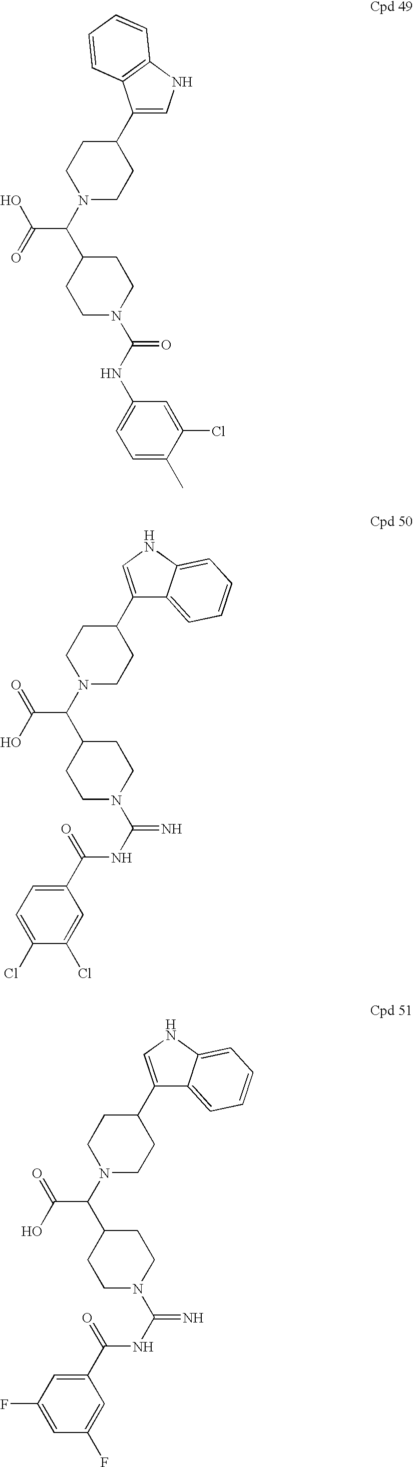 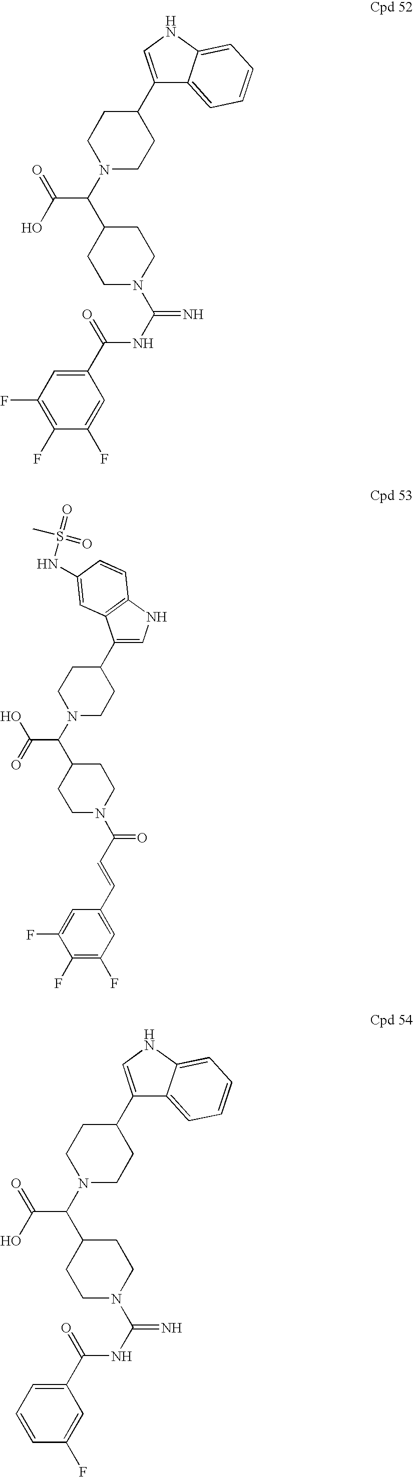 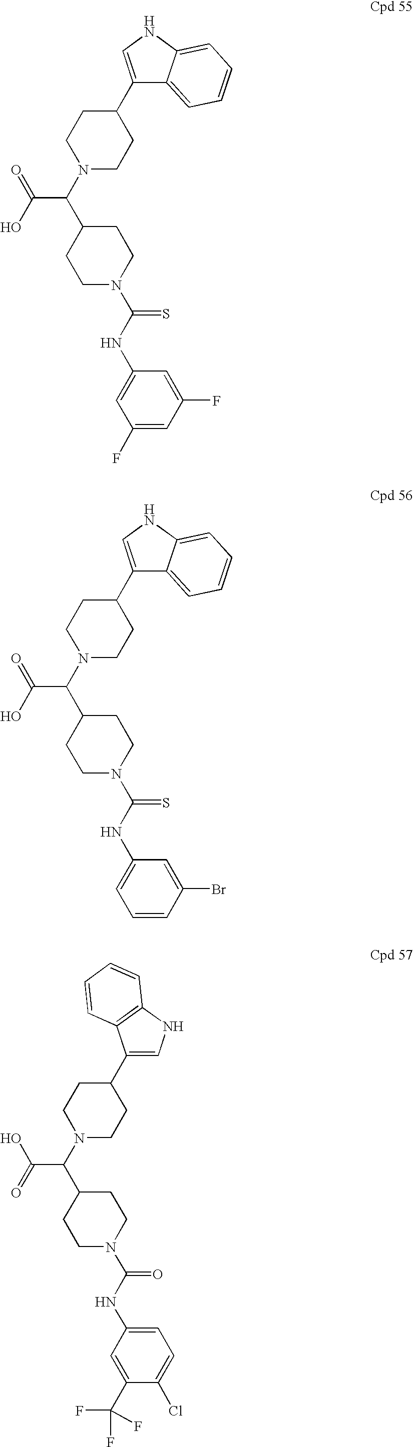 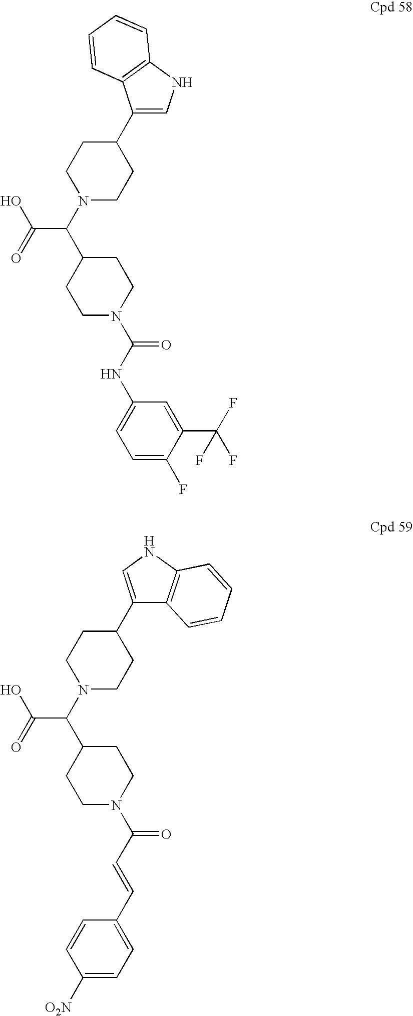 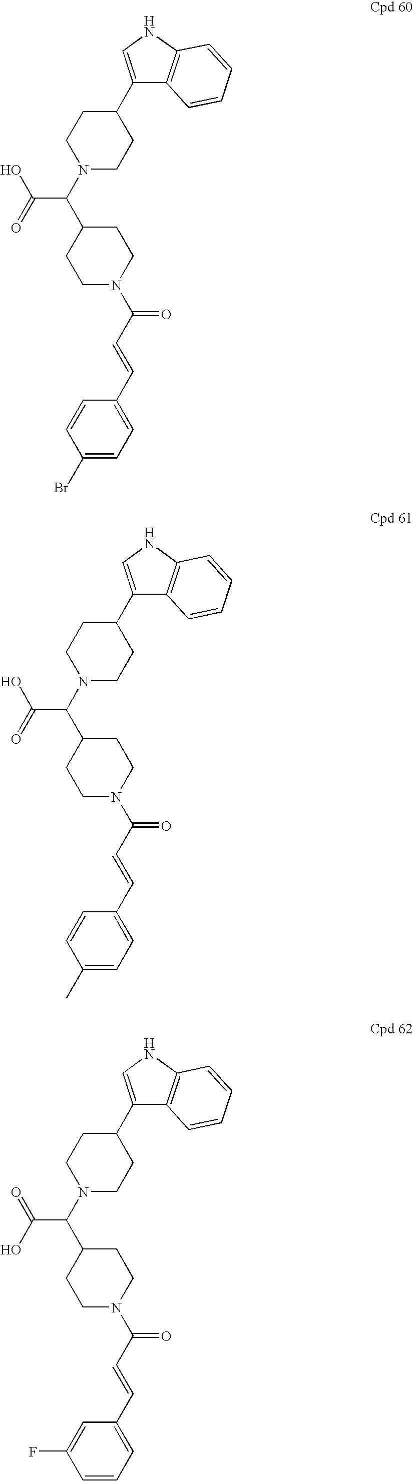 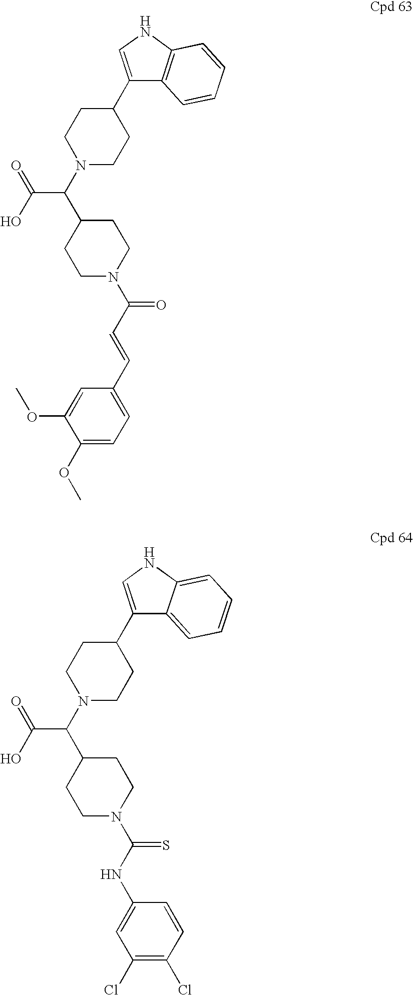 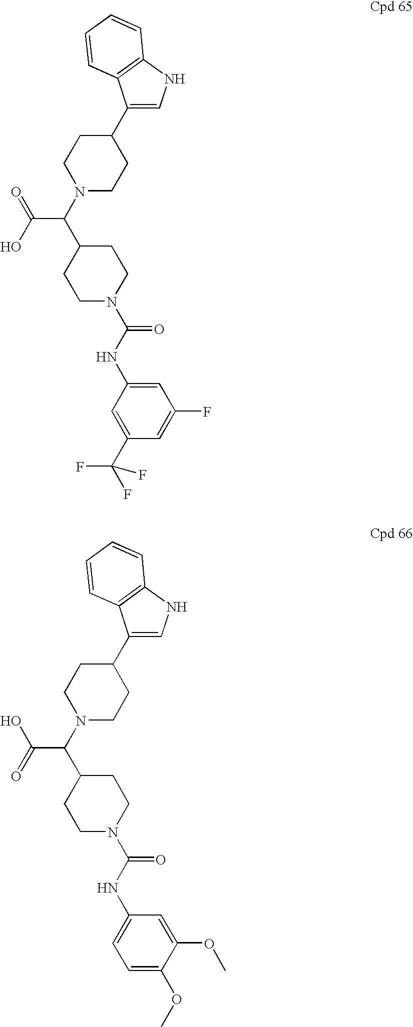 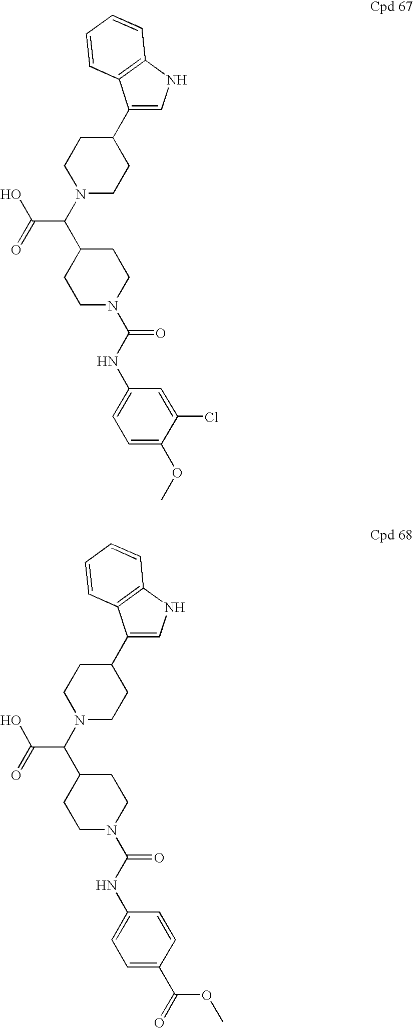 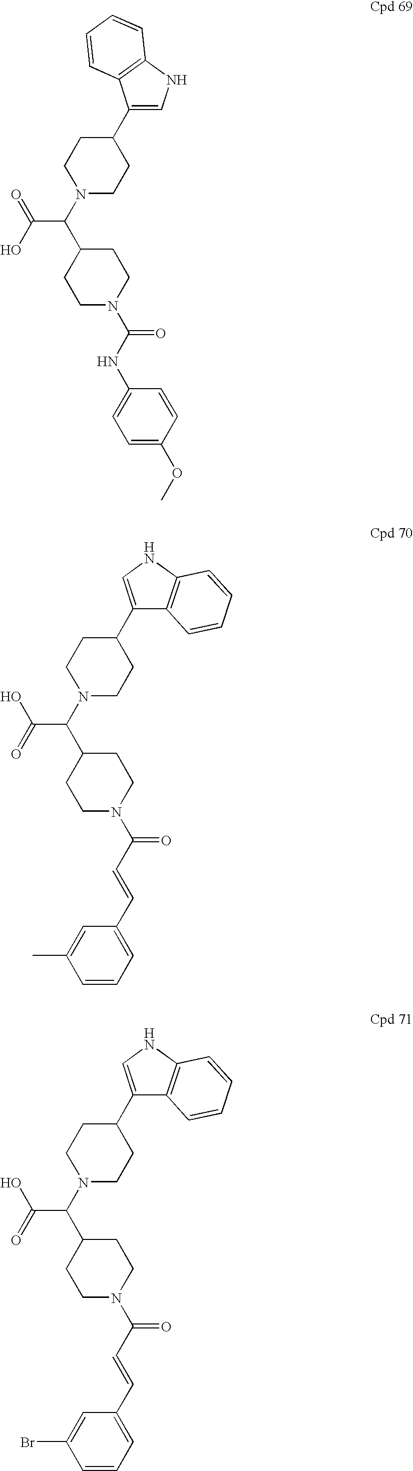 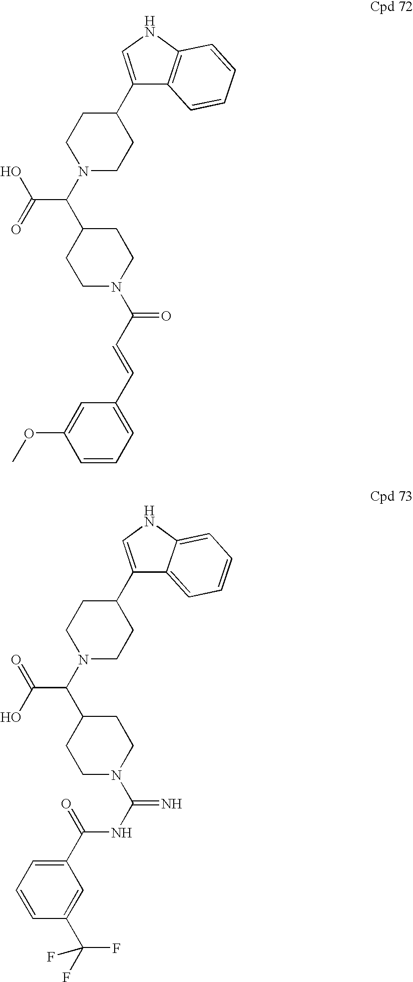 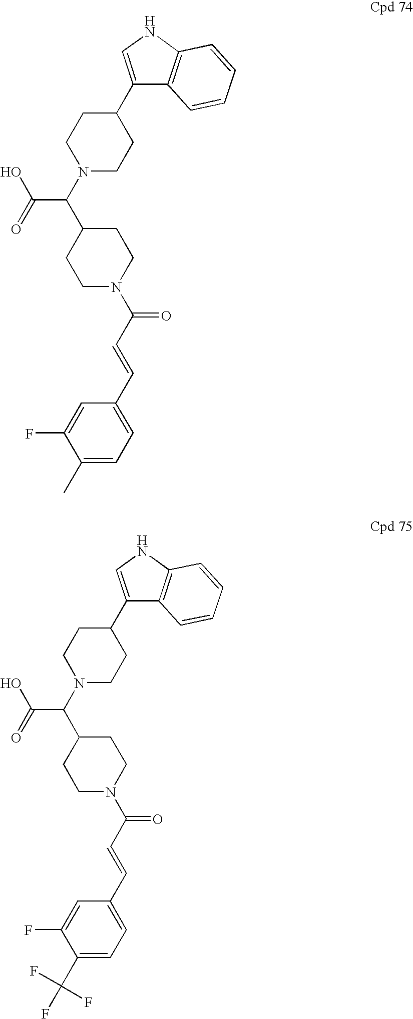 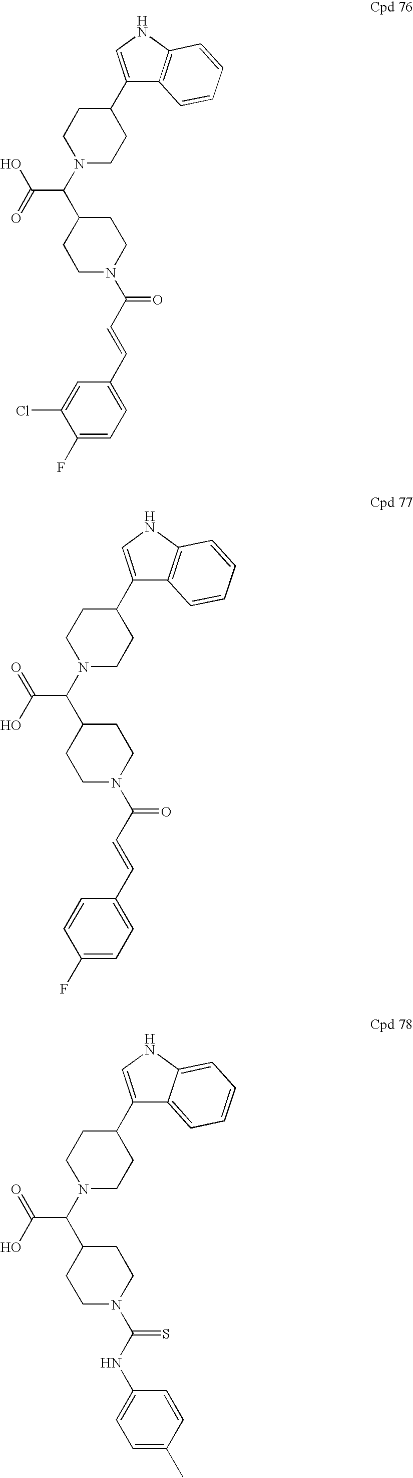 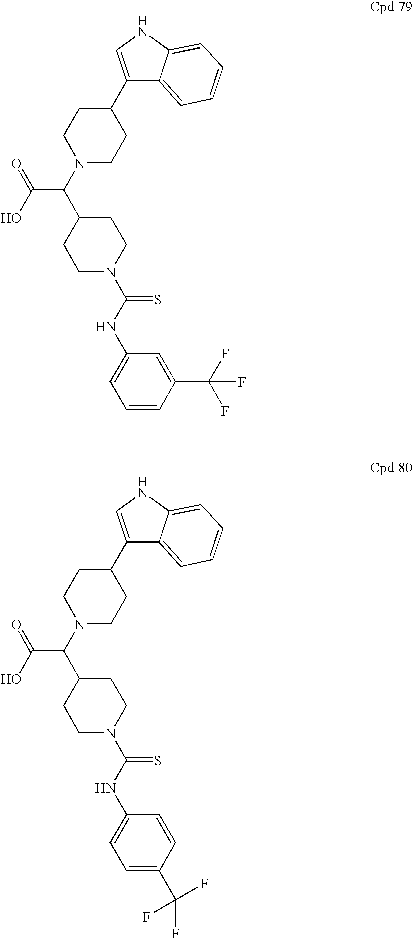 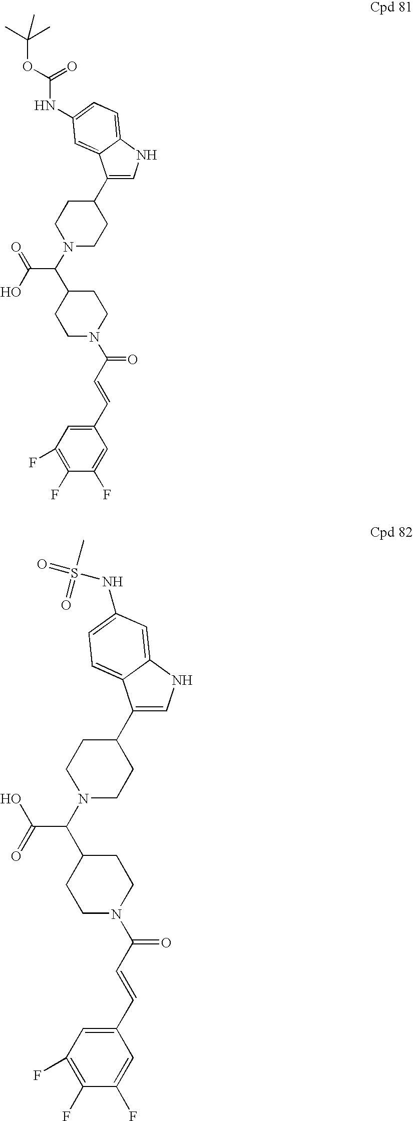 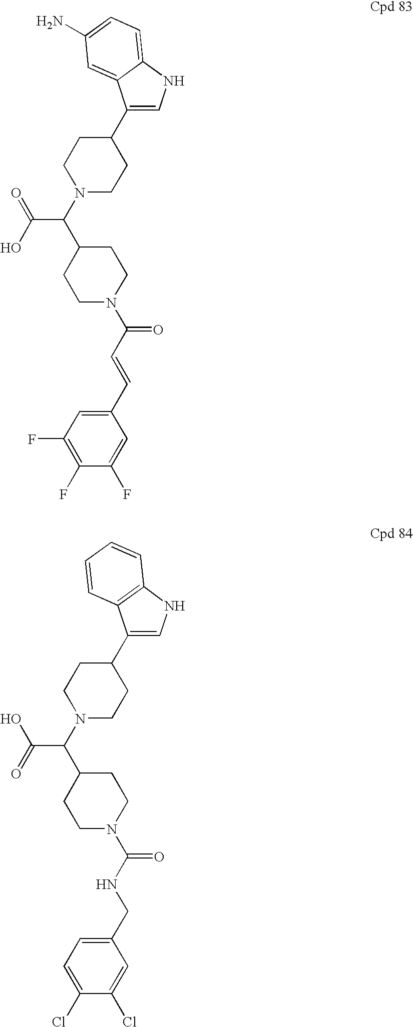 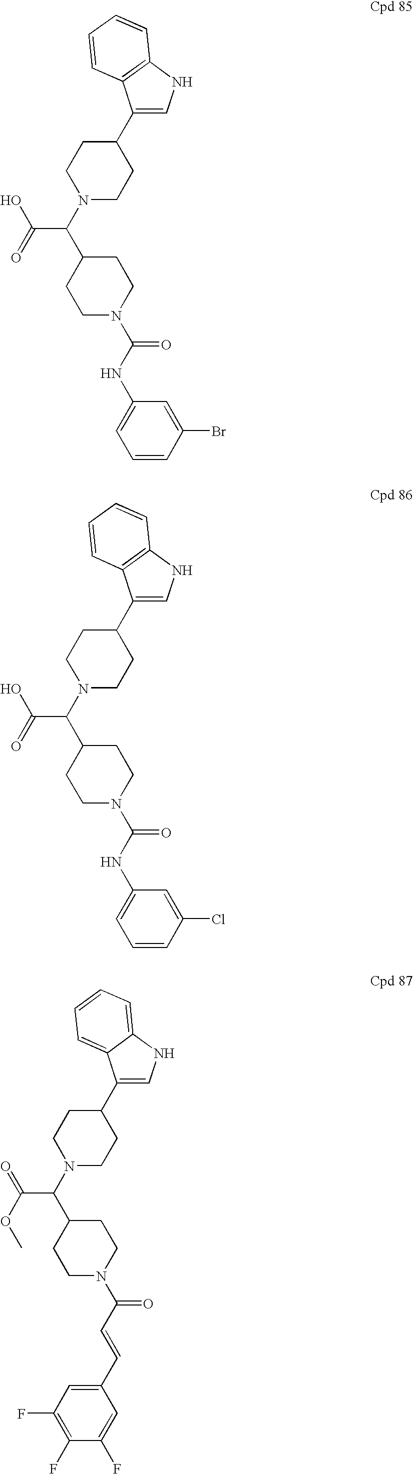 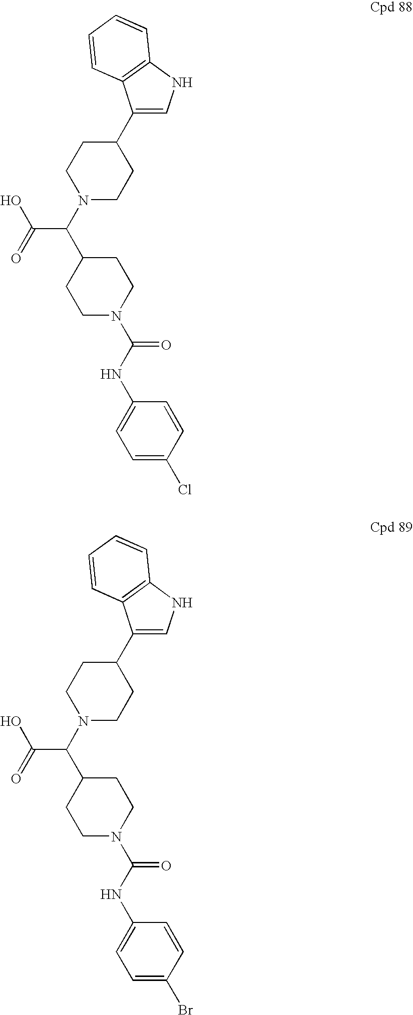 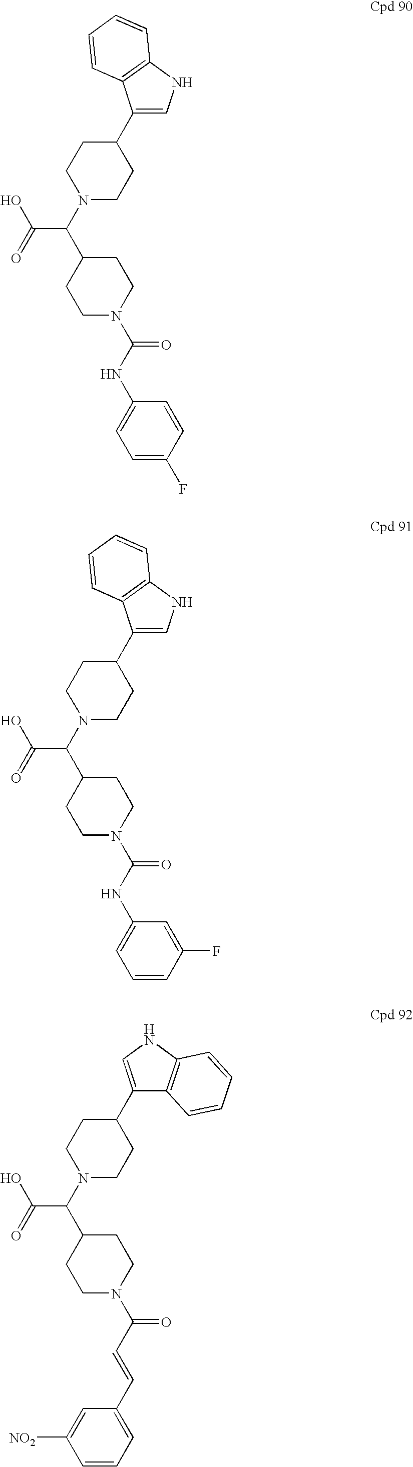 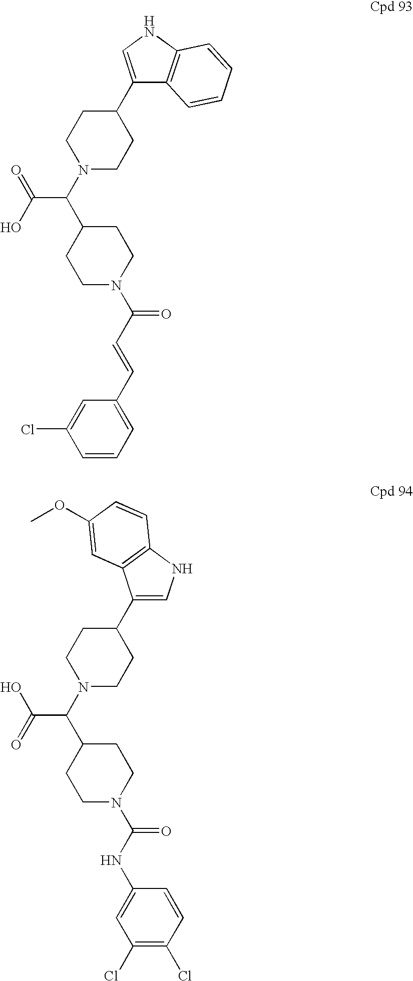 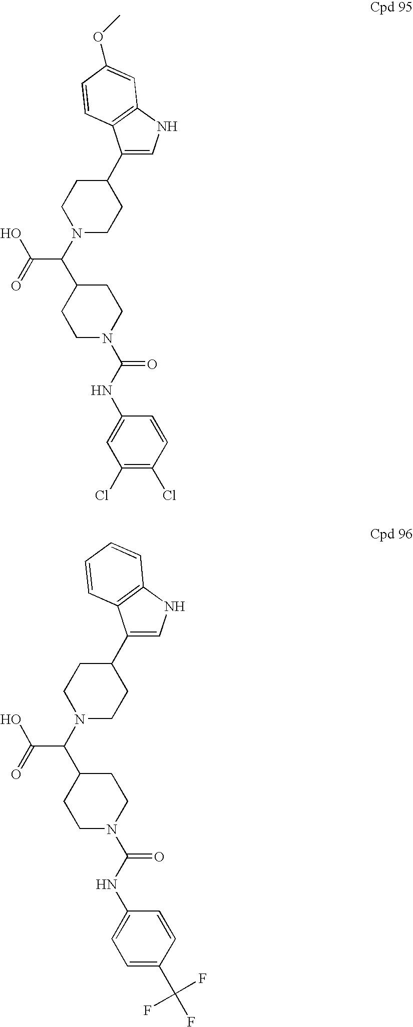 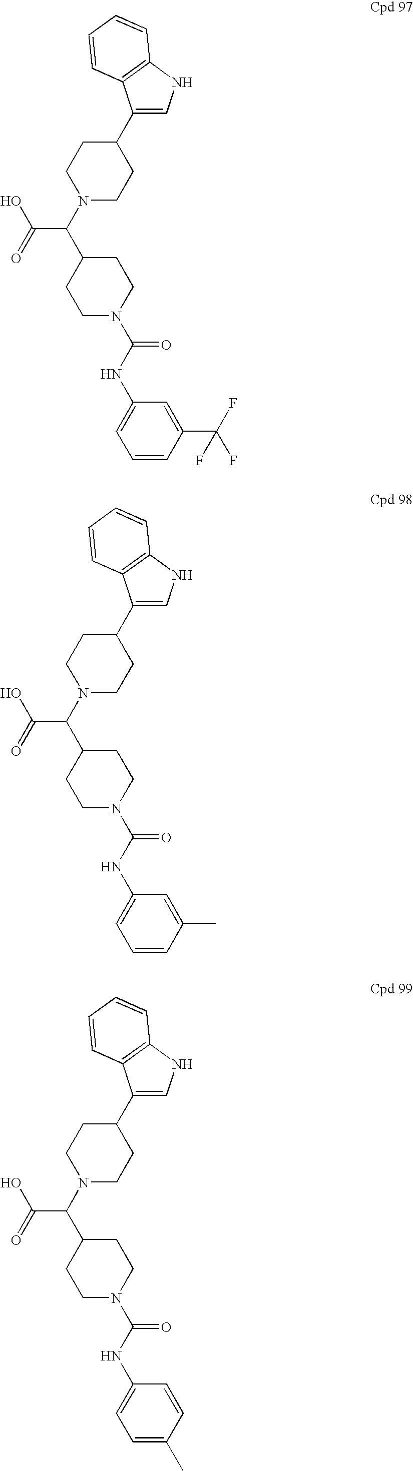 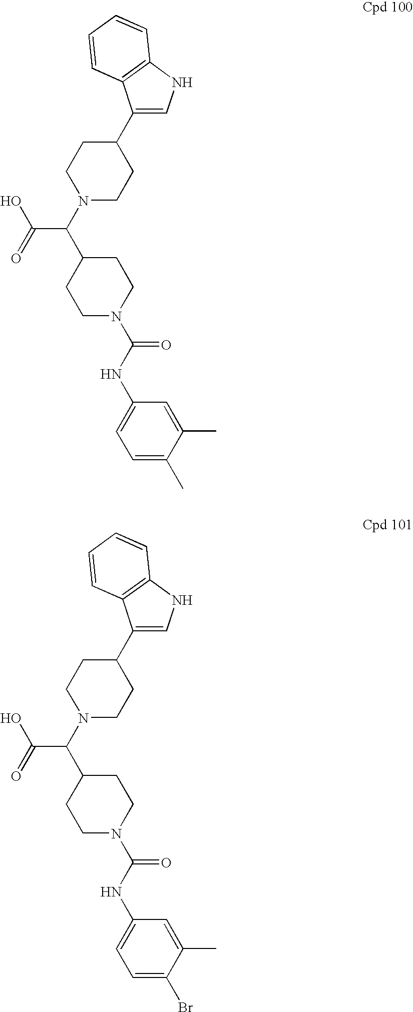 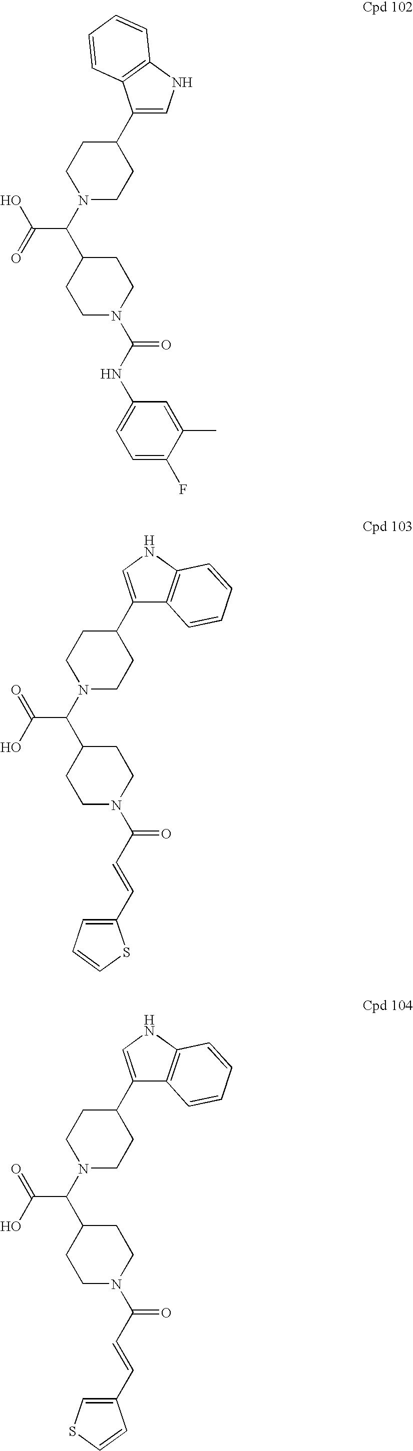 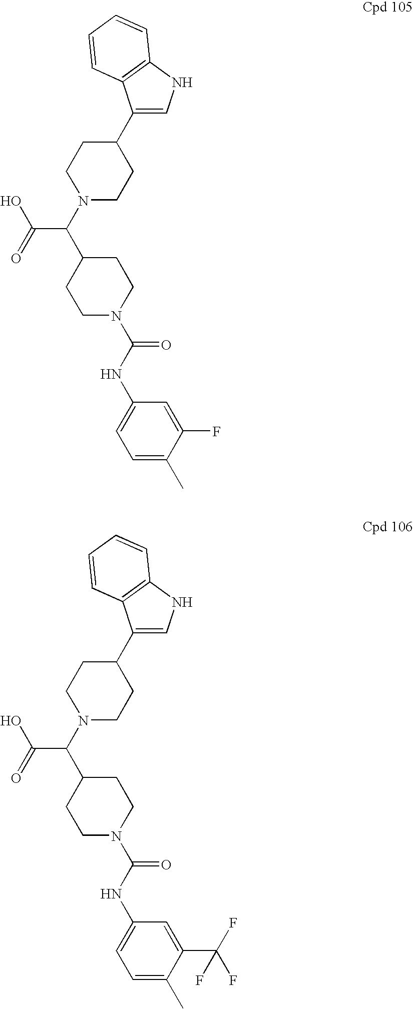 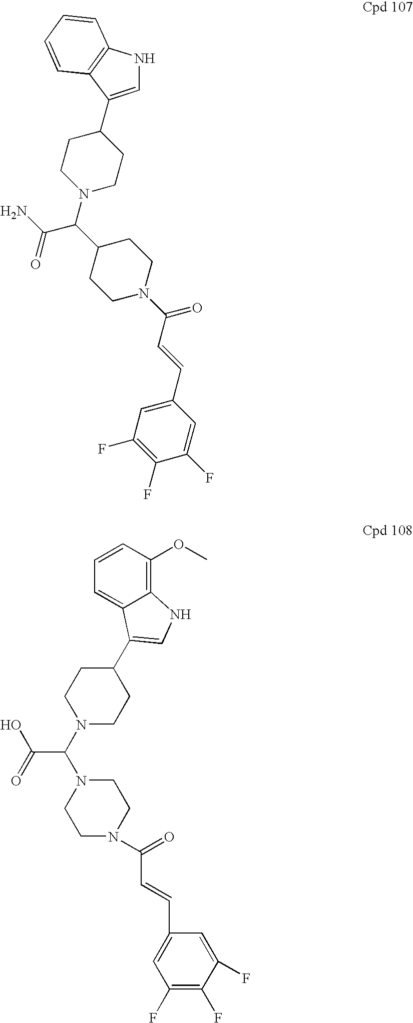 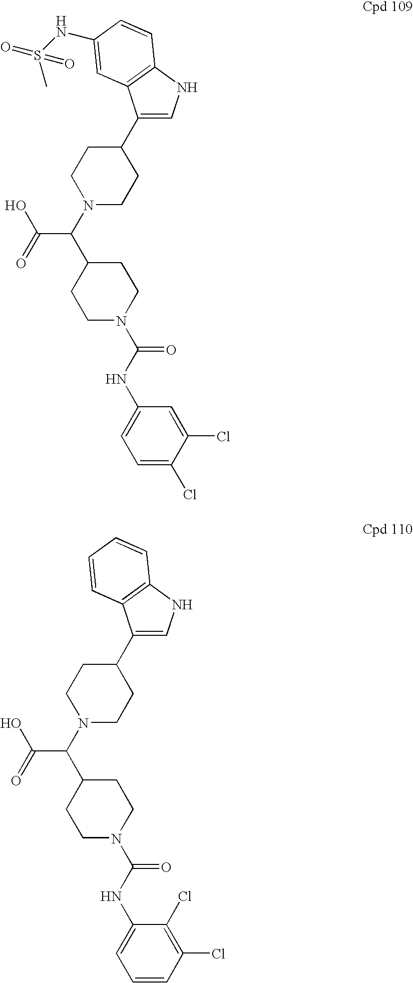 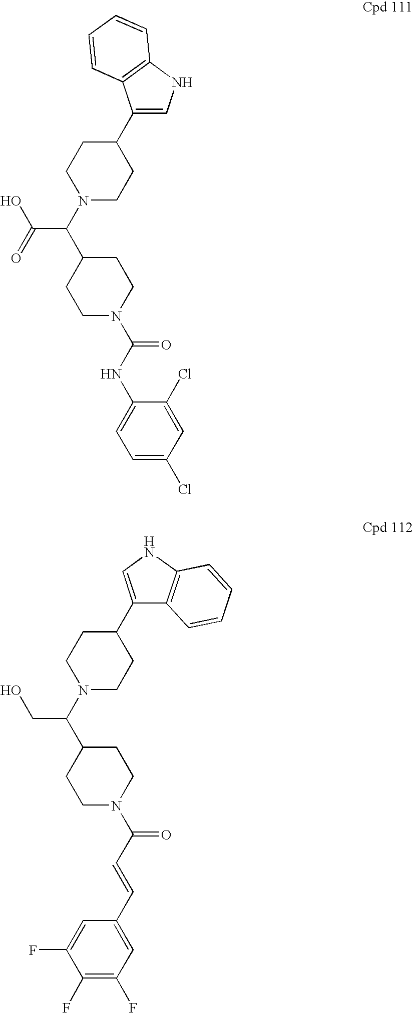 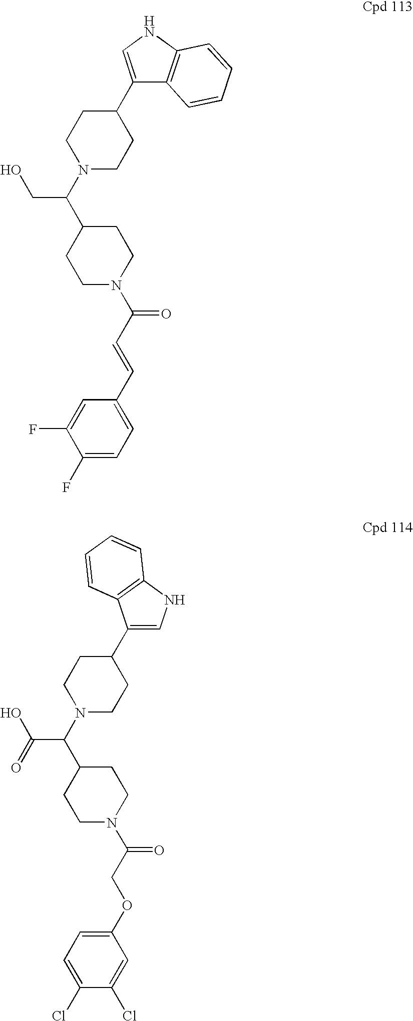 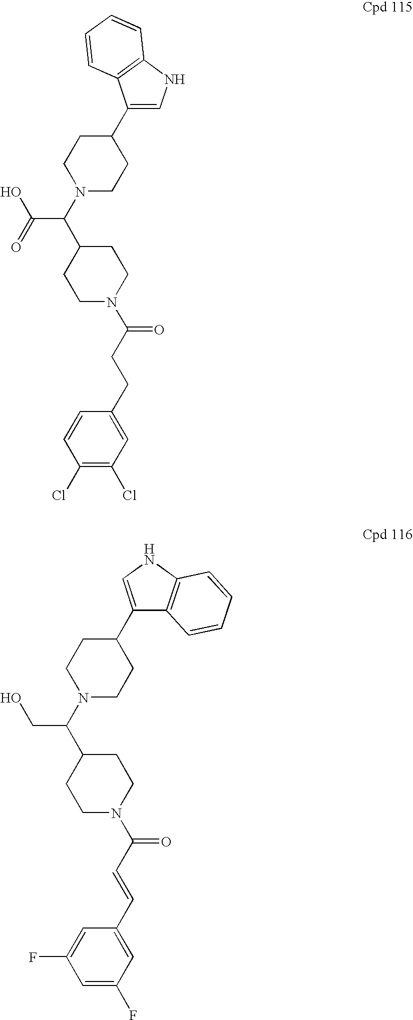 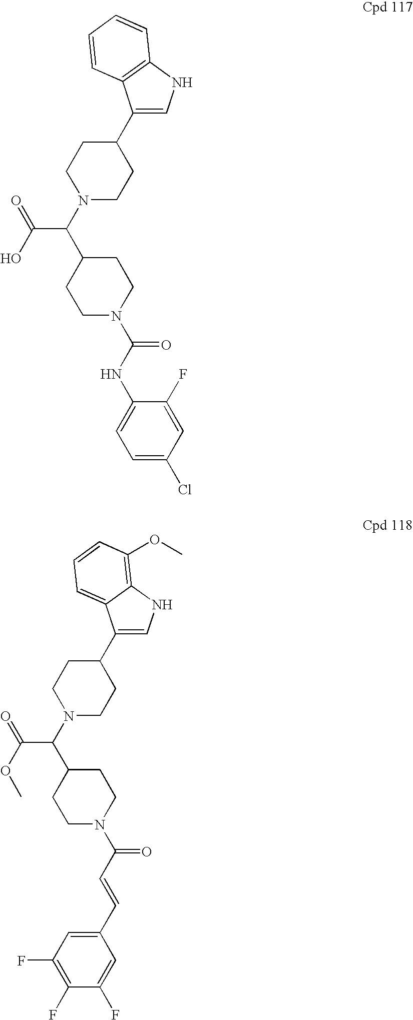 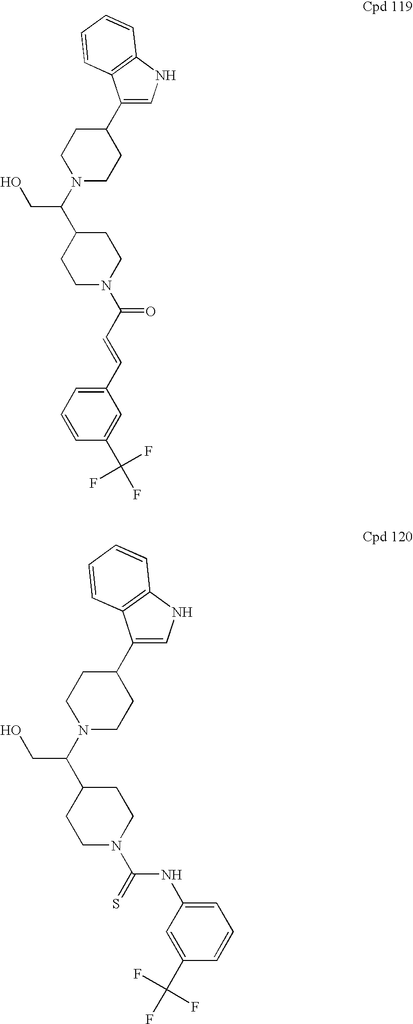 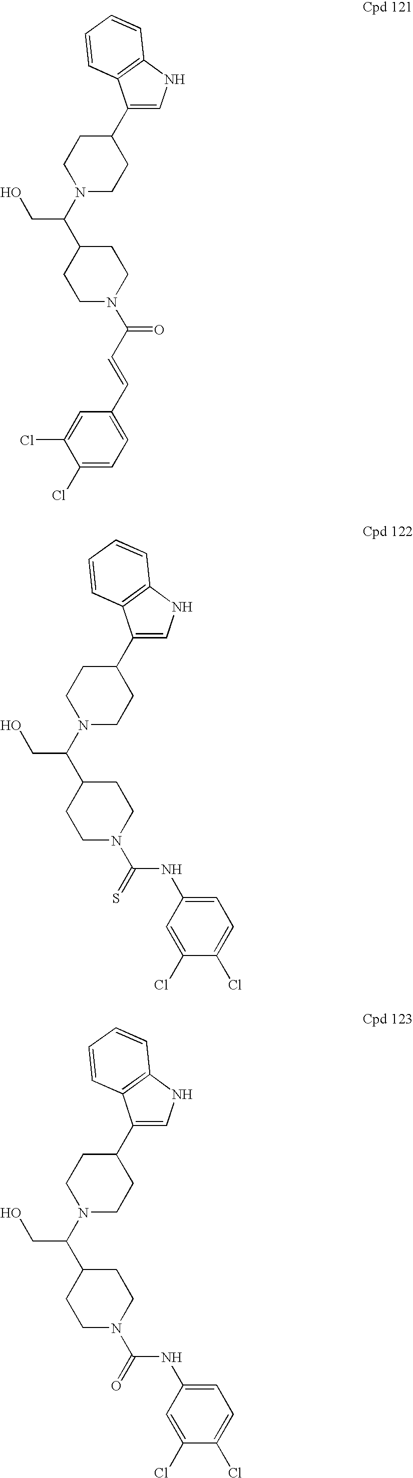 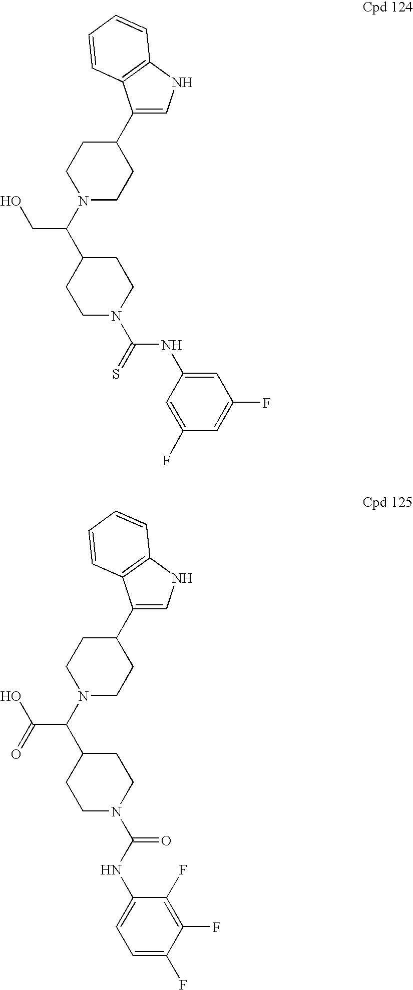 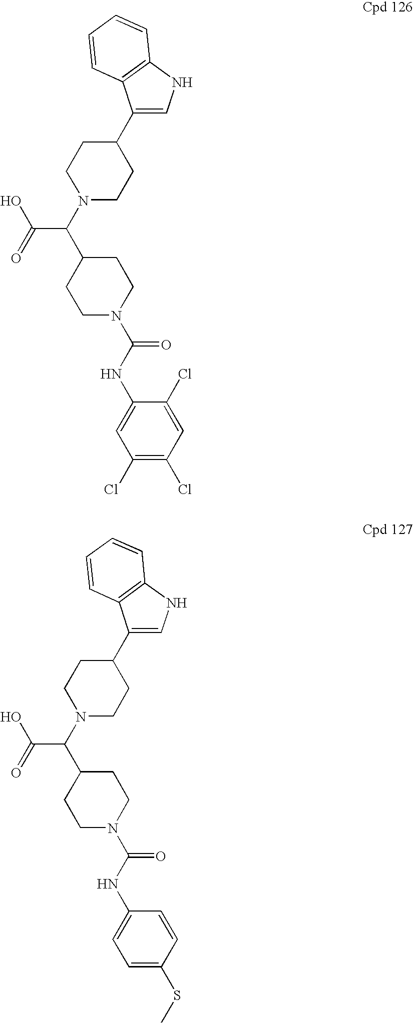 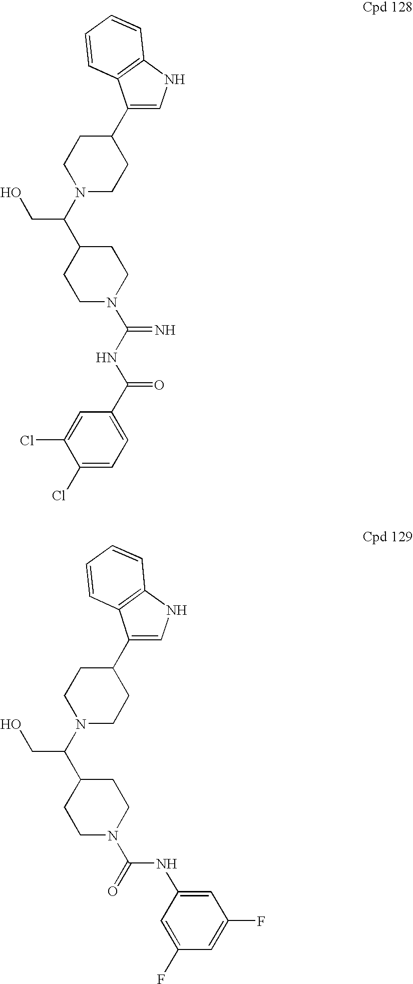 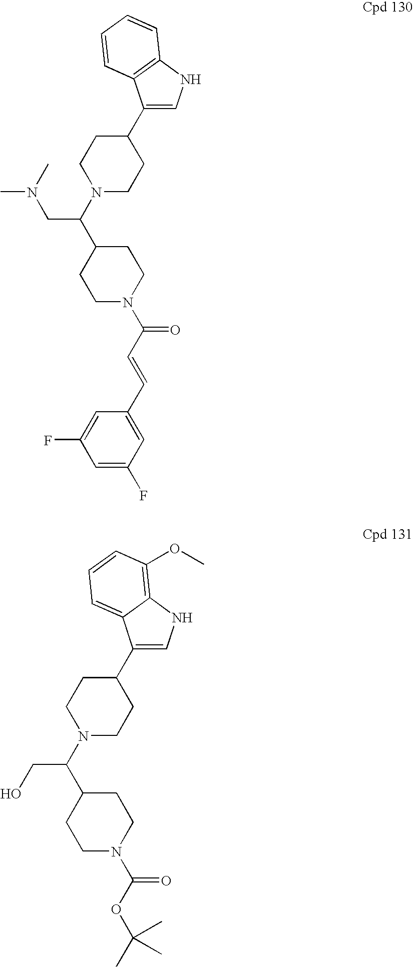 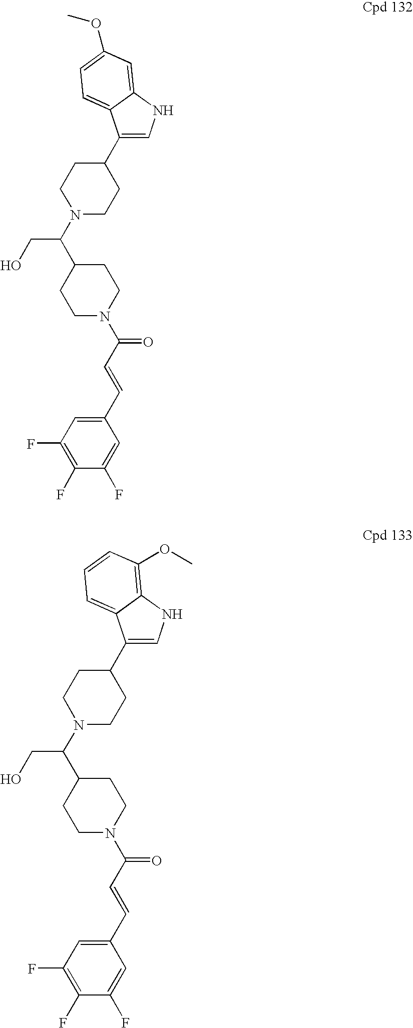 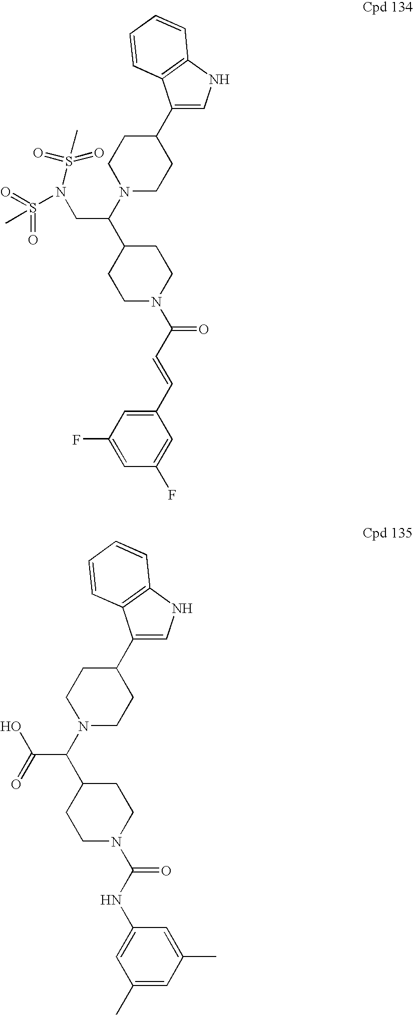 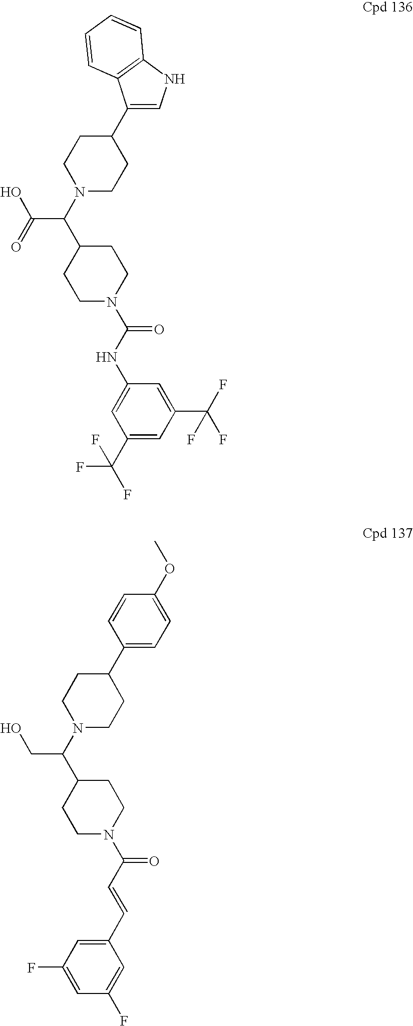 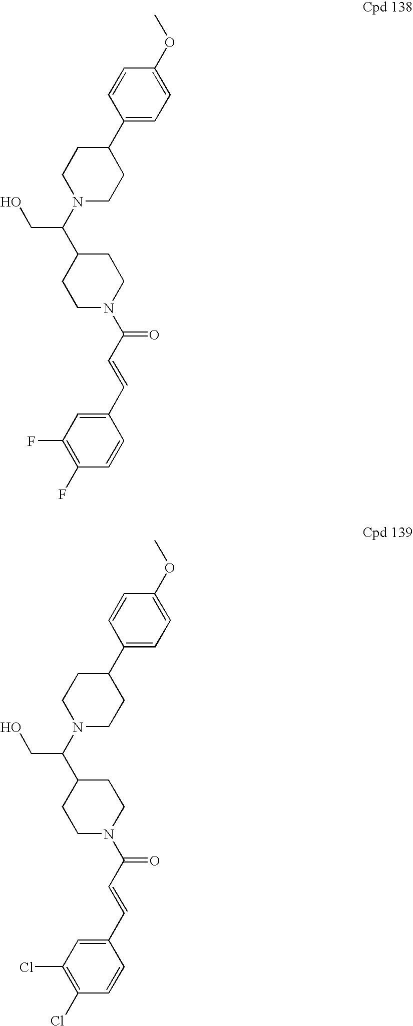 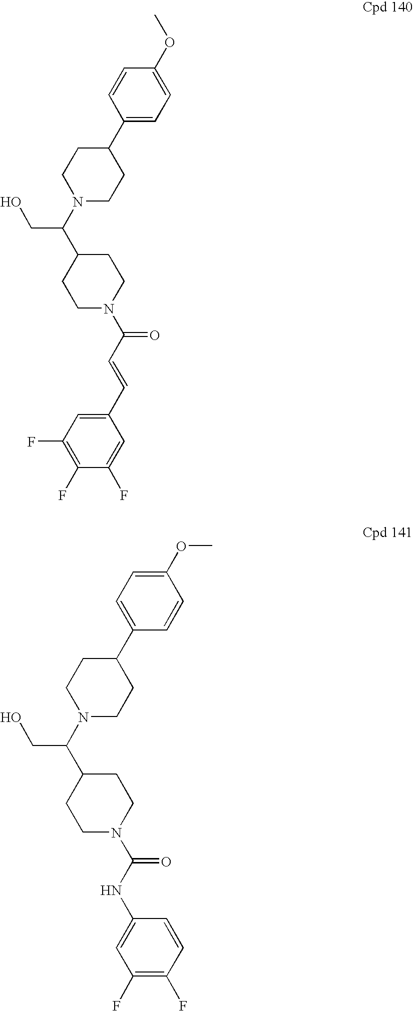 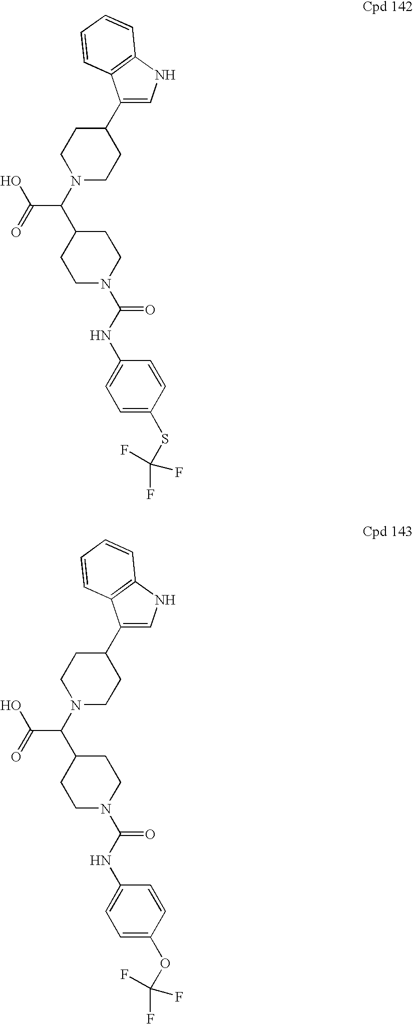 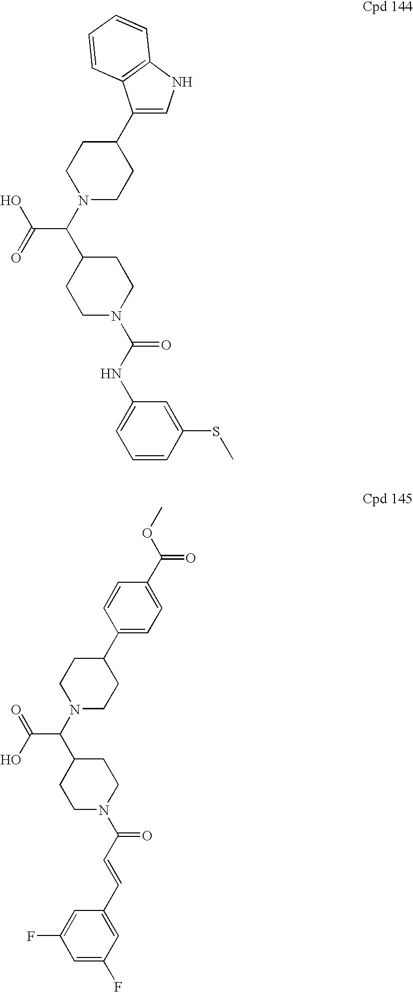 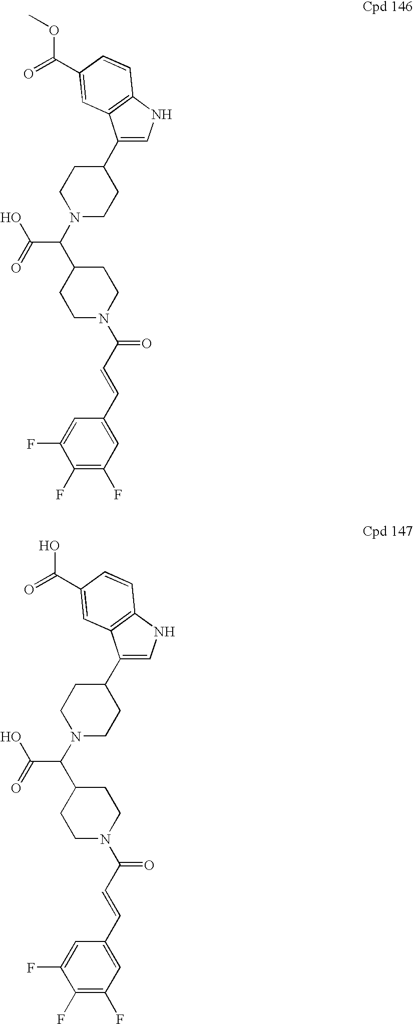 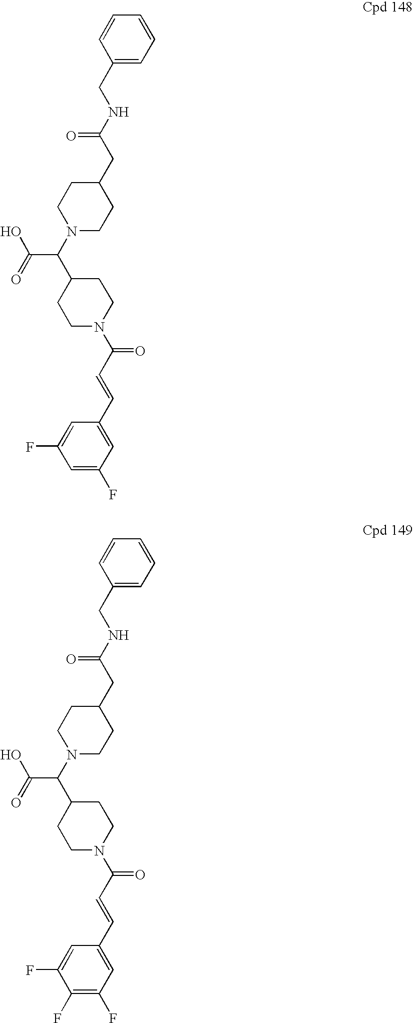 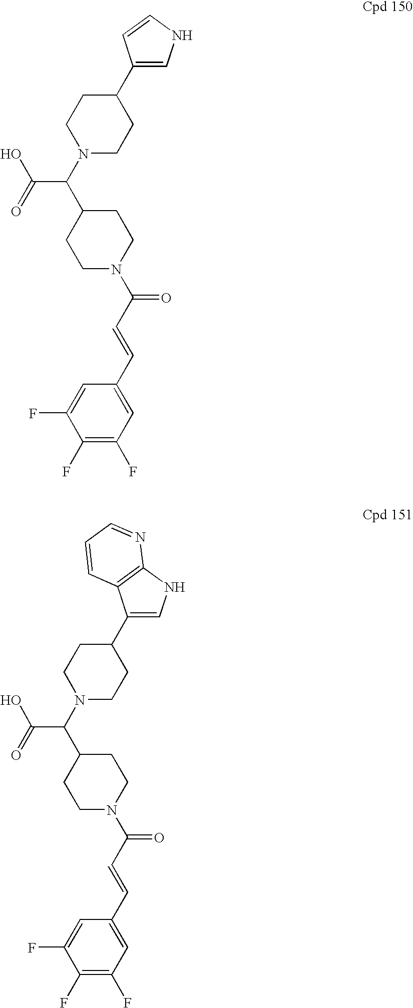 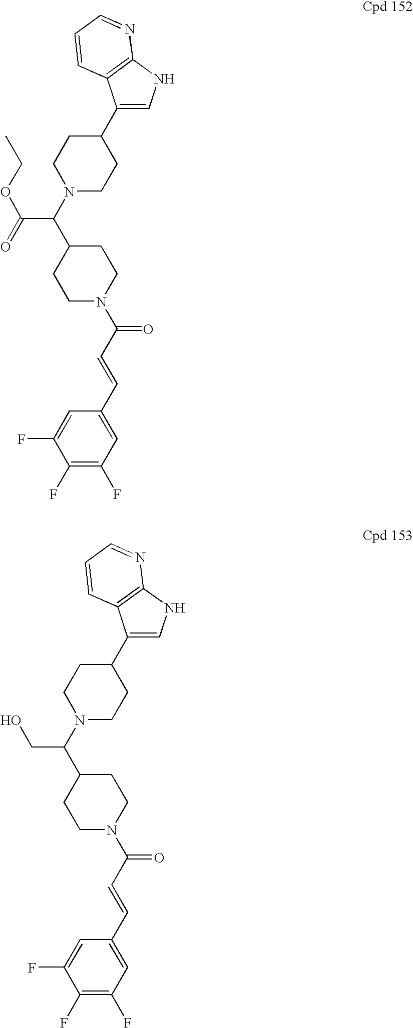 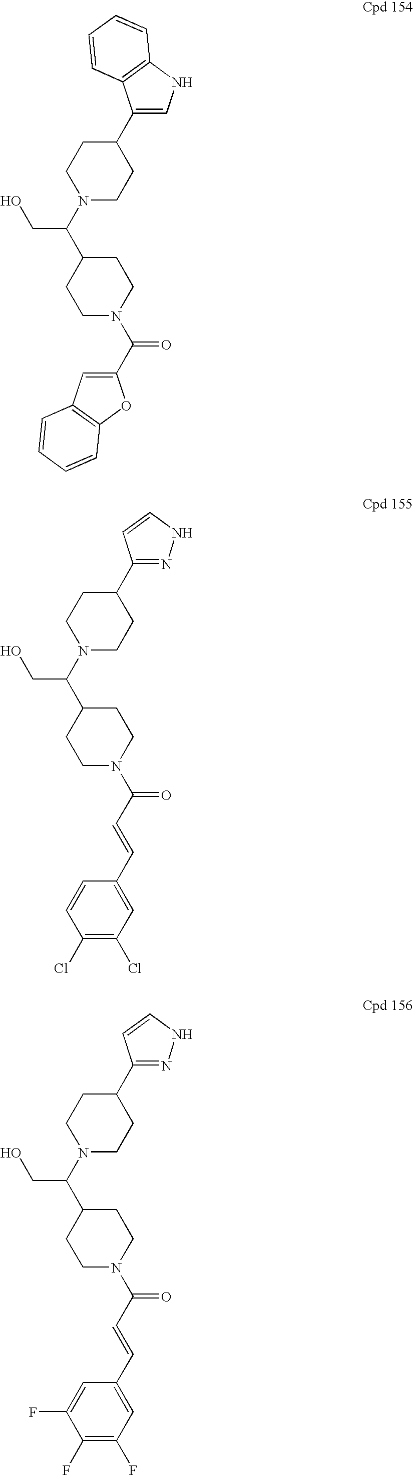 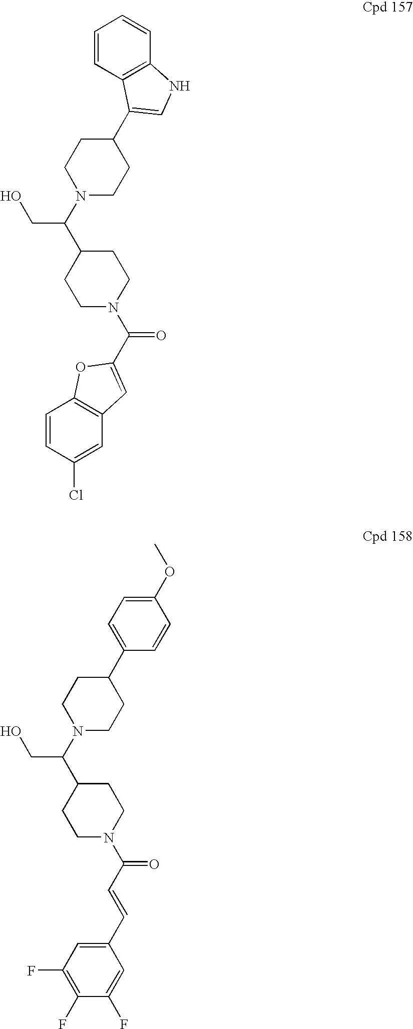 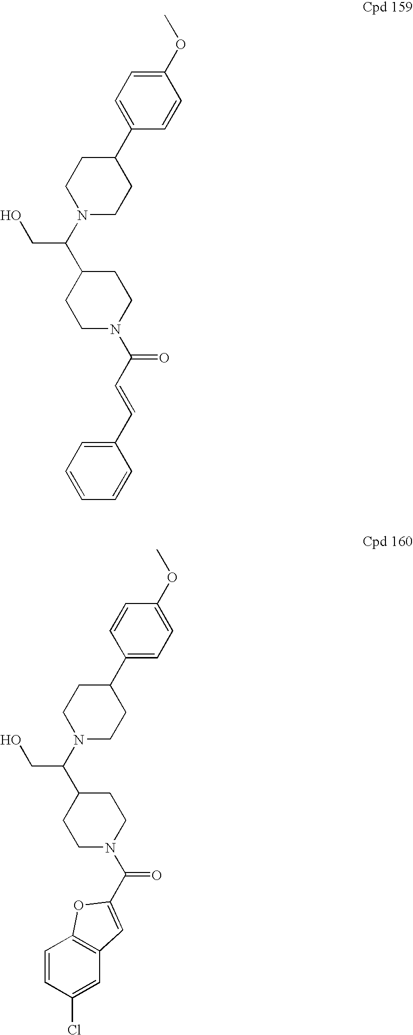 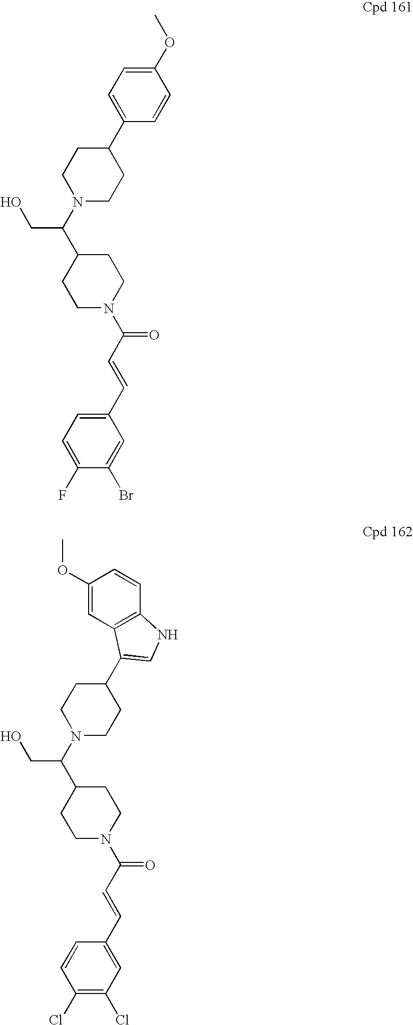 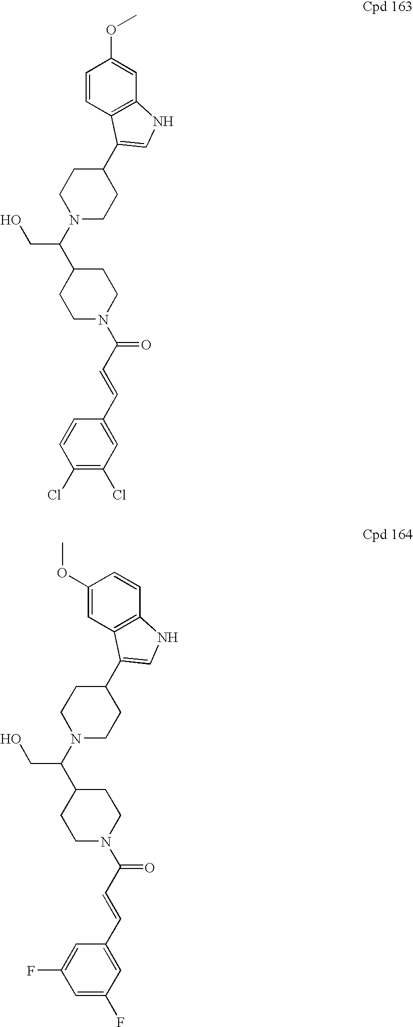 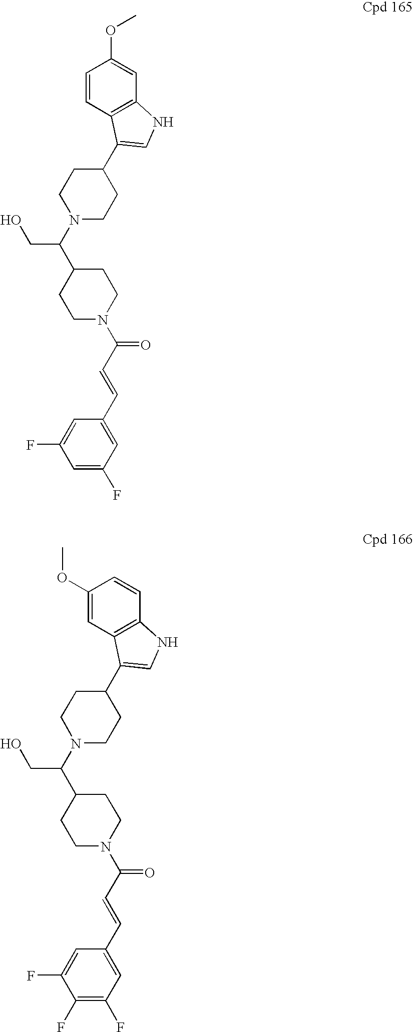 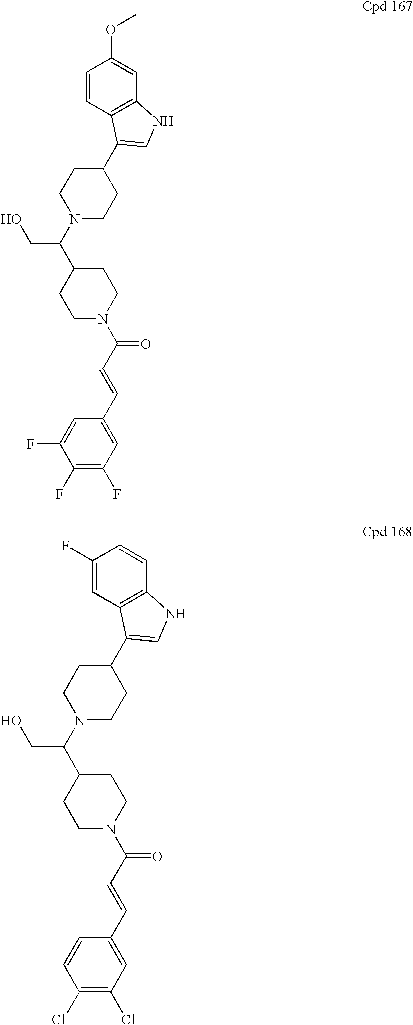 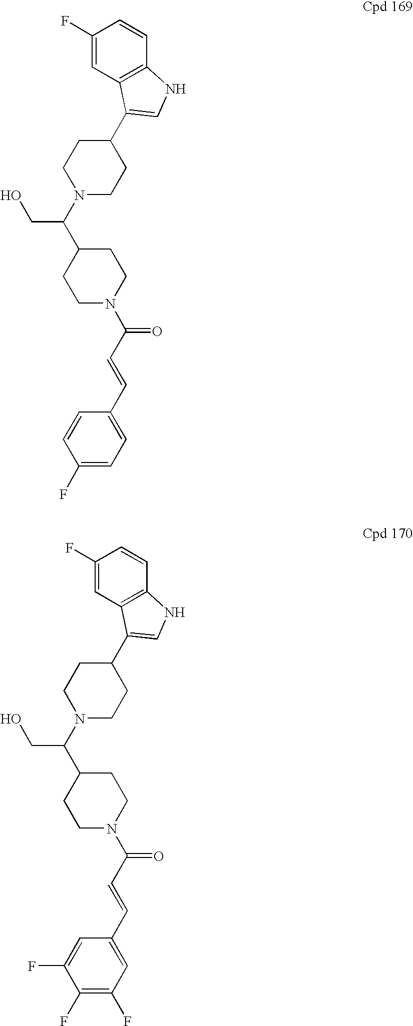 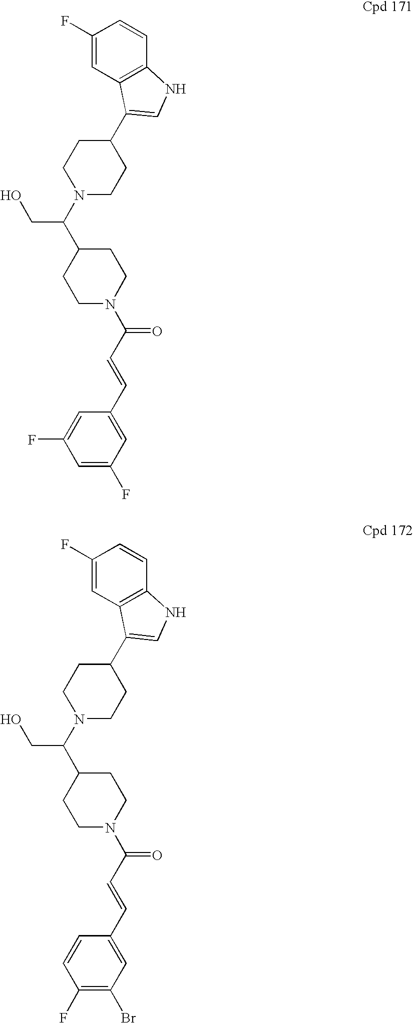 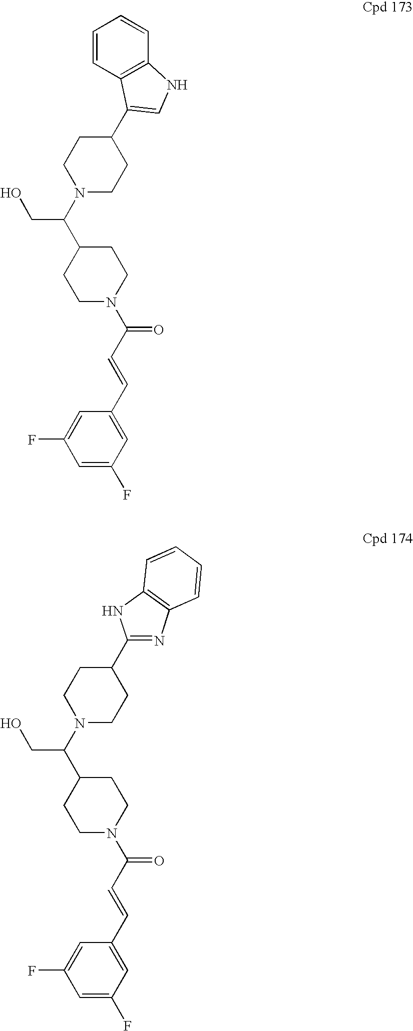 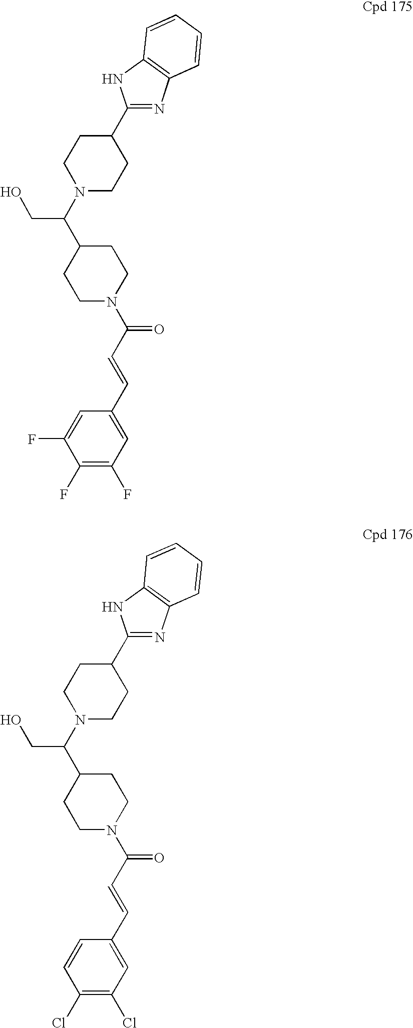 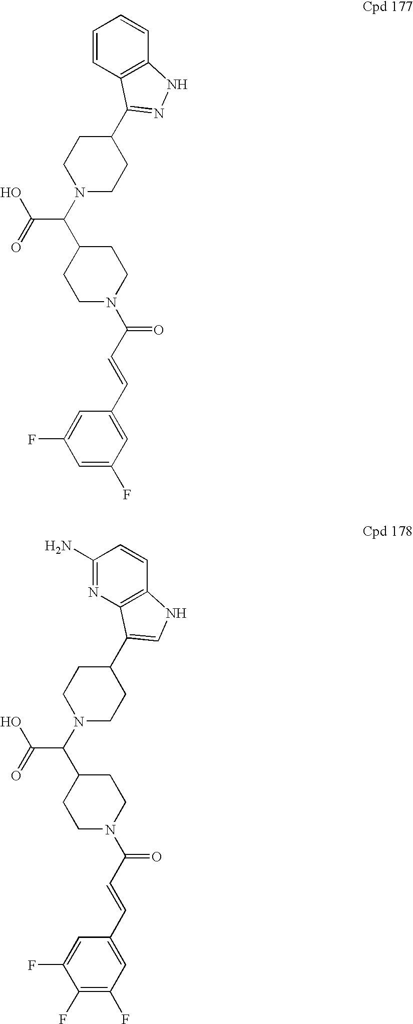 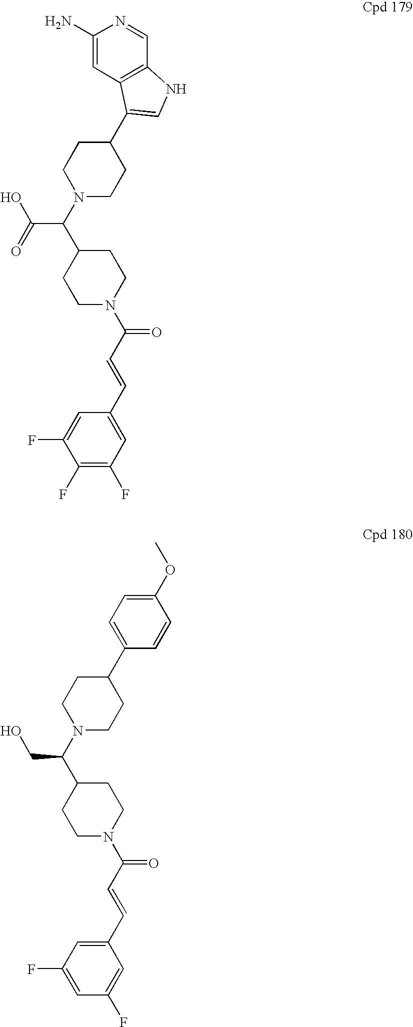 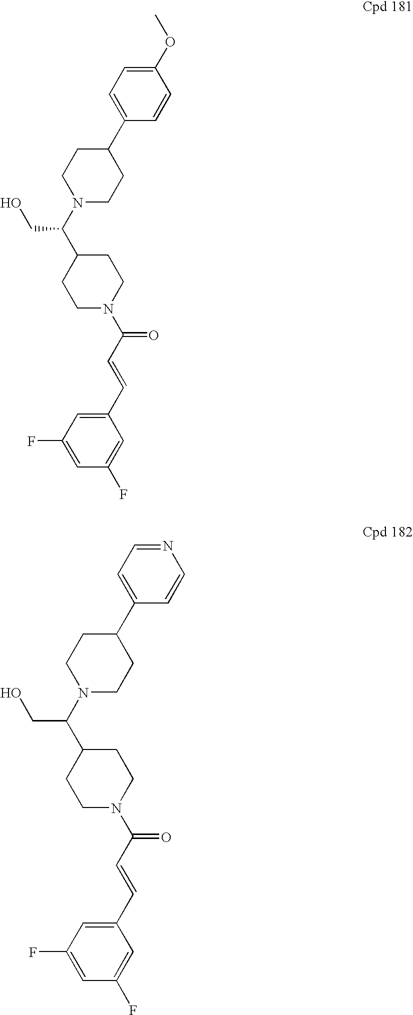 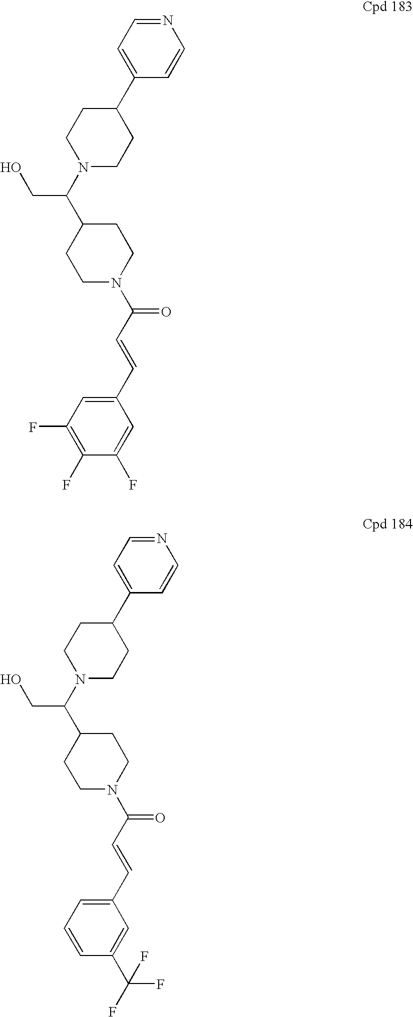 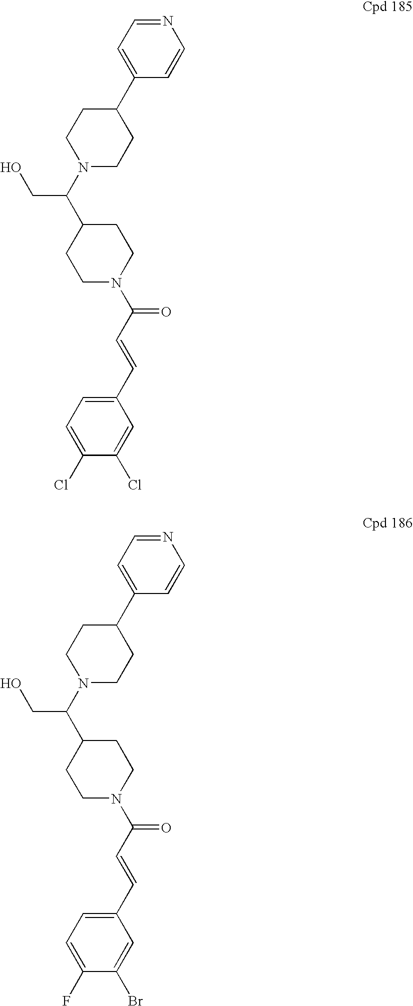 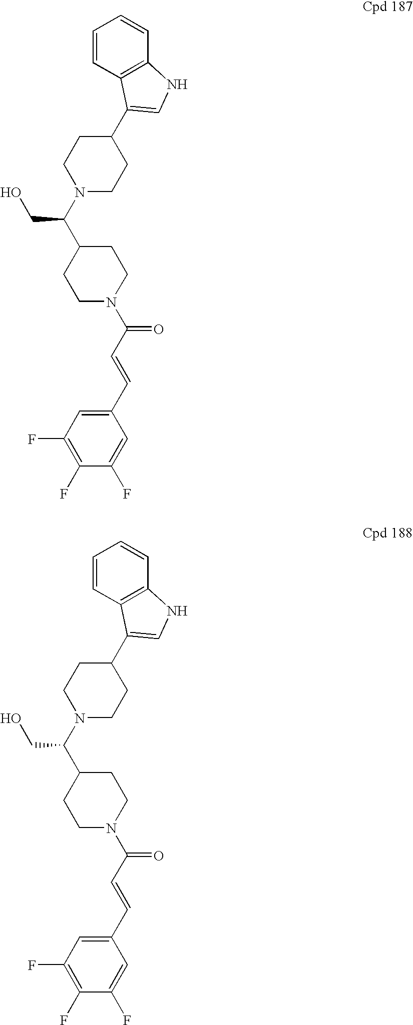 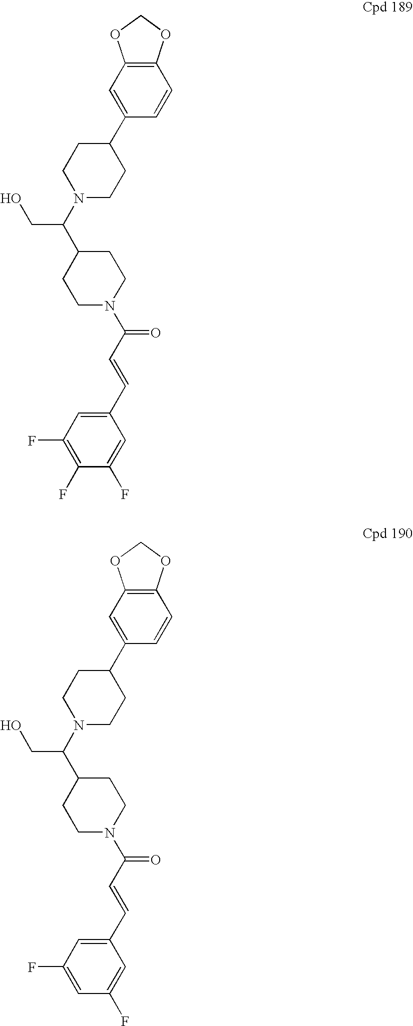 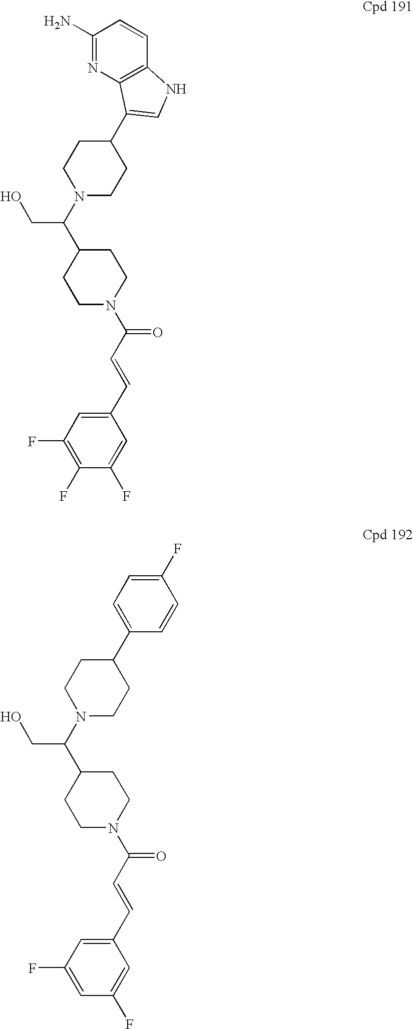 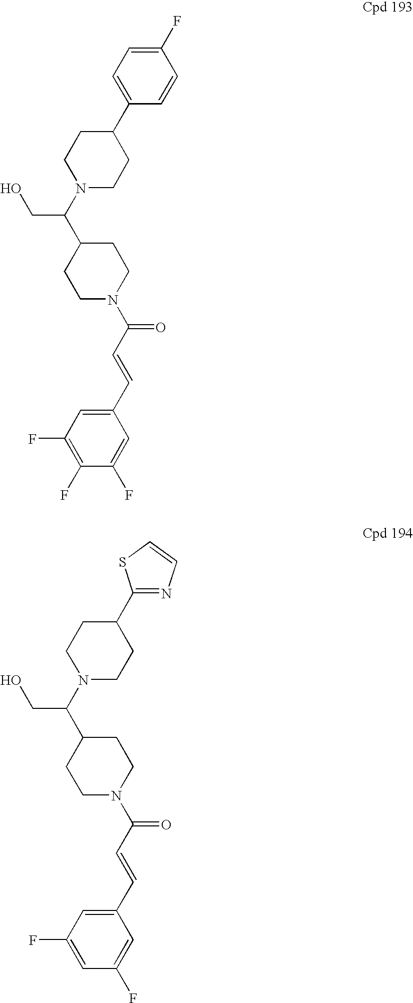 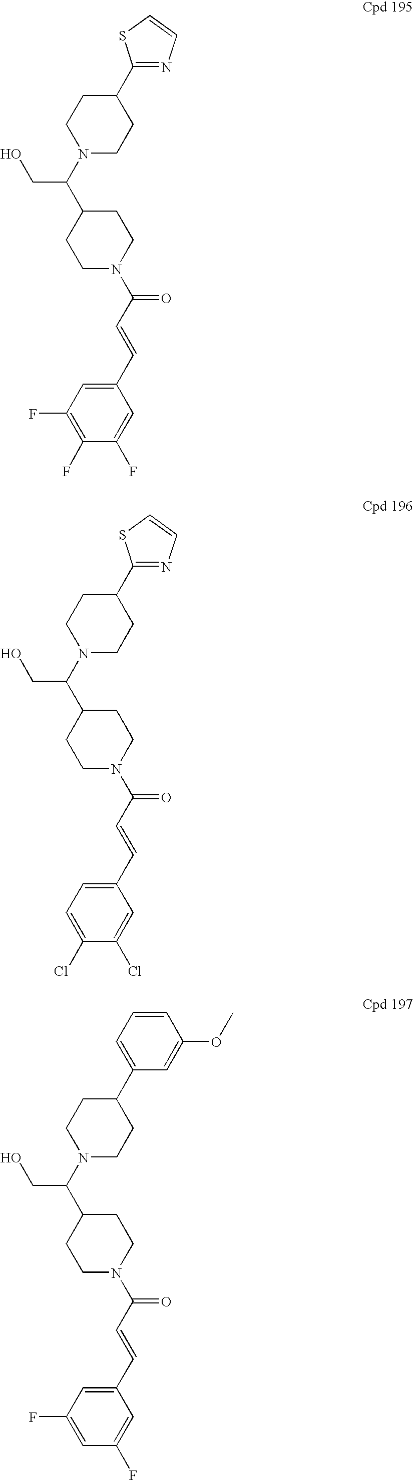 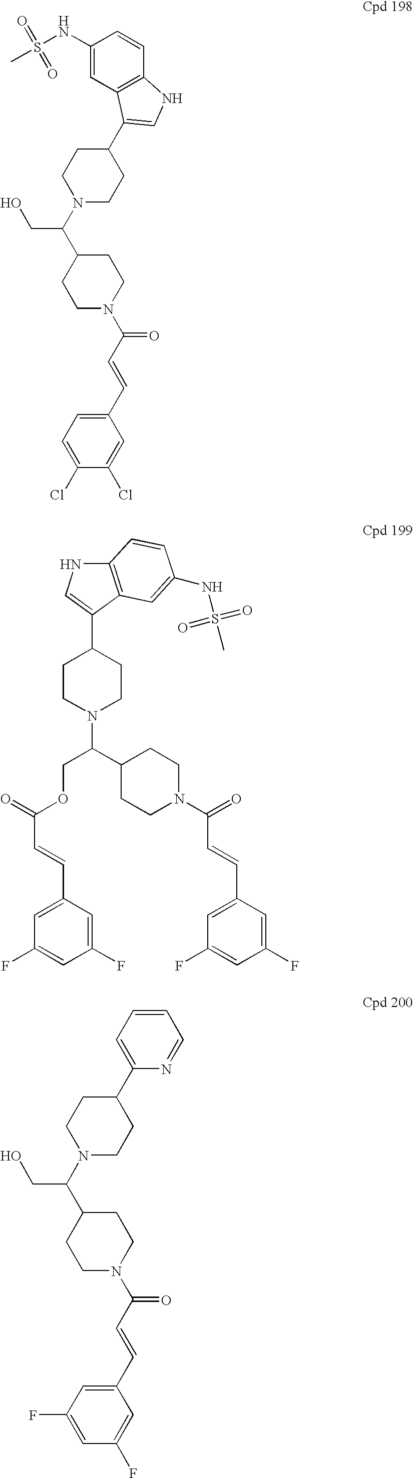 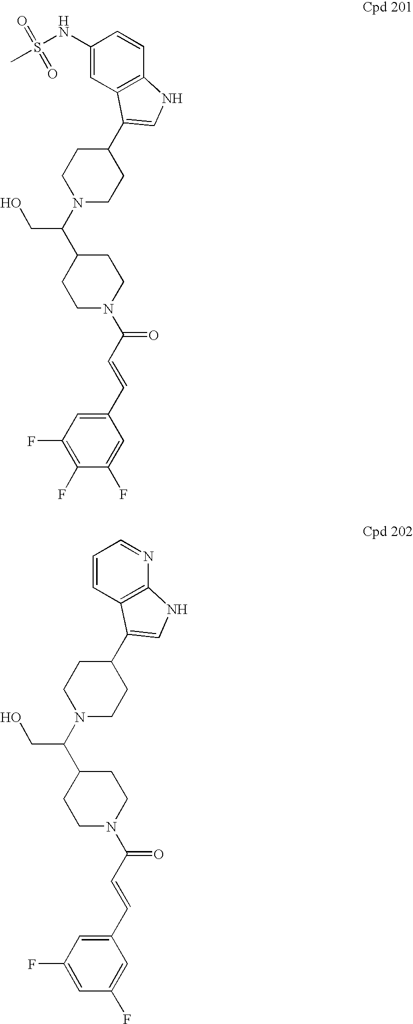 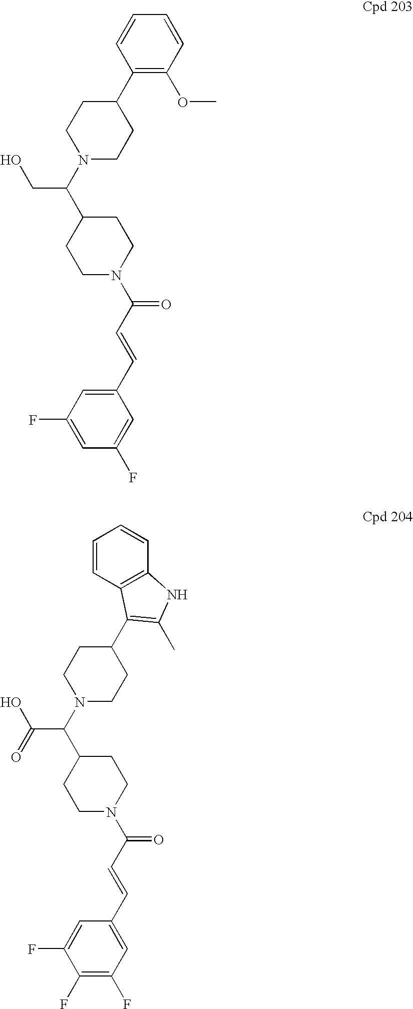 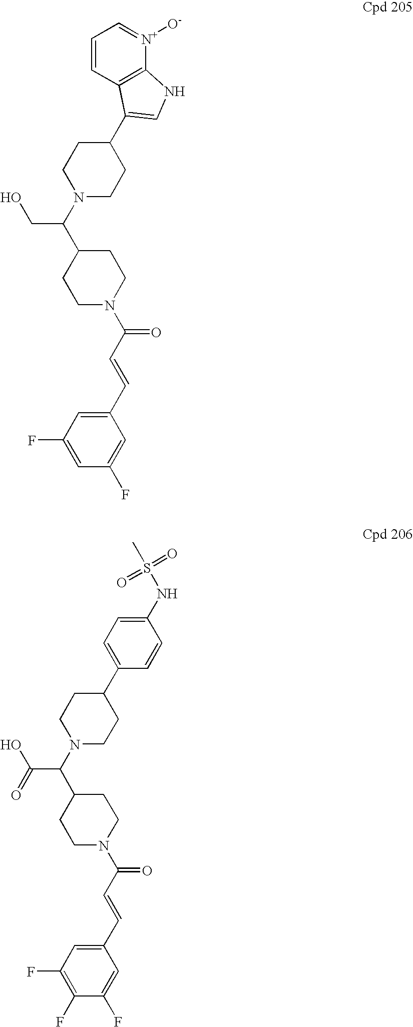 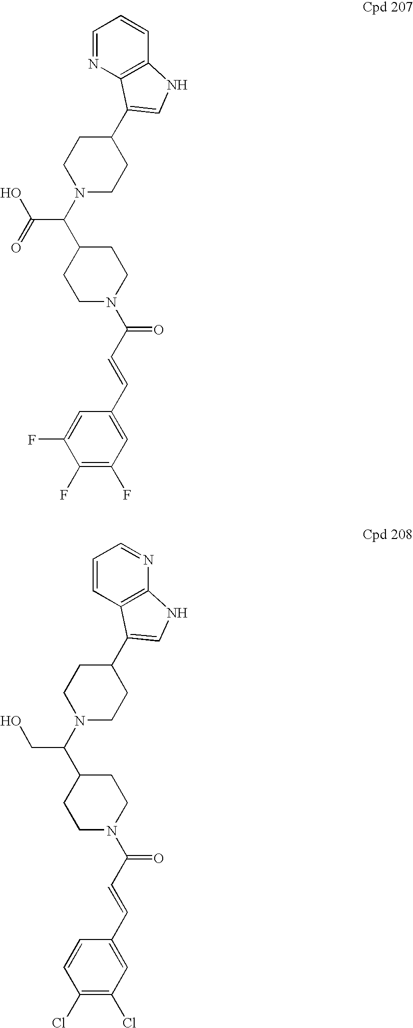 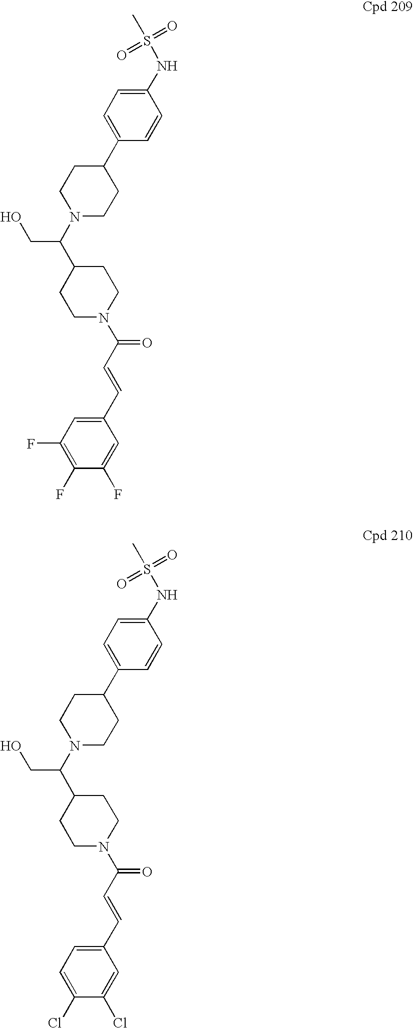 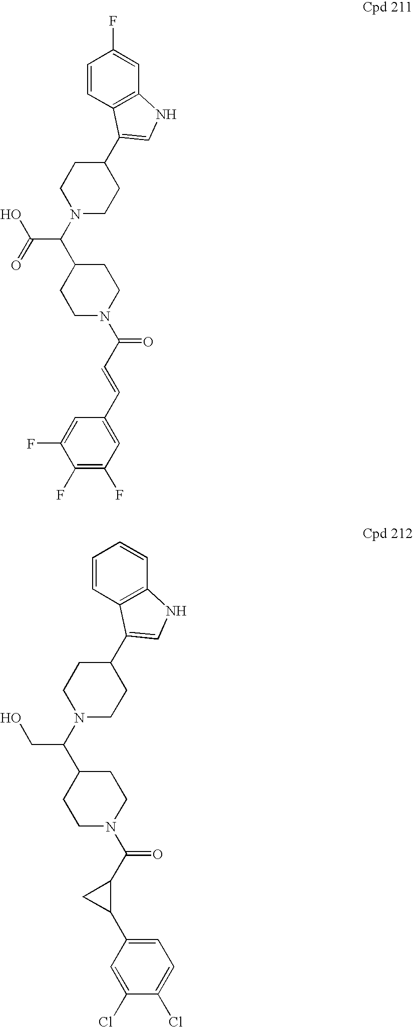 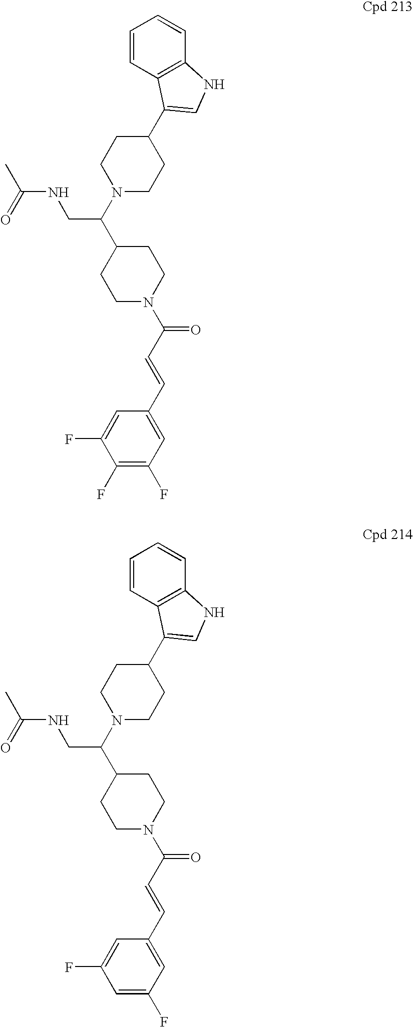
Chemical Definitions

As used herein, the following terms have the following meanings.

The term “alkyl” means a saturated aliphatic branched or straight-chain monovalent hydrocarbon radical or linking group substituent having from 1-8 carbon atoms, wherein the radical is derived by the removal of one hydrogen atom from a carbon atom and the linking group is derived by the removal of one hydrogen atom from each of two carbon atoms in the chain. The term includes, without limitation, methyl, methylene, ethyl, ethylene, propyl, propylene, isopropyl, isopropylene, n-butyl, n-butylene, t-butyl, t-butylene, pentyl, pentylene, hexyl, hexylene and the like. An alkyl substituent may be attached to a core molecule via a terminal carbon atom or via a carbon atom within the chain. Similarly, any number of substituent variables may be attached to an alkyl substituent when allowed by available valences. The term “lower alkyl” means an alkyl substituent having from 14 carbon atoms.

The term “alkenyl” means a partially unsaturated alkyl radical or linking group substituent having at least at least two carbon atoms and one double bond derived by the removal of one hydrogen atom from each of two adjacent carbon atoms in the chain. Atoms may be oriented about the double bond in either the cis (E) or trans (Z) conformation. The term includes, without limitation, methylidene, vinyl, vinylidene, allyl, allylidene, propylidene, isopropenyl, iso-propylidene, prenyl, prenylene (3-methyl-2-butenylene), methallyl, methallylene, allylidene (2-propenylidene), crotylene (2-butenylene), and the like. An alkenyl substituent may be attached to a core molecule via a terminal carbon atom or via a carbon atom within the chain. Similarly, any number of substituent variables may be attached to an alkenyl substituent when allowed by available valences. The term “lower alkenyl” means an alkenyl substituent having from 24 carbon atoms.

The term “alkynyl” means a partially unsaturated alkyl radical or linking group substituent having at least two carbon atoms and one triple bond derived by the removal of two hydrogen atom from each of two adjacent carbon atoms in the chain. The term includes, without limitation, ethinyl, ethinylidene, propargyl, propargylidene and the like. An alkynyl substituent may be attached to a core molecule via a terminal carbon atom or via a carbon atom within the chain. Similarly, any number of substituent variables may be attached to an alkynyl substituent when allowed by available valences. The term “lower alkynyl” means an alkynyl substituent having from 24 carbon atoms.

The term “alkoxy” means an alkyl radical or linking group substituent attached through an oxygen-linking atom, wherein a radical is of the formula —O-alkyl and a linking group is of the formula —O-alkyl-. The term includes, without limitation, methoxy, ethoxy, propoxy, butoxy and the like. An alkoxy substituent may be attached to a core molecule and further substituted where allowed.

The term “cycloalkyl” means a saturated or partially unsaturated monocyclic, polycyclic or bridged hydrocarbon ring system radical or linking group. A ring of 3 to 20 carbon atoms may be designated by C₃₋₂₀ cycloalkyl; a ring of 3 to 12 carbon atoms may be designated by C₃₋₁₂ cycloalkyl, a ring of 3 to 8 carbon atoms may be designated by C₃₋₈ cycloalkyl and the like.

The term cycloalkyl includes, without limitation, cyclopropyl, cyclobutyl, cyclopentyl, cyclohexyl, cyclohexenyl, cycloheptyl, cyclooctyl, indanyl, indenyl, 1,2,3,4-tetrahydro-naphthalenyl, 5,6,7,8-tetrahydro-naphthalenyl, 6,7,8,9-tetrahydro-5H-benzocycloheptenyl, 5,6,7,8,9,10-hexahydro-benzocyclooctenyl, fluorenyl, bicyclo[2.2.1]heptyl, bicyclo[2.2.1]heptenyl, bicyclo[2.2.2]octyl, bicyclo[3.1.1]heptyl, bicyclo[3.2.1]octyl, bicyclo[2.2.2]octenyl, bicyclo[3.2.1]octenyl, adamantanyl, octahydro-4,7-methano-1H-indenyl, octahydro-2,5-methano-pentalenyl (also referred to as hexahydro-2,5-methano-pentalenyl) and the like. A cycloalkyl substituent may be attached to a core molecule and further substituted where allowed.

The term “aryl” means an unsaturated, conjugated π electron monocyclic or polycyclic hydrocarbon ring system radical or linking group substituent of 6, 9, 10 or 14 carbon atoms. The term includes, without limitation, phenyl, naphthalenyl, fluorenyl, indenyl, azulenyl, anthracenyl and the like. An aryl substituent may be attached to a core molecule and further substituted where allowed.

The term “heterocyclyl” means a saturated, partially unsaturated (such as those named with the prefix dihydro, trihydro, tetrahydro, hexahydro and the like) or unsaturated monocyclic, polycyclic or bridged hydrocarbon ring system radical or linking group substituent, wherein at least one ring carbon atom has been replaced with one or more heteroatoms independently selected from N, O or S. A heterocyclyl substituent further includes a ring system having up to 4 nitrogen atom ring members or a ring system having from 0 to 3 nitrogen atom ring members and 1 oxygen or sulfur atom ring member. Alternatively, up to two adjacent ring members may be a heteroatom, wherein one heteroatom is nitrogen and the other is selected from N, O or S. A heterocyclyl radical is derived by the removal of one hydrogen atom from a single carbon or nitrogen ring atom. A heterocyclyl linking group is derived by the removal of one hydrogen atom from two of either a carbon or nitrogen ring atom. A heterocyclyl substituent may be attached to a core molecule by either a carbon atom ring member or by a nitrogen atom ring member and further substituted where allowed.

The term heterocyclyl includes, without limitation, furanyl, thienyl, 2H-pyrrolyl, 2-pyrrolinyl, 3-pyrrolinyl, pyrrolidinyl, pyrrolyl, 1,3-dioxolanyl, oxazolyl, thiazolyl, imidazolyl, 2-imidazolinyl (also referred to as 4,5-dihydro-1H-imidazolyl), imidazolidinyl, 2-pyrazolinyl, pyrazolidinyl, pyrazolyl, isoxazolyl, isothiazolyl, oxadiazolyl, triazolyl, thiadiazolyl, tetrazolyl, tetrazolinyl, tetrazolidinyl, 2H-pyranyl, 4H-pyranyl, thiopyranyl, pyridinyl, piperidinyl, 1,4-dioxanyl, morpholinyl, 1,4-dithianyl, thiomorpholinyl, pyridazinyl, pyrimidinyl, pyrazinyl, piperazinyl, azetidinyl, azepanyl, indolizinyl, indolyl, 4-aza-indolyl (also referred to as 1H-pyrrolo[3,2-b]pyridin-3-yl), 6-aza-indolyl (also referred to as 1H-pyrrolo[2,3-c]pyridin-3-yl), 7-aza-indolyl (also referred to as 1H-pyrrolo[2,3-b]pyridin-3-yl), isoindolyl, 3H-indolyl, indolinyl, benzo[b]furanyl, furo[2,3-b]pyridin-3-yl, benzo[b]thienyl, indazolyl (also referred to as 1H-indazolyl), benzoimidazolyl, benzothiazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl, quinuclidinyl, 2H-chromenyl, 3H-benzo[f]chromenyl, tetrahydro-furanyl, tetrahydro-thienyl, tetrahydro-pyranyl, tetrahydro-thiopyranyl, tetrahydro-pyridazinyl, hexahydro-1,4-diazepinyl, hexahydro-1,4-oxazepanyl, 2,3-dihydro-benzo[b]oxepinyl, 1,3-benzodioxolyl (also known as 1,3-methylenedioxyphenyl or benzo[1,3]dioxolyl), 2,3-dihydro-1,4-benzodioxinyl (also known as 1,4-ethylenedioxyphenyl or benzo[1,4]dioxinyl), benzo-dihydro-furanyl (also known as 2,3-dihydro-benzofuranyl), benzo-tetrahydro-pyranyl, benzo-dihydro-thienyl, 5,6,7,8-tetrahydro-4H-cyclohepta[b]thienyl, 5,6,7-trihydro-4H-cyclohexa[b]thienyl, 5,6-dihydro-4H-cyclopenta[b]thienyl, 2-aza-bicyclo[2.2.1]heptyl, 1-aza-bicyclo[2.2.2]octyl, 8-aza-bicyclo[3.2.1]octyl, 7-oxa-bicyclo[2.2.1]heptyl, pyrrolidinium, piperidinium, piperazinium, morpholinium and the like.

The term “acrylyl” means a linking group of the formula —C(O)C═C—.

The term “acyl” means a radical of the formula —C(O)-alkyl, or a linking group of the formula —C(O)-alkyl-.

The term “acyloxy” means a linking group of the formula —C(O)-alkyl-O—.

The term “alkoxycarbonylalkoxy” means a radical of the formula —O-alkyl-C(O)O-alkyl, or a linking group of the formula —O-alkyl-C(O)O-alkyl-.

The term “alkoxycarboxy” means a radical of the formula —O-alkyl-CO₂H or —O-alkyl-C(O)OH.

The term “alkylamino” means a radical of the formula -alkyl-NH₂, or a linking group of the formula -alkyl-NH—.

The term “alkylaminoalkyl” means a radical of the formula -alkyl-NH-alkyl or -alkyl-N(alkyl)₂, or a linking group of the formula -alkyl-NH-alkyl- or -alkyl-N(alkyl)-alkyl-.

The term “alkylcarbamoyl” means a radical of the formula -alkyl-C(O)NH₂, or a linking group of the formula -alkyl-C(O)NH—.

The term “alkylcarbamoylalkyl” means a radical of the formula -alkyl-C(O)NH-alkyl or -alkyl-C(O)N(alkyl)₂, or a linking group of the formula -alkyl-C(O)NH-alkyl- or —C(O)N(alkyl)-alkyl-.

The term “alkylcarbonylalkoxy” means a radical of the formula -alkyl-C(O)O-alkyl, or a linking group of the formula -alkyl-C(O)O-alkyl-.

The term “alkylcarboxy” means a radical of the formula -alkyl-CO₂H or -alkyl-C(O)OH.

The term “alkylsulfonylamino” means a radical of the formula -alkyl-SO₂—NH₂.

The term “alkylsulfonylaminoalkyl” means a radical of the formula -alkyl-SO₂—NH-alkyl or -alkyl-SO₂—N(alkyl)₂, or a linking group of the formula -alkyl-SO₂—NH-alkyl- or -alkyl-SO₂—N(alkyl)-alkyl-.

The term “amino” means a radical of the formula —NH₂.

The term “aminoacylamino” means a radical of the formula —NH—C(O)-alkyl-NH₂, or a linking group of the formula —NH—C(O)-alkyl-NH—.

The term “aminoacylaminoalkyl” means a radical of the formula —NH—C(O)-alkyl-NH-alkyl or —NH—C(O)-alkyl-N(alkyl)₂, or a linking group of the formula —NH—C(O)-alkyl-NH-alkyl- or —NH—C(O)-alkyl-N(alkyl)-alkyl-.

The term “aminoalkyl” means a radical of the formula —NH-alkyl or —N(alkyl)₂, or a linking group of the formula —NH-alkyl- or —N(alkyl)-alkyl-.

The term “carbamoyl” means a radical of the formula —C(O)NH₂, or a linking group of the formula —C(O)NH—.

The term “carbamoylalkyl” means a radical of the formula —C(O)NH-alkyl or —C(O)N(alkyl)₂, or a linking group of the formula —C(O)NH-alkyl- or —C(O)N(alkyl)-alkyl-.

The term “carbonyl” means a linking group of the formula —C(O)— or —C(═O)—.

The term “carbonylalkoxy” means a radical of the formula —C(O)O-alkyl, or a linking group of the formula —C(O)O-alkyl-.

The term “carboxy” means a radical of the formula —C(O)OH or —CO₂H.

The term “carboxyl” means a linking group of the formula —C(O)O—.

The term “halo” or “halogen” means fluoro, chloro, bromo or iodo.

The term “iminomethylaminocarbonyl” means a linking group having the formula —C(NH)NHC(O)— or —C(═NH)NHC(O)—.

The term “oxyacyl” means a radical of the formula —OC(O)-alkyl, or a linking group of the formula —OC(O)-alkyl-.

The term “oxyacylaryl” means a radical of the formula —OC(O)-alkyl-aryl.

The term “oxyacrylyl” means a radical of the formula —OC(O)-alkenyl, or a linking group of the formula —OC(O)-alkenyl-.

The term “oxyacrylylaryl” means a radical of the formula —OC(O)-alkenyl-aryl.

The term “oxycarbonylalkoxy” means a radical of the formula —OC(O)—O-alkyl, or a linking group of the formula —OC(O)—O-alkyl-.

The term “sulfonylalkyl” means a radical of the formula —SO₂-alkyl, or a linking group of the formula —SO₂-alkyl-.

The term “sulfonylamino” means a radical of the formula —SO₂—NH₂.

The term “sulfonylaminoalkyl” means a radical of the formula —SO₂—NH-alkyl or —SO₂—N(alkyl)₂, or a linking group of the formula —SO₂—NH-alkyl- or —SO₂—N(alkyl)-alkyl-.

The term “thioalkyl” means a radical of the formula —S-alkyl, or a linking group of the formula —S-alkyl-.

The term “thiocarbamyl” means a radical of the formula —C(S)NH₂ or —C(═S)NH₂, or a linking group of the formula—C(S)NH—.

The term “urea” means a radical of the formula —NH—C(O)—NH₂.

The term “ureaalkyl” means a radical of the formula —NH—C(O)—NH-alkyl or —NH—C(O)—N(alkyl)₂.

The term “substituted” means one or more hydrogen atoms on a core molecule have been replaced with one or more radicals Pr linking groups, wherein the linking group, by definition is also further substituted.

The term “dependently selected” means one or more substituent variables are present in a specified combination (e.g. groups of substituents commonly appearing in a tabular list).

The substituent nomenclature used in the disclosure of the present invention was derived using nomenclature rules well known to those skilled in the art (e.g., IUPAC).

Compound Forms

The compounds of the invention may be present in a form which may, alternatively or in addition to a compound of Formula (I), comprise a salt of a compound of Formula (I) or a prodrug or active metabolite of such a compound or salt.

The compounds of the invention may be present in a salt form. For use in medicines, the salts of the compounds of this invention refer to non-toxic. “pharmaceutically acceptable salts.” FDA-approved pharmaceutically acceptable salt forms include pharmaceutically acceptable acidic/anionic or basic/cationic salts.

Pharmaceutically acceptable acidic/anionic salts include, without limitation, acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, triethiodide trifluoroacetate salts and the like.

Organic or inorganic acids also include, and are not limited to, hydroiodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic, trifluoroacetic acid and the like.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate (SEH), sodium hydroxide, triethanolamine (TEA), zinc and the like.

The compounds of the invention may be present in the form of pharmaceutically acceptable prodrugs and metabolites thereof. In general, such prodrugs and metabolites will be functional derivatives of the compounds that are readily convertible in vivo into an active compound.

The term “prodrug” means a pharmaceutically acceptable form of a functional derivative of a compound of the invention (or a salt thereof), wherein the prodrug may be: 1) a relatively active precursor which converts in vivo to an active prodrug component; 2) a relatively inactive precursor which converts in vivo to an active prodrug component; or 3) a relatively less active component of the compound that contributes to therapeutic biological activity after becoming available in vivo (i.e., as a metabolite). Conventional procedures for the selection and preparation of suitable prodrug derivatives are described in, for example, “Design of Prodrugs”, ed. H. Bundgaard, Elsevier, 1985.

The term “metabolite” means a pharmaceutically acceptable form of a metabolic derivative of a compound of the invention (or a salt thereof), wherein the derivative is a relatively less active component of the compound that contributes to therapeutic biological activity after becoming available in vivo.

The present invention also contemplates compounds of Formula (I) in various stereoisomeric or tautomeric forms. The invention encompasses all such CCR2 inhibiting compounds, including active compounds in the form of essentially pure enantiomers, racemic mixtures and tautomers or pharmaceutically acceptable forms thereof.

The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (stereoisomers).

The term “stereoisomer” refers to isomers of identical constitution that differ in the arrangement of their atoms in space. Enantiomers and diastereomers are stereoisomers wherein an asymmetrically substituted carbon atom acts as a chiral center. The term “chiral” refers to a molecule that is not superposable on its mirror image, implying the absence of an axis and a plane or center of symmetry. The term “enantiomer” refers to one of a pair of molecular species that are mirror images of each other and are not superposable. The term “diastereomer” refers to stereoisomers that are not related as mirror images. The symbols “R” and “S” represent the configuration of substituents around a chiral carbon atom(s). The symbols “R*” and “S*” denote the relative configurations of substituents around a chiral carbon atom(s).

The term “racemate” or “racemic mixture” refers to a compound of equimolar quantities of two enantiomeric species, wherein the compound is devoid of optical activity. The term “optical activity” refers to the degree to which a chiral molecule or nonracemic mixture of chiral molecules rotates the plane of polarized light.

The term “geometric isomer” refers to isomers that differ in the orientation of substituent atoms in relationship to a carbon-carbon double bond, to a cycloalkyl ring or to a bridged bicyclic system. Substituent atoms (other than H) on each side of a carbon-carbon double bond may be in an E or Z configuration. In the “E” configuration, the substituents are on opposite sides in relationship to the carbon-carbon double bond; in the “Z” configuration, the substituents are oriented on the same side in relationship to the carbon-carbon double bond.

Substituent atoms (other than H) attached to a hydrocarbon ring may be in a cis or trans configuration. In the “cis” configuration, the substituents are on the same side in relationship to the plane of the ring; in the “trans” configuration, the substituents are on opposite sides in relationship to the plane of the ring. Compounds having a mixture of “cis” and “trans” species are designated “cis/trans”. Substituent atoms (other than H) attached to a bridged bicyclic system may be in an “endo” or “exo” configuration. In the “endo” configuration, the substituents attached to a bridge (not a bridgehead) point toward the larger of the two remaining bridges; in the “exo” configuration, the substituents attached to a bridge point toward the smaller of the two remaining bridges.

It is to be understood that the various substituent stereoisomers, geometric isomers and mixtures thereof used to prepare compounds of the present invention are either commercially available, can be prepared synthetically from commercially available starting materials or can be prepared as isomeric mixtures and then obtained as resolved isomers using techniques well-known to those of ordinary skill in the art.

The isomeric descriptors “R,” “S,” “S*,” “R*,” “E,” “Z,” “cis,” “trans,” “exo”, and “endo”, where used herein, indicate atom configurations relative to a core molecule and are intended to be used as defined in the literature.

The compounds of the present invention may be prepared as individual isomers by either isomer-specific synthesis or resolved from an isomeric mixture. Conventional resolution techniques include forming the free base of each isomer of an isomeric pair using an optically active salt (followed by fractional crystallization and regeneration of the free base), forming an ester or amide of each of the isomers of an isomeric pair (followed by chromatographic separation and removal of the chiral auxiliary) or resolving an isomeric mixture of either a starting material or a final product using various well known chromatographic methods.

Furthermore, compounds of the present invention may have a plurality of polymorph or amorphous crystalline forms and, as such, are intended to be included in the scope of the invention. In addition, some of the compounds may form a plurality of solvates with water (i.e., hydrates) or common organic solvents, such are also intended to be encompassed within the scope of this invention.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known in the art.

Therapeutic Use

Compounds of Formula (I) or a form, composition or medicament thereof in accordance with the present invention are CCR2 antagonists. A compound of Formula (I) or a form, composition or medicament thereof may have a mean inhibition constant (IC₅₀) against MCP-1 binding to CCR2 of between about 50 μM to about 0.01 nM; between about 25 μM to about 0.01 nM; between about 10 μM to about 0.01 nM; between about 5 μM to about 0.01 nM; between about 1 μM to about 0.01 nM; between about 800 nM to about 0.01 nM; between about 200 nM to about 0.01 nM; between about 100 nM to about 0.01 nM; or, between about 10 nM to about 0.01 nM.

A compound of Formula (I) or a composition or medicament thereof reduces MCP-1 induced monocyte chemotaxis. A compound of Formula (I) or a form, composition or medicament thereof may have an IC₅₀ for reduction in MCP-1 induced monocyte chemotaxis of between about 50 μM to about 0.01 nM; between about 25 μM to about 0.01 nM; between about 10 μM to about 0.01 nM; between about 5 μM to about 0.01 nM; between about 1 μM to about 0.01 nM; between about 800 nM to about 0.01 nM; between about 200 nM to about 0.01 nM; between about 100 nM to about 0.01 nM; or, between about 10 nM to about 0.01 nM.

A compound of Formula (I) or a composition or medicament thereof reduces MCP-1 intracellular calcium mobilization. A compound of Formula (I) or a form, composition or medicament thereof may have an IC₅₀ for reduction in MCP-1 induced intracellular calcium mobilization of between about 50 μM to about 0.01 nM; between about 25 μM to about 0.01 nM; between about 10 μM to about 0.01 nM; between about 5 μM to about 0.01 nM; between about 1 μM to about 0.01 nM; between about 800 nM to about 0.01 nM; between about 200 nM to about 0.01 nM; between about 100 nM to about 0.01 nM; or, between about 10 nM to about 0.01 nM.

Accordingly, a compound of Formula (I) or a form, composition or medicament thereof is useful in a method for preventing, treating or ameliorating a CCR2 mediated inflammatory syndrome, disorder or disease in a subject in need thereof comprising administering to the subject an effective amount of a compound of Formula (I) or form, composition or medicament thereof.

The present invention is directed to a method for preventing, treating or ameliorating a CCR2 mediated inflammatory syndrome, disorder or disease in a subject in need thereof comprising administering to the subject an effective amount of a compound of Formula (I) or a form, composition or medicament thereof.

The term “administering” with respect to the methods of the invention, means a method for therapeutically or prophylactically preventing, treating or ameliorating a syndrome, disorder or disease as described herein by using a compound of Formula (I) or a form, composition or medicament thereof. Such methods include administering an effective amount of said compound, compound form, composition or medicament at different times during the course of a therapy or concurrently in a combination form. The methods of the invention are to be understood as embracing all known therapeutic treatment regimens.

The term “subject” refers to a patient, which may be animal, typically a mammal, typically a human, which has been the object of treatment, observation or experiment and is at risk of (or susceptible to) developing a syndrome, disorder or disease that is associated with elevated MCP-1 expression or MCP-1 overexpression, or a patient with an inflammatory condition that accompanies syndromes, disorders or diseases associated with elevated MCP-1 expression or MCP-1 overexpression.

The term “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

The effective amount of a compound of the invention in such a therapeutic method is from about 0.1 ng/kg/day to about 300 mg/kg/day.

Examples of compounds of Formula (I) or a form, composition or medicament thereof useful in a method for preventing, treating or ameliorating a CCR2 mediated inflammatory syndrome, disorder or disease in a subject in need thereof is selected from the group consisting of:
6 [4-(1H-indol-3-yl)-piperidin-1-yl]-{1-[(2E)-3-(3,4,5-trifluoro-phenyl)-acryloyl]-piperidin-4-yl}-acetic acid;
7 {1-[(2E)-3-(3,5-difluoro-phenyl)-acryloyl]-piperidin-4-yl}-[4-(1H-indol-3-yl)-piperidin-1-yl]-acetic acid;
8 {1-[(2E)-3-(3,5-difluoro-phenyl)-acryloyl]-piperidin-4-yl}-[4-(5-fluoro-1H-indol-3-yl)-piperidin-1-yl]-acetic acid;
9 [4-(5-fluoro-1H-indol-3-yl)-piperidin-1-yl]-{1-[(2E)-3-(3,4,5-trifluoro-phenyl)-acryloyl]-piperidin-4-yl}-acetic acid;
13 (S)-{[4-(1H-indol-3-yl)-piperidin-1-yl]}-{1-[(2E)-3-(3,4,5-trifluoro-phenyl)-acryloyl]-piperidin-4-yl}-acetic acid;
15 [4-(5-hydroxy-1H-indol-3-yl)-piperidin-1-yl]-{1-[(2E)-3-(3,4,5-trifluoro-phenyl)-acryloyl]-piperidin-4-yl}-acetic acid;
16 {1-[(2E)-3-(3,5-difluoro-phenyl)-acryloyl]-piperidin-4-yl}-[4-(5-hydroxy-1H-indol-3-yl)-piperidin-1-yl]-acetic acid;
18 {1-[(2E)-3-(3,4-dichloro-phenyl)-acryloyl]-piperidin-4-yl}-[4-(1H-indol-3-yl)-piperidin-1-yl]-acetic acid;
19 {1-[(2E)-3-(3,4-dichloro-phenyl)-acryloyl]-piperidin-4-yl}-[4-(5-fluoro-1H-indol-3-yl)-piperidin-1-yl]-acetic acid;
20 [1-(3,4-dichloro-phenylcarbamoyl)-piperidin-4-yl]-[4-(1H-indol-3-yl)-piperidin-1-yl]-acetic acid;
22 {1-[(2E)-3-(3,4-difluoro-phenyl)-acryloyl]-piperidin-4-yl}-[4-(1H-indol-3-yl)-piperidin-1-yl]-acetic acid;
23 [4-(1H-indol-3-yl)-piperidin-1-yl]-{1-[(2E)-3-(4-trifluoromethyl-phenyl)-acryloyl]-piperidin-4-yl}-acetic acid;
24 {1-[(2E)-3-(3,5-difluoro-phenyl)-acryloyl]-piperidin-4-yl}-[4-(6-fluoro-1H-indol-3-yl)-piperidin-1-yl]-acetic acid;
25 [4-(6-chloro-1H-indol-3-yl)-piperidin-1-yl]-{1-[(2E)-3-(3,5-difluoro-phenyl)-acryloyl]-piperidin-4-yl}-acetic acid;
26 {1-[(2E)-3-(3,5-difluoro-phenyl)-acryloyl]-piperidin-4-yl}-[4-(5-methoxy-1H-indol-3-yl)-piperidin-1-yl]-acetic acid;
27 [4-(1H-indol-3-yl)-piperidin-1-yl]-{1-[(2E)-3-phenyl-acryloyl]-piperidin-4-yl}-acetic acid;
29 {1-[(2E)-3-(3,5-difluoro-phenyl)-acryloyl]-piperidin-4-yl}-[4-(5-methanesulfonylamino-1H-indol-3-yl)-piperidin-1-yl]-acetic acid;
30 [4-(5-methoxy-1H-indol-3-yl)-piperidin-1-yl]-{1-[(2E)-3-(3,4,5-trifluoro-phenyl)-acryloyl]-piperidin-4-yl}-acetic acid;
31 [4-(6-chloro-1H-indol-3-yl)-piperidin-1-yl]-{1-[(2E)-3-(3,4,5-trifluoro-phenyl)-acryloyl]-piperidin-4-yl}-acetic acid;
34 {1-[(2E)-3-(4-chloro-phenyl)-acryloyl]-piperidin-4-yl}-[4-(1H-indol-3-yl)-piperidin-1-yl]-acetic acid;
35 [4-(1H-indol-3-yl)-piperidin-1-yl]-{1-[(2E)-3-(3-trifluoromethyl-phenyl)-acryloyl]-piperidin-4-yl}-acetic acid;
36 {1-[(2E)-3-(3-bromo-4-fluoro-phenyl)-acryloyl]-piperidin-4-yl}-[4-(1H-indol-3-yl)-piperidin-1-yl]-acetic acid;
38 {1-[(2E)-3-(3,5-difluoro-phenyl)-acryloyl]-piperidin-4-yl}-[4-(6-methoxy-1H-indol-3-yl)-piperidin-1-yl]-acetic acid;
39 {1-[(2E)-3-(3,4-dichloro-phenyl)-acryloyl]-piperidin-4-yl}-[4-(6-fluoro-1H-indol-3-yl)-piperidin-1-yl]-acetic acid;
40 [1-(3,4-difluoro-phenylcarbamoyl)-piperidin-4-yl]-[4-(1H-indol-3-yl)-piperidin-1-yl]-acetic acid;
41 {1-[(2E)-3-(3,5-difluoro-phenyl)-acryloyl]-piperidin-4-yl}-[4-(4-methoxy-1H-indol-3-yl)-piperidin-1-yl]-acetic acid;
42 {1-[(2E)-3-(3,5-difluoro-phenyl)-acryloyl]-piperidin-4-yl}-[4-(7-methoxy-1H-indol-3-yl)-piperidin-1-yl]-acetic acid;
45 [1-(3,5-dichloro-phenylthiocarbamoyl)-piperidin-4-yl]-[4-(1H-indol-3-yl)-piperidin-1-yl]-acetic acid;
46 [4-(6-chloro-1H-indol-3-yl)-piperidin-1-yl]-{1-[(2E)-3-(3,4-dichloro-phenyl)-acryloyl]-piperidin-4-yl}-acetic acid;
47 {1-[(2E)-3-(3,4-dichloro-phenyl)-acryloyl]-piperidin-4-yl}-[4-(5-methoxy-1H-indol-3-yl)-piperidin-1-yl]-acetic acid;
48 [1-(3-chloro-4-fluoro-phenylcarbamoyl)-piperidin-4-yl]-[4-(1H-indol-3-yl)-piperidin-1-yl]-acetic acid;
49 [1-(3-chloro-4-methyl-phenylcarbamoyl)-piperidin-4-yl]-[4-(1H-indol-3-yl)-piperidin-1-yl]-acetic acid;
50 {1-[(3,4-dichloro-benzoylamino)-imino-methyl]-piperidin-4-yl}-[4-(1H-indol-3-yl)-piperidin-1-yl]-acetic acid;
52 {1 ([imino-(3,4,5-trifluoro-benzoylamino)-methyl]-piperidin-4-yl)}-[4-(1H-indol-3-yl)-piperidin-1-yl]-acetic acid;
53 [4-(5-methanesulfonylamino-1H-indol-3-yl)-piperidin-1-yl]-{1-[(2E)-3-(3,4,5-trifluoro-phenyl)-acryloyl]-piperidin-4-yl}-acetic acid;
57 [1-(4-chloro-3-trifluoromethyl-phenylcarbamoyl)-piperidin-4-yl]-[4-(1H-indol-3-yl)-piperidin-1-yl]-acetic acid;
59 [4-(1H-indol-3-yl)-piperidin-1-yl]-{1-[(2E)-3-(4-nitro-phenyl)-acryloyl]-piperidin-4-yl}-acetic acid;
60 {1-[(2E)-3-(4-bromo-phenyl)-acryloyl]-piperidin-4-yl)-[4-(1H-indol-3-yl)-piperidin-1-yl}-acetic acid;
62 {1-[(2E)-3-(3-fluoro-phenyl)-acryloyl]-piperidin-4-yl}-[4-(1H-indol-3-yl)-piperidin-1-yl]-acetic acid;
64 [1-(3,4-dichloro-phenylthiocarbamoyl)-piperidin-4-yl]-[4-(1H-indol-3-yl)-piperidin-1-yl]-acetic acid;
70 [4-(1H-indol-3-yl)-piperidin-1-yl]-{1-[(2E)-3-m-tolyl-acryloyl]-piperidin-4-yl}-acetic acid;
71 {1-[(2E)-3-(3-bromo-phenyl)-acryloyl]-piperidin-4-yl}-[4-(1H-indol-3-yl)-piperidin-1-yl]-acetic acid;
72 [4-(1H-indol-3-yl)-piperidin-1-yl]-{1-[(2E)-3-(3-methoxy-phenyl)-acryloyl]-piperidin-4-yl}-1-acetic acid;
74 {1-[(2E)-3-(3-fluoro-4-methyl-phenyl)-acryloyl]-piperidin-4-yl}-[4-(1H-indol-3-yl)-piperidin-1-yl]-acetic acid;
75 {1-[(2E)-3-(3-fluoro-4-trifluoromethyl-phenyl)-acryloyl]-piperidin-4-yl}-[4-(1H-indol-3-yl)-piperidin-1-yl]-acetic acid;
76 {1-[(2E)-3-(3-chloro-4-fluoro-phenyl)-acryloyl]-piperidin-4-yl}-[4-(1H-indol-3-yl)-piperidin-1-yl]-acetic acid;
77 {1-[(2E)-3-(4-fluoro-phenyl)-acryloyl]-piperidin-4-yl}-[4-(1H-indol-3-yl)-piperidin-1-yl]-acetic acid;
79 [4-(1H-indol-3-yl)-piperidin-1-yl]-[1-(3-trifluoromethyl-phenylthiocarbamoyl)-piperidin-4-yl]-acetic acid;
80 [4-(1H-indol-3-yl)-piperidin-1-yl]-[ ]1-(4-trifluoromethyl-phenylthiocarbamoyl)-piperidin-4-yl]-acetic acid;
81 [4-(1H-pyrrol-3-yl)-piperidin-1-yl]-{1-[(2E)-3-(3,4,5-trifluoro-phenyl)-acryloyl]-piperidin-4-yl}-1-acetic acid;
83 [4-(6-methanesulfonylamino-1H-indol-3-yl)-piperidin-1-yl]-{1-[(2E)-3-(3,4,5-trifluoro-phenyl)-acryloyl]-piperidin-4-yl}-1-acetic acid;
88 [1-(4-chloro-phenylcarbamoyl)-piperidin-4-yl]-[4-(1H-indol-3-yl)-piperidin 1-yl]-acetic acid;
92 [4-(1H-indol-3-yl)-piperidin-1-yl]-{1-[(2E)-3-(3-nitro-phenyl)-acryloyl]-piperidin-4-yl}-1-acetic acid;
93 {1-[(2E)-3-(3-chloro-phenyl)-acryloyl]-piperidin-4-yl}-[4-(1H-indol-3-yl)-piperidin-1-yl]-acetic acid;
94 [1-(3,4-dichloro-phenylcarbamoyl)-piperidin-4-yl]-[4-(5-methoxy-1H-indol-3-yl)-piperidin-1-yl]-acetic acid;
95 [1-(3,4-dichloro-phenylcarbamoyl)-piperidin-4-yl]-[4-(6-methoxy-1H-indol-3-yl)-piperidin-1-yl]-acetic acid;
96 [4-(1H-indol-3-yl)-piperidin-1-yl]-[1-(4-trifluoromethyl-phenylcarbamoyl)-piperidin-4-yl]-acetic acid;
101 [1-(4-bromo-3-methyl-phenylcarbamoyl)-piperidin-4-yl]-[4-(1H-indol-3-yl)-piperidin-1-yl]-acetic acid;
106 [4-(1H-indol-3-yl)-piperidin-1-yl]-[1-(4-methyl-3-trifluoromethyl-phenylcarbamoyl)-piperidin-4-yl]-acetic acid;
108 [4-(7-methoxy-1H-indol-3-yl)-piperidin-1-yl]-{1-[(2E)-3-(3,4,5-trifluoro-phenyl)-acryloyl]-piperidin-4-yl}-acetic acid;
109 [1-(3,4-dichloro-phenylcarbamoyl)-piperidin-4-yl]-[4-(5-methanesulfonylamino-1H-indol-3-yl)-piperidin-1-yl]-acetic acid;
112 (2E)-1-(4-{2-hydroxy-1-[4-(1H-indol-3-yl)-piperidin-1-yl]-ethyl}-piperidin-1-yl)-3-(3,4,5-trifluoro-phenyl)-propenone;
113 (2E)-3-(3,4-difluoro-phenyl)-1-(4-{2-hydroxy-1-[4-(1H-indol-3-yl)-piperidin 1-yl]-ethyl}-piperidin-1-yl)-propenone;
116 (2E)-3-(3,5-difluoro-phenyl)-1-(4-{2-hydroxy-1-[4-(1H-indol-3-yl)-piperidin-1-yl]-ethyl}-piperidin-1-yl)-propenone;
119 (2E)-1-(4-{2-hydroxy-1-[4-(1H-indol-3-yl)-piperidin-1-yl]-ethyl}-piperidin-1-yl)-3-(3-trifluoromethyl-phenyl)-propenone;
121 (2E)-3-(3,4-dichloro-phenyl)-1-(4-{2-hydroxy-1-[4-(1H-indol-3-yl)-piperidin-1-yl]-ethyl}-piperidin-1-yl)-propenone;
122 4-{2-hydroxy-1-[4-(1H-indol-3-yl)-piperidin-1-yl]-ethyl}-piperidine-1-carbothioic acid (3,4-dichloro-phenyl)-amide;
123 4-{2-hydroxy-1-[4-(1H-indol-3-yl)-piperidin-1-yl]-ethyl}-piperidine-1-carboxylic acid (3,4-dichloro-phenyl)-amide;
129 4-{2-hydroxy-1-[4-(1H-indol-3-yl)-piperidin-1-yl]-ethyl}-piperidine-1-carboxylic acid (3,5-difluoro-phenyl)-amide;
132 (2E)-1-(4-{2-hydroxy-1-[4-(6-methoxy-1H-indol-3-yl)-piperidin-1-yl]-ethyl}-piperidin-1-yl)-3-(3,4,5-trifluoro-phenyl)-propenone;
133 (2E)-1-(4-{2-hydroxy-1-[4-(7-methoxy-1H-indol-3-yl)-piperidin-1-yl]-ethyl}-piperidin-1-yl)-3-(3,4,5-trifluoro-phenyl)-propenone;
136 [1-(3,5-bis-trifluoromethyl-phenylcarbamoyl)-piperidin-4-yl]-[4-(1H-indol-3-yl)-piperidin-1-yl]-acetic acid;
137 (2E)-3-(3,5-difluoro-phenyl)-1-(4-{2-hydroxy-1-[4-(4-methoxy-phenyl)-piperidin-1-yl]-ethyl}-piperidin-1-yl)-propenone;
139 (2E)-3-(3,4-difluoro-phenyl)-1-(4-{2-hydroxy-1-[4-(4-methoxy-phenyl)-piperidin-1-yl]-ethyl}-piperidin-1-yl)-propenone;
142 [4-(1H-indol-3-yl)-piperidin-1-yl]-[1-(4-trifluoromethylsulfanyl-phenylcarbamoyl)-piperidin-4-yl]-acetic acid;
143 [4-(1H-indol-3-yl)-piperidin-1-yl]-[1-(4-trifluoromethoxy-phenylcarbamoyl)-piperidin-4-yl]-acetic acid;
144 [4-(1H-indol-3-yl)-piperidin-1-yl]-[1-(3-methylsulfanyl-phenylcarbamoyl)-piperidin-4-yl]-acetic acid;
146 3-[1-(carboxy-{1-[(2E)-3-(3,4,5-trifluoro-phenyl)-acryloyl]-piperidin-4-yl}-methyl)-piperidin-4-yl]-1H-indole-5-carboxylic acid methyl ester;
151 [4-(1H-pyrrolo[2,3-b]pyridin-3-yl)-piperidin-1-yl]-{1-[(2E)-3-(3,4,5-trifluoro-phenyl)-acryloyl]-piperidin-4-yl}-acetic acid;
153 (2E)-1-(4-{2-hydroxy-1-[4-(1H-pyrrolo[2,3-b]pyridin-3-yl)-piperidin-1-yl]-ethyl}-piperidin-1-yl)-3-(3,4,5-trifluoro-phenyl)-propenone;
158 (2E)-1-(4-{2-hydroxy-1-[4-(4-methoxy-phenyl)-piperidin-1-yl]-ethyl}-piperidin-1-yl)-3-(3,4,5-trifluoro-phenyl)-propenone;
162 (2E)-3-(3,4-dichloro-phenyl)-1-(4-{2-hydroxy-1-[4-(5-methoxy-1H-indol-3-yl)-piperidin-1-yl]-ethyl}-piperidin-1-yl)-propenone;
166 (2E)-1-(4-{2-hydroxy-1-[4-(5-methoxy-1H-indol-3-yl)-piperidin-1-yl]-ethyl}-piperidin-1-yl)-3-(3,4,5-trifluoro-phenyl)-propenone;
170 (2E)-1-(4-{1-[4-(5-fluoro-1H-indol-3-yl)-piperidin-1-yl]-2-hydroxy-ethyl}-piperidin-1-yl)-3-(3,4,5-trifluoro-phenyl)-propenone;
171 (2E)-3-(3,5-difluoro-phenyl)-1-(4-{1-[4-(5-fluoro-1H-indol-3-yl)-piperidin-1-yl]-2-hydroxy-ethyl}-piperidin-1-yl)-propenone;
180 (2E)-3-(3,5-difluoro-phenyl)-1-(4-{(1S)-2-hydroxy-1-[4-(4-methoxy-phenyl)-piperidin-1-yl]-ethyl}-piperidin-1-yl)-propenone;
181 (2E)-3-(3,5-difluoro-phenyl)-1-(4-{(1R)-2-hydroxy-1-[4-(4-methoxy-phenyl)-piperidin-1-yl]-ethyl}-1-piperidin-1-yl)-propenone;
187 (2E)-1-(4-{(1S)-2-hydroxy-1-[4-(1H-indol-3-yl)-piperidin-1-yl]-ethyl}-piperidin-1-yl)-3-(3,4,5-trifluoro-phenyl)-propenone;
188 (2E)-1-(4-{(1R)-2-hydroxy-1-[4-(1H-indol-3-yl)-piperidin-1-yl]-ethyl}-piperidin-1-yl)-3-(3,4,5-trifluoro-phenyl)-propenone;
198 N-{3-[1-(1-{1-[(2E)-3-(3,4-dichloro-phenyl)-acryloyl]-piperidin-4-yl}-2-hydroxy-ethyl)-piperidin-4-yl]-1H-indol-5-yl}-1-methanesulfonamide;
201 N-{3-[1-(2-hydroxy-1-{1-[(2E)-3-(3,4,5-trifluoro-phenyl)-acryloyl]-piperidin-4-yl}-ethyl)-piperidin-4-yl]-1H-indol-5-yl}-methanesulfonamide;
202 (2E)-3-(3,5-difluoro-phenyl)-1-(4-{2-hydroxy-1-[4-(1H-pyrrolo[2,3-b]pyridin-3-yl)-piperidin-1-yl]-ethyl}-1-piperidin-1-yl)-propenone;
205 (2E)-3-(3,5-difluoro-phenyl)-1-(4-{2-hydroxy-1-[4-(7-oxy-1H-pyrrolo[2,3-b]pyridin-3-yl)-piperidin-1-yl]-ethyl}-1-piperidin-1-yl)-propenone;
208 (2E)-3-(3,4-dichloro-phenyl)-1-(4-{2-hydroxy-1-[4-(1H-pyrrolo[2,3-b]pyridin-3-yl)-piperidin-1-yl]-ethyl}-1-piperidin-1-yl)-propenone;
211 [4-(6-fluoro-1H-indol-3-yl)-piperidin-1-yl]-{1-[(2E)-3-(3,4,5-trifluoro-phenyl)-acryloyl]-piperidin-4-yl}-acetic acid;
213 N-(2-[4-(1H-indol-3-yl)-piperidin-1-yl]-2-{1-[(2E)-3-(3,4,5-trifluoro-phenyl)-acryloyl]-piperidin-4-yl}-ethyl)-acetamide;
238 (2-[4-(1H-indol-3-yl)-piperidin-1-yl]-2-{1-[(2E)-3-(3,4,5-trifluoro-phenyl)-acryloyl]-piperidin-4-yl}-ethyl)-carbamic acid methyl ester;
243 acetic acid 2-{4-[5-(acetyl-methanesulfonyl-amino)-1H-indol-3-yl]-piperidin-1-yl)-2-{1-[(2E)-3-(3,5-difluoro-phenyl)-acryloyl]-piperidin-4-yl}-ethyl ester; and
259 (2E)-3-(3,5-difluoro-phenyl)-1-(4-{2-hydroxy-1-[4-(5-hydroxy-1H-indol-3-yl)-piperidin-1-yl]-ethyl}-piperidin-1-yl)-propenone.

The invention includes the use of an instant compound for the preparation of a composition or medicament for preventing, treating or ameliorating a CCR2 mediated inflammatory syndrome, disorder or disease in a subject in need thereof, wherein the composition or medicament comprises a mixture one or more compounds of the invention and an optional pharmaceutically acceptable carrier.

The term “composition” means a product comprising at least a compound of the invention, such as a product comprising the specified ingredients in the specified amounts, as well as any product which results, directly or indirectly, from such combinations of the specified ingredients in the specified amounts and one or more pharmaceutically acceptable carriers or any such alternatives to a compound of the invention and a pharmaceutically acceptable carrier therefor.

The term “medicament” means a product for use in preventing, treating or ameliorating a CCR2 mediated inflammatory syndrome, disorder or disease.

The term “pharmaceutically acceptable” means molecular entities and compositions that are of sufficient purity and quality for use in the formulation of a composition or medicament of the invention and that, when appropriately administered to an animal or a human, do not produce an adverse, allergic, or other untoward reaction. Since both human and veterinary use is included within the scope of the invention, a pharmaceutically acceptable formulation includes a compound of Formula (I) or a form, composition or medicament thereof for either human or veterinary use.

The term “CCR2 mediated inflammatory syndrome, disorder or disease” means, without limitation, syndromes, disorders or diseases associated with elevated MCP-1 expression, MCP-1 overexpression or inflammatory conditions that accompany syndromes, disorders or diseases associated with elevated MCP-1 expression or MCP-1 overexpression.

The terms “elevated MCP-1 expression” or “MCP-1 overexpression” mean unregulated or up-regulated CCR2 activation as a result of MCP-1 binding.

The term “unregulated” means unwanted CCR2 activation in a multicellular organism resulting in harm (such as discomfort or decreased life expectancy) to the multicellular organism.

The term “up-regulated” means: 1). increased or unregulated CCR2 activity or expression, or 2). increased CCR2 expression leading to unwanted monocyte and lymphocyte migration. The existence of an inappropriate or abnormal level of MCP-1 or activity of CCR2 is determined by procedures well known in the art.

CCR2 mediated inflammatory syndromes, disorders or diseases include, without limitation, ophthalmic disorders, uveitis, atherosclerosis, rheumatoid arthritis, psoriasis, psoriatic arthritis, atopic dermatitis, multiple sclerosis, Crohn's Disease, ulcerative colitis, nephritis, organ allograft rejection, fibroid lung, renal insufficiency, diabetes and diabetic complications, diabetic nephropathy, diabetic retinopathy, diabetic retinitis, diabetic microangiopathy, tuberculosis, chronic obstructive pulmonary disease, sarcoidosis, invasive staphyloccocia, inflammation after cataract surgery, allergic rhinitis, allergic conjunctivitis, chronic urticaria, asthma, allergic asthma, periodontal diseases, periodonitis, gingivitis, gum disease, diastolic cardiomyopathies, cardiac infarction, myocarditis, chronic heart failure, angiostenosis, restenosis, reperfusion disorders, glomerulonephritis, solid tumors and cancers, chronic lymphocytic leukemia, chronic myelocytic leukemia, multiple myeloma, malignant myeloma, Hodgkin's disease, and carcinomas of the bladder, breast, cervix, colon, lung, prostate, or stomach.

The term “uveitis” generically refers to any inflammatory disease involving the eye. Uveitis can be divided into clinically distinct subtypes based on the part of the eye in which the inflammation is present (percentages correspond to patients known to fit these categories): anterior (51%), intermediate (13%), posterior (20%), or panuveitis (16%) and, according to the course of the disease, as either acute (16%), recurring (26%), or chronic (58%). Those with anterior uveitis (·19%) eventually develop irreparable vision damage despite aggressive treatment such as unilateral blindness (9%), bilateral blindness (2%), or unilateral or bilateral vision impairment (8%). Most cases of uveitis are idiopathic, but known causes include infection (e.g., toxoplasmosis, cytomegalovirus, and the like) or development as a component of a systemic inflammatory and/or autoimmune disorder (e.g., juvenile RA, HLA-B27-associated spondyloarthropathies, sarcoidosis, and the like).

Patients with anterior uveitis have MCP-1 present in large quantities in the aqueous humor of the eye. The amount of MCP-1 correlates with the severity of the clinical symptoms and the large number of mononuclear cells present in the cellular infiltrate. Uveitis is also a potential complication resulting from cataract surgery and prophylactic use of antibiotics and corticosteroids is common for such patients. Currently, most patients with anterior uveitis are first treated with topical corticosteroids. Injected or oral steroids may be used in severe cases, or if the disease is recurrent or chronic. If steroids are ineffective, immunosuppressive agents (e.g., cyclosporine, methotrexate, azathioprine, cyclophosphamide, and the like) are used, particularly if the patient's vision is in danger. All of these drugs have potentially severe side-effects, particularly in children, and there is general agreement that there is an unmet medical need for safe and effective steroid substitutes or steroid-sparing agents.

An example of the invention is a method for preventing, treating or ameliorating CCR2 mediated ophthalmic disorders (such as uveitis, allergic conjunctivitis and the like), rheumatoid arthritis, psoriasis, psoriatic arthritis, atopic dermatitis, chronic obstructive pulmonary disease, allergic rhinitis, asthma, allergic asthma, periodontal diseases (such as periodonitis, gingivitis, gum disease and the like) in a subject in need thereof comprising administering to the subject an effective amount of a compound of Formula (I) or a form, composition or medicament thereof.

Another example of the invention is a method for preventing, treating or ameliorating CCR2 mediated uveitis, wherein uveitis includes, without limitation, acute, recurring or chronic uveitis (such as anterior uveitis, intermediate uveitis, posterior uveitis, panuveitis and the like) in a subject in need thereof comprising administering to the subject an effective amount of a compound of Formula (I) or a form, composition or medicament thereof.

An example of the invention is a method for preventing, treating or ameliorating CCR2 mediated acute uveitis, recurring uveitis, chronic uveitis, allergic conjunctivitis, rheumatoid arthritis, psoriasis, psoriatic arthritis, atopic dermatitis, chronic obstructive pulmonary disease, allergic rhinitis, asthma, allergic asthma, periodonitis, gingivitis or gum disease in a subject in need thereof comprising administering to the subject an effective amount of a compound of Formula (I) or a form, composition or medicament thereof.

The invention includes a method for preventing, treating or ameliorating a CCR2 mediated inflammatory syndrome, disorder or disease in a subject in need thereof comprising administering to the subject an effective amount of a compound of Formula (I) or a form, composition or medicament thereof in a combination product with one or more therapeutic agents.

The term “combination product” refers to a compound of Formula (I) or a form, composition or medicament thereof in admixture with a therapeutic agent and an optional carrier for preventing, treating or ameliorating a CCR2 mediated inflammatory syndrome, disorder or disease.

The term “therapeutic agent” refers to one or more anti-inflammatory agents (such as a small molecule, antibiotic, corticosteroid, steroid, and the like), anti-infective agents or immunosuppressive agents.

For preventing, treating or ameliorating a CCR2 mediated inflammatory syndrome, disorder or disease using a compound of Formula (I) or a form, composition or medicament thereof and a therapeutic agent in a combination product includes, without limitation, co-administration of the compound and the agent, sequential administration of the compound and the agent, administration of a composition containing of the compound and the agent or simultaneous administration of separate compositions containing of the compound and the agent.

As those skilled in the art will appreciate, the effective amounts of the components comprising the combination product may be independently optimized and combined to achieve a synergistic result whereby the pathology is reduced more than it would be if the components of the combination product were used alone.

Pharmaceutical Compositions

The present invention includes a pharmaceutical composition or medicament comprising one or more of the instant compounds and an optional pharmaceutically acceptable carrier.

The present invention further includes a process for making a pharmaceutical composition or medicament comprising mixing one or more of the instant compounds and an optional pharmaceutically acceptable carrier; and, includes those compositions or medicaments resulting from such a process. Contemplated processes include both conventional and unconventional pharmaceutical techniques.

The composition or medicament may take a wide variety of forms to effectuate mode of administration ocularly, intranasally (by inhalation or insufflation), sublingually, orally, parenterally or rectally including, without limitation, ocular (via a delivery device such as a contact lens and the like), intranasal (via a delivery device), transdermal, topical with or without occlusion, intravenous (both bolus and infusion), injection (intraperitoneally, subcutaneously, intramuscularly, intratumorally, or parenterally) and the like.

The composition or medicament may be in a dosage unit such as a tablet, pill, capsule, powder, granule, liposome, biodegradable carrier, ion exchange resin, sterile solution and the like (facilitating immediate release, timed release, or sustained release), parenteral solution or suspension, metered aerosol or liquid spray, drop, ampoule, auto-injector device or suppository.

Compositions or medicaments suitable for oral administration include solid forms such as pills, tablets, caplets, capsules (each including immediate release, timed release, and sustained release formulations), granules and powders and liquid forms such as solutions, syrups, elixirs, emulsions and suspensions. Forms useful for nasal administration include sterile solutions or nasal delivery devices. Forms useful for ocular administration include sterile solutions or ocular delivery devices. Forms useful for parenteral administration include sterile solutions, emulsions and suspensions.

Alternatively, the composition or medicament may be administered in a form suitable for once-weekly or once-monthly administration. For example, an insoluble salt of the active compound may be adapted to provide a depot preparation for intramuscular injection (e.g., a salt form) or to provide a solution for nasal or ocular administration (e.g., a quaternary ammonium salt).

The dosage form (tablet, capsule, powder, solution, contact lens, patch, liposome, ion exchange resin, suppository, teaspoonful, and the like) containing the composition or medicament thereof contains an effective amount of the active ingredient necessary to provide a therapeutic effect.

The composition or medicament may contain an effective amount of from about 0.0001 mg to about 5000 mg (preferably, from about 0.0001 to about 500 mg) of a compound of the present invention or a pharmaceutically acceptable form thereof and may be constituted into any form suitable for the mode of administration selected for a subject in need.

A contemplated range of the effective amount includes from about 0.0001 mg to about 300 mg/kg of body weight per day. A contemplated range also includes from about 0.0003 to about 100 mg/kg of body weight per day. Another contemplated range includes from about 0.0005 to about 15 mg/kg of body weight per day. The composition or medicament may be administered according to a dosage regimen of from about 1 to about 5 times per day.

For oral administration, the composition or medicament is preferably in the form of a tablet containing, e.g., 0.001, 0.005, 0.01, 0.05, 0.1, 0.5, 1.0, 2.5, 5.0, 10.0, 15.0, 25.0, 50.0, 100, 150, 200, 250, and 500 milligrams of the active ingredient for the symptomatic adjustment of the dosage to the patient to be treated.

Optimal dosages to be administered may be readily determined by those skilled in the art, and will vary with the particular compound used, the mode of administration, the strength of the preparation and the advancement of the disease condition. In addition, factors associated with the particular patient being treated, including patient's sex, age, weight, diet, time of administration and concomitant diseases, will result in the need to adjust dosages. The use of either daily administration or post-periodic dosing may be employed.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette.

Synthetic Methods

Representative compounds of the present invention can be synthesized in accordance with the general synthetic schemes described below and are illustrated more particularly in the specific examples that follow. The general schemes and specific examples are offered by way of illustration; the invention should not be construed as being limited by the chemical reactions and conditions expressed. The methods for preparing the various starting materials used in the schemes and examples are well within the skill of persons versed in the art.

The following abbreviations and formulas have the indicated meanings:

| | |
|---|---|
| Boc | tert-butoxy carbonyl or t-butoxy carbonyl |
| Ac₂O | acetic anhydride |
| CH₂Cl₂ or DCM | methylene chloride or dichloromethane |
| CHCl₃ | chloroform |
| CH₃CN or MeCN | acetonitrile |
| COPD | chronic obstructive pulmonary disease |
| Cpd | compound |
| DBU | 1,8-diazabicyclo[5.4.0]undec-7-ene |
| DIPEA | diisopropylethylamine |
| DMAP | 4-dimethylaminopyridine |
| DME | dimethoxyethane |
| DMF | N,N-dimethyl formamide |
| EDCI | 1-(3-dimethylaminopropyl)-3-ethylcarbodiimide |
| | hydrochloride |
| Et₂O | ether |
| EtOAc or | ethylacetate |
| CH₃CO₂Et | |
| FLIPR | fluorometric imaging plate reader |
| LiAlH₄ | lithium aluminum hydride |
| LHMDS | lithium bis(trimethylsilyl)amide |
| LiOH | lithium hydroxide |
| MeOH/CH₃OH | methanol |
| MsCl | methanesulfonyl chloride |
| min(s)/hr(s)/d(s) | minute(s)/hour(s)/day(s) |
| MS | mass spectrum, refers to data shown as m/z (M + H)⁺ |
| NH₄Cl | ammonium chloride |
| N(i-Pr)₂Et | dissopropylethylamine |
| NaH | sodium hydride |
| NaHCO₃ | sodium bicarbonate |
| NaN₃ | sodium azide |
| NaOH | sodium hydroxide |
| Na₂SO₄ | sodium sulfate |
| psi | pounds per square inch |
| PTLC | preparative thin layer chromatography |
| RPMI | Roswell Park Memorial Institute |
| RT/rt/r.t. | room temperature |
| SOCl₂ | thionyl chloride |
| TEA or Et₃N | triethylamine |
| TFA | trifluoroacetic acid |
| THF | tetrahydrofuran |
| TMSCl | chlorotrimethylsilane or trimethylsilyl chloride |

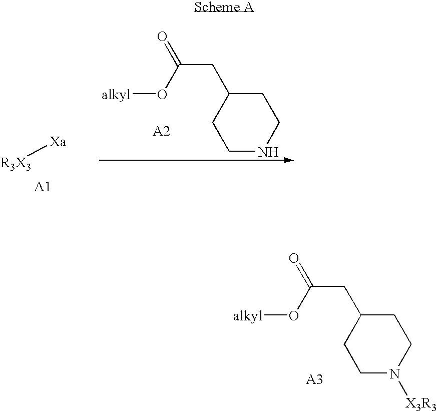

Compound A1 (wherein Xa is a suitable leaving group such as halogen) is reacted with a solution of Compound A2 (in a solvent or mixture of solvents such as TEA, methylene chloride and the like) at about 0° C. and stirred for about 8-10 hrs at room temperature to give a disubstituted piperidine Compound A3 (representative of an intermediate compound of Formula (I) wherein X₂ is absent and R₂ is carbonylalkoxy).

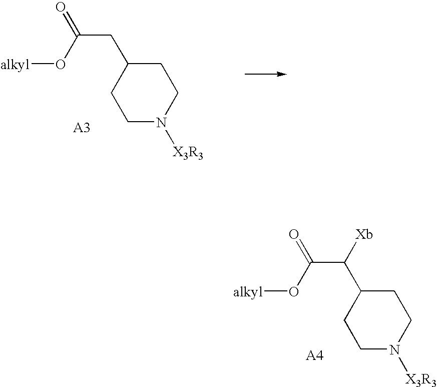

A solution of Compound A3 is added dropwise to a reagent solution (such as LHMDS in a solvent such as THF and the like) at about −78° C. and is stirred for about 3-4 hrs at about −78° C. A reagent (such as TMSCl and the like) is added dropwise to the mixture at about −78° C. The mixture is stirred for about 1 hr, then a halogen reagent solution is added (such as NBS, NCS, bromine and the like in a solvent such as THF and the like) dropwise at about −78° C. The mixture is stirred for about 2 hrs, then transferred to an ice-water bath and stirred for about 30 min. to provide Compound A4 as a racemate (wherein Xb is a suitable leaving group such as halogen).

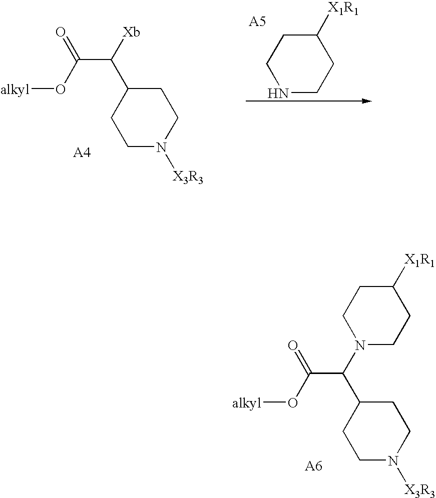

A solution of Compound A5 (commercially available or prepared according to methods well known to one skilled in the art; in a solvent such as CH₃CN and the like) and TEA are reacted at reflux for about 5 hrs with a solution of Compound A4 (in a solvent such as acetonitrile and the like) to provide a racemate Compound A6 (representative of a compound of Formula (I) wherein X₂ is absent and R₂ is carbonylalkoxy). The racemate Compound A6 may be chromatographically separated using conventional resolution techniques known to those skilled in the art.

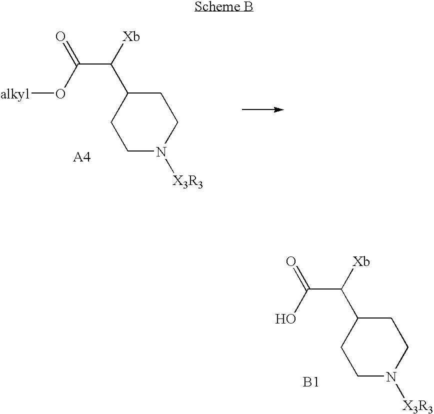

A solution of Compound A4 (wherein Xb is a suitable leaving group such as halogen) is reacted with an aqueous reagent solution (such as LiOH in a solvent such as THF, MeOH, and the like or mixtures thereof) at about room temperature. The reaction mixture is stirred at about room temperature for about 4 hrs then acidified (using an acid such as HCl and the like) to provide Compound B1.

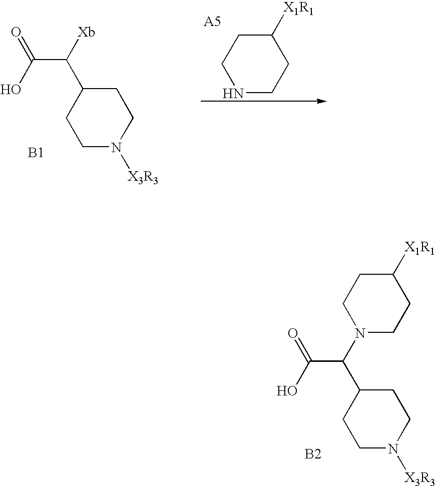

Using the procedure of Scheme A, Compound By is used in place of Compound A4. Compound By is reacted with Compound A5 to provide a racemate Compound B2 (representative of a compound of Formula (I) wherein X₂ is absent and R₂ is carboxy).

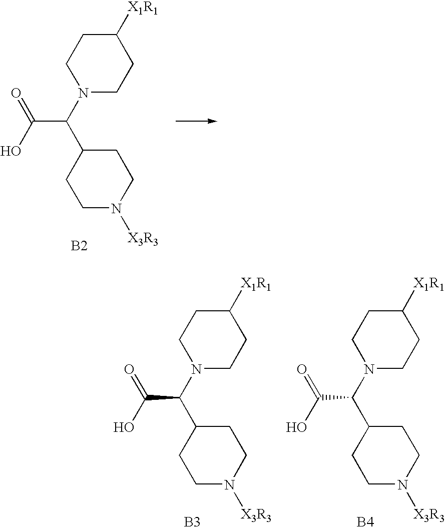

The racemate Compound B2 may be chromatographically separated using conventional resolution techniques known to those skilled in the art to provide the separate enantiomers Compound B3 and Compound B4.

For Compound B2, B3 or B4, substitutions with other functional groups may be made using techniques known to those skilled in the art to provide compounds that are representative of the scope of the present invention.

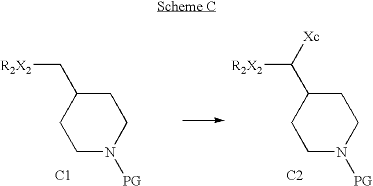

Using the procedure of Scheme A, Compound C1 (wherein PG is a protecting group, representing that X₃ is carbonylalkoxy and R₃ is not present and the like) is used in place of Compound A3.

Compound C1 is reacted with a halogen reagent solution to provide Compound C2 (wherein Xc is a suitable leaving group such as halogen) as a racemate. The racemate Compound C2 may be separated into two enantiomers using conventional resolution techniques known to those skilled in the art.

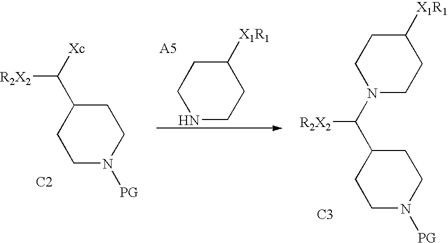

Using the procedure of Scheme A, Compound C2 is used in place of Compound A4.

Compound C2 is reacted with Compound A5 to provide Compound C3 as a racemate.

Compound C3 (wherein X₂ is absent and R₂ is selected from carbonylalkoxy or carboxy) is reacted with a reducing agent (such as lithium aluminum hydride and the like) to provide intermediates wherein X₂ is alkyl and R₂ is hydroxy.

The racemate Compound C3 may be separated into two enantiomers using conventional resolution techniques known to those skilled in the art.

For Compound C3, either before or after resolution, conversions to other functional groups may be made using techniques known to those skilled in the art to provide compounds that are representative of the scope of the present invention.

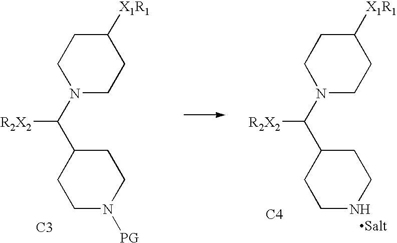

At a suitable point, the protecting group may be removed and converted to a salt form using means known to those skilled in the art to provide an intermediate Compound C4 made amendable for further substitution.

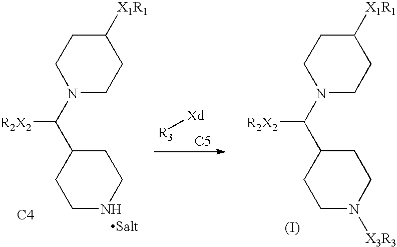

A solution of Compound C4 (in a suitable solvent such as CH₂Cl₂, CH₃CN, DMF and the like or mixtures thereof) in the presence of a suitable base (such as Et₃N, DIPEA and the like) is reacted under suitable conditions with an Xd substituted Compound C5 (wherein Xd is a suitable reaction group such as isocyanato, isothiocyanato, N-(imino-pyrazol-1-yl-methyl)-aminocarbonyl, acrylylchloride and the like, wherein certain portions of Xd are incorporated into X₃ as a product of the reaction) to provide a compound of Formula (I).

Included within the scope of the present invention are art known functional group transformations for any of the foregoing intermediates or compounds described in the present invention.

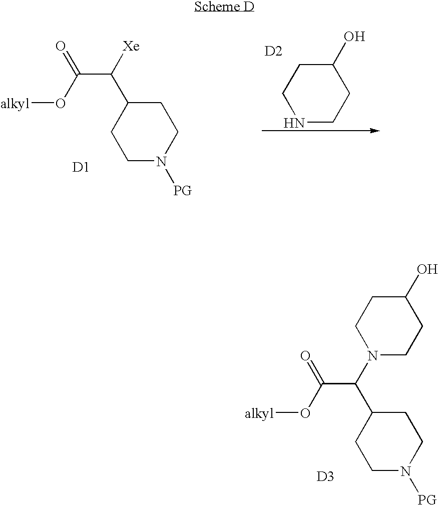

A solution of commercially available Compound D2 and Compound D1 (wherein Xe is a suitable leaving group such as halogen) is refluxed (in a solvent such as acetonitrile and the like) in the presence of a reagent (such as DIPEA and the like) to provide Compound D3 as a racemate.

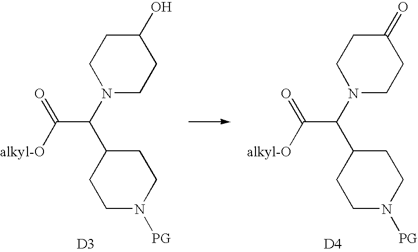

A solution of Compound D3 is oxidized (using an oxidizing agent such as oxalyl chloride, DMSO and TEA in CH₂Cl₂ and the like) to provide Compound D4.

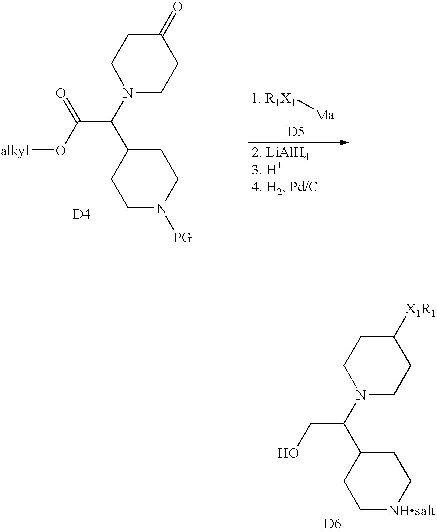

In Step 1 of the reaction sequence, Compound D4 is reacted with a Compound D5 (wherein X₁ is absent or alkyl and Ma represents a magnesium halide or other metal or metal halide group and the like) to provide an R₁ substituted intermediate (wherein a tertiary hydroxyl group is present at the point of attachment of X₁R₁ on the piperidine ring).

In Step 2 of the reaction sequence, the Compound D4 R₂ ester group is reacted with a reducing reagent (such as lithium aluminum hydride and the like), whereby the ester is converted to a hydroxymethyl group.

In Step 3 of the reaction sequence, the Compound D4 protecting group is removed and converted to an acid salt form and the tertiary hydroxyl is simultaneously eliminated with an acid (such as trifluoroacetic acid or hydrochloric acid and the like).

In Step 4 of the reaction sequence, a Compound D4 double bond resulting from the tertiary hydroxyl elimination is hydrogenated in the presence of a suitable catalyst (such as palladium on carbon and the like).

Using the procedure of Scheme C and Compound D6 in place of Compound C4 enables one skilled in the art to prepare other compounds representative of the scope of the present invention.

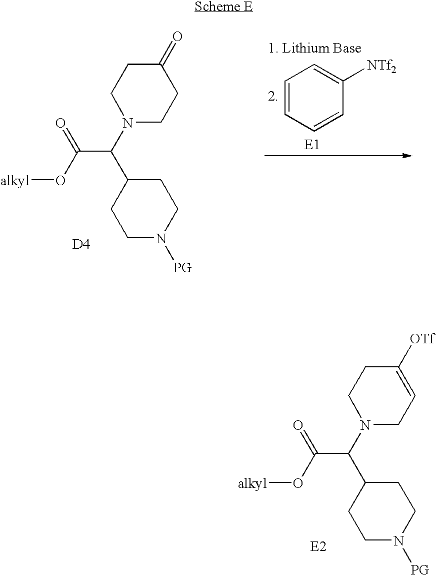

In Step 1 of the reaction sequence, Compound D4 is enolized using a suitable lithiated amine base (such as LHMDS and the like in a solvent such as THF and the like) at −78° C.

In Step 2 of the reaction sequence, the enolized intermediate is reacted with N-phenyl-trifluoromethanesulfonimide to provide the vinyl triflate Compound E2.

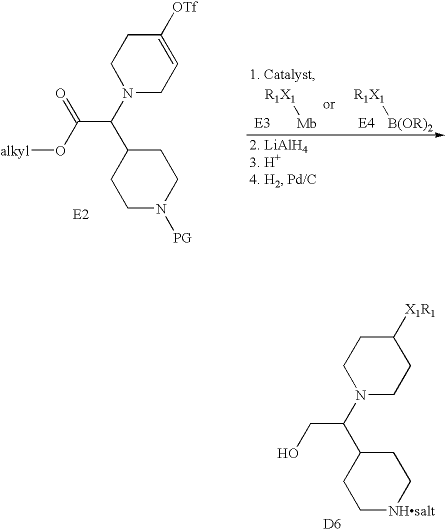

In Step 1 of the reaction sequence, Compound E2 is coupled with either Compound E3 (wherein X₁ is absent or —CH₂— and Mb represents a zinc halide or other metalated group and the like) or Compound E4 (wherein X₁ is absent and B(OR)₂ represents a boronic ester or acid 20 group and the like) in the presence of a transition metal catalyst (such as tetrakis (triphenylphosphine)palladium and the like) to provide an intermediate product which is then carried forward in Reactions 24, according to the procedure of Scheme D, to provide Compound D6 (wherein X₁ is as defined respectively for Compound E3 or Compound E4).

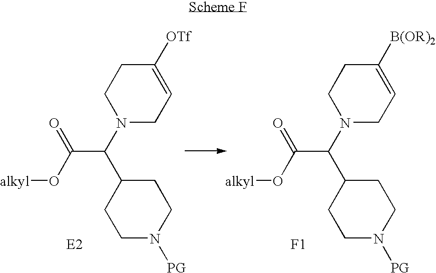

Compound E2 is reacted with a diborane [such as 4,4,5,5,4′,4′,5′,5′-octamethyl-[2,2′]bi[[1,3,2]dioxaborolanyl] (also referred to as bis-pinacolato-diboron) and the like] and a palladium catalyst (such as dichloro[1,1′-bis(diphenylphosphino)ferrocene]palladium and the like) to provide Compound F1.

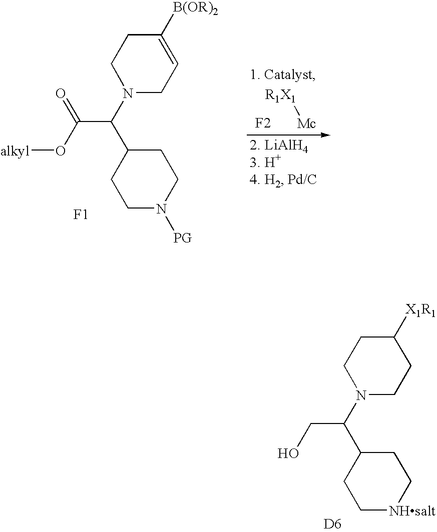

In Reaction 1, Compound F1 is coupled with Compound F2 (wherein X₁ is absent and Mc represents triflate, halide and the like) in the presence of a transition metal catalyst (such as tetrakis (triphenylphosphine)palladium and the like) to provide an intermediate product which is then carried forward in Reactions 2-4, according to the procedure of Scheme D, to provide Compound D6 (wherein X₁ is absent).

The invention is further defined by reference to the following examples, which are merely intended to be illustrative and not limiting.

### EXAMPLE 1

### [4-(1H-indol-3-yl)-piperidin-1-yl]-{1-[(2E)-3-(3,4,5-trifluoro-phenyl)-acryloyl]-piperidin-4-yl}-acetic acid (Cpd 6)

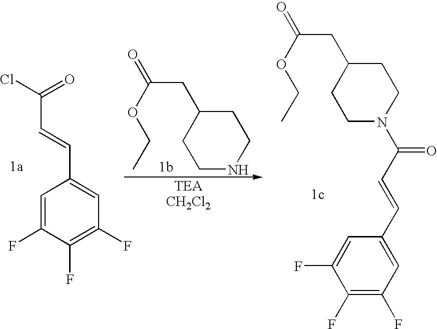

3-(3,4,5-trifluoro-phenyl)-acryloyl chloride Compound 1a (1.50 g, 6.80 mmol) was added to the solution of piperidin-4-yl-acetic acid ethyl ester Compound 1b (1.28 g, 7.49 mmol) and TEA (triethylamine) (1.89 mL, 13.56 mmol) in CH₂Cl₂ (30 mL) at 0° C. The mixture was stirred overnight at room temperature, diluted with methylene chloride (20 mL) and washed with 1 N HCl (10 mL) and water (10 mL), then dried over Na₂SO₄ and concentrated. The crude product was purified by chromatography (50% EtOAc/hexane) to give {1-[3,4,5-trifluoro-phenyl)acryloyl]-piperidin-4-yl}-acetic acid ethyl ester Compound 1c (1.80 g, 75% yield). MS: m/z 356 (M+H)⁺.

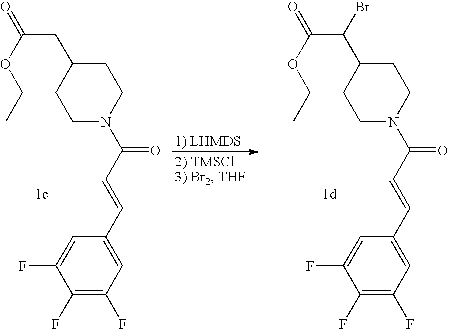

To a solution of LHMDS in THF (1.0 M, 4.9 mL) at −78° C. was added dropwise a solution of Compound 1c (0.96 g, 2.70 mmol) in THF (8 mL). The resulting reaction mixture was stirred at −78° C. for 3.5 hrs. TMSCl (0.62 mL, 4.88 mmol) was added dropwise to the reaction mixture at −78° C., then the mixture was stirred for 1 hr and Br₂ (0.17 mL, 3.3 mmol) was added dropwise at the same temperature. The reaction mixture was stirred at −78° C. for 2 hrs, then stirred in an ice-water bath for 0.5 hr. The reaction mixture was poured into a mixture of EtOAc (100 mL) and NaHCO₃ (100 mL). The organic layer was washed with water (1×100 mL) and brine (1×100 mL), then dried over Na₂SO₄, filtered and concentrated. The resulting crude product was purified on a silica gel column with 50% EtOAc/hexane to give bromo-{1-[3-(3,4,5-trifluoro-phenyl)-acryloyl]-piperidin-4-yl}-acetic acid ethyl ester Compound 1d (0.7 g, 59.8%). MS: m/z 434 (M+H)⁺.

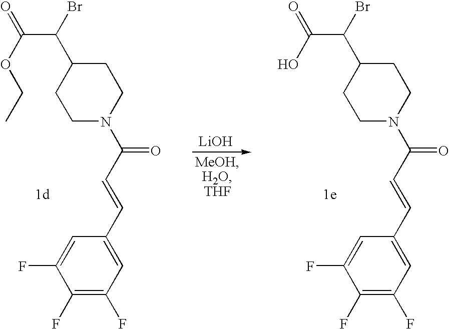

To a solution of Compound 1d (0.7 g, 1.62 mmol) in MeOH (18 mL) and THF (6 mL) at room temperature was added LiOH (0.2 g, 8.3 mmol) in water (6 mL). The resulting reaction mixture was stirred at room temperature for 4 hrs and concentrated by evaporating the MeOH and THF solvents. The aqueous solution was acidified to pH 1 with 1M HCl solution and extracted with EtOAc. The organic layer was washed with brine (1×100 mL), dried over Na₂SO₄, then filtered and concentrated to give bromo-{1-[3-(3,4,5-trifluoro-phenyl)-acryloyl]-piperidin-4-yl}-acetic acid Compound 1e (0.64 g, 98%). MS: m/z 406 (M+H)⁺.

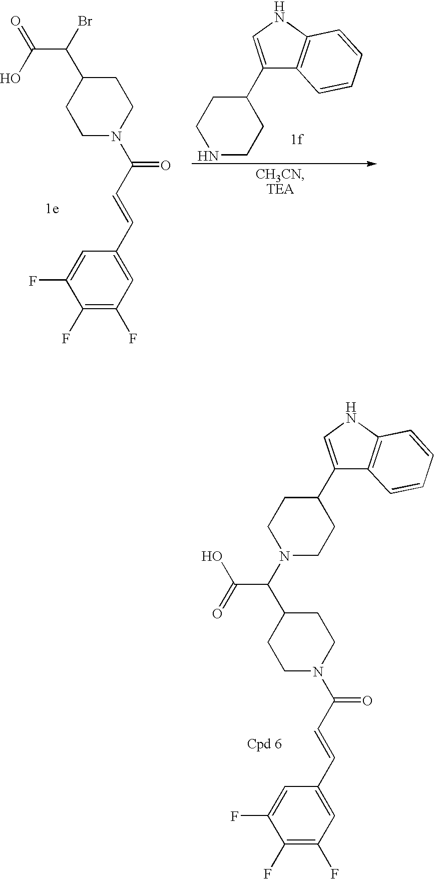

To a solution of Compound 1e (0.26 g, 0.64 mmol) in acetonitrile (10 mL) was added 3-piperidin-4-yl-1H-indole Compound 1f (152 mg, 0.64 mmol) and TEA (0.18 mL, 1.29 mmol). The resulting reaction mixture was refluxed for 5 hrs, then concentrated and cooled to provide a white precipitate. The precipitate was washed with EtOAc and water to give Compound 6 (0.23 g, 67%) as a racemate. MS m/z 526 (M+H)⁺. ¹H NMR (DMSO-d6, 400 MHz) δ 12.11 (br s, 1H), 10.85 (s, 1H), 7.81 (q, J=7.2 Hz, 2H), 7.55 (d, J=8.0 Hz, 1H), 7.37 (m, 2H), 7.32 (d, J=8.0 Hz, 1H), 7.04 (m, 2H), 6.95 (q, J=7.0 Hz, 1H), 4.47 (m, 1H), 4.31 (m, 1H), 3.10 (m, 1H), 2.96 (d, J=10.8 Hz, 1H), 2.88 (m, 2H), 2.65 (m, 3H), 2.35 (m, 1H), 2.06 (m, 1H), 1.94 (m, 1H), 1.61 (m, 2H), 1.09 (m, 2H).

Using the procedure of Example 1 and known appropriate reagents and starting materials, the following compounds of the invention were prepared:

| Cpd | Name | MS |
|---|---|---|
| 1 | [4-(4-chloro-phenyl)-piperidin-1-yl]-{1-[(2E)-3-(3,4-dichloro-phenyl)- | 535 |
| | acryloyl]-piperidin-4-yl}-acetic acid | |
| 2 | {1-[(2E)-3-(3,5-difluoro-phenyl)-acryloyl]-piperidin-4-yl}-[4-(4- | 499 |
| | methoxy-phenyl)-piperidin-1-yl]-acetic acid | |
| 4 | [4-(4-chloro-phenyl)-piperidin-1-yl]-{1-[(2E)-3-(3,4,5-trifluoro-phenyl)- | 521 |
| | acryloyl]-piperidin-4-yl}-acetic acid | |
| 5 | [4-(4-methoxy-phenyl)-piperidin-1-yl]-{1-[(2E)-3-(3,4,5-trifluoro- | 517 |
| | phenyl)-acryloyl]-piperidin-4-yl}-acetic acid | |
| 7 | {1-[(2E)-3-(3,5-difluoro-phenyl)-acryloyl]-piperidin-4-yl}-[4-(1H-indol- | 508 |
| | 3-yl)-piperidin-1-yl]-acetic acid | |
| 8 | {1-[(2E)-3-(3,5-difluoro-phenyl)-acryloyl]-piperidin-4-yl}-[4-(5-fluoro- | 526 |
| | 1H-indol-3-yl)-piperidin-1-yl]-acetic acid | |
| 9 | [4-(5-fluoro-1H-indol-3-yl)-piperidin-1-yl]-{1-[(2E)-3-(3,4,5-trifluoro- | 544 |
| | phenyl)-acryloyl]-piperidin-4-yl}-acetic acid | |
| 10 | (4-indol-1-yl-piperidin-1-yl)-{1-[(2E)-3-(3,4,5-trifluoro-phenyl)- | 526 |
| | acryloyl]-piperidin-4-yl}-acetic acid | |
| 11 | {1-[(2E)-3-(3,5-difluoro-phenyl)-acryloyl]-piperidin-4-yl}-[4-(1H-indol- | 522 |
| | 3-ylmethyl)-piperidin-1-yl]-acetic acid | |
| 12 | [4-(1H-indol-3-ylmethyl)-piperidin-1-yl]-{1-[(2E)-3-(3,4,5-trifluoro- | 540 |
| | phenyl)-acryloyl]-piperidin-4-yl}-acetic acid | |
| 15 | [4-(5-hydroxy-1H-indol-3-yl)-piperidin-1-yl]-{1-[(2E)-3-(3,4,5- | 542 |
| | trifluoro-phenyl)-acryloyl]-piperidin-4-yl}-acetic acid | |
| 16 | {1-[(2E)-3-(3,5-difluoro-phenyl)-acryloyl]-piperidin-4-yl}-[4-(5- | 524 |
| | hydroxy-1H-indol-3-yl)-piperidin-1-yl]-acetic acid | |
| 17 | [4-(5-acetylamino-1H-indol-3-yl)-piperidin-1-yl]-{1-[(2E)-3-(3,4,5- | 583 |
| | trifluoro-phenyl)-acryloyl]-piperidin-4-yl}-acetic acid | |
| 18 | {1-[(2E)-3-(3,4-dichloro-phenyl)-acryloyl]-piperidin-4-yl}-[4-(1H-indol- | 540 |
| | 3-yl)-piperidin-1-yl]-acetic acid | |
| 19 | {1-[(2E)-3-(3,4-dichloro-phenyl)-acryloyl]-piperidin-4-yl}-[4-(5-fluoro- | 558 |
| | 1H-indol-3-yl)-piperidin-1-yl]-acetic acid | |
| 22 | {1-[(2E)-3-(3,4-difluoro-phenyl)-acryloyl]-piperidin-4-yl}-[4-(1H-indol- | 508 |
| | 3-yl)-piperidin-1-yl]-acetic acid | |
| 23 | [4-(1H-indol-3-yl)-piperidin-1-yl]-{1-[(2E)-3-(4-trifluoromethyl- | 540 |
| | phenyl)-acryloyl]-piperidin-4-yl}-acetic acid | |
| 24 | {1-[(2E)-3-(3,5-difluoro-phenyl)-acryloyl]-piperidin-4-yl}-[4-(6-fluoro- | 526 |
| | 1H-indol-3-yl)-piperidin-1-yl]-acetic acid | |
| 25 | [4-(6-chloro-1H-indol-3-yl)-piperidin-1-yl]-{1-[(2E)-3-(3,5-difluoro- | 542 |
| | phenyl)-acryloyl]-piperidin-4-yl}-acetic acid | |
| 26 | {1-[(2E)-3-(3,5-difluoro-phenyl)-acryloyl]-piperidin-4-yl}-[4-(5- | 538 |
| | methoxy-1H-indol-3-yl)-piperidin-1-yl]-acetic acid | |
| 27 | [4-(1H-indol-3-yl)-piperidin-1-yl]-{1-[(2E)-3-phenyl-acryloyl]- | 472 |
| | piperidin-4-yl}-acetic acid | |
| 29 | {1-[(2E)-3-(3,5-difluoro-phenyl)-acryloyl]-piperidin-4-yl}-[4-(5- | 601 |
| | methanesulfonylamino-1H-indol-3-yl)-piperidin-1-yl]-acetic acid | |
| 30 | [4-(5-methoxy-1H-indol-3-yl)-piperidin-1-yl]-{1-[(2E)-3-(3,4,5- | 556 |
| | trifluoro-phenyl)-acryloyl]-piperidin-4-yl}-acetic acid | |
| 31 | [4-(6-chloro-1H-indol-3-yl)-piperidin-1-yl]-{1-[(2E)-3-(3,4,5-trifluoro- | 560 |
| | phenyl)-acryloyl]-piperidin-4-yl}-acetic acid | |
| 34 | {1-[(2E)-3-(4-chloro-phenyl)-acryloyl]-piperidin-4-yl}-[4-(1H-indol-3- | 506 |
| | yl)-piperidin-1-yl]-acetic acid | |
| 35 | [4-(1H-indol-3-yl)-piperidin-1-yl]-{1-[(2E)-3-(3-trifluoromethyl- | 540 |
| | phenyl)-acryloyl]-piperidin-4-yl}-acetic acid | |
| 36 | {1-[(2E)-3-(3-bromo-4-fluoro-phenyl)-acryloyl]-piperidin-4-yl}-[4-(1H- | 568 |
| | indol-3-yl)-piperidin-1-yl]-acetic acid | |
| 37 | [4-(1H-indol-3-yl)-piperidin-1-yl]-{1-[(2E)-3-(4-methoxy-phenyl)- | 502 |
| | acryloyl]-piperidin-4-yl}-acetic acid | |
| 38 | {1-[(2E)-3-(3,5-difluoro-phenyl)-acryloyl]-piperidin-4-yl}-[4-(6- | 538 |
| | methoxy-1H-indol-3-yl)-piperidin-1-yl]-acetic acid | |
| 39 | {1-[(2E)-3-(3,4-dichloro-phenyl)-acryloyl]-piperidin-4-yl}-[4-(6-fluoro- | 558 |
| | 1H-indol-3-yl)-piperidin-1-yl]-acetic acid | |
| 41 | {1-[(2E)-3-(3,5-difluoro-phenyl)-acryloyl]-piperidin-4-yl}-[4-(4- | 538 |
| | methoxy-1H-indol-3-yl)-piperidin-1-yl]-acetic acid | |
| 42 | {1-[(2E)-3-(3,5-difluoro-phenyl)-acryloyl]-piperidin-4-yl}-[4-(7- | 538 |
| | methoxy-1H-indol-3-yl)-piperidin-1-yl]-acetic acid | |
| 46 | [4-(6-chloro-1H-indol-3-yl)-piperidin-1-yl]-{1-[(2E)-3-(3,4-dichloro- | 574 |
| | phenyl)-acryloyl]-piperidin-4-yl}-acetic acid | |
| 47 | {1-[(2E)-3-(3,4-dichloro-phenyl)-acryloyl]-piperidin-4-yl}-[4-(5- | 570 |
| | methoxy-1H-indol-3-yl)-piperidin-1-yl]-acetic acid | |
| 53 | [4-(5-methanesulfonylamino-1H-indol-3-yl)-piperidin-1-yl]-{1-[(2E)-3- | 619 |
| | (3,4,5-trifluoro-phenyl)-acryloyl]-piperidin-4-yl}-acetic acid | |
| 59 | [4-(1H-indol-3-yl)-piperidin-1-yl]-{1-[(2E)-3-(4-nitro-phenyl)-acryloyl]- | 517 |
| | piperidin-4-yl}-acetic acid | |
| 60 | {1-[(2E)-3-(4-bromo-phenyl)-acryloyl]-piperidin-4-yl}-[4-(1H-indol-3- | 550 |
| | yl)-piperidin-1-yl]-acetic acid | |
| 61 | [4-(1H-indol-3-yl)-piperidin-1-yl]-{1-[(2E)-3-p-tolyl-acryloyl]- | 486 |
| | piperidin-4-yl}-acetic acid | |
| 62 | {1-[(2E)-3-(3-fluoro-phenyl)-acryloyl]-piperidin-4-yl}-[4-(1H-indol-3- | 490 |
| | yl)-piperidin-1-yl]-acetic acid | |
| 63 | {1-[(2E)-3-(3,4-dimethoxy-phenyl)-acryloyl]-piperidin-4-yl}-[4-(1H- | 532 |
| | indol-3-yl)-piperidin-1-yl]-acetic acid | |
| 70 | [4-(1H-indol-3-yl)-piperidin-1-yl]-{1-[(2E)-3-m-tolyl-acryloyl]- | 486 |
| | piperidin-4-yl}-acetic acid | |
| 71 | {1-[(2E)-3-(3-bromo-phenyl)-acryloyl]-piperidin-4-yl}-[4-(1H-indol-3- | 550 |
| | yl)-piperidin-1-yl]-acetic acid | |
| 72 | [4-(1H-indol-3-yl)-piperidin-1-yl]-{1-[(2E)-3-(3-methoxy-phenyl)- | 502 |
| | acryloyl]-piperidin-4-yl}-acetic acid | |
| 74 | {1-[(2E)-3-(3-fluoro-4-methyl-phenyl)-acryloyl]-piperidin-4-yl}-[4-(1H- | 504 |
| | indol-3-yl)-piperidin-1-yl]-acetic acid | |
| 75 | {1-[(2E)-3-(3-fluoro-4-trifluoromethyl-phenyl)-acryloyl]-piperidin-4- | 558 |
| | yl}-[4-(1H-indol-3-yl)-piperidin-1-yl]-acetic acid | |
| 76 | {1-[(2E)-3-(3-chloro-4-fluoro-phenyl)-acryloyl]-piperidin-4-yl}-[4-(1H- | 524 |
| | indol-3-yl)-piperidin-1-yl]-acetic acid | |
| 77 | {1-[(2E)-3-(4-fluoro-phenyl)-acryloyl]-piperidin-4-yl}-[4-(1H-indol-3- | 490 |
| | yl)-piperidin-1-yl]-acetic acid | |
| 81 | [4-(1H-pyrrol-3-yl)-piperidin-1-yl]-{1-[(2E)-3-(3,4,5-trifluoro-phenyl)- | 641 |
| | acryloyl]-piperidin-4-yl}-acetic acid | |
| 82 | [4-(5-tert-butoxycarbonylamino-1H-indol-3-yl)-piperidin-1-yl]-{1-[(2E)- | 619 |
| | 3-(3,4,5-trifluoro-phenyl)-acryloyl]-piperidin-4-yl}-acetic acid | |
| 83 | [4-(6-methanesulfonylamino-1H-indol-3-yl)-piperidin-1-yl]-{1-[(2E)-3- | 541 |
| | (3,4,5-trifluoro-phenyl)-acryloyl]-piperidin-4-yl}-acetic acid | |
| 92 | [4-(1H-indol-3-yl)-piperidin-1-yl]-{1-[(2E)-3-(3-nitro-phenyl)-acryloyl]- | 517 |
| | piperidin-4-yl}-acetic acid | |
| 93 | {1-[(2E)-3-(3-chloro-phenyl)-acryloyl]-piperidin-4-yl}-[4-(1H-indol-3- | 506 |
| | yl)-piperidin-1-yl]-acetic acid | |
| 103 | [4-(1H-indol-3-yl)-piperidin-1-yl]-{1-[(2E)-3-thiophen-2-yl-acryloyl]- | 478 |
| | piperidin-4-yl}-acetic acid | |
| 104 | [4-(1H-indol-3-yl)-piperidin-1-yl]-{1-[(2E)-3-thiophen-3-yl-acryloyl]- | 478 |
| | piperidin-4-yl}-acetic acid | |
| 108 | [4-(7-methoxy-1H-indol-3-yl)-piperidin-1-yl]-{1-[(2E)-3-(3,4,5- | 556 |
| | trifluoro-phenyl)-acryloyl]-piperidin-4-yl}-acetic acid | |
| 114 | {1-[2-(3,4-dichloro-phenoxy)-acetyl]-piperidin-4-yl}-[4-(1H-indol-3-yl)- | 544 |
| | piperidin-1-yl]-acetic acid | |
| 115 | {1-[3-(3,4-dichloro-phenyl)-propionyl]-piperidin-4-yl}-[4-(1H-indol-3- | 542 |
| | yl)-piperidin-1-yl]-acetic acid | |
| 145 | 4-[1-(carboxy-{1-[(2E)-3-(3,5-difluoro-phenyl)-acryloyl]-piperidin-4- | 527 |
| | yl}-methyl)-piperidin-4-yl]-benzoic acid methyl ester | |
| 146 | 3-[1-(carboxy-{1-[(2E)-3-(3,4,5-trifluoro-phenyl)-acryloyl]-piperidin-4- | 584 |
| | yl}-methyl)-piperidin-4-yl]-1H-indole-5-carboxylic acid methyl ester | |
| 147 | 3-[1-(carboxy-{1-[(2E)-3-(3,4,5-trifluoro-phenyl)-acryloyl]-piperidin-4- | 570 |
| | yl}-methyl)-piperidin-4-yl]-1H-indole-5-carboxylic acid | |
| 151 | [4-(1H-pyrrolo[2,3-b]pyridin-3-yl)-piperidin-1-yl]-{1-[(2E)-3-(3,4,5- | 527 |
| | trifluoro-phenyl)-acryloyl]-piperidin-4-yl}-acetic acid | |
| 177 | {1-[(2E)-3-(3,5-difluoro-phenyl)-acryloyl]-piperidin-4-yl}-[4-(1H- | 509 |
| | indazol-3-yl)-piperidin-1-yl]-acetic acid | |
| 178 | [4-(5-amino-1H-pyrrolo[3,2-b]pyridin-3-yl)-piperidin-1-yl]-{1-[(2E)-3- | 542 |
| | (3,4,5-trifluoro-phenyl)-acryloyl]-piperidin-4-yl}-acetic acid | |
| 179 | [4-(5-amino-1H-pyrrolo[2,3-c]pyridin-3-yl)-piperidin-1-yl]-{1-[(2E)-3- | 542 |
| | (3,4,5-trifluoro-phenyl)-acryloyl]-piperidin-4-yl}-acetic acid | |
| 204 | [4-(2-methyl-1H-indol-3-yl)-piperidin-1-yl]-{1-[(2E)-3-(3,4,5-trifluoro- | 540 |
| | phenyl)-acryloyl]-piperidin-4-yl}-acetic acid | |
| 206 | [4-(4-methanesulfonylamino-phenyl)-piperidin-1-yl]-{1-[(2E)-3-(3,4,5- | 580 |
| | trifluoro-phenyl)-acryloyl]-piperidin-4-yl}-acetic acid | |
| 207 | [4-(1H-pyrrolo[3,2-b]pyridin-3-yl)-piperidin-1-yl]-{1-[(2E)-3-(3,4,5- | 527 |
| | trifluoro-phenyl)-acryloyl]-piperidin-4-yl}-acetic acid | |
| 211 | [4-(6-fluoro-1H-indol-3-yl)-piperidin-1-yl]-{1-[(2E)-3-(3,4,5-trifluoro- | 544 |
| | phenyl)-acryloyl]-piperidin-4-yl}-acetic acid | |

### EXAMPLE 2

### (S)-{[4-(1H-indol-3-yl)-piperidin-1-yl]}-{1-[(2E)-3-(3,4,5-trifluoro-phenyl)-acryloyl]-piperidin-4-yl}-acetic acid (Cpd 13)

### (R)-{[4-(1H-indol-3-yl)-piperidin-1-yl}-{1-[(2E)-3-(3,4,5-trifluoro-phenyl)-acryloyl]-piperidin-4-yl}-acetic acid (Cpd 14)

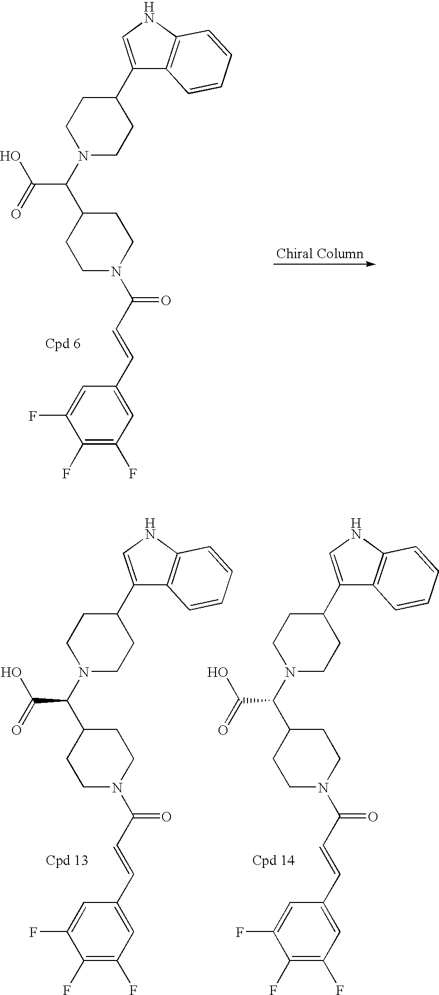 The racemate [4-(1H-indol-3-yl)-piperidin-1-yl]-{1-[3-(3,4,5-trifluoro-phenyl)-acryloyl]-piperidin-4-yl}-acetic acid Compound 6 (255 mg, 0.49 mmol) was separated into two enantiomers Compound 13 (110 mg, 86.3%) and Compound 14 (110 mg, 86.3%) with a chiralpak AD column (eluted with CH₃CN/CH₃OH 85/15).

Compound 13: MS m/z 526 (M+H)⁺, 548 (M+Na)⁺. ¹H NMR (DMSO-d6, 400 MHz) δ 11.95 (br s, 1H), 10.78 (s, 1H), 7.81 (m, 2H), 7.55 (d, J=8.0 Hz, 1H), 7.37 (m, 2H), 7.32 (d, J=8.0 Hz, 1H), 7.04 (m, 2H), 6.95 (q, J=7.0 Hz, 1H), 4.47 (m, 1H), 4.31 (m, 1H), 3.10 (m, 1H), 2.90 (m, 3H), 2.65 (m, 3H), 2.35 (m, 1H), 2.06 (m, 1H), 1.94 (m, 3H), 1.69 (m, 1H), 1.61 (m, 2H), 1.09 (m, 2H).

Compound 14: MS m/z 526 (M+H)⁺, 548 (M+Na)⁺. ¹H NMR (DMSO-d6, 400 MHz) δ 12.02 (br s, 1H), 10.73 (s, 1H), 7.81 (m, 2H), 7.53 (d, J=8.0 Hz, 1H), 7.37 (m, 2H), 7.32 (d, J=8.0 Hz, 1H), 7.04 (m, 2H), 6.95 (q, J=7.0 Hz, 1H), 4.46 (m, 1H), 4.31 (m, 1H), 3.10 (m, 1H), 2.90 (m, 3H), 2.65 (m, 3H), 2.35 (m, 1H), 2.06 (m, 1H), 1.94 (m, 3H), 1.69 (m, 1H), 1.61 (m, 2H), 1.09 (m, 2H).

### EXAMPLE 3

### [4-(1H-indol-3-yl)-piperidin-1-yl]-{1-[(2E)-3-(3,4,5-trifluoro-phenyl)-acryloyl]-piperidin-4-yl}-acetic acid methyl ester (Cpd 87)

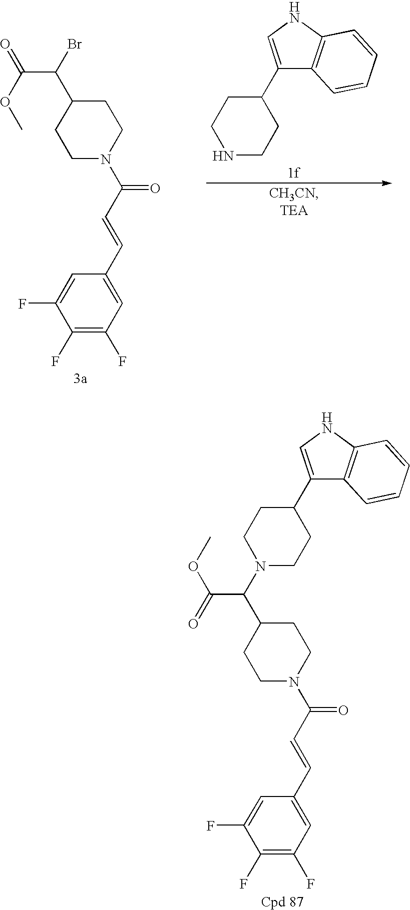

The procedure of Example 1 and piperidin-4-yl-acetic acid methyl ester was used in place of piperidin-4-yl-acetic acid ethyl ester Compound 1f to provide bromo-{1-[(2E)3-(3,4,5-trifluoro-phenyl)-acryloyl]-piperidin-4-yl}-acetic acid methyl ester Compound 3a.

3-piperidin-4-yl-1H-indole Compound 1f (1.0 g, 5.0 mmol) and TEA (0.6 g, 5.9 mmol) were added to a solution of Compound 3a (2.1 g, 5.0 mmol) in acetonitrile (70 mL). The mixture was refluxed for 48 hrs and then concentrated in vacuo. The residue was chromatographed (5% CH₃OH/CHCl₃) to give Compound 87 (1.5 g, 56%). MS m/z 540 (M+H)⁺; ¹H NMR (CDCl₃, 300 MHz) δ 7.98 (br s, 1H), 7.63 (d, J=7.8 Hz, 1H), 7.48 (d, J=15.4 Hz, 1H), 7.36 (d, J=8.0 Hz, 1H), 7.10 (m, 4H), 6.96 (br s, 1H), 6.81 (m, 1H), 4.69 (m, 1H), 4.08 (m, 1H), 3.76 (s, 3H), 3.13 (m, 1H), 2.93 (m, 2H), 2.82 (m, 3H), 2.59 (m, 1H), 2.29 (m, 1H), 2.08 (m, 4H), 1.79 (m, 1H), 1.65 (m, 2H), 1.21 (m, 2H).

Using the procedure of Example 3 and known appropriate reagents and starting materials, the following compounds of the invention were prepared:

| Cpd | Name | MS |
|---|---|---|
| 3 | {1-[(2E)-3-(3,5-difluoro-phenyl)-acryloyl]-piperidin-4-yl}- | 513 |
| | [4-(4-methoxy-phenyl)-piperidin-1-yl]-acetic acid methyl | |
| | ester | |
| 118 | [4-(7-methoxy-1H-indol-3-yl)-piperidin-1-yl]-{1-[(2E)-3- | 570 |
| | (3,4,5-trifluoro-phenyl)-acryloyl]-piperidin-4-yl}-acetic | |
| | acid methyl ester | |
| 152 | [4-(1H-pyrrolo[2,3-b]pyridin-3-yl)-piperidin-1-yl]-{1- | 555 |
| | [(2E)-3-(3,4,5-trifluoro-phenyl)-acryloyl]-piperidin-4- | |
| | yl}-acetic acid ethyl ester | |

### EXAMPLE 4

### 2-[4-(1H-indol-3-yl)-piperidin-1-yl]-2-{1-[(2E)-3-(3,4,5-trifluoro-phenyl)-acryloyl]-piperidin-4-yl}-acetamide (Cpd 107)

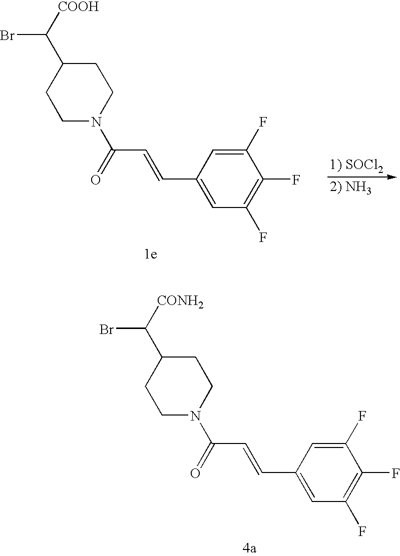

To a solution of bromo-{1-[3-(3,4,5-trifluoro-phenyl)-acryloyl]-piperidin-4-yl}-acetic acid Compound 1e (0.38 g, 0.93 mmol) in CH₂Cl₂ (4 mL) was added SOCl₂ (1 mL). The resulting reaction mixture was refluxed for 3 hrs, the concentrated in vacuo to give an acid chloride intermediate (0.39 g, 98.9%). A solution of the intermediate (0.39 g, 0.92 mmol) in acetone (10 mL) was added dropwise to a solution of ammonium hydroxide (39 mL). The reaction mixture was stirred at room temperature for 2 hrs and extracted with EtOAc (100 mL). The organic layer was washed with water (50 mL) and brine (50 mL), dried over Na₂SO₄, then filtered and concentrated to give 2-bromo-2-{1-[3-(3,4,5-trifluoro-phenyl)-acryloyl]-piperidin-4-yl}-acetamide Compound 4a (0.38 g, 94%). MS m/z 405 (M+H)⁺.

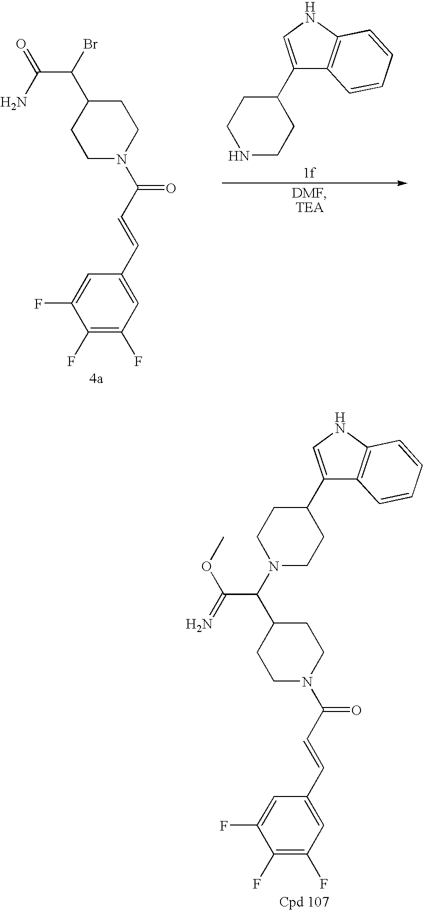

To a solution of Compound 4a (25 mg, 0.065 mmol) in DMF (4 mL) was added 3-piperidin-4-yl-1H-indole Compound 1f (13 mg, 0.065 mmol) and TEA (0.05 mL, 0.36 mmol). The reaction mixture was refluxed for 4 hrs and then concentrated in vacuo. The residue was purified using preparative TLC (70% CH₃CO₂Et/hexane) to give Compound 107 (8 mg, 25%). MS m/z 525 (M+H)⁺; ¹H NMR (CD₃OD, 300 MHz) δ: 7.38-7.61 (m, 5H), 7.18-7.31 (m, 2H), 6.92-7.10 (m, 4H), 4.62 (m, 1H), 4.39 (m, 1H), 4.12 (m, 1H), 3.79 (m, 1H), 3.10-3.40 (m, 4H), 2.79 (m, 1H), 2.61 (m, 1H), 2.08-2.39 (m, 4H), 1.81 (m, 2H), 1.25-1.49 (m, 2H).

Using the procedure of Example 4 and known appropriate reagents and starting materials, the following compounds of the invention were prepared:

| Cpd | Name | MS |
|---|---|---|
| 227 | 2-[4-(1H-pyrrolo[2,3-b]pyridin-3-yl)-piperidin-1-yl]- | 526 |
| | 2-{1-[(2E)-3-(3,4,5-trifluoro-phenyl)-acryloyl]-piperidin-4- | |
| | yl}-acetamide | |

### EXAMPLE 5

### [1-(4-fluoro-3-methyl-phenylcarbamoyl)-piperidin-4-yl]-[4-(1H-indol-3-yl)-piperidin-1-yl]-acetic acid (Cpd 102)

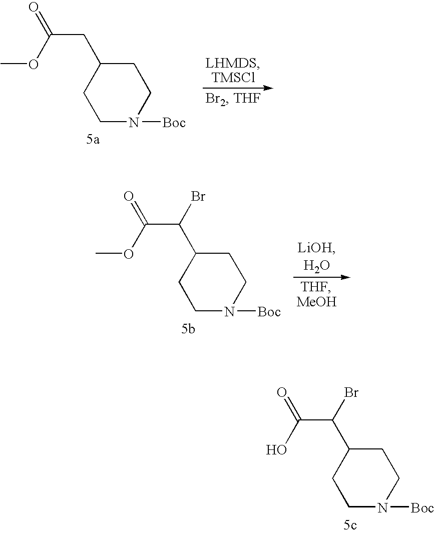

A solution of 4-methoxycarbonylmethyl-piperidine-1-carboxylic acid tert-butyl ester Compound 5a (1.0 g, 3.9 mmol) in THF (5 mL) was added to LHMDS (1.0 M in THF) (7.0 mL, 7.0 mmol) at −78° C. and the reaction mixture was stirred at −78° C. for 3 hrs. TMSCl (0.89 mL, 7.0 mmol) was added dropwise and the mixture was stirred for 1 hr at −78° C. then Br₂ (0.24 mL, 4.7 mmol) was added dropwise. The mixture was stirred at −78° C. for 2 hrs, then allowed to warm to 0° C. and stirred for an additional 30 min. The mixture was diluted with ethyl acetate and washed with saturated NaHCO₃ solution, then washed with H₂O. The organics were dried over Na₂SO₄, then the drying agent was filtered and solvent removed in vacuo to yield a yellow solid. The crude product was purified by flash column chromatography (50% EtOAc/hexane) to yield 4-(bromo-methoxycarbonyl-methyl)-piperidine-1-carboxylic acid tert-butyl ester Compound 5b as a pale yellow oil (1.0 g, 77%). MS m/z 358 (M+Na)⁺; ¹H NMR (400 MHz, CDCl₃) δ 4.15 (br, 2H), 4.01 (d, J=8.5 Hz, 1H), 3.80 (s, 3H), 2.65-2.78 (br s, 2H), 2.04 (m, 2H), 1.61 (m, 1H), 1.45 (s, 9H), 1.21 (m, 2H).

An aqueous LiOH solution (0.624 g, 14.87 mmol in 7 mL H₂O) was added to a solution of Compound 5b (1.0 g, 2.97 mmol) in MeOH (21 mL) and THF (7 mL). The reaction mixture was stirred overnight at room temperature. The solvent was removed in vacuo to provide a white solid, which was acidified with 1 N HCl. A crude product was extracted with ethyl acetate and the organics were washed with brine and dried over Na₂SO₄. The drying agent was filtered and the solvent removed in vacuo, yielding 4-(bromo-carboxy-methyl)-piperidine-1-carboxylic acid tert-butyl ester Compound 5c (0.663 g, 66%) as a white solid. The product (>90% purity by NMR) was used in the next step without further purification. MS m/z 344; 346 (M+Na)⁺; ¹H NMR (300 MHz, CDCl₃) δ 4.04.2 (m, 3H), 2.6-2.8 (br s, 2H), 1.9-2.1 (m, 2H), 1.64-1.75 (m, 1H), 1.45 (s, 9H), 1.2-1.3 (m, 2H).

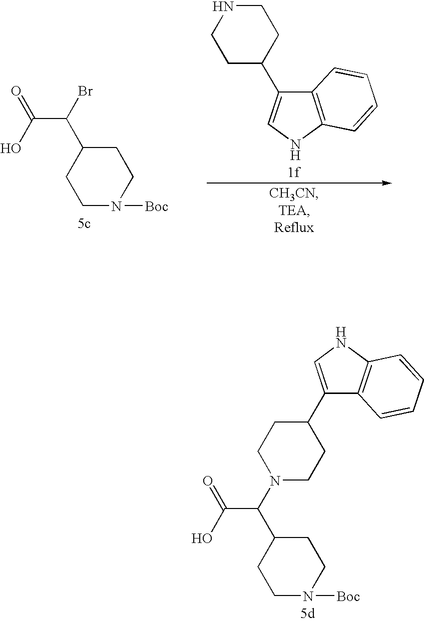

A solution of Compound 5c (0.335 g, 1.040 mmol), 3-piperidin-4-yl-1H-indole Compound 1f (0.208 g, 1.040 mmol) and TEA (0.29 mL, 2.080 mmol) in CH₃CN was refluxed for 5 hrs. The solvent was removed in vacuo to provide a yellow solid. The product was washed with a minimal amount of methanol to removing residual starting material to obtain 4-{carboxy-[4-(1H-indol-3-yl)-piperidin-1-yl]-methyl}-piperidine-1-carboxylic acid tert-butyl ester Compound 5d (27%, 0.459 g) as a white solid. MS m/z 442 (M+H)⁺.

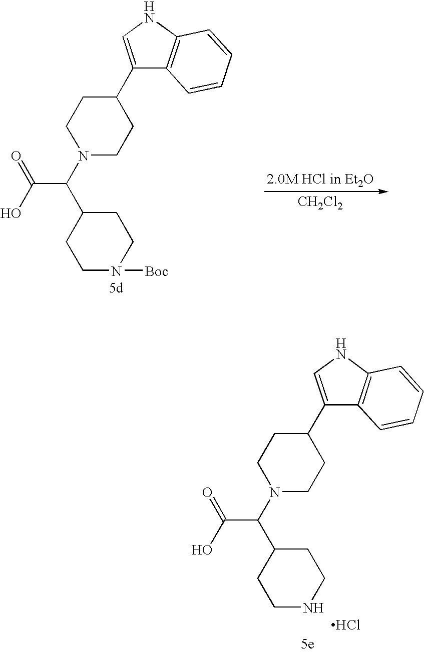

2.0 M HCl in Et₂O (5 mL, 10 mmol) was added to a solution of Compound 5d (0.125 g, 0.283 mmol) in CH₂Cl₂ (10 mL). The reaction mixture was stirred overnight at room temperature. The solvent was removed in vacuo to provide a tan solid product. The product was washed with CH₂Cl₂ to obtain [4-(1H-indol-3-yl)-piperidin-1-yl]-piperidin-4-yl-acetic acid Compound 5e (0.108 g, 100%) as a tan solid. MS m/z 342 (M+H)⁺.

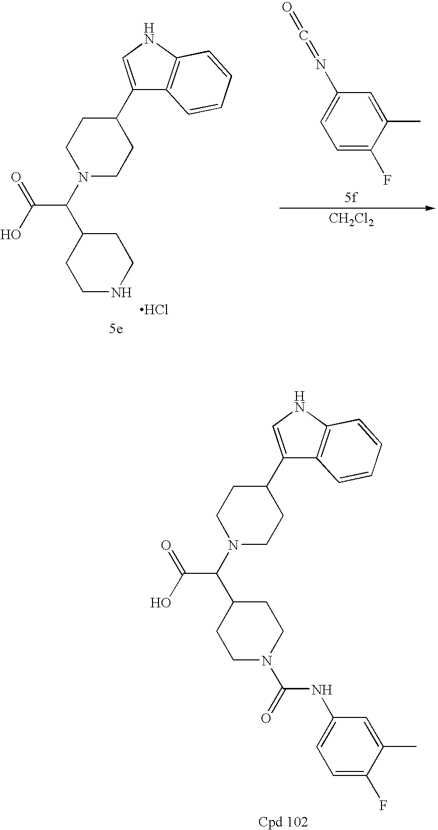

To a solution of Compound 5e (28.8 mg, 0.07 mmol) and Et₃N (0.02 mL, 0.14 mmol) in CH₂Cl₂ at 0° C. was added 1-fluoro-4-isocyanato-2-methyl-benzene Compound 5f (10.6 mg, 0.07 mmol) dropwise. The reaction mixture was warmed to room temperature and stirred overnight. The solvent was removed in vacuo, leaving an off-white solid. The solid was washed with H₂O, which was decanted and then with 50% EtOAc/hexane, which was decanted to provide Compound 102 (76%, 0.026 g) as an off-white solid. MS m/z 493 (M+H)⁺; ¹H NMR (400 MHz, DMSO-d6) δ 10.70 (s, 1H), 8.40 (s, 1H), 7.55 (m, 1H), 7.35 (m, 2H), 7.25 (m, 1H), 7.05 (m, 4H), 4.15 (m, 2H), 2.60-3.05 (m, 8H), 2.20 (s, 3H), 1.85-2.05 (m, 4H), 1.65 (m, 5H), 1.15 (m, 2H).

Using the procedure of Example 5 and known appropriate reagents and starting materials, the following compounds of the invention were prepared:

| Cpd | Name | MS |
|---|---|---|
| 20 | [1-(3,4-dichloro-phenylcarbamoyl)-piperidin-4-yl]-[4-(1H-indol-3-yl)- | 529 |
| | piperidin-1-yl]-acetic acid | |
| 28 | [1-(3,5-difluoro-phenylcarbamoyl)-piperidin-4-yl]-[4-(1H-indol-3-yl)- | 497 |
| | piperidin-1-yl]-acetic acid | |
| 32 | [4-(1H-indol-3-yl)-piperidin-1-yl]-(1-phenylcarbamoyl-piperidin-4-yl)- | 461 |
| | acetic acid | |
| 33 | [1-(3,5-dichloro-phenylcarbamoyl)-piperidin-4-yl]-[4-(1H-indol-3-yl)- | 529 |
| | piperidin-1-yl]-acetic acid | |
| 40 | [1-(3,4-difluoro-phenylcarbamoyl)-piperidin-4-yl]-[4-(1H-indol-3-yl)- | 497 |
| | piperidin-1-yl]-acetic acid | |
| 48 | [1-(3-chloro-4-fluoro-phenylcarbamoyl)-piperidin-4-yl]-[4-(1H-indol-3- | 513 |
| | yl)-piperidin-1-yl]-acetic acid | |
| 49 | [1-(3-chloro-4-methyl-phenylcarbamoyl)-piperidin-4-yl]-[4-(1H-indol-3- | 509 |
| | yl)-piperidin-1-yl]-acetic acid | |
| 57 | [1-(4-chloro-3-trifluoromethyl-phenylcarbamoyl)-piperidin-4-yl]-[4- | 563 |
| | (1H-indol-3-yl)-piperidin-1-yl]-acetic acid | |
| 58 | [1-(4-fluoro-3-trifluoromethyl-phenylcarbamoyl)-piperidin-4-yl]-[4-(1H- | 547 |
| | indol-3-yl)-piperidin-1-yl]-acetic acid | |
| 65 | [1-(3-fluoro-5-trifluoromethyl-phenylcarbamoyl)-piperidin-4-yl]-[4-(1H- | 547 |
| | indol-3-yl)-piperidin-1-yl]-acetic acid | |
| 66 | [1-(3,4-dimethoxy-phenylcarbamoyl)-piperidin-4-yl]-[4-(1H-indol-3-yl)- | 521 |
| | piperidin-1-yl]-acetic acid | |
| 67 | [1-(3-chloro-4-methoxy-phenylcarbamoyl)-piperidin-4-yl]-[4-(1H-indol- | 525 |
| | 3-yl)-piperidin-1-yl]-acetic acid | |
| 68 | 4-[(4-{carboxy-[4-(1H-indol-3-yl)-piperidin-1-yl]-methyl}-piperidine-1- | 519 |
| | carbonyl)-amino]-benzoic acid methyl ester | |
| 69 | [4-(1H-indol-3-yl)-piperidin-1-yl]-[1-(4-methoxy-phenylcarbamoyl)- | 491 |
| | piperidin-4-yl]-acetic acid | |
| 84 | [1-(3,4-dichloro-benzylcarbamoyl)-piperidin-4-yl]-[4-(1H-indol-3-yl)- | 543 |
| | piperidin-1-yl]-acetic acid | |
| 85 | [1-(3-bromo-phenylcarbamoyl)-piperidin-4-yl]-[4-(1H-indol-3-yl)- | 539 |
| | piperidin-1-yl]-acetic acid | |
| 86 | [1-(3-chloro-phenylcarbamoyl)-piperidin-4-yl]-[4-(1H-indol-3-yl)- | 495 |
| | piperidin-1-yl]-acetic acid | |
| 88 | [1-(4-chloro-phenylcarbamoyl)-piperidin-4-yl]-[4-(1H-indol-3-yl)- | 495 |
| | piperidin-1-yl]-acetic acid | |
| 89 | [1-(4-bromo-phenylcarbamoyl)-piperidin-4-yl]-[4-(1H-indol-3-yl)- | 539 |
| | piperidin-1-yl]-acetic acid | |
| 90 | [1-(4-fluoro-phenylcarbamoyl)-piperidin-4-yl]-[4-(1H-indol-3-yl)- | 479 |
| | piperidin-1-yl]-acetic acid | |
| 91 | [1-(3-fluoro-phenylcarbamoyl)-piperidin-4-yl]-[4-(1H-indol-3-yl)- | 479 |
| | piperidin-1-yl]-acetic acid | |
| 94 | [1-(3,4-dichloro-phenylcarbamoyl)-piperidin-4-yl]-[4-(5-methoxy-1H- | 559 |
| | indol-3-yl)-piperidin-1-yl]-acetic acid | |
| 95 | [1-(3,4-dichloro-phenylcarbamoyl)-piperidin-4-yl]-[4-(6-methoxy-1H- | 559 |
| | indol-3-yl)-piperidin-1-yl]-acetic acid | |
| 96 | [4-(1H-indol-3-yl)-piperidin-1-yl]-[1-(4-trifluoromethyl- | 529 |
| | phenylcarbamoyl)-piperidin-4-yl]-acetic acid | |
| 97 | [4-(1H-indol-3-yl)-piperidin-1-yl]-[1-(3-trifluoromethyl- | 529 |
| | phenylcarbamoyl)-piperidin-4-yl]-acetic acid | |
| 98 | [4-(1H-indol-3-yl)-piperidin-1-yl]-(1-m-tolylcarbamoyl-piperidin-4-yl)- | 475 |
| | acetic acid | |
| 99 | [4-(1H-indol-3-yl)-piperidin-1-yl]-(1-p-tolylcarbamoyl-piperidin-4-yl)- | 475 |
| | acetic acid | |
| 100 | [1-(3,4-dimethyl-phenylcarbamoyl)-piperidin-4-yl]-[4-(1H-indol-3-yl)- | 489 |
| | piperidin-1-yl]-acetic acid | |
| 101 | [1-(4-bromo-3-methyl-phenylcarbamoyl)-piperidin-4-yl]-[4-(1H-indol-3- | 553 |
| | yl)-piperidin-1-yl]-acetic acid | |
| 105 | [1-(3-fluoro-4-methyl-phenylcarbamoyl)-piperidin-4-yl]-[4-(1H-indol-3- | 493 |
| | yl)-piperidin-1-yl]-acetic acid | |
| 106 | [4-(1H-indol-3-yl)-piperidin-1-yl]-[1-(4-methyl-3-trifluoromethyl- | 543 |
| | phenylcarbamoyl)-piperidin-4-yl]-acetic acid | |
| 109 | [1-(3,4-dichloro-phenylcarbamoyl)-piperidin-4-yl]-[4-(5- | 622 |
| | methanesulfonylamino-1H-indol-3-yl)-piperidin-1-yl]-acetic acid | |
| 110 | [1-(2,3-dichloro-phenylcarbamoyl)-piperidin-4-yl]-[4-(1H-indol-3-yl)- | 529 |
| | piperidin-1-yl]-acetic acid | |
| 111 | [1-(2,4-dichloro-phenylcarbamoyl)-piperidin-4-yl]-[4-(1H-indol-3-yl)- | 529 |
| | piperidin-1-yl]-acetic acid | |
| 117 | [1-(4-chloro-2-fluoro-phenylcarbamoyl)-piperidin-4-yl]-[4-(1H-indol-3- | 513 |
| | yl)-piperidin-1-yl]-acetic acid | |
| 125 | [4-(1H-indol-3-yl)-piperidin-1-yl]-[1-(2,3,4-trifluoro-phenylcarbamoyl)- | 515 |
| | piperidin-4-yl]-acetic acid | |
| 126 | [4-(1H-indol-3-yl)-piperidin-1-yl]-[1-(2,4,5-trichloro-phenylcarbamoyl)- | 563 |
| | piperidin-4-yl]-acetic acid | |
| 127 | [4-(1H-indol-3-yl)-piperidin-1-yl]-[1-(4-methylsulfanyl- | 507 |
| | phenylcarbamoyl)-piperidin-4-yl]-acetic acid | |
| 135 | [1-(3,5-dimethyl-phenylcarbamoyl)-piperidin-4-yl]-[4-(1H-indol-3-yl)- | 489 |
| | piperidin-1-yl]-acetic acid | |
| 136 | [1-(3,5-bis-trifluoromethyl-phenylcarbamoyl)-piperidin-4-yl]-[4-(1H- | 597 |
| | indol-3-yl)-piperidin-1-yl]-acetic acid | |
| 142 | [4-(1H-indol-3-yl)-piperidin-1-yl]-[1-(4-trifluoromethylsulfanyl- | 561 |
| | phenylcarbamoyl)-piperidin-4-yl]-acetic acid | |
| 143 | [4-(1H-indol-3-yl)-piperidin-1-yl]-[1-(4-trifluoromethoxy- | 545 |
| | phenylcarbamoyl)-piperidin-4-yl]-acetic acid | |
| 144 | [4-(1H-indol-3-yl)-piperidin-1-yl]-[1-(3-methylsulfanyl- | 507 |
| | phenylcarbamoyl)-piperidin-4-yl]-acetic acid | |

### EXAMPLE 6

### [1-(3,5-dichloro-phenylthiocarbamoyl)-piperidin-4-yl]-[4-(1H-indol-3-yl)-piperidin-1-yl]-acetic acid (Cpd 45)

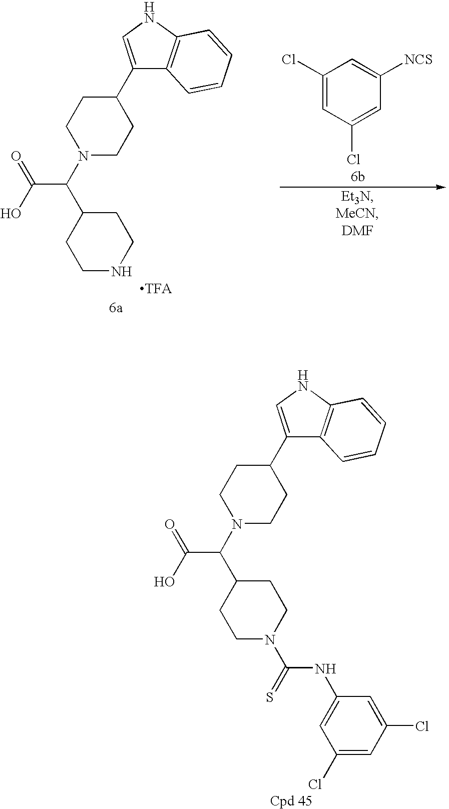

A solution of a TFA salt of [4-(1H-indol-3-yl)-piperidin-1-yl]-piperidin-4-yl-acetic acid Compound 6a (35 mg, 0.076 mmol, 1 eq) and Et₃N (32 μL, 0.23 mmol, 3 eq) in DMF (1 mL) and MeCN (1 mL) was treated with 3,5-dichloro-phenylisothiocyanate Compound 6b (22 mg, 0.11 mmol, 1.5 eq). The mixture was stirred for 16 hrs and then diluted with MeCN resulting in the formation of a tan precipitate. The precipitate was collected by filtration, washed with MeCN and dried to provide Compound 45 (30 mg, 73%) as a tan solid. MS: m/z 545 (M+H)⁺; ¹H NMR (d⁶-DMSO, 400 MHz) δ: 10.76 (1H, s), 9.41 (1H, s), 7.55 (1H, d, J=7.7 Hz), 7.43 (1H, s), 7.43 (1H, s), 7.32 (1H, d, J=8.3 Hz), 7.27 (1H, app t, J=1.6 Hz), 7.08 (1H, d, J=2.0 Hz), 7.05 (1H, app t, J=6.9 Hz), 6.95 (1H, app t, J=7.4 Hz), 4.70 (2H, m), 3.14 (3H, m), 2.93 (3H, m), 2.75 (1H, m), 2.62 (1H, app t, J=12.8 Hz), 2.36 (1H, app t, J=11.2 Hz), 2.13 (1H, m), 1.95 (3H, m), 1.73 (1H, m), 1.63 (2H, m), 1.26 (2H, m).

Using the procedure of Example 6 and known appropriate reagents and starting materials, the following compounds of the invention were prepared:

| Cpd | Name | MS |
|---|---|---|
| 43 | [4-(1H-indol-3-yl)-piperidin-1-yl]-(1-phenylthiocarbamoyl-piperidin-4- | 477 |
| | yl)-acetic acid | |
| 44 | [1-(2,4-difluoro-phenylthiocarbamoyl)-piperidin-4-yl]-[4-(1H-indol-3- | 513 |
| | yl)-piperidin-1-yl]-acetic acid | |
| 55 | [1-(3,5-difluoro-phenylthiocarbamoyl)-piperidin-4-yl]-[4-(1H-indol-3- | 513 |
| | yl)-piperidin-1-yl]-acetic acid | |
| 56 | [1-(3-bromo-phenylthiocarbamoyl)-piperidin-4-yl]-[4-(1H-indol-3-yl)- | 555 |
| | piperidin-1-yl]-acetic acid | |
| 64 | [1-(3,4-dichloro-phenylthiocarbamoyl)-piperidin-4-yl]-[4-(1H-indol-3- | 545 |
| | yl)-piperidin-1-yl]-acetic acid | |
| 78 | [4-(1H-indol-3-yl)-piperidin-1-yl]-(1-p-tolylthiocarbamoyl-piperidin-4- | 491 |
| | yl)-acetic acid | |
| 79 | [4-(1H-indol-3-yl)-piperidin-1-yl]-[1-(3-trifluoromethyl- | 545 |
| | phenylthiocarbamoyl)-piperidin-4-yl]-acetic acid | |
| 80 | [4-(1H-indol-3-yl)-piperidin-1-yl]-[1-(4-trifluoromethyl- | 545 |
| | phenylthiocarbamoyl)-piperidin-4-yl]-acetic acid | |

### EXAMPLE 7

### {1-[(3,5-difluoro-benzoylamino)-imino-methyl]-piperidin-4-yl}-[4-(1H-indol-3-yl)-piperidin-1-yl]-acetic acid (Cpd 51)

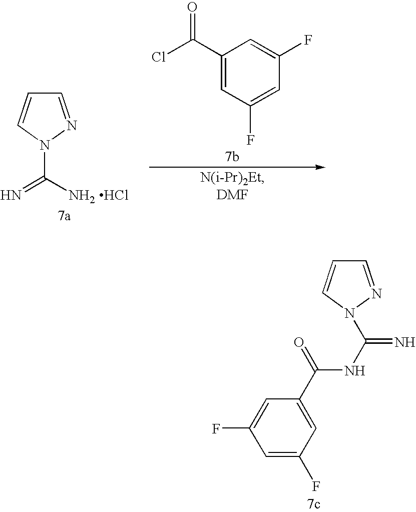

DIPEA (348 μL, 2.00 mmol, 2 eq) was added to a solution of pyrazole-1-carboxamidine Compound 7a (146 mg, 1.00 mmol, 1 eq) in DMF (2 mL), then 3,5-difluoro-benzoyl-chloride Compound 7b (126 μL, 1.00 mmol, 1 eq) was added with stirring. After 48 hrs, the mixture was poured into EtOAc and a dilute NH₄Cl solution. The aqueous layer was removed, the organic layer was washed twice with brine then dried over anhydrous Na₂SO₄. The solid was removed by filtration and the filtrate was evaporated to provide an off-white solid. The crude product was heated in a minimal amount of 3:2:1 CH₂Cl₂:hexanes:EtOAc and then cooled to room temperature. A precipitate formed and was collected by filtration to provide 3,5-difluoro-N-(imino-pyrazol-1-yl-methyl)-benzamide Compound 7c (105 mg, 42%) as a white solid. MS m/z 251 (M+H)⁺.

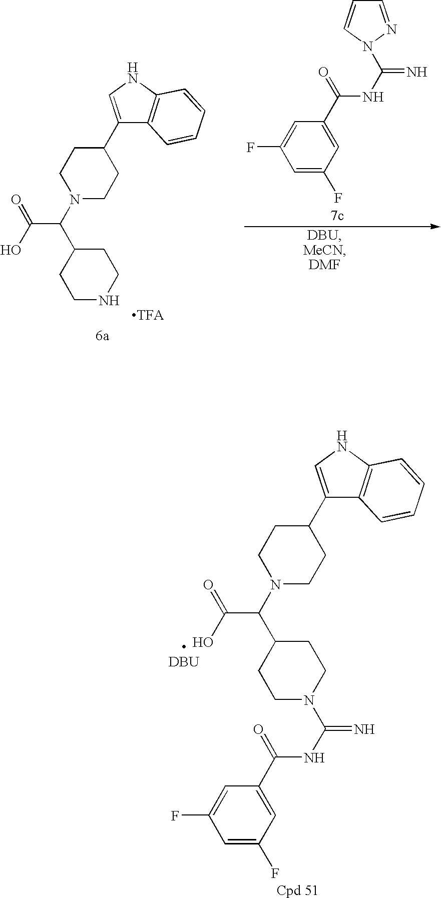

A solution of the TFA salt of [4-(1H-indol-3-yl)-piperidin-1-yl]-piperidin-4-yl-acetic acid Compound 6a (34 mg, 0.075 mmol, 1 eq) and DBU (26 μL, 0.17 mmol, 2.2 eq) in DMF (1 mL) and MeCN (1 mL) was treated with Compound 7c (19 mg, 0.075 mmol, 1 eq) and stirred for 24 hrs. The reaction was then diluted with MeCN, resulting in the formation of a tan precipitate. The precipitate was collected by filtration, washed with MeCN and dried to provide a DBU salt of Compound 51 (28 mg, 55%) as a tan solid. MS m/z 524 (M+H)⁺; 546 (M+Na)⁺; ¹H NMR (d⁶-DMSO, 400 MHz) δ: 10.76 (1H, s), 7.63 (1H, d, J=8.8 Hz), 7.62 (1H, d, J=8.6 Hz), 7.51 (1H, d, J=7.8 Hz), 7.33 (1H, m), 7.31 (1H, d, J=7.9 Hz), 7.01-7.05 (2H, m), 6.94 (1H, app t, J=7.2 Hz), 3.49 (2H, m), 3.42 (2H, m), 3.24 (2H, m), 2.94-2.80 (3H, m), 2.77-2.59 (5H, m), 2.49 (obscured)-2.40 (1H, m), 1.99-1.82 (6H, m), 1.74 (1H, m), 1.70-1.48 (8H, m), 1.08 (2H, m).

Using the procedure of Example 7 and known appropriate reagents and starting materials, the following compounds of the invention were prepared:

| Cpd | Name | MS |
|---|---|---|
| 50 | {1-[(3,4-dichloro-benzoylamino)-imino-methyl]- | 556 |
| | piperidin-4-yl}-[4-(1H-indol-3-yl)-piperidin-1-yl]-acetic | |
| | acid | |
| 52 | {1-[imino-(3,4,5-trifluoro-benzoylamino)-methyl]- | 542 |
| | piperidin-4-yl}-[4-(1H-indol-3-yl)-piperidin-1-yl]-acetic | |
| | acid | |
| 54 | {1-[(3-fluoro-benzoylamino)-imino-methyl]- | 506 |
| | piperidin-4-yl}-[4-(1H-indol-3-yl)-piperidin-1-yl]-acetic | |
| | acid | |
| 73 | {1-[imino-(3-trifluoromethyl-benzoylamino)-methyl]- | 556 |
| | piperidin-4-yl}-[4-(1H-indol-3-yl)-piperidin-1-yl]-acetic | |
| | acid | |

### EXAMPLE 8

### [4-(1-acetyl-1H-indol-3-yl)-piperidin-1-yl]-{1-[(2E)-3-(3,5-difluoro-phenyl)-acryloyl]-piperidin-4-yl}-acetic acid (Cpd 21)

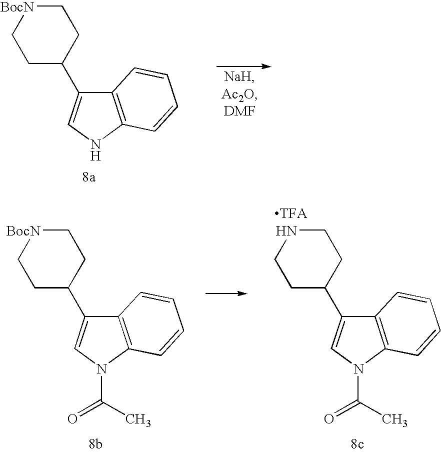

A solution of 4-(1H-indol-3-yl)-piperidine-1-carboxylic acid tert-butyl ester Compound 8a (95 mg, 0.32 mmol, 1 eq) in DMF (3 mL) was treated with NaH (17 mg, 0.35 mmol, 1.1 eq) and stirred for 30 min. Acetic anhydride (33 μL, 0.35 mmol, 1.1 eq) was added and the reaction mixture was stirred for 3 hrs. The mixture was partitioned between EtOAc and water and the aqueous layer was discarded. The organic layer was washed with brine, dried over Na₂SO₄, then filtered and the filtrate was evaporated. Purification of the crude residue by silica gel chromatography (2:1 hexanes:EtOAc) provided 4-(]-acetyl-1H-indol-3-yl)-piperidine-1-carboxylic acid tert-butyl ester Compound 8b (98 mg, 89%) as an oil. MS: m/z 365 (M+Na)⁺.

A solution of Compound 8b (59 mg, 0.17 mmol, 1 eq) in CH₂Cl₂ (1.5 mL) was cooled to 0° C. and treated with TFA (0.5 mL) with stirring. After stirring for 4 hrs, the reaction mixture was allowed to warm to room temperature, the volatiles were removed to provide a TFA salt of 4-(1-acetyl-1H-indol-3-yl)-piperidine Compound 8c as an oil, which was used in the next step without further purification. MS m/z 243 (M+H⁺).

Using the procedure of Example 1, Compound 8c was used in place of Compound 1f and carried forward to provide Compound 21. MS m/z 550 (M+H)⁺.

### EXAMPLE 9

### (2E)-1-(4-{2-hydroxy-1-[4-(1H-indol-3-yl)-piperidin-1-yl]-ethyl}-piperidin-1-yl)-3-(3,4,5-trifluoro-phenyl)-propenone (Cpd 112)

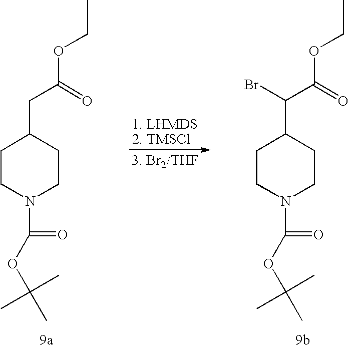

4-ethoxycarbonylmethyl-piperidine-1-carboxylic acid tert-butyl ester Compound 9a (12.4 g, 45.7 mmol, 1 eq) was dissolved in THF (40 mL) and cooled to −78° C. LHMDS (1M solution in THF, 82 mL, 82.3 mmol, 1.8 eq) was added dropwise with stirring. After 45 min, TMSCl (10.4 mL, 82.3 mmol, 1.8 eq) was added to the lithium enolate, and the resulting solution was stirred at −78° C. for 1 hr. Bromine (2.3 mL, 45.7 mmol, 1 eq) was then added, and the reaction was stirred for 2 hrs at −78° C. The mixture was then warmed to room temperature over 30 min, quenched with saturated aqueous NaHCO₃ and partitioned between EtOAc and saturated aqueous NaHCO₃. The aqueous layer was removed and extracted again with EtOAc. The organic layers were combined and washed twice with brine. The organic layer was dried over anhydrous sodium sulfate, the filtered and evaporated to provide a dark orange oil which was purified by silica gel chromatography (4:1 to 1:1 hexanes:EtOAc) to provide 4-(bromo-ethoxycarbonyl-methyl)-piperidine-1-carboxylic acid tert-butyl ester Compound 9b (12.3 g, 82%) as a pale yellow oil. ¹H NMR (CDCl₃, 400 MHz) δ: 4.06 (2H, q, J=6.9 Hz); 3.96 (2H, broad m); 3.81 (1H, d, J=8.5 Hz); 2.53 (2H, m); 1.86 (2H, m); 1.47 (1H, m); 1.28 (9H, s); 1.13 (3H, t, J=6.9 Hz); 1.14-0.96 (2H, m).

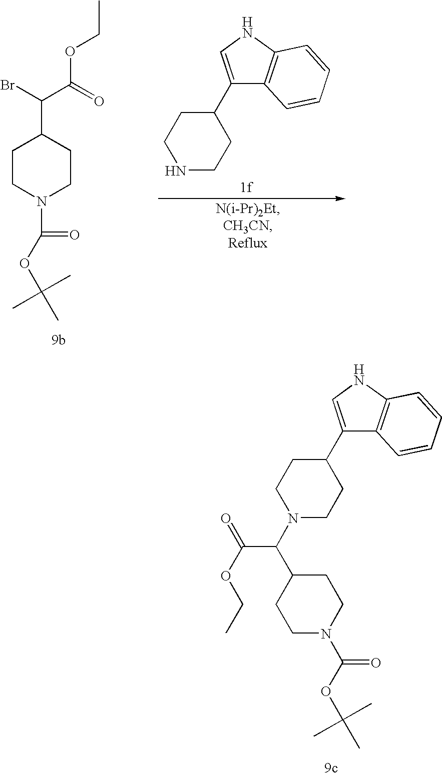

Compound 9b (7.25 g, 20.7 mmol, 1 eq), 3-piperidin-4-yl-1H-indole Compound 1f (4.14 g, 20.7 mmol, 1 eq) and diisopropylethylamine (10.8 mL, 62.1 mmol, 3 eq) were added to MeCN (60 mL) and the resulting solution was heated at reflux for 48 hrs. The reaction was then cooled to room temperature to precipitate unreacted Compound 1f from the solution. The precipitate was removed by filtration and the filtrate evaporated. Silica gel chromatography (3:2:1 to 3:1.5:1 CH₂Cl₂:hexanes:EtOAc) provided 4-{ethoxycarbonyl-[4-(1H-indol-3-yl)-piperidin-1-yl]-methyl}-piperidine-1-carboxylic acid tert-butyl ester Compound 9c (4.73 g, 49%) as a pale foam. MS: m/z 470 (M+H)⁺, 492 (M+Na)⁺.

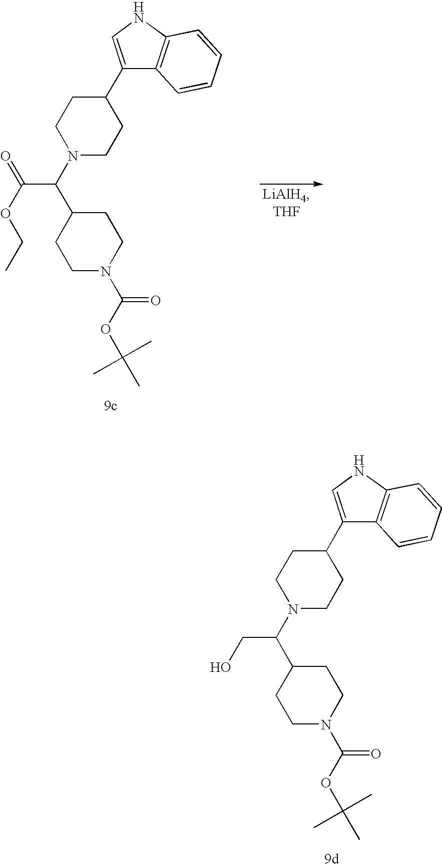

Compound 9c (646 mg, 1.38 mmol, 1 eq) was dissolved in THF (12 mL) and the solution was cooled to 0° C. A 1M solution of LiAlH₄ (2.06 mmol, 1.5 eq) in THF (2 mL) was added dropwise to the solution of Compound 9c. The mixture was stirred for 1.5 hrs, additional LiAlH₄ solution (0.5 mL) was added and the reaction mixture was stirred for an additional 1 hr. The reaction was quenched by sequential addition of water (0.1 mL), 15% NaOH (0.1 mL) and water (0.3 mL). The mixture was stirred for 30 min to form a precipitate. The precipitate was removed by filtration through a pad of celite. The pad was then washed with EtOAc, and the resulting filtrate washed twice with brine. The organic layer was dried over anhydrous sodium sulfate, filtered and the filtrate was evaporated to provide 4-{2-hydroxy-1-[4-(1H-indol-3-yl)-piperidin-1-yl]-ethyl}-piperidine-1-carboxylic acid tert-butyl ester Compound 9d (492 mg, 83%) as a white foam, used in the next step without further purification. MS m/z 428 (M+H)⁺.

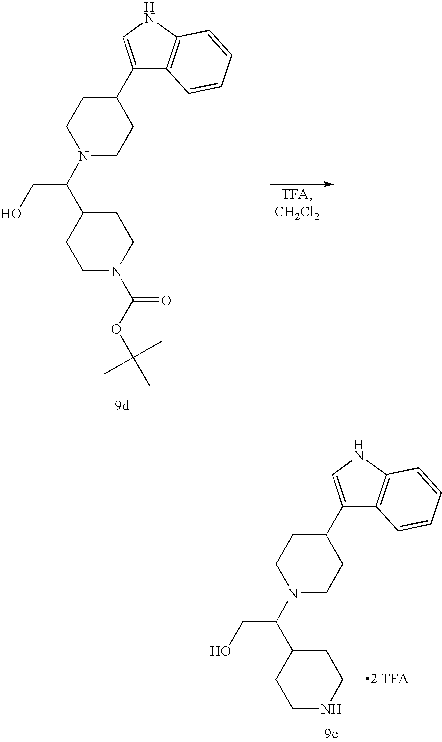

Compound 9d (273 mg, 0.64 mmol, 1 eq) was dissolved in CH₂Cl₂ (1.5 mL) and cooled to 0° C. TFA (0.5 mL) was added dropwise with stirring and the reaction was allowed to slowly warm to room temperature. After 3 hrs, the volatiles were removed in vacuo to provide the bis-trifluoroacetate salt of 2-[4-(1H-indol-3-yl)-piperidin-1-yl]-2-piperidin-4-yl-ethanol Compound 9e as an orange oil that was used in the next step without further purification. MS m/z 328 (M+H)⁺.

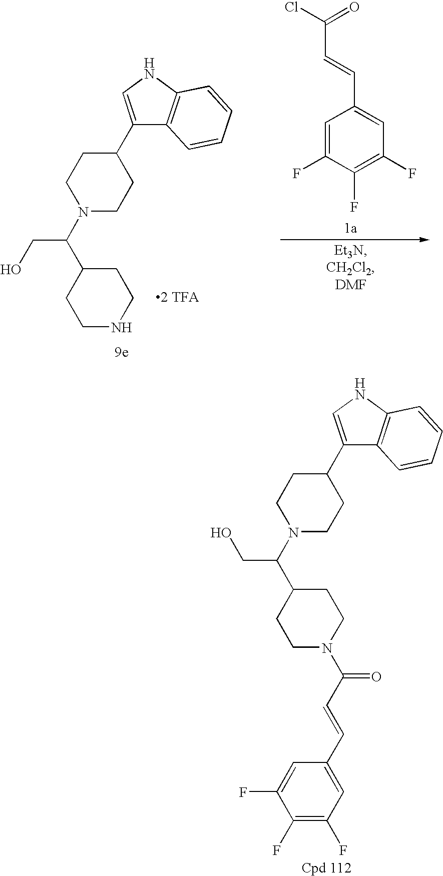

Compound 9e (805 mg, 1.45 mmol, 1 eq) was dissolved in CH₂Cl₂ (10 mL) and DMF (2 mL) and cooled to 0° C. TEA (0.8 mL, 5.80 mmol, 4 eq) was added, followed by slow addition of a solution of 3,4,5-trifluoro-cinnamoyl chloride Compound 1a (320 mg, 1.45 mmol, 1 eq) in CH₂Cl₂ (2 mL) and DMF (3 mL). After stirring overnight, the reaction was allowed to warm to room temperature, the volatiles were removed in vacuo and the resulting residue dissolved in CH₂Cl₂. The solution was washed with saturated aqueous NaHCO₃ and brine. The organic layer was dried with anhydrous Na₂SO₄ and filtered to remove the solid. The filtrate was evaporated and the resulting residue chromatographed using PTLC (8% MeOH in CH₂Cl₂). Isolation of the product band was followed by elution with 10-15% MeOH in CH₂Cl₂. The solvent was removed in vacuo and the residue triturated with methanol to provide Compound 112 (154 mg, 21%) as a white solid. MS m/z 512 (M+H)⁺; 534 (M+Na)⁺; ¹H NMR (d⁶-DMSO, 400 MHz) δ: 10.74 (1H, s), 7.81 (2H, m), 7.52 (1H, d, J=7.9 Hz), 7.39 (2H, s), 7.32 (1H, d, J=7.8 Hz), 7.09-7.01 (2H, m), 6.95 (1H, app t, J=7.4 Hz), 4.47 (1H, broad t, J=11.3 Hz), 4.35-4.27 (2H, m), 3.70-3.62 (1H, m), 3.62-3.54 (1H, m), 3.05 (1H, m), 2.94-2.81 (2H, m), 2.77-2.59 (3H, m), 2.55 (1H, t (partially obscured), J=11.3 Hz), 2.22 (1H, m), 2.03 (1H, m), 1.96-1.74 (4H, m), 1.70-1.50 (2H, m), 1.25-0.99 (2H, m).

Using the procedure of Example 9 and known appropriate reagents and starting materials, the following compounds of the invention were prepared:

| Cpd | Name | MS |
|---|---|---|
| 113 | (2E)-3-(3,4-difluoro-phenyl)-1-(4-{2-hydroxy-1-[4-(1H-indol-3-yl)- | 494 |
| | piperidin-1-yl]-ethyl}-piperidin-1-yl)-propenone | |
| 116 | (2E)-3-(3,5-difluoro-phenyl)-1-(4-{2-hydroxy-1-[4-(1H-indol-3-yl)- | 494 |
| | piperidin-1-yl]-ethyl}-piperidin-1-yl)-propenone | |
| 119 | (2E)-1-(4-{2-hydroxy-1-[4-(1H-indol-3-yl)-piperidin-1-yl]-ethyl}- | 526 |
| | piperidin-1-yl)-3-(3-trifluoromethyl-phenyl)-propenone | |
| 121 | (2E)-3-(3,4-dichloro-phenyl)-1-(4-{2-hydroxy-1-[4-(1H-indol-3-yl)- | 526 |
| | piperidin-1-yl]-ethyl}-piperidin-1-yl)-propenone | |
| 123 | 4-{2-hydroxy-1-[4-(1H-indol-3-yl)-piperidin-1-yl]-ethyl}-piperidine-1- | 515 |
| | carboxylic acid (3,4-dichloro-phenyl)-amide | |
| 129 | 4-{2-hydroxy-1-[4-(1H-indol-3-yl)-piperidin-1-yl]-ethyl}-piperidine-1- | 483 |
| | carboxylic acid (3,5-difluoro-phenyl)-amide | |
| 131 | 4-{2-hydroxy-1-[4-(7-methoxy-1H-indol-3-yl)-piperidin-1-yl]-ethyl}- | 458 |
| | piperidine-1-carboxylic acid tert-butyl ester | |
| 132 | (2E)-1-(4-{2-hydroxy-1-[4-(6-methoxy-1H-indol-3-yl)-piperidin-1-yl]- | 542 |
| | ethyl}-piperidin-1-yl)-3-(3,4,5-trifluoro-phenyl)-propenone | |
| 133 | (2E)-1-(4-{2-hydroxy-1-[4-(7-methoxy-1H-indol-3-yl)-piperidin-1-yl]- | 542 |
| | ethyl}-piperidin-1-yl)-3-(3,4,5-trifluoro-phenyl)-propenone | |
| 137 | (2E)-3-(3,5-difluoro-phenyl)-1-(4-{2-hydroxy-1-[4-(4-methoxy- | 485 |
| | phenyl)-piperidin-1-yl]-ethyl}-piperidin-1-yl)-propenone | |
| 138 | (2E)-3-(3,4-difluoro-phenyl)-1-(4-{2-hydroxy-1-[4-(4-methoxy- | 485 |
| | phenyl)-piperidin-1-yl]-ethyl}-piperidin-1-yl)-propenone | |
| 139 | (2E)-3-(3,4-dichloro-phenyl)-1-(4-{2-hydroxy-1-[4-(4-methoxy- | 517 |
| | phenyl)-piperidin-1-yl]-ethyl}-piperidin-1-yl)-propenone | |
| 140 | (2E)-1-(4-{2-hydroxy-1-[4-(4-methoxy-phenyl)-piperidin-1-yl]-ethyl}- | 503 |
| | piperidin-1-yl)-3-(2,4,5-trifluoro-phenyl)-propenone | |
| 141 | 4-{2-hydroxy-1-[4-(4-methoxy-phenyl)-piperidin-1-yl]-ethyl}- | 474 |
| | piperidine-1-carboxylic acid (3,4-difluoro-phenyl)-amide | |
| 153 | (2E)-1-(4-{2-hydroxy-1-[4-(1H-pyrrolo[2,3-b]pyridin-3-yl)-piperidin- | 513 |
| | 1-yl]-ethyl}-piperidin-1-yl)-3-(3,4,5-trifluoro-phenyl)-propenone | |
| 154 | benzofuran-2-yl-(4-{2-hydroxy-1-[4-(1H-indol-3-yl)-piperidin-1-yl]- | 472 |
| | ethyl}-piperidin-1-yl)-methanone | |
| 155 | (2E)-3-(3,4-dichloro-phenyl)-1-(4-{2-hydroxy-1-[4-(1H-pyrazol-3-yl)- | 477 |
| | piperidin-1-yl]-ethyl}-piperidin-1-yl)-propenone | |
| 156 | (2E)-1-(4-{2-hydroxy-1-[4-(1H-pyrazol-3-yl)-piperidin-1-yl]-ethyl}- | 463 |
| | piperidin-1-yl)-3-(3,4,5-trifluoro-phenyl)-propenone | |
| 157 | (5-chloro-benzofuran-2-yl)-(4-{2-hydroxy-1-[4-(1H-indol-3-yl)- | 506 |
| | piperidin-1-yl]-ethyl}-piperidin-1-yl)-methanone | |
| 158 | (2E)-1-(4-{2-hydroxy-1-[4-(4-methoxy-phenyl)-piperidin-1-yl]-ethyl}- | 503 |
| | piperidin-1-yl)-3-(3,4,5-trifluoro-phenyl)-propenone | |
| 159 | (2E)-1-(4-{2-hydroxy-1-[4-(4-methoxy-phenyl)-piperidin-1-yl]-ethyl}- | 449 |
| | piperidin-1-yl)-3-phenyl-propenone | |
| 160 | (5-chloro-benzofuran-2-yl)-(4-{2-hydroxy-1-[4-(4-methoxy-phenyl)- | 497 |
| | piperidin-1-yl]-ethyl}-piperidin-1-yl)-methanone | |
| 161 | (2E)-3-(3-bromo-4-fluoro-phenyl)-1-(4-{2-hydroxy-1-[4-(4-methoxy- | 545 |
| | phenyl)-piperidin-1-yl]-ethyl}-piperidin-1-yl)-propenone | |
| 162 | (2E)-3-(3,4-dichloro-phenyl)-1-(4-{2-hydroxy-1-[4-(5-methoxy-1H- | 556 |
| | indol-3-yl)-piperidin-1-yl]-ethyl}-piperidin-1-yl)-propenone | |
| 163 | (2E)-3-(3,4-dichloro-phenyl)-1-(4-{2-hydroxy-1-[4-(6-methoxy-1H- | 556 |
| | indol-3-yl)-piperidin-1-yl]-ethyl}-piperidin-1-yl)-propenone | |
| 164 | (2E)-3-(3,5-difluoro-phenyl)-1-(4-{2-hydroxy-1-[4-(5-methoxy-1H- | 524 |
| | indol-3-yl)-piperidin-1-yl]-ethyl}-piperidin-1-yl)-propenone | |
| 165 | (2E)-3-(3,5-difluoro-phenyl)-1-(4-{2-hydroxy-1-[4-(6-methoxy-1H- | 524 |
| | indol-3-yl)-piperidin-1-yl]-ethyl}-piperidin-1-yl)-propenone | |
| 166 | (2E)-1-(4-{2-hydroxy-1-[4-(5-methoxy-1H-indol-3-yl)-piperidin-1-yl]- | 542 |
| | ethyl}-piperidin-1-yl)-3-(3,4,5-trifluoro-phenyl)-propenone | |
| 167 | (2E)-1-(4-{2-hydroxy-1-[4-(6-methoxy-1H-indol-3-yl)-piperidin-1-yl]- | 542 |
| | ethyl}-piperidin-1-yl)-3-(3,4,5-trifluoro-phenyl)-propenone | |
| 168 | (2E)-3-(3,4-dichloro-phenyl)-1-(4-{1-[4-(5-fluoro-1H-indol-3-yl)- | 544 |
| | piperidin-1-yl]-2-hydroxy-ethyl}-piperidin-1-yl)-propenone | |
| 169 | (2E)-1-(4-{1-[4-(5-fluoro-1H-indol-3-yl)-piperidin-1-yl]-2-hydroxy- | 494 |
| | ethyl}-piperidin-1-yl)-3-(4-fluoro-phenyl)-propenone | |
| 170 | (2E)-1-(4-{1-[4-(5-fluoro-1H-indol-3-yl)-piperidin-1-yl]-2-hydroxy- | 530 |
| | ethyl}-piperidin-1-yl)-3-(3,4,5-trifluoro-phenyl)-propenone | |
| 171 | (2E)-3-(3,5-difluoro-phenyl)-1-(4-{1-[4-(5-fluoro-1H-indol-3-yl)- | 512 |
| | piperidin-1-yl]-2-hydroxy-ethyl}-piperidin-1-yl)-propenone | |
| 172 | (2E)-3-(3-bromo-4-fluoro-phenyl)-1-(4-{1-[4-(5-fluoro-1H-indol-3- | 572 |
| | yl)-piperidin-1-yl]-2-hydroxy-ethyl}-piperidin-1-yl)-propenone | |
| 173 | (2E)-3-(3,5-difluoro-phenyl)-1-(4-{2-hydroxy-1-[4-(1H-indazol-3-yl)- | 495 |
| | piperidin-1-yl]-ethyl}-piperidin-1-yl)-propenone | |
| 174 | (2E)-1-(4-{1-[4-(1H-benzoimidazol-2-yl)-piperidin-1-yl]-2-hydroxy- | 495 |
| | ethyl}-piperidin-1-yl)-3-(3,5-difluoro-phenyl)-propenone | |
| 175 | (2E)-1-(4-{1-[4-(1H-benzoimidazol-2-yl)-piperidin-1-yl]-2-hydroxy- | 513 |
| | ethyl}-piperidin-1-yl)-3-(3,4,5-trifluoro-phenyl)-propenone | |
| 176 | (2E)-1-(4-{1-[4-(1H-benzoimidazol-2-yl)-piperidin-1-yl]-2-hydroxy- | 527 |
| | ethyl}-piperidin-1-yl)-3-(3,4-dichloro-phenyl)-propenone | |
| 182 | (2E)-3-(3,5-difluoro-phenyl)-1-{4-[2-hydroxy-1-(3,4,5,6-tetrahydro- | 456 |
| | 2H-[4,4′]bipyridinyl-1-yl)-ethyl]-piperidin-1-yl}-propenone | |
| 183 | (2E)-1-{4-[2-hydroxy-1-(3,4,5,6-tetrahydro-2H-[4,4′]bipyridinyl-1-yl)- | 474 |
| | ethyl]-piperidin-1-yl}-3-(3,4,5-trifluoro-phenyl)-propenone | |
| 184 | (2E)-1-{4-[2-hydroxy-1-(3,4,5,6-tetrahydro-2H-[4,4′]bipyridinyl-1-yl)- | 488 |
| | ethyl]-piperidin-1-yl}-3-(3-trifluoromethyl-phenyl)-propenone | |
| 185 | (2E)-3-(3,4-dichloro-phenyl)-1-{4-[2-hydroxy-1-(3,4,5,6-tetrahydro- | 488 |
| | 2H-[4,4′]bipyridinyl-1-yl)-ethyl]-piperidin-1-yl}-propenone | |
| 186 | (2E)-3-(3-bromo-4-fluoro-phenyl)-1-{4-[2-hydroxy-1-(3,4,5,6- | 516 |
| | tetrahydro-2H-[4,4′]bipyridinyl-1-yl)-ethyl]-piperidin-1-yl}-propenone | |
| 191 | (2E)-1-(4-{1-[4-(5-amino-1H-pyrrolo[3,2-b]pyridin-3-yl)-piperidin-1- | 528 |
| | yl]-2-hydroxy-ethyl}-piperidin-1-yl)-3-(3,4,5-trifluoro-phenyl)- | |
| | propenone | |
| 198 | N-{3-[1-(1-{1-[(2E)-3-(3,4-dichloro-phenyl)-acryloyl]-piperidin-4-yl}- | 619 |
| | 2-hydroxy-ethyl)-piperidin-4-yl]-1H-indol-5-yl}-methanesulfonamide | |
| 201 | N-{3-[1-(2-hydroxy-1-{1-[(2E)-3-(3,4,5-trifluoro-phenyl)-acryloyl]- | 605 |
| | piperidin-4-yl}-ethyl)-piperidin-4-yl]-1H-indol-5-yl}- | |
| | methanesulfonamide | |
| 202 | (2E)-3-(3,5-difluoro-phenyl)-1-(4-{2-hydroxy-1-[4-(1H-pyrrolo[2,3- | 495 |
| | b]pyridin-3-yl)-piperidin-1-yl]-ethyl}-piperidin-1-yl)-propenone | |
| 205 | (2E)-3-(3,5-difluoro-phenyl)-1-(4-{2-hydroxy-1-[4-(7-oxy-1H- | 511 |
| | pyrrolo[2,3-b]pyridin-3-yl)-piperidin-1-yl]-ethyl}-piperidin-1-yl)- | |
| | propenone | |
| 208 | (2E)-3-(3,4-dichloro-phenyl)-1-(4-{2-hydroxy-1-[4-(1H-pyrrolo[2,3- | 527 |
| | b]pyridin-3-yl)-piperidin-1-yl]-ethyl}-piperidin-1-yl)-propenone | |
| 209 | N-{4-[1-(2-hydroxy-1-{1-[(2E)-3-(3,4,5-trifluoro-phenyl)-acryloyl]- | 566 |
| | piperidin-4-yl}-ethyl)-piperidin-4-yl]-phenyl}-methanesulfonamide | |
| 210 | N-{4-[1-(1-{1-[(2E)-3-(3,4-dichloro-phenyl)-acryloyl]-piperidin-4-yl}- | 580 |
| | 2-hydroxy-ethyl)-piperidin-4-yl]-phenyl}-methanesulfonamide | |
| 212 | [2-(3,4-dichloro-phenyl)-cyclopropyl]-(4-{2-hydroxy-1-[4-(1H-indol- | 540 |
| | 3-yl)-piperidin-1-yl]-ethyl}-piperidin-1-yl)-methanone | |
| 244 | (2E)-1-(4-{1-[4-(4-chloro-phenyl)-piperidin-1-yl]-2-hydroxy-ethyl}- | 521 |
| | piperidin-1-yl)-3-(3,4-dichloro-phenyl)-propenone | |
| 246 | (2E)-1-(4-{1-[4-(4-chloro-phenyl)-piperidin-1-yl]-2-hydroxy-ethyl}- | 507 |
| | piperidin-1-yl)-3-(3,4,5-trifluoro-phenyl)-propenone | |
| 251 | (2E)-3-(4-nitro-phenyl)-acrylic acid 2-[4-(1H-indol-3-yl)-piperidin-1- | 678 |
| | yl]-2-{1-[(2E)-3-(4-nitro-phenyl)-acryloyl]-piperidin-4-yl}-ethyl ester | |
| 253 | 1-(4-{2-hydroxy-1-[4-(4-methoxy-phenyl)-piperidin-1-yl]-ethyl}- | 501 |
| | piperidin-1-yl)-3-(3,4,5-trifluoro-phenyl)-propynone | |
| 254 | (2E)-3-(3,4-difluoro-phenyl)-1-(4-{1-[4-(5-fluoro-1H-indol-3-yl)- | 512 |
| | piperidin-1-yl]-2-hydroxy-ethyl}-piperidin-1-yl)-propenone | |
| 256 | (2E)-1-(4-{1-[4-(5,6-dichloro-1H-benzoimidazol-2-yl)-piperidin-1-yl]- | 563 |
| | 2-hydroxy-ethyl}-piperidin-1-yl)-3-(3,5-difluoro-phenyl)-propenone | |
| 257 | (2E)-1-(4-{1-[4-(5,6-dichloro-1H-benzoimidazol-2-yl)-piperidin-1-yl]- | 581 |
| | 2-hydroxy-ethyl}-piperidin-1-yl)-3-(3,4,5-trifluoro-phenyl)-propenone | |
| 258 | (2E)-3-(4-chloro-phenyl)-1-(4-{1-[4-(4-chloro-phenyl)-piperidin-1-yl]- | 487 |
| | 2-hydroxy-ethyl}-piperidin-1-yl)-propenone | |
| 259 | (2E)-3-(3,5-difluoro-phenyl)-1-(4-{2-hydroxy-1-[4-(5-hydroxy-1H- | 510 |
| | indol-3-yl)-piperidin-1-yl]-ethyl}-piperidin-1-yl)-propenone | |

### EXAMPLE 10

### 4-{2-hydroxy-1-[4-(1H-indol-3-yl)-piperidin-1-yl]-ethyl}-piperidine-1-carbothioic acid (3-trifluoromethyl-phenyl)-amide (Cpd 120)

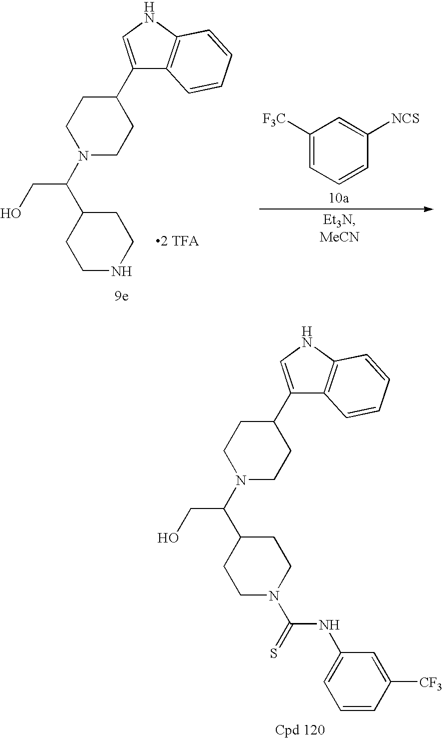

2-[4-(1H-indol-3-yl)-piperidin-1-yl]-2-piperidin-4-yl-ethanol, bis-trifluoroacetate salt Compound 9e (61 mg, 0.11 mmol, 1 eq) and TEA (46 μL, 0.33 mmol, 3 eq) were dissolved in acetonitrile (1 mL). 3-trifluoromethyl-phenylisothiocyanate Compound 10a (17 μL, 0.11 mmol, 1 eq) was added and the mixture stirred overnight at room temperature. The reaction mixture was diluted with CH₂Cl₂, washed once with saturated aqueous NaHCO₃ and washed twice with brine. The organic layer was dried over anhydrous Na₂SO₄. The solids were removed by filtration and the filtrate evaporated to provide an oil that was chromatographed using PTLC (8% MeOH in CH₂Cl₂). Isolation of the product band was followed by elution with 10-15% MeOH in CH₂Cl₂. The solvent was removed in vacuo to provide Compound 120 (35 mg, 60%) as a yellow solid. MS m/z 531 (M+H)⁺.

Using the procedure of Example 10 and known appropriate reagents and starting materials, the following compounds of the invention were prepared:

| Cpd | | MS |
|---|---|---|
| 122 | 4-{2-hydroxy-1-[4-(1H-indol-3-yl)-piperidin-1-yl]-ethyl}- | 531 |
| | piperidine-1-carbothioic acid (3,4-dichloro-phenyl)-amide | |
| 124 | 4-{2-hydroxy-1-[4-(1H-indol-3-yl)-piperidin-1-yl]-ethyl}- | 499 |
| | piperidine-1-carbothioic acid (3,5-difluoro-phenyl)-amide | |

### EXAMPLE 11

### 3,4-dichloro-N-[(4-{2-hydroxy-1-[4-(1H-indol-3-yl)-piperidin-1-yl]-ethyl}-piperidin-1-yl)-imino-methyl]-benzamide (Cpd 128)

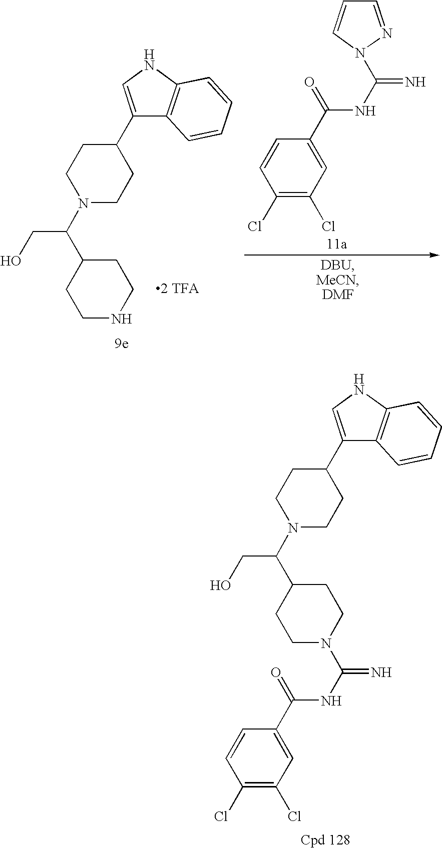

2-[4-(1H-indol-3-yl)-piperidin-1-yl]-2-piperidin-4-yl-ethanol, bis-trifluoroacetate salt Compound 9e (56 mg, 0.10 mmol, 1 eq) and DBU (49 μL, 0.33 mmol, 3.3 eq) were dissolved in DMF (1 mL). 3,4-dichloro-N-(imino-pyrazol-1-yl-methyl)-benzamide Compound 1a (31 mg, 0.11 mmol, 1.1 eq) was added, and the mixture was stirred overnight at room temperature. The volatiles were removed in vacuo and the resulting residue was dissolved in CH₂Cl₂. The solution was washed with saturated aqueous NaHCO₃ and twice with brine. The organic layer was dried with anhydrous Na₂SO₄ then filtered to remove the solid. The filtrate was evaporated and the resulting residue chromatographed, using PTLC (8% MeOH in CH₂Cl₂). Isolation of the product band was followed by elution with 10-15% MeOH in CH₂Cl₂. The solvent was removed in vacuo to provide Compound 128 as an oil.

The oil was dissolved with CH₂Cl₂ and 4N HCl in dioxane was added to form a precipitate which was collected by filtration and washed with dichloromethane to provide the hydrochloride salt of Compound 128 (28 mg, 48%) as a white solid. MS z/z 542 (M+H)⁺.

### EXAMPLE 12

### (2E)-3-(3,5-difluoro-phenyl)-1-(4-{2-dimethylamino-1-[4-(1H-indol-3-yl)-piperidin-1-yl]-ethyl}-piperidin-1-yl)-propenone (Cpd 130)

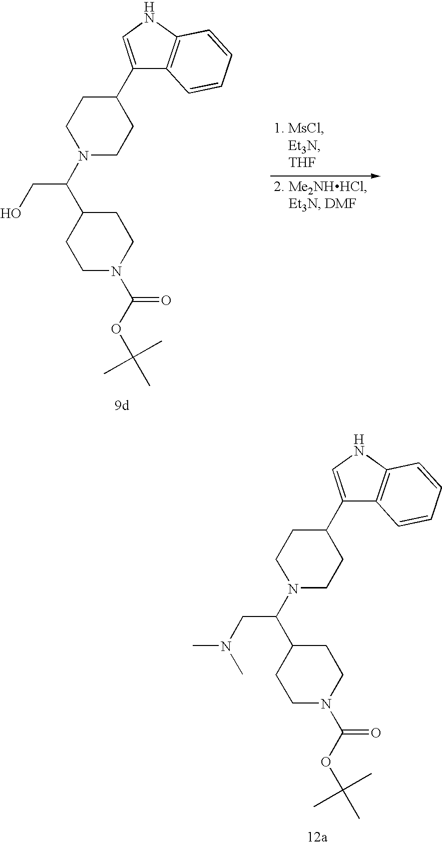

4-{2-hydroxy-1-[4-(1H-indol-3-yl)-piperidin-1-yl]-ethyl}-piperidine-1-carboxylic acid tert-butyl ester Compound 9d (91 mg, 0.21 mmol, 1 eq) and Et₃N (88 μL, 0.63 mmol, 3 eq) were dissolved in THF (2 mL) and cooled to 0° C. MsCl (18 mL, 0.23 mmol, 1.1 eq) was added dropwise and the reaction mixture was stirred for 1.5 hrs. The solvent was removed in vacuo and the residue dissolved in DMF (2 mL). Et₃N (88 μL, 0.63 mmol, 3 eq) and dimethylamine hydrochloride (43 mg, 0.53 mmol, 2.5 eq) were added, and the mixture was stirred for 16 hrs. The volatiles were removed in vacuo and the resulting residue was dissolved in CH₂Cl₂. After washing with saturated aqueous NaHCO₃ and brine, the organic layer was dried over Na₂SO₄ and filtered. The filtrate was evaporated to provide a crude oil which was purified by silica gel column chromatography (10% 2N methanolic ammonia in CH₂Cl₂) to provide 4-{2-dimethylamino-1-[4-(1H-indol-3-yl)-piperidin-1-yl]-ethyl}-piperidine-1-carboxylic acid tert-butyl ester Compound 12a (59 mg, 62%) as an oil. MS m/z 455 (M+H)⁺.

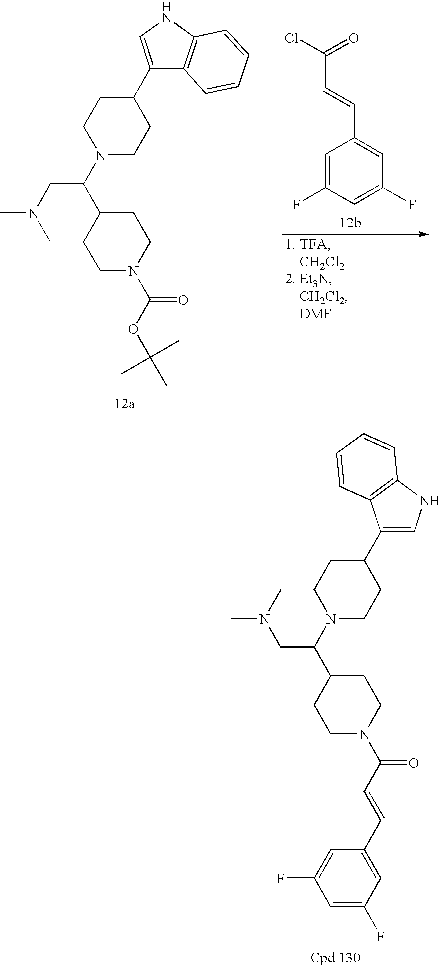

Compound 12a (59 mg, 0.13 mmol, 1 eq) was dissolved in CH₂Cl₂ (1.5 mL) and cooled to 0° C. TFA (0.5 mL) was added dropwise with stirring and the reaction was allowed to warm to room temperature over 3 hrs. The volatiles were removed in vacuo and the resulting residue dissolved in DMF (1 mL) and CH₂Cl₂ (1 mL). Et₃N (54 μL, 0.39 mmol, 3 eq) was added and the solution was cooled to 0° C. 3-(3,5-difluoro-phenyl)-acryloyl chloride Compound 12b (26 mg, 0.13 mmol, 1 eq) was added and the mixture was stirred for 48 hrs. The reaction mixture was allowed to warm to room temperature, the solvents were removed in vacuo and the resulting residue was dissolved in CH₂Cl₂. The solution was washed with saturated aqueous NaHCO₃ and twice with brine, then the organic layer was dried over anhydrous Na₂SO₄ and filtered. The filtrate was evaporated to provide a crude oil, which was purified by silica gel chromatography (10-15% 2N methanolic ammonia in CH₂Cl₂) to provide Compound 130 (30 mg, 44%) as a pale foam. MS m/z 521 (M+H)⁺; ¹H NMR (CDCl₃, 400 MHz) δ 7.98 (1H, s), 7.64 (1H, d, J=7.9 Hz), 7.53 (1H, d, J=15.4 Hz), 7.36 (1H, d, J=0.1 Hz), 7.18 (1H, ddd, J=1.1, 8.2, 8.2 Hz), 7.10 (1H, ddd, J=1.0, 8.2, 8.2), 7.04-6.94 (3H, m), 6.78 (1H, m), 4.69 (1H, broad s), 4.08 (1H, d, J=12.7 Hz), 3.11 (1H, app t, J=12.3 Hz), 2.95 (1H, m), 2.87-2.75 (3H, m), 2.70 (1H, m), 2.61-2.43 (2H, m), 2.43-2.32 (1H, m), 2.23 (6H, s), 2.27-2.15 (1H, m), 2.12-1.94 (3H, m), 1.93-1.82 (1H, m), 1.80-1.58 (3H, m), 1.47-1.23 (2H, m).

### EXAMPLE 13

### N-{2-{1-[(2E)-3-(3,5-difluoro-phenyl)-acryloyl]-piperidin-4-yl}-2-[4-(1H-indol-3-yl)-piperidin-1-yl]-ethyl}-N-methanesulfonyl-methanesulfonamide (Cpd 134)

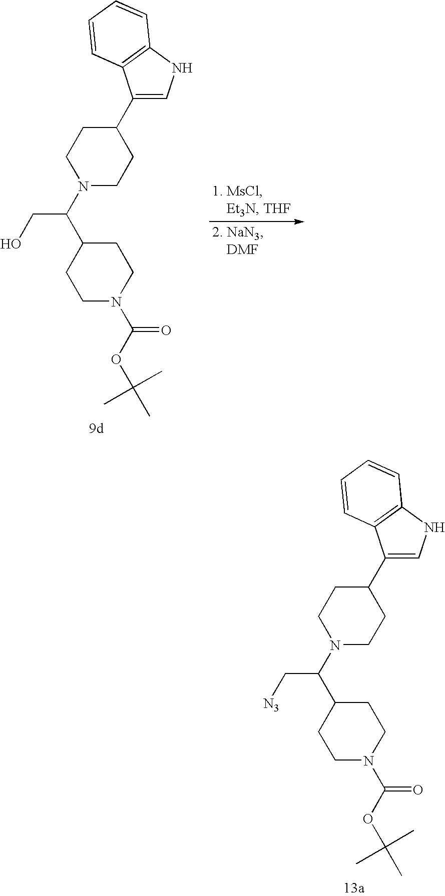

4-{2-hydroxy-1-[4-(1H-indol-3-yl)-piperidin-1-yl]-ethyl}-piperidine-1-carboxylic acid tert-butyl ester Compound 9d (869 mg, 2.03 mmol, 1 eq) and Et₃N (854 μL, 6.09 mmol, 3 eq) were dissolved in THF (21 mL) and cooled to 0° C. MsCl (172 mL, 2.22 mmol, 1.1 eq) was added dropwise and the mixture was stirred for 2 hrs. The solvent was removed in vacuo and the residue dissolved in DMF (7 mL). Sodium azide (330 mg, 5.08 mmol, 2.5 eq) was added and the reaction mixture was stirred for 16 hrs at room temperature. The solvent was removed in vacuo and the resulting residue dissolved in CH₂Cl₂. The solution was washed with saturated aqueous NaHCO₃ and brine, then the organic layer was dried over Na₂SO₄ and filtered. The filtrate was evaporated to provide a crude oil, which was purified by silica gel column chromatography (3:1.5:1 to 3:1:1.5 CH₂Cl₂:hexanes:EtOAc) to provide 4-{2-azido-1-[4-(1H-indol-3-yl)-piperidin-1-yl]-ethyl}-piperidine-1-carboxylic acid tert-butyl ester Compound 13a (560 mg, 61%) as a pale foam. MS m/z 453 (M+H)⁺.

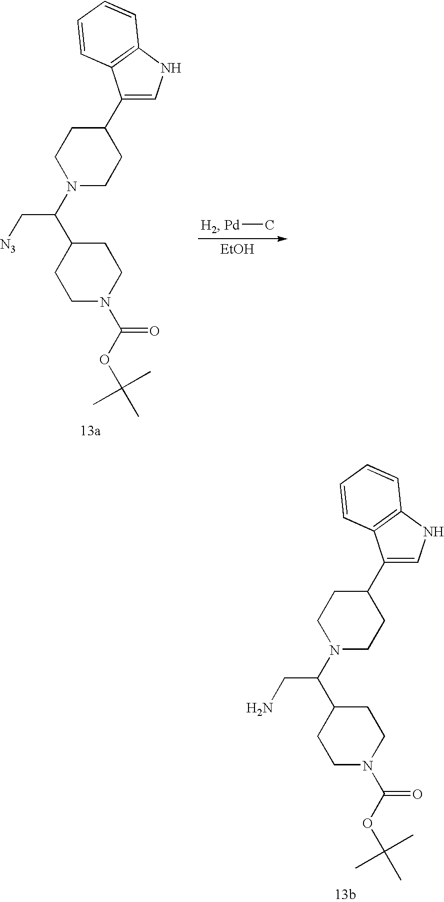

A solution of Compound 13a (560 mg, 1.24 mmol, 1 eq) in absolute ethanol (20 mL) in a bottle was purged with nitrogen for 10 min. Pd-C (palladium on carbon) (10% by weight, 264 mg, 0.25 mmol, 0.2 eq) was added and the bottle was pressurized to 60 psi with hydrogen. The pressure was released and the bottle was refilled again to 60 psi with hydrogen. The pressurization and release was repeated twice more, then the bottle was shaken at 60 psi H₂ for 4 hrs at room temperature. After release of the hydrogen pressure, the solution was purged with nitrogen and filtered through celite. Evaporation of the solvent in vacuo provided 4-{2-amino-1-[4-(1H-indol-3-yl)-piperidin-1-yl]-ethyl}-piperidine-1-carboxylic acid tert-butyl ester Compound 13b (510 mg, 96%) as a pale foam, used in the next step without further purification. MS m(z 427 (M+H)⁺.

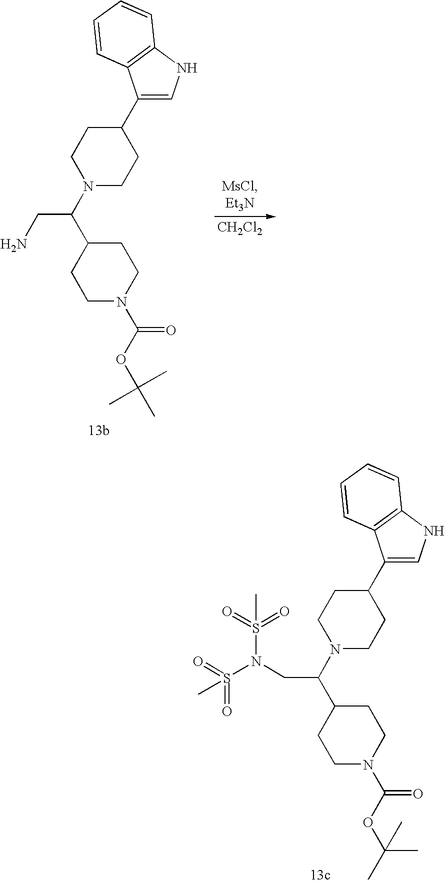

Compound 13b (79 mg, 0.19 mmol, 1 eq) and Et₃N (53 μL, 0.38 mmol, 2 eq) were dissolved in CH₂Cl₂ (1 mL). The mixture was cooled to 0° C. and MsCl (16 μL, 0.20 mmol, 1.1 eq) was added dropwise with stirring. The reaction mixture was stirred for 48 hrs, then the volatiles were removed in vacuo and the residue subjected to silica gel chromatography (3:1:1 CH₂Cl₂:EtOAc:hexanes) to provide 4-{1-[4-(1H-indol-3-yl)-piperidin-1-yl]-2-(dimethanesulfonyl)-amino-ethyl}-piperidine-1-carboxylic acid tert-butyl ester Compound 13c (81 mg, 73%) as a yellow foam. ¹H NMR (CDCl₃, 300 MHz) δ 7.97 (1H, s), 7.61 (1H, d, J=7.8 Hz), 7.36 (1H, d, J=8.0 Hz), 7.19 (1H, app dt, J=0.9, 7.8, 7.8 Hz), 7.10 (1H, app dt, J=0.9, 7.8, 7.8 Hz), 6.94 (1H, d, J=2.0 Hz), 4.25-4.07 (1H, broad m), 4.05 (1H, dd, J=15.4, 11.1 Hz), 3.46 (6H, s), 3.17 (1H, d, J=10.4 Hz), 2.97 (1H, app t, J=11.7), 2.92-2.78 (3H, m), 2.78-2.59 (2H, m), 2.45 (1H, t, J=10.1 Hz), 2.19-2.04 (2H, app t), 1.99-1.84 (1H, m), 1.81-1.50 (5H, m), 1.51-1.37 (1H, m (obscured by 9H singlet)), 1.47 (9H, s), 1.35-1.17 (2H, m).

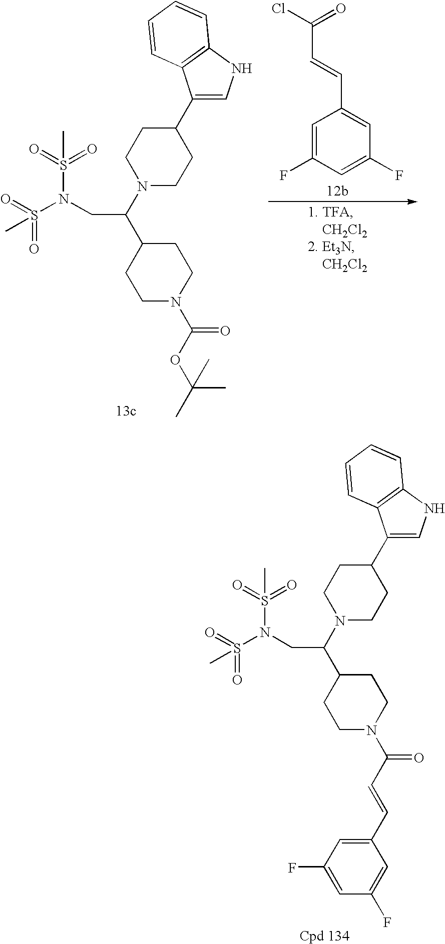

Compound 13c (75 mg, 0.13 mmol, 1 eq) was dissolved in CH₂Cl₂ (3 mL) and cooled to 0° C. TFA (1 mL) was added dropwise with stirring and the reaction was allowed to warm to room temperature over 3 hrs. The volatiles were removed in vacuo to provide an oil that was used in the subsequent reaction without further purification. The deprotected Compound 13c (41 mg, 0.065 mmol, 1 eq) was dissolved in CH₂Cl₂ (1 mL). Et₃N (27 μL, 0.20 mmol, 3 eq) was added to the solution followed by 3-(3,5-difluoro-phenyl)-acryloyl chloride Compound 12b (17 mg, 0.085 mmol, 1.3 eq). After stirring overnight, the reaction was diluted with CH₂Cl₂ and washed with saturated aqueous NaHCO₃ and brine. The organic layer was dried over anhydrous Na₂SO₄ then filtered and the filtrate was evaporated to provide a crude oil, which was chromatographed using PTLC (3:2.5:1 CH₂Cl₂:EtOAc:hexanes). Isolation of the product band was followed by elution with 3:2 CH₂Cl₂:EtOAc. The solvent was removed in 20 vacuo to provide Compound 134 (21 mg) as a pale foam. MS m/z 649 (M+H)⁺; ¹H NMR (CDCl₃, 400 MHz) δ 8.00 (1H, s), 7.61 (1H, d, J=7.8 Hz), 7.56 (1H, d, J=15.4 Hz), 7.37 (1H, d, J=8.1 Hz), 7.19 (1H, ddd, J=7.1, 7.1, 1.1 Hz), 7.10 (1H, ddd, J=7.8, 7.8, 1.1 Hz), 7.02 (2H, m), 6.95 (1H, d, J=2.2 Hz), 6.89 (1H, d, 15.2 Hz), 6.80 (1H, m), 4.76 (1H, broad t, J=11.5 Hz), 4.20-4.10 (1H, m), 4.06 (1H, dd, J=14.9, 10.9 Hz), 3.45 (6H, s), 3.25-3.08 (2H, m), 3.04-2.78 (4H, m), 2.68 (1H, m), 2.48 (1H, m), 2.11 (3H, m), 1.85 (1H, m), 1.81-1.61 (3H, m), 1.55 (1H, m), 1.35 (1H, m).

### EXAMPLE 14

### 4-{2-acetoxy-1-[4-(1H-indol-3-yl)-piperidin-1-yl]-ethyl}-piperidine-1-carboxylic acid tert-butyl ester (Cpd 234)

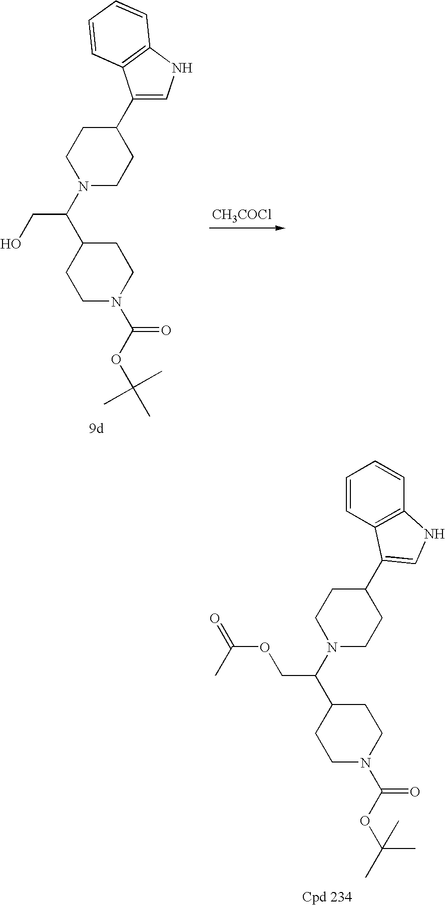

TEA (0.2 g, 2.0 mmol) and acetyl chloride (0.1 mL, 1.4 mmol) were added to a solution of 4-{2-hydroxy-1-[4-(1H-indol-3-yl)-piperidin-1-yl]-ethyl}-piperidine-1-carboxylic acid tert-butyl ester Compound 9d (0.43 g, 1.0 mmol) in methylene chloride (15.0 mL), The mixture was stirred for 2 hrs at r.t. then the reaction was quenched with water. The organic layer was washed with 0.5N HCl (5.0 mL), water (5.0 mL) and brine (5.0 mL), then dried over Na₂SO₄. The methylene chloride was evaporated to provide Compound 234 (0.47 g, 99%) as a white solid. MS m/z 470 (M+H)⁺.

### EXAMPLE 15

### acetic acid 2-[4-(1H-indol-3-yl)-piperidin-1-yl]-2-{1-[(2E)-3-(3,4,5-trifluoro-phenyl)-acryloyl]-piperidin-4-yl}-ethyl ester (Cpd 236)

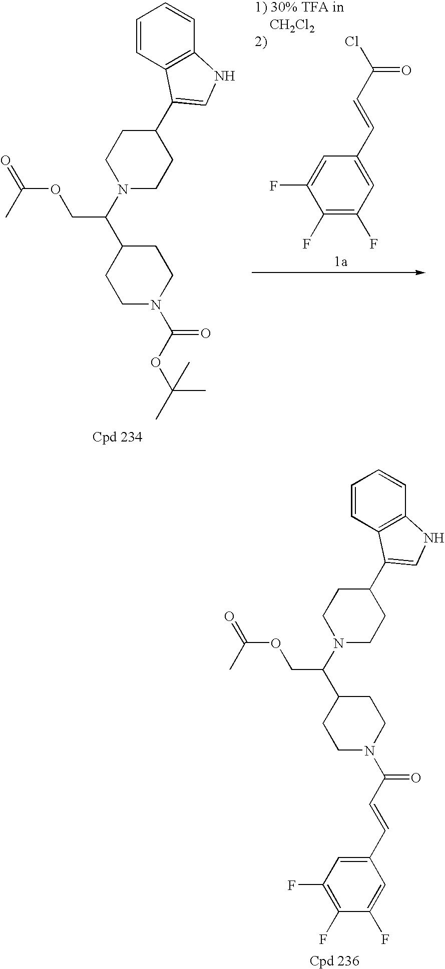

TFA (3.0 mL) was added to a solution of Compound 234 (0.1 g, 0.21 mmol) in methylene chloride (7.0 mL). The mixture was stirred for 2 hrs and then concentrated in vacuo. The resulting residue was dissolved in methylene chloride (10.0 mL) and TEA (0.1 g) and 3-(3,4,5-trifluoro-phenyl)-acryloyl chloride Compound 1a (0.05 g, 0.23 mmol) was added. A crude product was prepared then purified with chromatography (eluted with 50% EtOAc in hexane) to provide Compound 236 (0.08 g, 68%). MS m/z 554 (M+H)⁺.

Using the procedure of Example 15 and known appropriate reagents and starting materials, the following compounds of the invention were prepared:

| Cpd | Name | MS |
|---|---|---|
| 199 | (2E)-3-(3,5-difluoro-phenyl)-acrylic acid 2-{1-[(2E)-3-(3,5-difluoro- | 753 |
| | phenyl)-acryloyl]-piperidin-4-yl}-2-[4-(5-methanesulfonylamino-1H- | |
| | indol-3-yl)-piperidin-1-yl]-ethyl ester | |
| 235 | acetic acid 2-{1-[(2E)-3-(3,5-difluoro-phenyl)-acryloyl]-piperidin-4-yl}- | 536 |
| | 2-[4-(1H-indol-3-yl)-piperidin-1-yl]-ethyl ester | |
| 237 | acetic acid 2-{1-[(2E)-3-(3,4-dichloro-phenyl)-acryloyl]-piperidin-4-yl}- | 568 |
| | 2-[4-(1H-indol-3-yl)-piperidin-1-yl]-ethyl ester | |
| 242 | acetic acid 2-[1-(3,4-dichloro-phenylcarbamoyl)-piperidin-4-yl]-2-[4- | 557 |
| | (1H-indol-3-yl)-piperidin-1-yl]-ethyl ester | |
| 243 | acetic acid 2-{4-[5-(acetyl-methanesulfonyl-amino)-1H-indol-3-yl]- | 671 |
| | piperidin-1-yl}-2-{1-[(2E)-3-(3,5-difluoro-phenyl)-acryloyl]-piperidin-4- | |
| | yl}-ethyl ester | |

### EXAMPLE 16

### (2E)-1-(4-{1-[4-(4-chloro-phenyl)-piperidin-1-yl]-2-hydroxy-ethyl}-piperidin-1-yl)-3-(3,5-difluoro-phenyl)-propenone (Cpd 249)

### carbonic acid 2-[4-(4-chloro-phenyl)-piperidin-1-yl]-2-{1-[(2E)-3-(3,5-difluoro-phenyl)-acryloyl]-piperidin-4-yl}-ethyl ester methyl ester (Cpd 250)

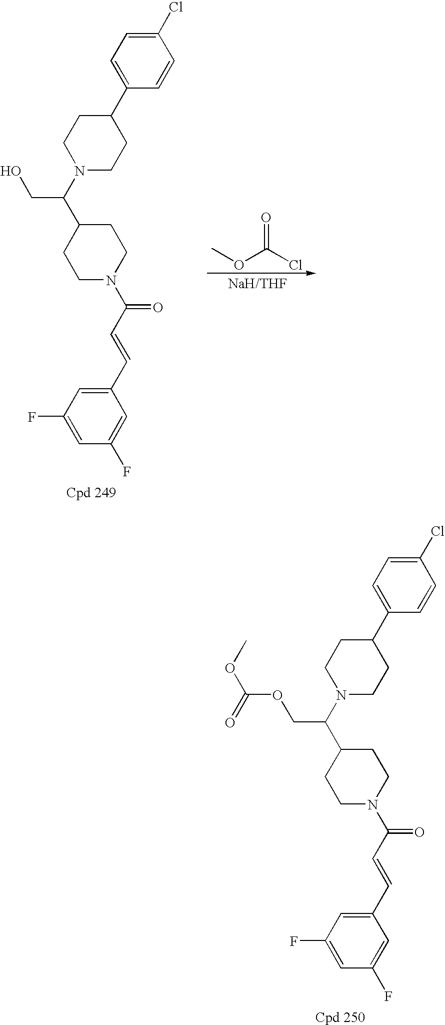

Compound 249 was prepared using the procedure of Example 9 and 4-(4-chloro-phenyl)-piperidine in place of 3-piperidin-4-yl-1H-indole Compound 1f. MS m/z 489 (M+H)⁺.

NaH (5 mg, 0.21 mmol) and methyl chloroformate (10 mg, 0.11 mmol) were added to a solution of Compound 249 (40 mg, 0.082 mmol) in THF (8 mL). The mixture was refluxed for 24 hrs, then concentrated in vacuo for 0.5 hrs. The resulting residue was purified via preparative TLC (in 50% EtOAc/Hexane) to provide Compound 250 (15 mg, 33%). MS m/z 547 (M+H)⁺.

### EXAMPLE 17

### (2E)-3-(3,5-difluoro-phenyl)-1-(4-{2-methoxy-1-[4-(4-methoxy-phenyl)-piperidin-1-yl]-ethyl}-piperidin-1-yl)-propenone (Cpd 255)

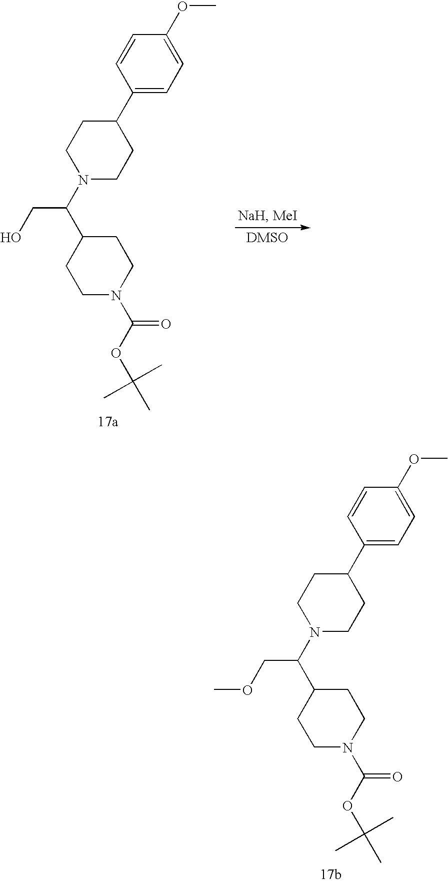

4-{2-hydroxy-1-[4-(4-methoxy-phenyl)-piperidin-1-yl]-ethyl}-piperidine-1-carboxylic acid tert-butyl ester Compound 17a was prepared using the procedure of Example 9 and 4-(4-methoxy-phenyl)-piperidine in place of 3-piperidin-4-yl-1H-indole Compound 1f.

Compound 17a (150 mg, 0.36 mmol, 1 eq) was dissolved in DMSO (3 mL) under nitrogen. Sodium hydride (50% in mineral oil, 22 mg, 0.47 mmol, 1.3 eq) was added at r.t. and the resulting suspension was stirred for 30 mins. Methyl iodide (29 μL, 0.47 mmol, 1.3 eq) was added and the solution was stirred for 16 hrs. An additional amount of sodium hydride (22 mg, 1.3 eq) was added, followed by additional methyl iodide (29 μL, 0.47 mmol, 1.3 eq) and the mixture was stirred for 1 hr. A final portion of sodium hydride (22 mg, 1.3 eq) was added and the suspension was stirred for 1 hr. The reaction mixture was partitioned between brine and EtOAc. The organic layer was removed and the aqueous layer was extracted with EtOAc. The combined organic layers were washed with dilute brine and dried over sodium sulfate, then filtered and evaporated. The residue was purified via silica gel (1:1 hexanes:EtOAc to 100% EtOAc) to provide 4-{2-methoxy-1-[4-(4-methoxy-phenyl)-piperidin-1-yl]-ethyl}-piperidine-1-carboxylic acid tert-butyl ester Compound 17b (47 mg, 30%) as a viscous oil. MS m/Z 433 (M+H)⁺.

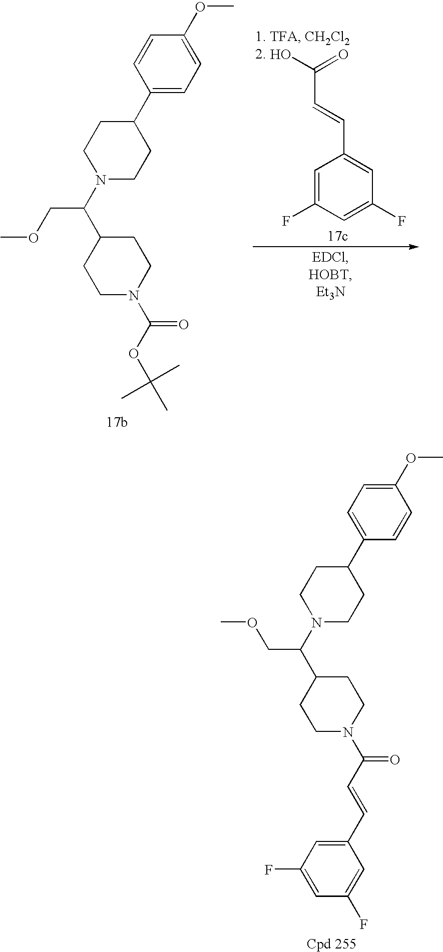

Compound 17b (47 mg, 0.11 mmol, 1 eq) was dissolved in CH₂Cl₂ (2 mL) and treated dropwise with TFA (500 μL). The mixture was stirred for 2 hrs and the solvent was evaporated to provide a crude residue that was used in the next step without further purification. The residue was dissolved in CH₂Cl₂ (1 mL) and DMF (100 μL). The solution was cooled to 0° C. and 3-(3,5-difluoro-phenyl)-acrylic acid Compound 17c (20 mg, 0.11 mmol, 1 eq) was added, followed by HOBt (16 mg, 0.12 mmol, 1.1 eq), Et₃N (46 μL, 0.33 mmol, 3 eq) and EDCI (23 mg, 0.12 mmol, 1.1 eq). The reaction was allowed to slowly warm to r.t. and stirred for 3 days. The solvent was evaporated to provide a residue that was partitioned between CH₂Cl₂ and sat. NaHCO₃. The organic layer was removed, then washed with brine and dried over anhydrous Na₂SO₄. The solution was filtered, then the filtrate was concentrated and purified via silica gel chromatography (1:1 to 1:3 hexanes:EtOAc) to provide Compound 255 (41 mg, 82%) as a pale foam. MS m/z 499 (M+H)⁺.

### EXAMPLE 18

### (2E)-1-{4-[1-(4-benzo[1,3]dioxol-5-yl-piperidin-1-yl)-2-hydroxy-ethyl]-piperidin-1-yl}-3-(3,4,5-trifluoro-phenyl)-propenone (Cpd 189)

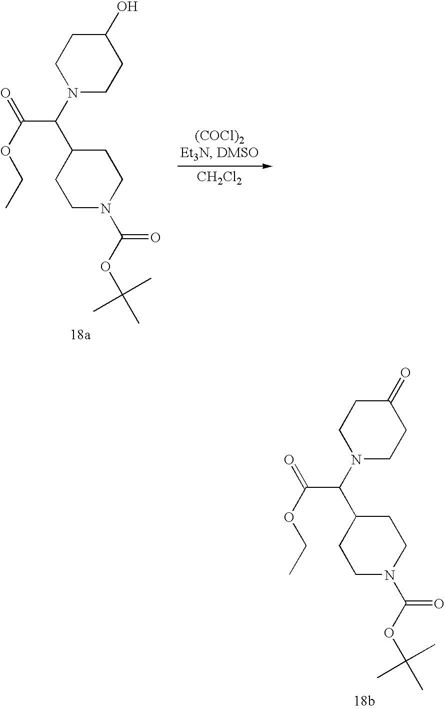

A solution of DMSO (493 μL, 6.95 mmol, 4.4 eq) in CH₂Cl₂ (10 mL) was cooled to −78° C. Oxalyl chloride (276 μL, 3.16 mmol, 2 eq) was added dropwise and the mixture was stirred for 25 mins.

4-[ethoxycarbonyl-(4-hydroxy-piperidin-1-yl)-methyl]-piperidine-1-carboxylic acid tert-butyl ester Compound 18a was prepared using the procedure of Example 9 and piperidin-4-ol in place of 3-piperidin-4-yl-1H-indole Compound 1f.

A solution of Compound 18a (586 mg, 1.58 mmol, 1 eq) in CH₂Cl₂ (5 mL) was added dropwise to the solution of oxalyl chloride in DMSO at −78° C. The mixture was stirred for 20 mins and Et₃N (1.3 mL, 9.48 mmol, 6 eq) was added dropwise. The mixture was warmed to room temperature and then partitioned between CH₂Cl₂ and brine. The organic layer was removed and the aqueous layer was made more basic with 2.5N NaOH and extracted twice with CH₂Cl₂. The combined organic layers were washed with brine and dried over sodium sulfate, then filtered and evaporated to provide a crude residue that was purified by silica gel chromatography (3:1 hexanes:EtOAc to 2:3 hexanes:EtOAc) to provide 4-[ethoxycarbonyl-(4-oxo-piperidin-1-yl)-methyl]-piperidine-1-carboxylic acid tert-butyl ester Compound 18b (503 mg, 86%) as a crystalline solid. MS m/z 387 (M+H+ H₂O)⁺.

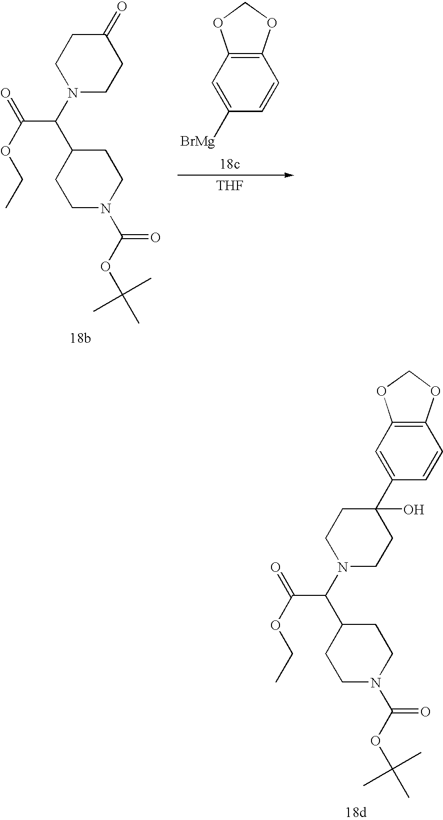

A solution of benzo[1,3]dioxol-5-yl magnesium bromide Compound 18c (1M in 1:1 toluene:THF, 1.03 mL, 1.03 mmol, 1 eq) was added dropwise to a stiffed solution of Compound 18b (378 mg, 1.03 mmol, 1 eq) in THF (6 mL) at 0° C. After 1 hr, additional Compound 18c (600 μL) was added and the mixture was stirred for another 30 mins. The reaction was quenched with saturated NH₄Cl and partitioned between saturated NaHCO₃ and EtOAc. The organic layer was removed and the aqueous layer was extracted with EtOAc. The organic layers were combined, washed with brine and dried over anhydrous sodium sulfate, then filtered and evaporated to provide a crude product which was purified via silica gel chromatography (2:1 hexanes:EtOAc to 50:50 hexanes:EtOAc) to provide 4-[(4-benzo[1,3]dioxol-5-yl-4-hydroxy-piperidin-1-yl)-ethoxycarbonyl-methyl]-piperidine-1-carboxylic acid tert-butyl ester Compound 18d (335 mg, 66%). MS m/z 491 (M+H)⁺.

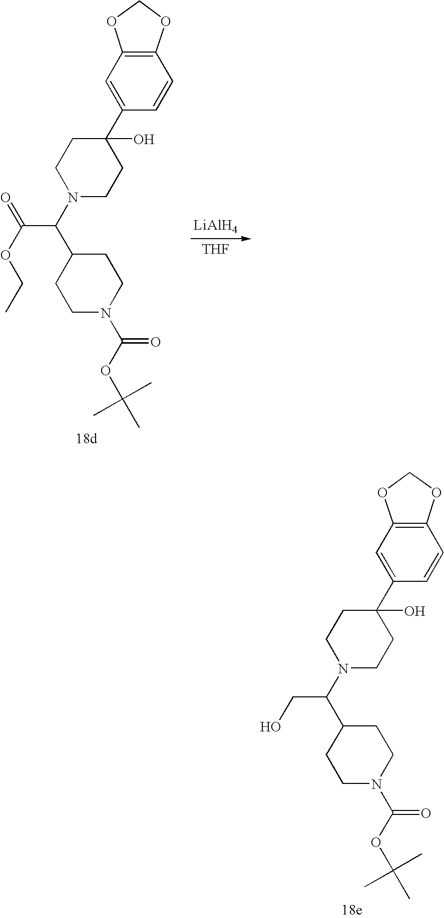

A solution of Compound 18d (163 mg, 0.33 mmol, 1 eq) in T}IF (2.5 mL) was cooled to 0° C. and treated with LiAlH₄ (1M in THF, 500 μL, 0.50 mmol, 1.5 eq). The mixture was stirred for 2 hrs, during which time the ice bath melted, and the reaction was sequentially quenched with water (22 μL), 15% NaOH (22 μL) and water (66 μL). The quenched reaction mixture was stirred for 30 mins, then the solids were removed by filtration through celite and subsequent washing with EtOAc. The filtrate was evaporated and the crude residue was purified via silica gel chromatography (5% to 10% 2M MeOH/NH₃ in CH₂Cl₂) to provide 4-[1-(4-benzo[1,3]dioxol-5-yl-4-hydroxy-piperidin-1-yl)-2-hydroxy-ethyl]-piperidine-1-carboxylic acid tert-butyl ester Compound 18e (72 mg, 49%) as an oil. MS m/z 449 (M+H)⁺.

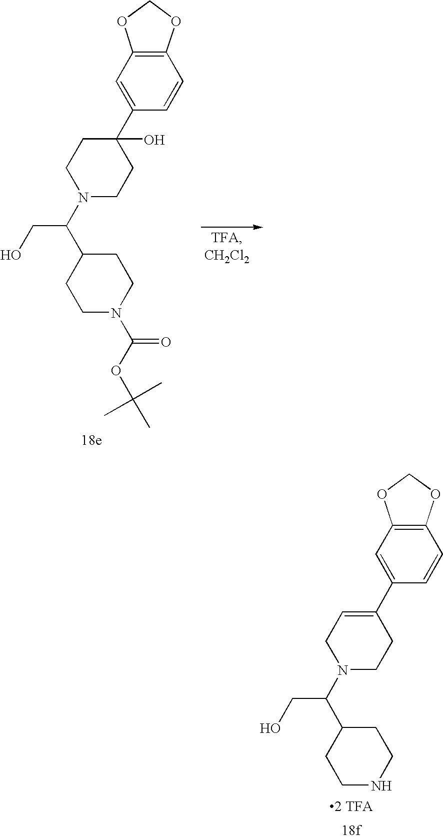

TFA (0.5 mL) was added to a solution of Compound 18e (72 mg, 0.16 mmol) in CH₂Cl₂ (1 mL). The mixture was stirred for 30 min, then evaporated to provide a bis-trifluoroacetate salt of 2-(4-benzo[1,3]dioxol-5-yl-3,6-dihydro-2H-pyridin-1-yl)-2-piperidin-4-yl-ethanol Compound 18f (89 mg, quant) as a yellow oil that was used in the next step without further purification. MS m/z 331 (M+H)⁺.

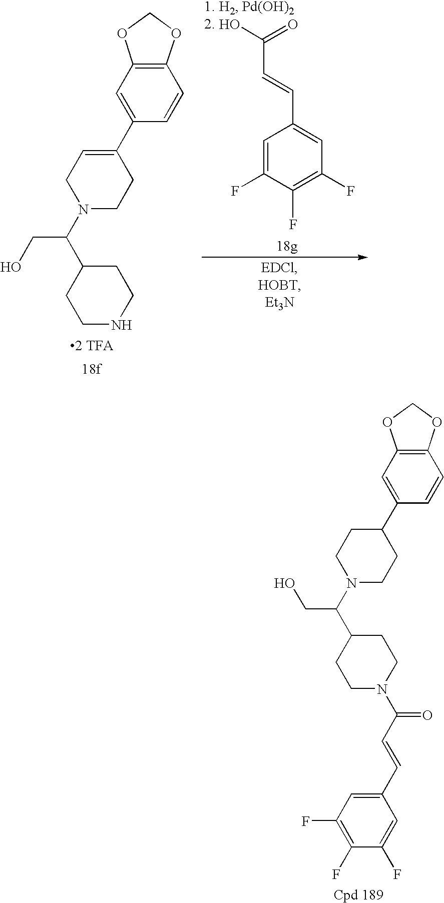

A solution of Compound 18f (89 mg, 0.16 mmol, 1 eq) was dissolved in methanol (10 mL) and charged with palladium hydroxide (20% on carbon, 50% w/w with water, 40 mg, 0.028 mmol, 0.2 eq). The mixture was sequentially purged with nitrogen and hydrogen, then shaken under hydrogen (50 psi) for 4 hrs. After purging with nitrogen, the mixture was filtered through celite and the filtrate was evaporated to provide a viscous oil. A portion of the crude product (45 mg, 0.08 mmol, 1 eq) was dissolved in CH₂Cl₂ (0.5 mL) and DMF (0.5 mL). 3-(3,4,5-trifluoro-phenyl)-acrylic acid Compound 18g (16 mg, 0.08 mmol, 1 eq) was added, followed by HOBt (12 mg, 0.088 mmol, 1.1 eq), Et₃N (45 μL, 0.32 mmol, 4 eq) and EDCI (17 mg, 0.088 mmol, 1.1 eq). The reaction mixture was stirred at room temperature for 16 hrs, then the solvents were evaporated. The resulting residue was partitioned between CH₂Cl₂ and sat. NaHCO₃. The organic layer was removed and the aqueous layer was extracted again with CH₂Cl₂. The combined organic layers were dried over Na₂SO₄, then filtered and evaporated. The resulting residue was purified via silica gel chromatography (4% to 12% 2M NH₃.MeOH in CH₂Cl₂) to provide Compound 189 (24 mg, 58%) as a tan foam. MS m/z 517 (M+H)⁺.

Using the procedure of Example 18 and known appropriate reagents and starting materials, the following compounds of the invention were prepared:

| Cpd | Name | MS |
|---|---|---|
| 190 | (2E)-1-{4-[1-(4-benzo[1,3]dioxol-5-yl- | 499 |
| | piperidin-1-yl)-2-hydroxy-ethyl]-piperidin-1- | |
| | yl}-3-(3,5-difluoro-phenyl)-propenone | |

### EXAMPLE 19

### (2E)-3-(3,5-difluoro-phenyl)-1-(4-{1-[4-(4-fluoro-phenyl)-piperidin-1-yl]-2-hydroxy-ethyl}-piperidin-1-yl)-propenone (Cpd 192)

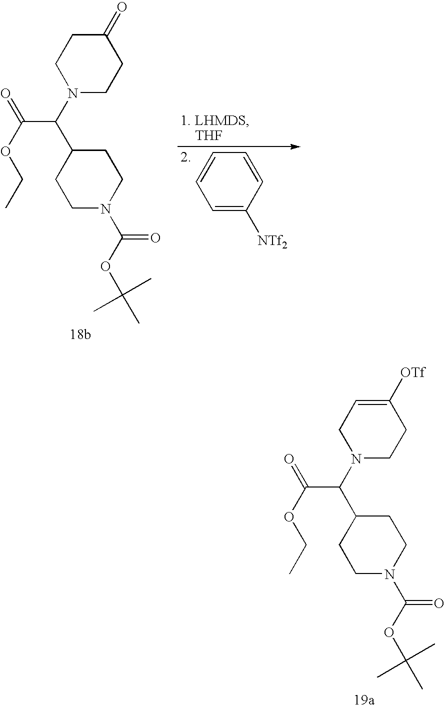

4-[ethoxycarbonyl-(4-oxo-piperidin-1-yl)-methyl]-piperidine-1-carboxylic acid tert-butyl ester Compound 18b (503 mg, 1.37 mmol, 1 eq) was dissolved in THF 10.5 mL) and cooled to −78° C. Lithium bis(trimethylsilyl)amide (1M in THF, 1.5 mL, 1.5 mmol, 1.1 eq) was added dropwise to the Compound 18b solution and stirred for 20 mins at −78° C. A solution of N-phenyl-trifluoromethanesulfonimide (536 mg, 1.5 mmol, 1.5 eq) in THF (5 mL) was added dropwise with stirring. The resulting mixture was warmed to 0° C. and stirred for 3 hrs at 0° C. The solvents were removed in vacuo, and the resulting residue purified by chromatography on neutral alumina (3:1 hexanes:EtOAc) to provide 4-[ethoxycarbonyl-(4-trifluoromethanesulfonyloxy-3,6-dihydro-2H-pyridin-1-yl)-methyl]-piperidine-1-carboxylic acid tert-butyl ester Compound 19a (432 mg, 63%) as a viscous oil. MS m/z 523 (M+Na)⁺.

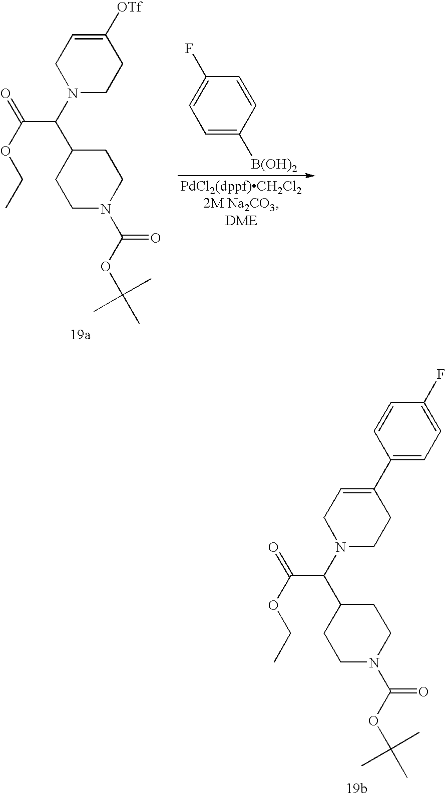

A solution of Compound 19a (170 mg, 0.34 mmol, 1 eq) and 4-fluoro-phenyl boronic acid (52 mg, 0.37 mmol, 1.1 eq) in DME (3.3 mL) was charged with 2M Na₂CO₃ (0.68 mL) and dichloro[1,1′-bis(diphenylphosphino)ferrocene]palladium dichloromethane adduct (20 mg, 0.027 mmol, 0.08 eq). The mixture was heated to reflux for 2.5 hrs, then cooled and partitioned between EtOAc and brine. The organic layer was removed and the aqueous layer was extracted again with EtOAc. The combined organic layers were dried over anhydrous sodium sulfate, filtered and evaporated, then purified via silica gel chromatography (4:1 hexanes:EtOAc to 1:1 hexanes:EtOAc) to provide 4-{ethoxycarbonyl-[4-(4-fluoro-phenyl)-3,6-dihydro-2H-pyridin-1-yl]-methyl}-piperidine-1-carboxylic acid tert-butyl ester Compound 19b (79 mg, 52%) as a viscous oil. MS m/z 447 (M+H)⁺.

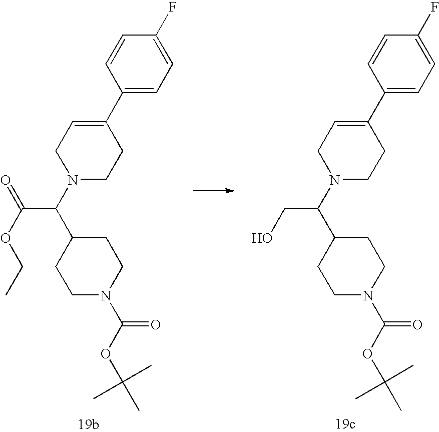

A solution of Compound 19b (79 mg, 0.18 mmol, 1 eq) in THF (1.4 mL) was treated with LiAlH₄ (1M in THF, 270 μL, 0.27 mmol, 1.5 eq) and stirred for 2 hrs, then water (13 μL), 15% NaOH (13 μL) and water (39 μL) were sequentially added. The reaction mixture was stirred for 1 hr, then the quenched reaction mixture was filtered through a celite pad and the pad was washed with EtOAc. The combined filtrates were evaporated to provide 4-{1-[4-(4-fluoro-phenyl)-3,6-dihydro-2H-pyridin-1-yl]-2-hydroxy-ethyl}-piperidine-1-carboxylic acid tert-butyl ester Compound 19c (65 mg (89%), which was used in the next step without further purification. MS nz/z 405 (M+H)⁺.

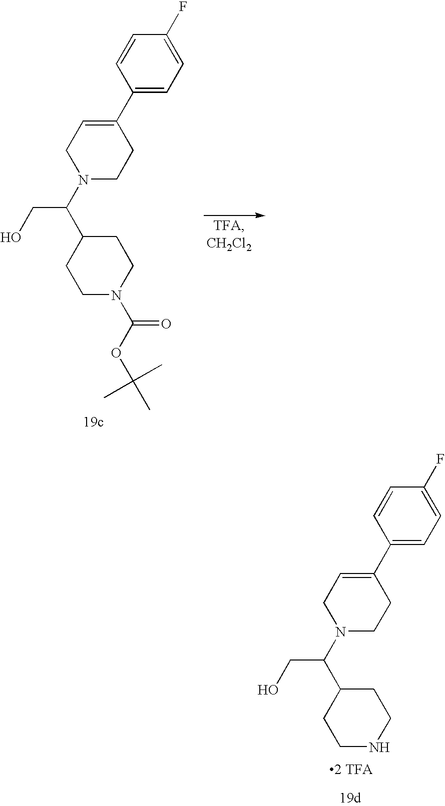

A solution of Compound 19c (65 mg, 0.16 mmol) in CH₂Cl₂ (1 mL) was treated with TFA (0.5 mL). The mixture was stirred for 3 hrs, then the solvent was removed in vacuo to provide the bis-trifluoroacetate salt of 2-[4-(4-fluoro-phenyl)-3,6-dihydro-2H-pyridin-1-yl]-2-piperidin-4-yl-ethanol Compound 19d (88 mg, quant.) as a viscous oil that was used without further purification. MS m/z 305 (M+H)⁺.

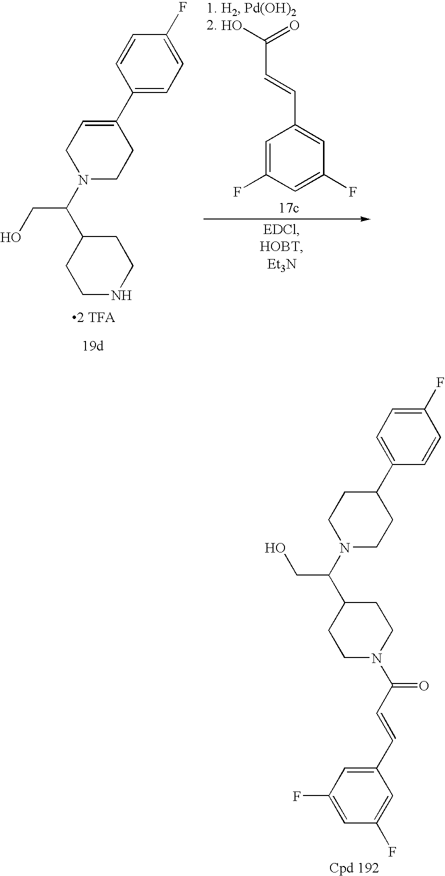

A solution of Compound 19d (88 mg, 0.16 mmol, 1 eq.) and palladium hydroxide (40 mg, 0.029 mmol, 0.18 eq) in methanol (10 mL) was sequentially purged with nitrogen and hydrogen, then shaken under hydrogen (50 psi) for 16 hrs. After nitrogen purging, the reaction mixture was filtered through celite and the filtrate was evaporated to provide the bis-trifluoroacetate salt of 2-[4-(4-fluoro-phenyl)-piperidin-1-yl]-2-piperidin-4-yl-ethanol Compound 19e, which was used in the next step without further purification. A portion of Compound 19e (43 mg, 0.08 mmol, 1 eq) was dissolved in CH₂Cl₂ (0.5 mL) and DMF (0.5 mL). 3-(3,5-difluoro-phenyl)-acrylic acid Compound 17c (15 mg, 0.08 mmol, 1 eq) was added, followed by HOBt (12 mg, 0.088 mmol, 1.1 eq), Et₃N (45 μL, 0.32 mmol, 4 eq) and EDCI (17 mg, 0.088 mmol, 1.1 eq). The mixture was stirred at room temperature for 72 hrs. The solvent was evaporated to provide a residue that was partitioned between CH₂Cl₂ and sat. NaHCO₃. The organic layer was removed and the aqueous layer was extracted again with CH₂Cl₂. The combined organic layers were dried over anhydrous Na₂SO₄, then filtered and evaporated. The resulting residue was purified by silica gel chromatography (4% to 12% 2M NH₃.MeOH in CH₂Cl₂) to provide Compound 192 (11 mg, 29%) as a tan foam. MS m/z 473 (M+H)⁺.

Using the procedure of Example 19 and known appropriate reagents and starting materials, the following compounds of the invention were prepared:

| Cpd | Name | MS |
|---|---|---|
| 193 | (2E)-1-(4-{1-[4-(4-fluoro-phenyl)-piperidin-1- | 491 |
| | yl]-2-hydroxy-ethyl}-piperidin-1-yl)-3- | |
| | (3,4,5-trifluoro-phenyl)-propenone | |
| 197 | (2E)-3-(3,5-difluoro-phenyl)-1-(4-{2-hydroxy- | 507 |
| | 1-[4-(3-methoxy-phenyl)-piperidin-1- | (M + Na) |
| | yl]-ethyl}-piperidin-1-yl)-propenone | |

### EXAMPLE 20

### (2E)-3-(3,5-difluoro-phenyl)-1-{4-[2-hydroxy-1-(4-thiazol-2-yl-piperidin-1-yl)-ethyl]-piperidin-1-yl}-propenone

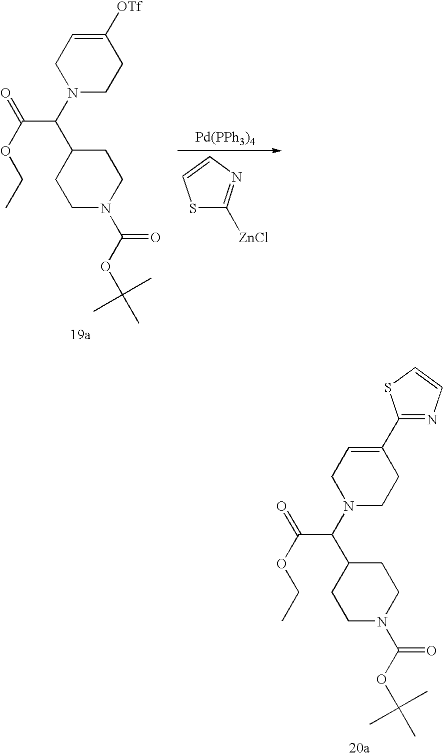

A solution of n-butyl lithium (1.05M in hexanes, 695 mL, 1.7 eq) was added dropwise to a solution of thiazole (43 μL, 0.60 mmol, 1.4 eq) in THF (1 mL) at −78° C. and the mixture was stirred for 20 mins. Freshly powdered zinc chloride (246 mg, 1.81 mmol, 4.2 eq) was added and the mixture was warmed to room temperature with stirring. A solution of 4-[ethoxycarbonyl-(4-trifluoromethanesulfonyloxy-3,6-dihydro-2H-pyridin-1-yl)-methyl]-piperidine-1-carboxylic acid tert-butyl ester Compound 19a (216 mg, 0.43 mmol, 1 eq) in THF (2 mL) and tetrakis triphenylphosphine palladium (50 mg, 0.043 mmol, 0.1 eq) were added to the solution. The mixture was heated at reflux for 1 hr, then cooled and partitioned between EtOAc and saturated NaHCO₃. The organic layer was removed and the aqueous layer was extracted with EtOAc. The organic layers were combined and dried over anhydrous sodium sulfate, then filtered and evaporated. The resulting residue was purified via silica gel chromatography (3:2 to 2:3 hexanes:EtOAc) to provide 4-[ethoxycarbonyl-(4-thiazol-2-yl-3,6-dihydro-2H-pyridin-1-yl)-methyl]-piperidine-1-carboxylic acid tert-butyl ester Compound 20a (174 mg, 93%) as a yellow foam. MS m/z 438 (M+H)⁺.

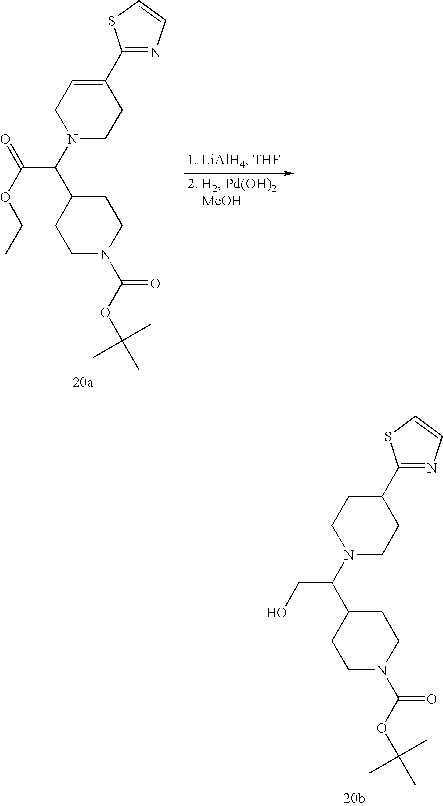

A solution of Compound 20a (165 mg, 0.38 mmol, 1 eq) in THF (3 mL) was cooled to 0° C. and treated with LiAlH₄ (1M in THF, 570 μL, 1.5 eq) with stirring. The mixture was stirred for 1 hr, then warmed to room temperature and stirred for an additional 1 hr. The reaction was sequentially quenched with water (30 μL), 15% NaOH (30 mL) and water (90 mL). The quenched reaction mixture was stirred for 30 mins, then filtered through a celite pad and the pad was washed with EtOAc. The filtrate was evaporated and the resulting residue purified via silica gel chromatography (4% to 12% 2M MeOH.NH₃ in CH₂Cl₂) to provide an inseparable mixture of crude products. The product mixture was dissolved in of MeOH and Pd(OH)₂ (35 mg, 0.025 mmol, 0.12 eq) and purged with nitrogen. Hydrogen was bubbled through the mixture, and the mixture was stirred under hydrogen for 3 hrs. The mixture was purged with nitrogen, then filtered through celite and evaporated to provide (in 2 steps) 4-[2-hydroxy-1-(4-thiazol-2-yl-piperidin-1-yl)-ethyl]-piperidine-1-carboxylic acid tert-butyl ester Compound 20b (82 mg, 55%) as a pale foam that was used in the next step without further purification. MS m/z 396 (M+H)⁺.

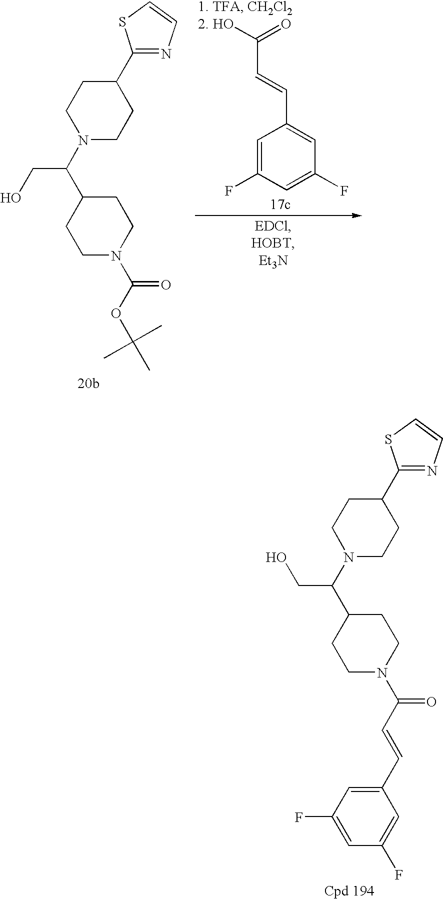

Compound 20b (82 mg, 0.21 mmol, 1 eq) was dissolved in CH₂Cl₂ (2 mL) and cooled to 0° C. with stirring. TFA (0.5 mL) was added dropwise and the mixture was stirred for 3 hrs while warming to room temperature. The solvent was removed in vacuo to provide a crude residue, which was used in the next step without further purification. A portion of the residue (37 mg, 0.07 mmol, 1 eq) was dissolved in CH₂Cl₂ (0.5 mL) and DMF (0.5 mL). 3-(3,5-difluoro-phenyl)-acrylic acid Compound 17b (13 mg, 0.07 mmol, 1 eq) was added, followed by HOBt (10 mg, 0.077 mmol, 1.1 eq), Et₃N (39 μL, 0.28 mmol, 4 eq) and EDCI (15 mg, 0.077 mmol, 1.1 eq). The mixture was stirred at room temperature for 16 hrs, then the solvent was evaporated. The resulting residue was partitioned between CH₂Cl₂ and sat. NaHCO₃. The organic layer was removed and the aqueous layer was extracted again with CH₂Cl₂. The combined organic layers were dried over anhydrous Na₂SO₄, then filtered and evaporated. The resulting residue was purified via silica gel chromatography (2% to 10% 2M NH₃.MeOH in CH₂Cl₂) to provide Compound 194 (11 mg, 34%) as a tan foam. MS m/z 462 (M+H)⁺.

Using the procedure of Example 20 and known appropriate reagents and starting materials, the following compounds of the invention were prepared:

| Cpd | Name | MS |
|---|---|---|
| 195 | (2E)-1-{4-[2-hydroxy-1-(4-thiazol-2-yl- | 480 |
| | piperidin-1-yl)-ethyl]-piperidin-1-yl}-3- | |
| | (3,4,5-trifluoro-phenyl)-propenone | |
| 196 | (2E)-3-(3,4-dichloro-phenyl)-1-{4-[2- | 494 |
| | hydroxy-1-(4-thiazol-2-yl-piperidin-1-yl)- | |
| | ethyl]-piperidin-1-yl}-propenone | |

### EXAMPLE 21

### (2E)-3-(3,5-difluoro-phenyl)-1-(4-{2-hydroxy-1-[4-(2-methoxy-phenyl)-piperidin-1-yl]-ethyl}-piperidin-1-yl)-propenone (Compound 203)

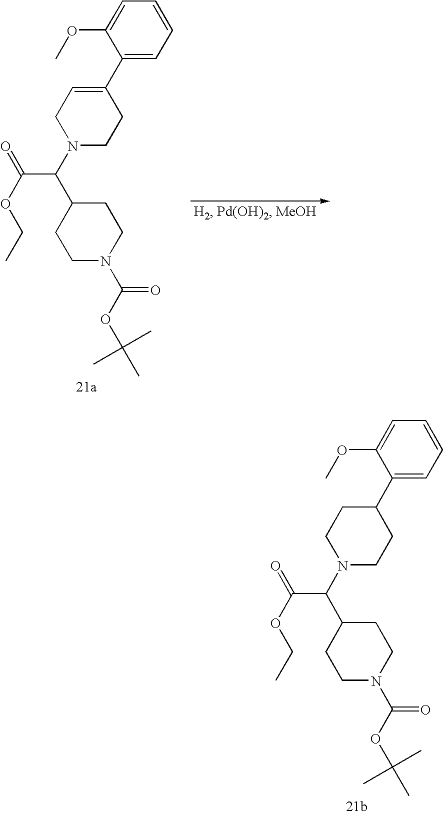

The procedure of Example 20 and 2-methoxy-phenyl and zinc iodide in place of thiazol-2-yl and zinc chloride were used to prepare 4-{ethoxycarbonyl-[4-(2-methoxy-phenyl)-3,6-dihydro-2H-pyridin-1-yl]-methyl}-piperidine-1-carboxylic acid tert-butyl ester Compound 21a.

A solution of Compound 21a (200 mg, 0.44 mmol, 1 eq) and palladium hydroxide (20% on carbon, 50 wt. % H₂O, 70 mg, 0.05 mmol, 0.11 eq) in methanol (3 mL) was sequentially purged with nitrogen and hydrogen, then pressurized under hydrogen (50 psi), the mixture was shaken for 24 hrs. After purging with nitrogen, the reaction mixture was filtered through celite and the filtrate was evaporated. The resulting residue was filtered through a plug of silica (3:2:1 to 3:1:1 CH₂Cl₂:hexanes:EtOAc) to provide 4-{ethoxycarbonyl-[4-(2-methoxy-phenyl)-piperidin-1-yl]-methyl}-piperidine-1-carboxylic acid tert-butyl ester Compound 21b (58 mg, 29%) as a viscous oil. MS m/z 462 (M+H)⁺.

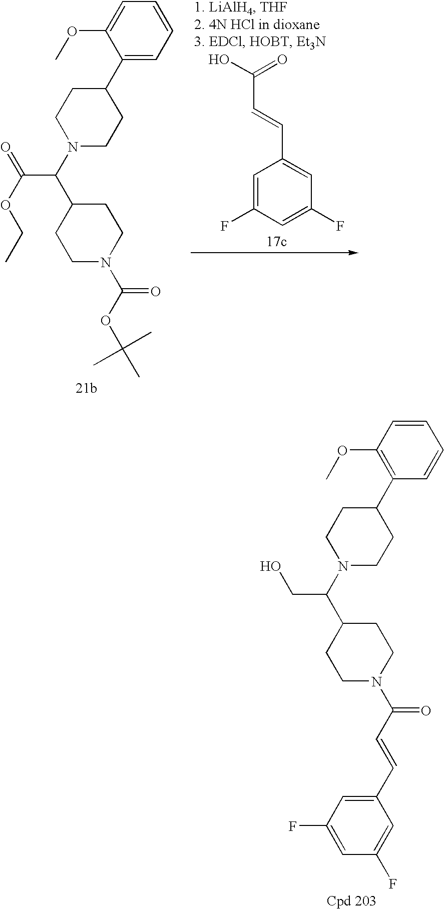

A solution of Compound 21b (58 mg, 0.13 mmol, 1 eq) in THF (1 mL) was cooled to 0° C. and treated with LiAlH₄ (1M in THF, 190 μL, 1.5 eq) with stirring. After 1 hr, the mixture was warmed to room temperature and stirred for an additional 1 hr. The reaction was sequentially quenched with water (9 μL), 15% NaOH (9 μL) and water (27 μL). The mixture was stirred for 30 mins, then filtered through a celite pad and the pad was washed with EtOAc. The filtrates were evaporated and dissolved in methanol (2 mL). A solution of 4N HCl in dioxane was added dropwise with stirring. The mixture was stirred for 3 hrs, then the solvent was removed in vacuo and the residue dissolved in DMF (1 mL). 3-(3,5-difluoro-phenyl)-acrylic acid Compound 17c (20 mg, 0.11 mmol, 1 eq) was added, followed by HOBt (16 mg, 0.12 mmol, 1.1 eq), Et₃N (46 μL, 0.33 mmol, 3 eq) and EDCI (23 mg, 0.12 mmol, 1.1 eq). The mixture was stirred at room temperature for 16 hrs. The solvent was evaporated to provide a residue that was partitioned between CH₂Cl₂ and sat. NaHCO₃. The organic layer was removed and the aqueous layer was extracted again with CH₂Cl₂. The combined organic layers were dried over anhydrous Na₂SO₄, then filtered and evaporated. The resulting residue was purified by silica gel chromatography (2% to 10% 2M NH₃.MeOH in CH₂Cl₂) to provide Compound 203 (12 mg, 23%) as a pale foam. MS m/z 485 (M+H)⁺.

Using the procedure of Example 21 and known appropriate reagents and starting materials, the following compounds of the invention were prepared:

| Cpd | Name | MS |
|---|---|---|
| 200 | 3-(3,5-difluoro-phenyl)-1-{4-[2-hydroxy-1- | 456 |
| | (3′,4′,5′,6′-tetrahydro-2′H- | |
| | [2,4′]bipyridinyl-1′-yl)-ethyl]- | |
| | piperidin-1-yl}-propenone | |

### EXAMPLE 22

### N-{2-{1-[(2E)-3-(3,5-difluoro-phenyl)-acryloyl]-piperidin-4-yl}-2-[4-(1H-indol-3-yl)-piperidin-1-yl]-ethyl}-acetamide (Cpd 214)

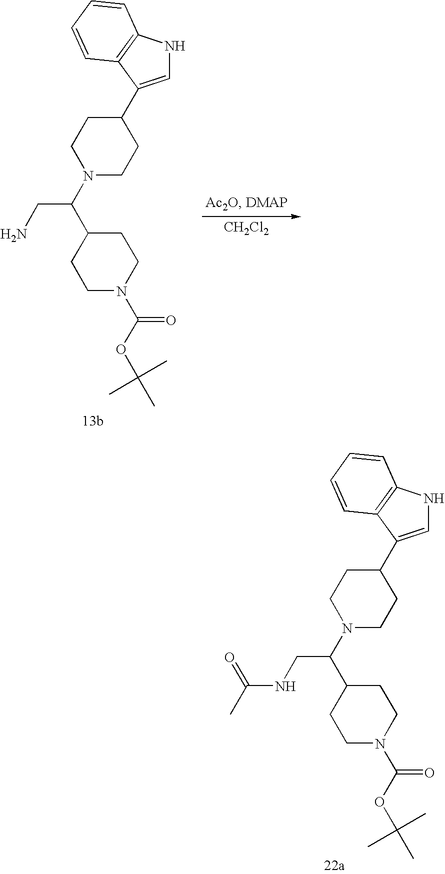

A solution of 4-{2-amino-1-[4-(1H-indol-3-yl)-piperidin-1-yl]-ethyl}-piperidine-1-carboxylic acid tert-butyl ester Compound 13b (431 mg, 1.01 mmol, 1 eq) in CH₂Cl₂ (5 mL) was treated with dropwise addition of acetic anhydride (572 μL, 6.06 mmol, 6 eq) followed by addition of DMAP (12 mg, 0.1 mmol, 0.1 eq). After stirring overnight at room temperature, the volatiles were removed in vacuo and the resulting residue dissolved in CH₂Cl₂. After washing with saturated sodium bicarbonate, the organic layer was dried over anhydrous sodium sulfate, filtered, and evaporated. The crude residue was subjected to silica gel chromatography (2% to 10% 2M MeOH.NH₃ in CH₂Cl₂) to provide 4-{2-acetylamino-1-[4-(1H-indol-3-yl)-piperidin-1-yl]-ethyl}-piperidine-1-carboxylic acid tert-butyl ester Compound 22a (385 mg, 81%) as a white foam. MS m/z 469 (M+H)⁺.

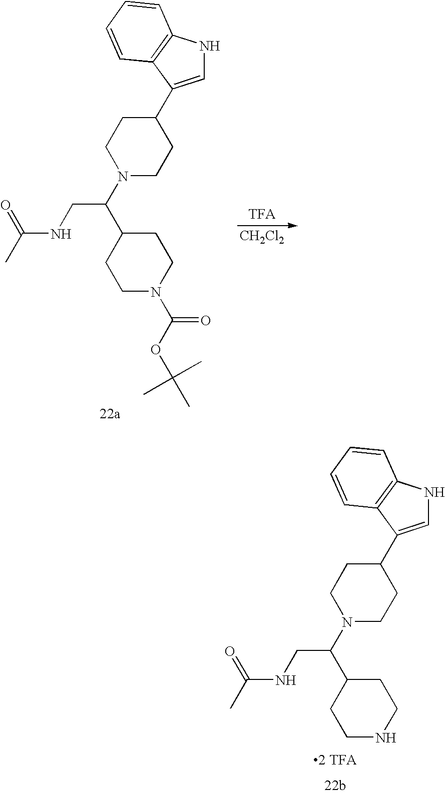

A solution of Compound 22a (352 mg, 0.75 mmol) in CH₂Cl₂ (6 mL) was treated with TFA (1 mL) and the reaction mixture was stirred for 4 hrs at room temperature. The mixture was evaporated to dryness to provide N-{2-[4-(1H-indol-3-yl)-piperidin-1-yl]-2-piperidin-4-yl-ethyl}-acetamide, bis-trifluoroacetate salt Compound 22b (442 mg, 99%) as a dark oil that was used in the next step without further purification. MS m/z 369 (M+H)⁺.

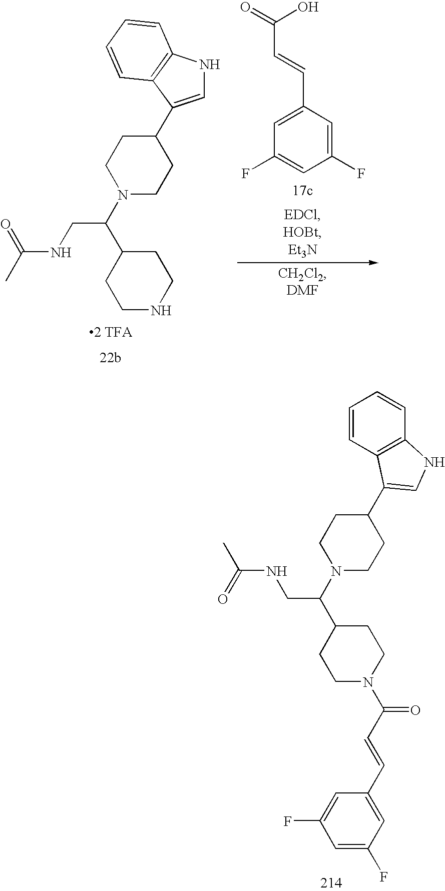

A solution of Compound 22b (66 mg, 0.11 mmol, 1 eq) and 3-(3,5-difluoro-phenyl)-acrylic acid Compound 17c (24 mg, 0.12 mmol, 1.1 eq) in CH₂Cl₂ (1 mL) and DMF (0.5 mL) was treated with triethylamine (61 μL, 0.44 mmol, 4 eq), HOBt (16 mg, 0.12 mmol, 1.1 eq), and EDCI (23 mg, 0.12 mmol, 1.1 eq) and the reaction was stirred for 16 hrs at room temperature. The solvents were removed in vacuo, and the resulting residue partitioned between CH₂Cl₂ and saturated NaHCO₃. The organic layer was removed, and the aqueous layer extracted with CH₂Cl₂. The organic extracts were combined, dried over anhydrous sodium sulfate, filtered, and evaporated to provide a crude residue that was purified via silica gel chromatography (2% to 10% gradient of 2M MeOH.NH₃ in CH₂Cl₂) to afford Compound 214 (29 mg, 49%) as a tan foam. MS m/z 535 (M+H)⁺.

Using the procedure of Example 22 and known appropriate reagents and starting materials, the following compounds of the invention were prepared:

| Cpd | Name | MS |
|---|---|---|
| 213 | N-(2-[4-(1H-indol-3-yl)-piperidin-1-yl]-2-{1-[(2E)-3-(3,4,5-trifluoro- | 553 |
| | phenyl)-acryloyl]-piperidin-4-yl}-ethyl)-acetamide | |
| 215 | N-{2-{1-[(2E)-3-(3,4-dichloro-phenyl)-acryloyl]-piperidin-4-yl}-2-[4- | 567 |
| | (1H-indol-3-yl)-piperidin-1-yl]-ethyl}-acetamide | |
| 216 | N-({2-[4-(1H-indol-3-yl)-piperidin-1-yl]-2-{1-[(2E)-3-m-tolyl- | 513 |
| | acryloyl]-piperidin-4-yl}-ethyl)-acetamide | |
| 217 | 4-{2-acetylamino-1-[4-(1H-indol-3-yl)-piperidin-1-yl]-ethyl}- | 556 |
| | piperidine-1-carboxylic acid (3,4-dichloro-phenyl)-amide | |
| 218 | N-(2-[4-(1H-indol-3-yl)-piperidin-1-yl]-2-{1-[(2E)-3-(3- | 567 |
| | trifluoromethyl-phenyl)-acryloyl]-piperidin-4-yl}-ethyl)-acetamide | |
| 219 | N-(2-[4-(1H-indol-3-yl)-piperidin-1-yl]-2-{1-[(2E)-3-thiophen-3-yl- | 505 |
| | acryloyl]-piperidin-4-yl}-ethyl)-acetamide | |
| 220 | N-(2-[4-(1H-indol-3-yl)-piperidin-1-yl]-2-{1-[(2E)-3-(3,4,5-trifluoro- | 539 |
| | phenyl)-acryloyl]-piperidin-4-yl}-ethyl)-formamide | |
| 221 | N-{2-{1-[(2E)-3-(3,5-difluoro-phenyl)-acryloyl]-piperidin-4-yl}-2-[4- | 521 |
| | (1H-indol-3-yl)-piperidin-1-yl]-ethyl}-formamide | |
| 222 | N-{2-{1-[(2E)-3-(3,4-dichloro-phenyl)-acryloyl]-piperidin-4-yl}-2-[4- | 553 |
| | (1H-indol-3-yl)-piperidin-1-yl]-ethyl}-formamide | |
| 223 | N-(2-[4-(1H-indol-3-yl)-piperidin-1-yl]-2-{1-[(2E)-3-m-tolyl- | 499 |
| | acryloyl]-piperidin-4-yl}-ethyl)-formamide | |
| 224 | N-(2-[4-(1H-indol-3-yl)-piperidin-1-yl]-2-{1-[(2E)-3-(3- | 553 |
| | trifluoromethyl-phenyl)-acryloyl]-piperidin-4-yl}-ethyl)-formamide | |
| 225 | N-(2-[4-(1H-indol-3-yl)-piperidin-1-yl]-2-{1-[(2E)-3-thiophen-3-yl- | 491 |
| | acryloyl]-piperidin-4-yl}-ethyl)-formamide | |
| 226 | 4-{2-formylamino-1-[4-(1H-indol-3-yl)-piperidin-1-yl]-ethyl}- | 542 |
| | piperidine-1-carboxylic acid (3,4-dichloro-phenyl)-amide | |
| 228 | 1-ethyl-3-(2-[4-(1H-indol-3-yl)-piperidin-1-yl]-2-{1-[(2E)-3-(3,4,5- | 582 |
| | trifluoro-phenyl)-acryloyl]-piperidin-4-yl}-ethyl)-urea | |
| 229 | 1-{2-{1-[(2E)-3-(3,5-difluoro-phenyl)-acryloyl]-piperidin-4-yl}-2-[4- | 564 |
| | (1H-indol-3-yl)-piperidin-1-yl]-ethyl}-3-ethyl-urea | |
| 230 | 1-{2-{1-[(2E)-3-(3,4-dichloro-phenyl)-acryloyl]-piperidin-4-yl}-2-[4- | 596 |
| | (1H-indol-3-yl)-piperidin-1-yl]-ethyl}-3-ethyl-urea | |
| 231 | 1-ethyl-3-(2-[4-(1H-indol-3-yl)-piperidin-1-yl]-2-{1-[(2E)-3-m-tolyl- | 542 |
| | acryloyl]-piperidin-4-yl}-ethyl)-urea | |
| 232 | 1-ethyl-3-(2-[4-(1H-indol-3-yl)-piperidin-1-yl]-2-{1-[(2E)-3-(3- | 596 |
| | trifluoromethyl-phenyl)-acryloyl]-piperidin-4-yl}-ethyl)-urea | |
| 233 | 1-{2-{1-[(2E)-3-(3-bromo-4-fluoro-phenyl)-acryloyl]-piperidin-4-yl}- | 624 |
| | 2-[4-(1H-indol-3-yl)-piperidin-1-yl]-ethyl}-3-ethyl-urea | |
| 238 | (2-[4-(1H-indol-3-yl)-piperidin-1-yl]-2-{1-[(2E)-3-(3,4,5-trifluoro- | 569 |
| | phenyl)-acryloyl]-piperidin-4-yl}-ethyl)-carbamic acid methyl ester | |
| 239 | {2-{1-[(2E)-3-(3,5-difluoro-phenyl)-acryloyl]-piperidin-4-yl}-2-[4- | 551 |
| | (1H-indol-3-yl)-piperidin-1-yl]-ethyl}-carbamic acid methyl ester | |
| 240 | {2-{1-[(2E)-3-(3,4-dichloro-phenyl)-acryloyl]-piperidin-4-yl}-2-[4- | 583 |
| | (1H-indol-3-yl)-piperidin-1-yl]-ethyl}-carbamic acid methyl ester | |
| 241 | (2-[4-(1H-indol-3-yl)-piperidin-1-yl]-2-{1-[(2E)-3-m-tolyl-acryloyl]- | 529 |
| | piperidin-4-yl}-ethyl)-carbamic acid methyl ester | |
| 252 | N-{2-{1-[(2E)-3-(3,5-difluoro-phenyl)-acryloyl]-piperidin-4-yl}-2-[4-(1H- | 578 |
| | indol-3-yl)-piperidin-1-yl]-ethyl}-2-dimethylamino-acetamide | |

### EXAMPLE 23

### [4-(1H-pyrrol-3-yl)-piperidin-1-yl]-{1-[(2E)-3-(3,4,5-trifluoro-phenyl)-acryloyl]-piperidin-4-yl}-acetic acid (Cpd 150)

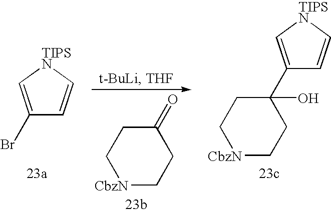

A solution of 3-bromo-1-triisopropylsilanyl-1H-pyrrole Compound 23a (2.42 g, 8.00 mmol, 1 eq) in THF (80 mL) was cooled to −78° C. tert-butyl lithium (1.7M in pentane, 9.6 mL, 16.00 mmol, 2 eq) was added dropwise with stirring. The mixture was stirred for 20 min and 4-oxo-piperidine-1-carboxylic acid benzyl ester Compound 23b (1.87 g, 8.00 mmol, 1 eq) was added and the mixture was stirred for an additional 20 mins. The solution was warmed to room temperature with stirring for 1.5 hrs. The reaction was partitioned between EtOAc and water and the aqueous layer was removed. Extraction of the aqueous layer with EtOAc was followed by combination of the organic layers, and washing twice with brine. The organic layer was dried over anhydrous Na₂SO₄, then filtered. The filtrate was evaporated and the crude product was purified via silica gel chromatography (2:1 hexanes:EtOAc) to provide 4-hydroxy-4-(1-triisopropylsilanyl-1H-pyrrol-3-yl)-piperidine-1-carboxylic acid benzyl ester Compound 23c (2.71 g, 74%) as a clear oil. ¹H NMR (CDCl₃, 400 MHz) δ: 7.39-7.28 (5H, m), 6.72 (1H, dd, J=2.6, 2.6 Hz), 6.68 (1H, dd, J=1.7, 1.7 Hz), 6.27 (1H, dd, J=3.0, 1.5 Hz), 5.14 (2H, s), 3.84 (2H, broad s), 3.46 (2H, app t, J=10.3 Hz), 2.04-1.81 (4H, m), 1.42 (3H, m), 1.08 (18H, d, J=7.5 Hz).

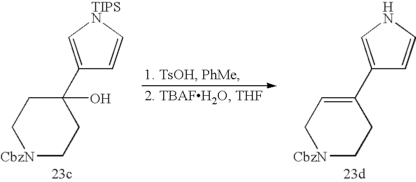

A solution of Compound 23c (557 mg, 1.21 mmol, 1 eq) in toluene (36 mL) was treated with TsOH.H₂O (19 mg, 0.098 mmol, 0.08 eq) and stirred for 30 mins at room temperature. The reaction was then partitioned between EtOAc and saturated aqueous NaHCO₃ and the aqueous layer was discarded. The organic layer was washed twice with brine, dried over anhydrous Na₂SO₄ and filtered. The filtrate was evaporated to provide a brown oil that was used without further purification. The oil (0.61 mmol, 1 eq) was dissolved in THF (10 mL) and treated with TBAF.H₂O (190 mg, 0.73 mmol, 1.2 eq). The mixture was stirred for 30 mins at room temperature, then between EtOAc and water. The aqueous layer was discarded and the organic layer was washed with brine. The organic layer was dried over anhydrous Na₂SO₄ and filtered. The filtrate was evaporated to provide a tan oil that was purified by silica gel chromatography (3:2 hexanes:EtOAc) to provide 4-(1H-pyrrol-3-yl)-3,6-dihydro-2H-pyridine-1-carboxylic acid benzyl ester Compound 23d (150 mg, 87%) in two steps as an oil. ¹H NMR (CD₃OD, 400 MHz) δ: 7.38-7.26 (5H, m), 6.75 (1H, s), 6.67 (1H, dd, J=2.0, 2.7 Hz), 6.23 (1H, dd, J=1.4, 2.8 Hz), 5.78 (1H, s), 5.13 (2H, s), 4.05 (2H, s), 3.63 (2H, s), 2.40 (2H, s).

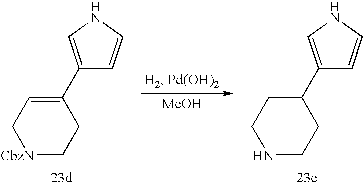

A solution of Compound 23d (64 mg, 0.23 mmol, 1 eq) and Pd(OH)₂ (20 wt. % on carbon, 40 mg, 0.057 mmol, 0.25 eq) in MeOH (13 mL) was sequentially purged with nitrogen (10 mins) and hydrogen, then pressurized with hydrogen (60 psi) and shaken for 16 hrs. The pressure was released and the solution was purged with nitrogen, then filtered through Celite and evaporated to provide 4-(1H-pyrrol-3-yl)-piperidine Compound 23d (32 mg, 94%) as a white solid. ¹H NMR (CD₃OD, 400 MHz) δ: 6.63 (1H, s), 6.53 (1H, s), 5.99 (1H, s), 3.13 (2H, m), 2.78 (2H, m), 2.62 (1H, m), 1.93 (2H, m), 1.58 (2H, m).

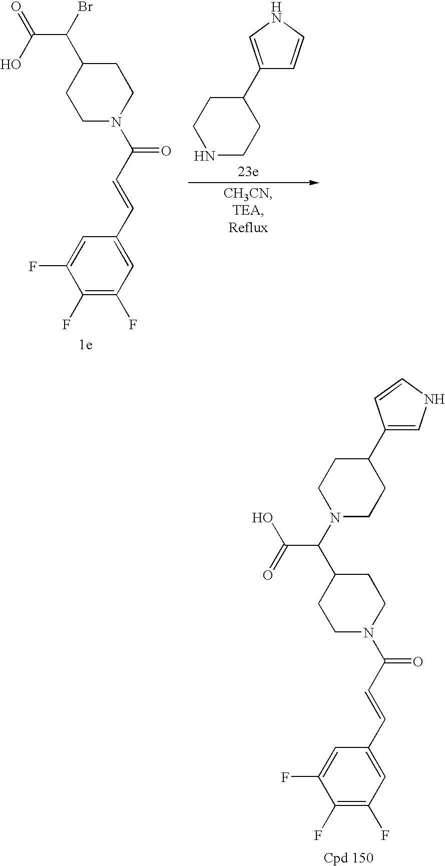

The procedure of Example 1 and Compound 23d in place of bromo-{1-[3-(3,4,5-trifluoro-phenyl)-acryloyl]-piperidin-4-yl}-acetic acid Compound 1e were used to provide Compound 150. MS m/z 476 (M+H)⁺.

### EXAMPLE 24

### (2E)-1-{4-[1-(4-furo[2,3-b]pyridin-3-yl-piperidin-1-yl)-2-hydroxy-ethyl]-piperidin-1-yl}-3-(3,4,5-trifluoro-phenyl)-propenone (Cpd 248)

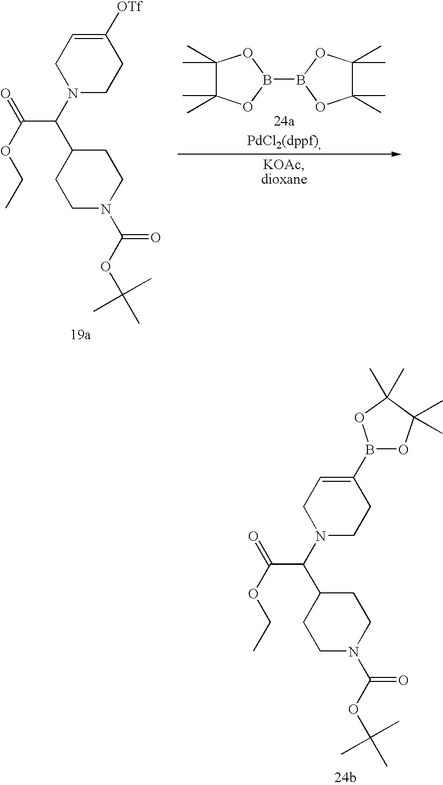

A solution of 4-[ethoxycarbonyl-(4-trifluoromethanesulfonyloxy-3,6-dihydro-2H-pyridin-1-yl)-methyl]-piperidine-1-carboxylic acid tert-butyl ester Compound 19a (200 mg, 0.40 mmol, 1 eq), 4,4,5,5,4′,4′,5′,5′-octamethyl-[2,2′]bi[[1,3,2]dioxaborolanyl] (also referred to as bis-pinacolato-diboron) Compound 24a (112 mg, 0.44 mmol, 1.1 eq), potassium acetate (118 mg, 1.20 mmol, 3 eq) and dichloro[1,1′-bis(diphenylphosphino)ferrocene]palladium dichloromethane adduct (10 mg, 0.012 mmol, 0.03 eq) in 1,4-dioxane (3 mL) was heated at 80° C. for 4 hrs. The reaction mixture was cooled and partitioned between EtOAc and brine. The organic layer was removed and the aqueous layer was extracted with EtOAc. The organic layers were combined, dried over anhydrous sodium sulfate, then filtered and evaporated to provide a crude residue that was purified via silica gel chromatography (3:1 to 2:1 hexanes:EtOAc) to provide 4-{ethoxycarbonyl-[4-(4,4,5,5-tetramethyl-[1,3,2]dioxaborolan-2-yl)-3,6-dihydro-2H-pyridin-1-yl]-methyl}-piperidine-1-carboxylic acid tert-butyl ester Compound 24a (137 mg, 72%) as a viscous oil. MS m/z 479 (M+H)⁺.

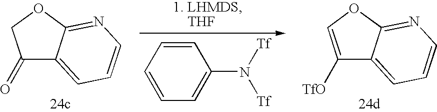

A solution of furo[2,3-b]pyridin-3-one Compound 24c (124 mg, 0.92 mmol, 1 eq) in THF (7.5 mL) was cooled to −78° C. and treated with dropwise addition of LHMDS (1M in THF, 1 mL, 1.01 mmol, 1.1 eq). The mixture was stirred for 30 min, then N-phenyl-trifluoromethanesulfonimide (361 mg, 1.01 mmol, 1.1 eq) was added and the reaction was warmed to 0° C. The mixture was then stirred for 1 hr at 0° C., then evaporated to dryness. The resulting crude residue was purified by neutral alumina chromatography (3:1 hexanes:EtOAc) to provide trifluoro-methanesulfonic acid furo[2,3-b]pyridin-3-yl ester Compound 24d, which was used immediately in the next step.

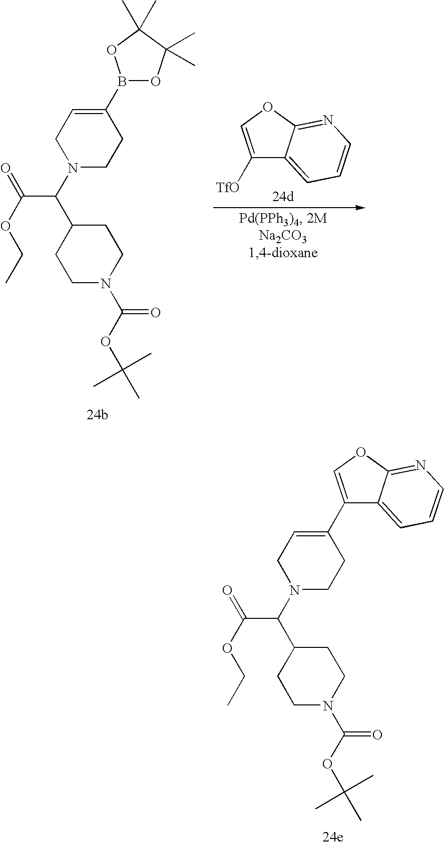

A solution of Compound 24b (94 mg, 0.20 mmol, 1 eq), Compound 24d (70 mg, 0.26 mmol, 1.3 eq), and tetrakis(triphenylphosphine) palladium (10 mg, 0.0087 mmol, 0.04 eq) in 2M sodium carbonate (0.4 mL) and 1,4-dioxane (2 mL) were added to a microwave reaction vessel. The solution was subjected to microwave irradiation (250 W pMax, 110° C., 4.5 min ramp, 5 min hold) and then cooled. The reaction was partitioned between EtOAc and saturated NaHCO3 and the organic layer removed. The aqueous layer was extracted with EtOAc and the organic layers were combined and dried over anhydrous sodium sulfate, then filtered and evaporated. The resulting residue was subjected to silica gel chromatography (1:1 hexanes:EtOAc) to provide 4-[ethoxycarbonyl-(4-furo[2,3-b]pyridin-3-yl-3,6-dihydro-2H-pyridin-1-yl)-methyl]-piperidine-1-carboxylic acid tert-butyl ester Compound 24e (51 mg, 54%). MS m/z 470 (M+H)⁺.

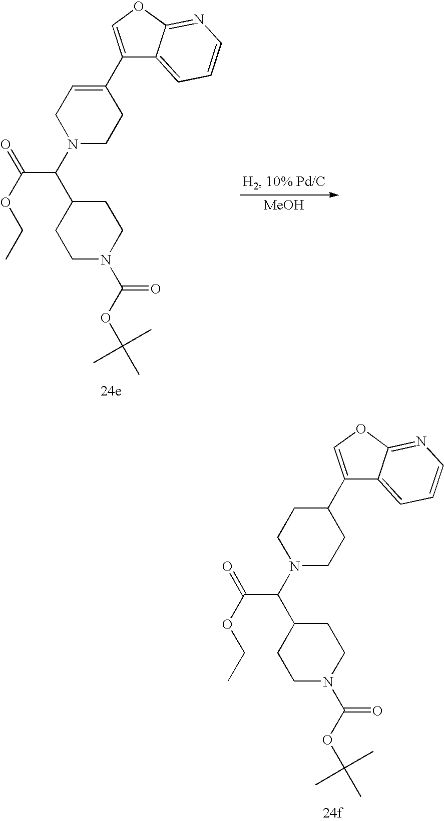

A solution of Compound 24e (51 mg, 0.11 mmol, 1 eq) and 10% palladium on carbon (50 mg, 0.047 mmol, 0.43 eq) in MeOH (2 mL) was sequentially purged with nitrogen and hydrogen and stirred under a balloon atmosphere of hydrogen for 16 hrs. The reaction mixture was purged with nitrogen, filtered through celite, then evaporated and subjected to silica gel chromatography (1:1:1 CH₂Cl₂:hexanes:EtOAc) to provide 4-[ethoxycarbonyl-(4-furo[2,3-b]pyridin-3-yl-piperidin-1-yl)-methyl]-piperidine-1-carboxylic acid tert-butyl ester Compound 24f (14 mg, 27%) as an oil. MS m/z 472 (M+H)⁺.

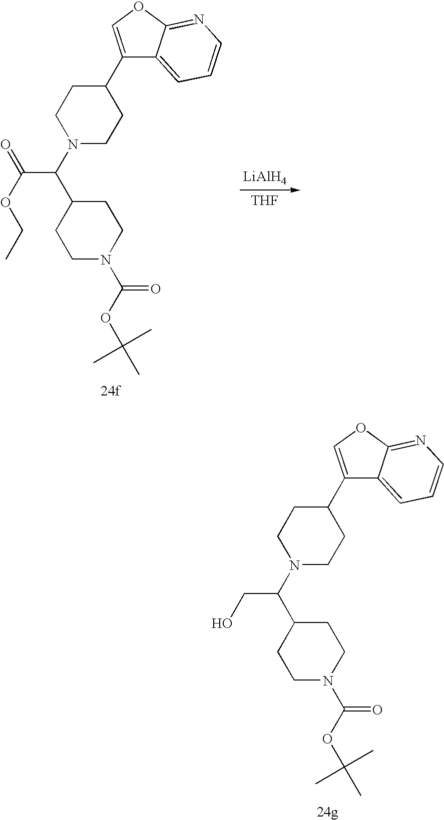

Compound 24f (14 mg, 0.030 mmol, 1 eq) was dissolved in THF and cooled to 0° C. A solution of lithium aluminum hydride (1M in THF, 0.045 mL, 0.045 mmol, 1.5 eq) was added dropwise with stirring, followed by additional lithium aluminum hydride solution (0.075 mL) over a 2 hr period. The reaction was quenched by successive addition of water (5 μL), 15% NaOH (5 mL), and water (15 μL). The solution was stirred for 1 hr, then filtered through celite and the solids were washed with EtOAc. The combined filtrates were evaporated to provide 4-[1-(4-furo[2,3-b]pyridin-3-yl-piperidin-1-yl)-2-hydroxy-ethyl]-piperidine-1-carboxylic acid tert-butyl ester Compound 24g (13 mg, quant) as a clear film that was used in the next step without further purification. MS m/z 430 (M+H)⁺.

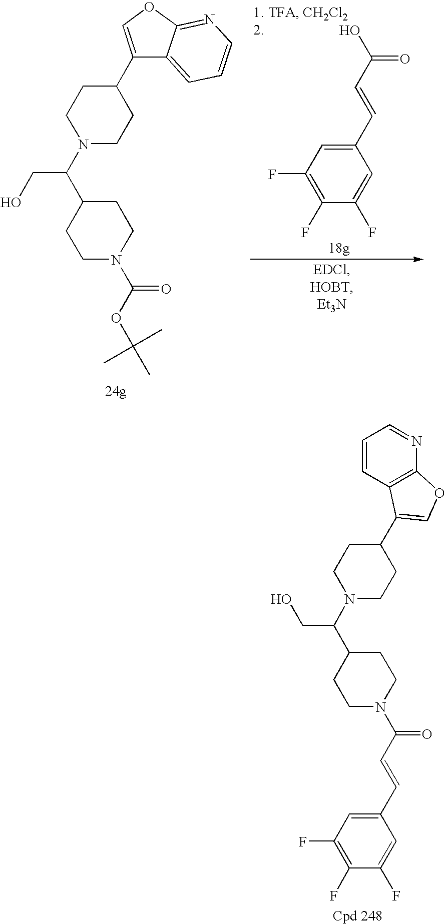

A solution of Compound 24g (13 mg, 0.030 mmol, 1 eq) in CH₂Cl₂ (4 mL) was cooled to 0° C. TFA (1 mL) was added and the reaction mixture was stirred at 0° C. for 1 hr, then room temperature for 2 hrs. The solvents were removed in vacuo and the resulting residue was dissolved in CH₂Cl₂ (1 mL) and DMF (0.2 mL). Triethylamine (0.017 mL, 0.12 mmol, 4 eq), HOBt (4 mg, 0.033 mmol, 1.1 eq), and 3-(3,4,5-trifluoro-phenyl)-acrylic acid Compound 18 g (6 mg, 0.030 mmol, 1 eq) were added and the reaction was cooled to 0° C. EDCI (7 mg, 0.036 mmol, 1.2 eq) was added and the reaction mixture was stirred for 16 hrs, slowly warming to room temperature. The solvents were removed in vacuo, then the resulting residue was dissolved in CH₂Cl₂ and partitioned with saturated NaHCO3. The organic layer was removed and the aqueous layer was extracted with CH₂Cl₂. The organic layers were combined, dried over anhydrous sodium sulfate, then filtered and evaporated to provide a crude residue, which was purified via silica gel chromatography to provide Compound 248 (7 mg, 45%) as a pale foam.

### EXAMPLE 25

### (2E)-1-(4-{(1S)-2-hydroxy-1-[4-(1H-indol-3-yl)-piperidin-1-yl]-ethyl}-piperidin-1-yl)-3-(3,4,5-trifluoro-phenyl)-propenone (Cpd 187)

### (2E)-1-(4-{(1R)-2-hydroxy-1-[4-(1H-indol-3-yl)-piperidin-1-yl]-ethyl}-piperidin-1-yl)-3-(3,4,5-trifluoro-phenyl)-propenone (Cpd 188)

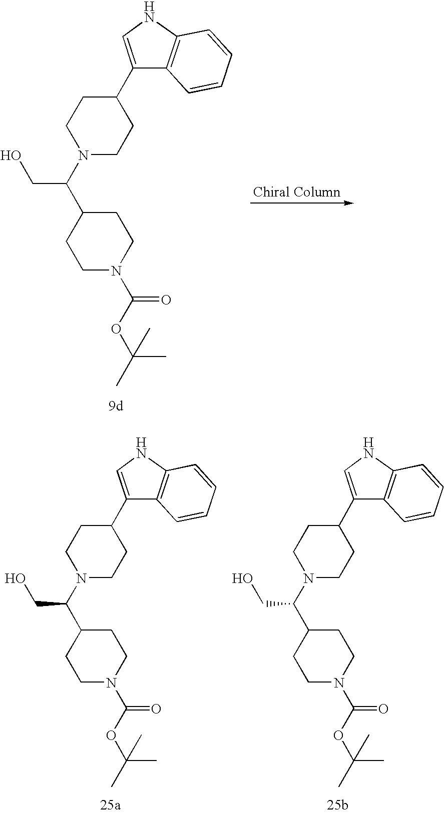

The racemic 4-{2-hydroxy-1-[4-(1H-indol-3-yl)-piperidin-1-yl]-ethyl}-piperidine-1-carboxylic acid tert-butyl ester Compound 9d (220 mg) was enantiomerically separated to provide a 4-{(1S)-2-hydroxy-1-[4-(1H-indol-3-yl)-piperidin-1-yl]-ethyl}-piperidine-1-carboxylic acid tert-butyl ester Compound 25a (60 mg, 55%) and a 4-{(1R)-2-hydroxy-1-[4-(1H-indol-3-yl)-piperidin-1-yl]-ethyl}-piperidine-1-carboxylic acid tert-butyl ester Compound 25b (60 mg, 55%) via chiral HPLC chromatography using a Chiralpak AD column (Mobile phase: 15% heptane in ethanol). MS m/z 428 (M+H)+(for each enantiomer).

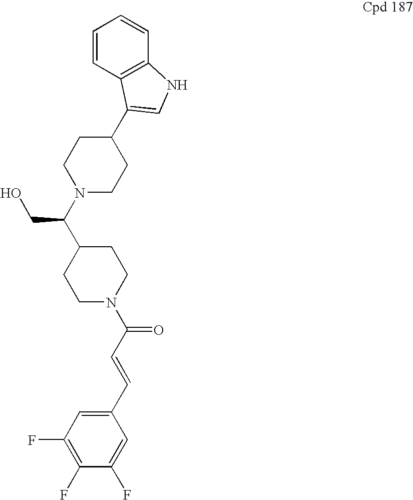

The procedure of Example 9 and Compound 25a in place of Compound 9d were used to provide Compound 187. MS m/z 512 (M+H)⁺.

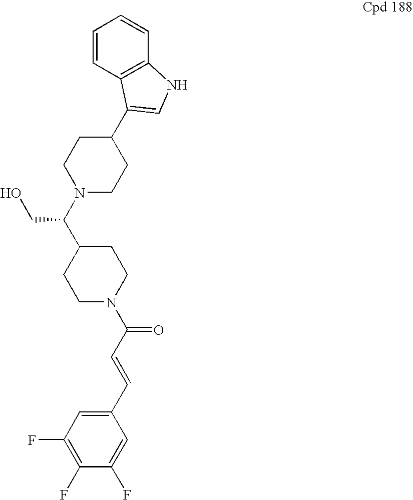

The procedure of Example 9 and Compound 25b in place of Compound 9d were used to provide Compound 188. MS m/z 512 (M+H)⁺.

Using the procedure of Example 25 (with the exception of the mobile phase being changed from 15% heptane in ethanol to 15% ethanol in heptane) and known appropriate reagents and starting materials, the following compounds of the invention were prepared:

| Cpd | Name | MS |
|---|---|---|
| 180 | (2E)-3-(3,5-difluoro-phenyl)-1-(4-{(1S)-2- | 485 |
| | hydroxy-1-[4-(4-methoxy-phenyl)-piperidin- | |
| | 1-yl]-ethyl}-piperidin-1-yl)-propenone | |
| 181 | (2E)-3-(3,5-difluoro-phenyl)-1-(4-{(1R)-2- | 485 |
| | hydroxy-1-[4-(4-methoxy-phenyl)-piperidin-1- | |
| | yl]-ethyl}-piperidin-1-yl)-propenone | |

### EXAMPLE 26

### [4-(benzylcarbamoyl-methyl)-piperidin-1-yl]-{1-[(2E)-3-(3,5-difluoro-phenyl)-acryloyl]-piperidin-4-yl}-acetic acid (Cpd 148)

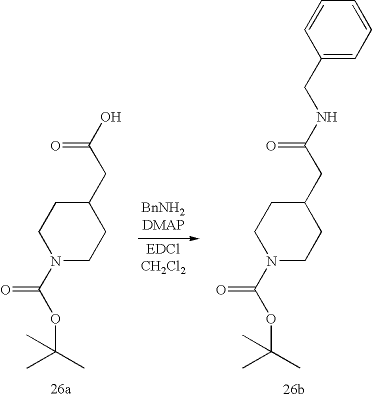

A solution of benzylamine (655 mL, 6.00 mmol, 3 eq), 4-carboxymethyl-piperidine-1-carboxylic acid tert-butyl ester Compound 26a (487 mg, 2.00 mmol, 1 eq) and DMAP (24 mg, 0.20 mmol, 0.1 eq) in CH₂Cl₂ (5 mL) was treated with EDCI (422 mg, 2.20 mmol, 1.1 eq). The mixture was stirred for 16 hrs, then the reaction mixture was poured into EtOAc and sequentially washed with 1N HCl, brine, saturated NaHCO₃ and brine. The organic layer was dried over anhydrous sodium sulfate, then filtered and evaporated to provide 4-(benzylcarbamoyl-methyl)-piperidine-1-carboxylic acid tert-butyl ester Compound 26b (455 mg, 69%) as a white solid that was used in the next step without further purification. MS m/z 355 (M+H)⁺.

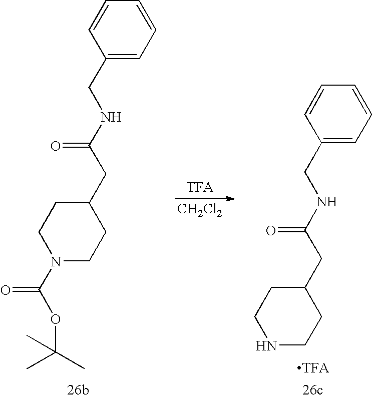

A solution of Compound 26b (93 mg, 0.28 mmol) in CH₂Cl₂ (1.5 mL) was cooled to 0° C. with stirring. TFA (0.5 mL) was added dropwise and the reaction mixture was stirred for 4 hrs. The solvents were removed in vacuo to provide N-benzyl-2-piperidin-4-yl-acetamide, trifluoroacetate salt Compound 26c (96 mg, 99%) as a clear oil that was used in the next step without further purification.

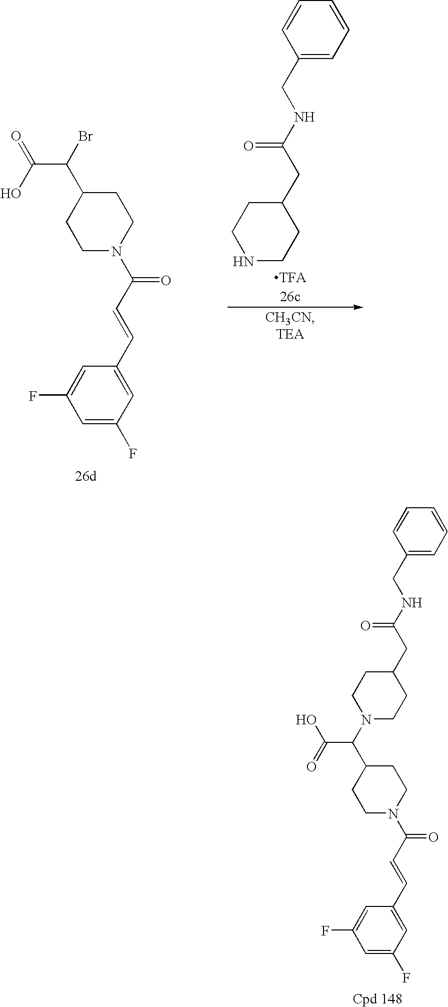

The procedure of Example 1 and 3-(3,5-difluoro-phenyl)-acryloyl chloride Compound 12b in place of 3-(3,4,5-trifluoro-phenyl)-acryloyl chloride Compound 1a was used to prepare bromo-{1-[3-(3,5-difluoro-phenyl)-acryloyl]-piperidin-4-yl}-acetic acid Compound 26d.

The procedure of Example 1, Compound 26c in place of bromo-{1-[3-(3,4,5-trifluoro-phenyl)-acryloyl]-piperidin-4-yl}-acetic acid Compound 1e and Compound 26c in place of 3-piperidin-4-yl-1H-indole Compound 1f were used to provide Compound 148. MS m/z 540 (M+H)⁺.

Using the procedure of Example 26 and known appropriate reagents and starting materials, the following compounds of the invention were prepared:

| Cpd | Name | MS |
|---|---|---|
| 149 | [4-(benzylcarbamoyl-methyl)-piperidin-1- | 558 |
| | yl]-{1-[(2E)-3-(3,4,5-trifluoro-phenyl)- | |
| | acryloyl]-piperidin-4-yl}-acetic acid | |

### EXAMPLE 27

### (2E)-1-(4-{2-chloro-1-[4-(4-chloro-phenyl)-piperidin-1-yl]-ethyl}-piperidin-1-yl)-3-(4-trifluoromethyl-phenyl)-propenone (Cpd 247)

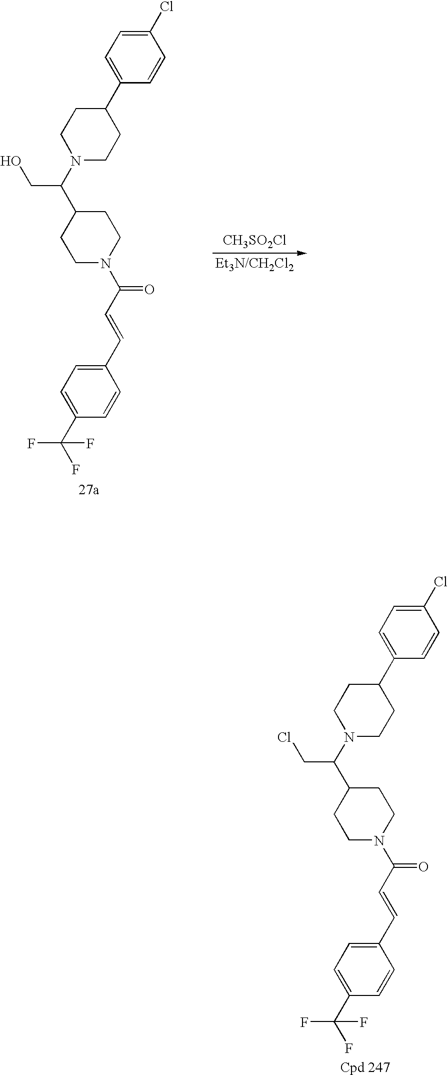

Et₃N (0.02 mL, 0.14 mmol) and methanesulfonyl chloride (10 mg, 0.088 mmol) were added to a solution of Compound 27a (20 mg, 0.041 mmol) in DCM (3 mL). The mixture was stirred at room temperature for 2 hrs, then concentrated in vacuo for 0.5 hrs. The resulting residue was purified via preparative TLC with 50% EtOAc/Hexane to provide Compound 247 (7 mg, 32%). MS m/z 539 (M+H)⁺.

Using the procedure of Example 27 and known appropriate reagents and starting materials, the following compounds of the invention were prepared:

| Cpd | Name | MS |
|---|---|---|
| 245 | (2E)-1-(4-{2-chloro-1-[4-(4-chloro-phenyl)- | 539 |
| | piperidin-1-yl]-ethyl}-piperidin-1-yl)-3- | |
| | (3,4-dichloro-phenyl)-propenone | |

Biological Activity

Compounds of the invention were subjected to various representative biological tests.

The results of these tests are intended to illustrate the invention in a non-limiting fashion.

### EXAMPLE 28

MCP-1 Receptor Binding Assay in THP-1 Cells

THP-1 cells were obtained from American Type Culture Collection (Manassas, Va., USA). The THP-1 cells were grown in RPMI-1640 supplemented with 10% fetal bovine serum in a humidified 5% CO₂ atmosphere at 37° C. The cell density was maintained between 0.5×10⁶ cells/mL.

TH-1 cells were incubated with 0.5 nM ¹²⁵I labeled MCP-1 (Perkin-Elmer Life Sciences, Inc. Boston, Mass.) in the presence of varying concentrations of either unlabeled MCP-1 (R & D Systems, Minneapolis, Minn.) or test compound for 2 hours at 30° C. in a 96 well plate. Cells were then harvested onto a filter plate, dried, and 20 μL of Microscint 20 was added to each well. Plates were counted in a TopCount NXT, Microplate Scintillation & Luminescence Counter (Perkin-Elmer Life Sciences, Inc. Boston, Mass.). Blank values (buffer only) were subtracted from all values and drug treated values were compared to vehicle treated values. 1 μM cold MCP-1 was used for nonspecific binding.

Table 1 lists IC₅₀ values for inhibition of MCP-1 binding to CCR2 obtained for test compounds of the invention. Where an IC₅₀ value was not obtained for a particular compound, the percent inhibition is provided at a test concentration of 25 μM.

TABLE 1
Inhibition of MCP-1 Binding IC₅₀ (μM)

| Cpd | IC₅₀ |
|---|---|
| 1 | 0.253 |
| 2 | 1.83 |
| 3 | 3.8 |
| 4 | 0.37 |
| 5 | 0.84 |
| 6 | 0.002 |
| 7 | 0.02 |
| 8 | 0.065 |
| 9 | 0.035 |
| 10 | 8.6 |
| 11 | 2.167 |
| 12 | 0.41 |
| 13 | 0.001 |
| 14 | 0.364 |
| 15 | 0.015 |
| 16 | 0.03 |
| 17 | 0.16 |
| 18 | 0.004 |
| 19 | 0.01 |
| 20 | 0.024 |
| 21 | 3.4 |
| 22 | 0.025 |
| 23 | 0.015 |
| 24 | 0.01 |
| 25 | 0.007 |
| 26 | 0.02 |
| 27 | 0.08 |
| 28 | 0.1 |
| 29 | 0.024 |
| 30 | 0.017 |
| 31 | 0.008 |
| 32 | 1.1 |
| 33 | 0.72 |
| 34 | 0.01 |
| 35 | 0.008 |
| 36 | 0.008 |
| 37 | 0.655 |
| 38 | 0.02 |
| 39 | 0.002 |
| 40 | 0.05 |
| 41 | 0.014 |
| 42 | 0.007 |
| 43 | 1.1 |
| 44 | 2.7 |
| 45 | 0.14 |
| 46 | 0.001 |
| 47 | 0.01 |
| 48 | 0.03 |
| 49 | 0.025 |
| 50 | 0.03 |
| 51 | 0.3 |
| 52 | 0.03 |
| 53 | 0.006 |
| 54 | 1.4 |
| 55 | 0.115 |
| 56 | 0.06 |
| 57 | 0.02 |
| 58 | 0.09 |
| 59 | 0.21 |
| 60 | 0.04 |
| 61 | 0.12 |
| 62 | 0.08 |
| 63 | 1.61 |
| 64 | 0.02 |
| 65 | 0.353 |
| 66 | 17.70 |
| 67 | 0.845 |
| 68 | 3.55 |
| 69 | 14.2 |
| 70 | 0.003 |
| 71 | 0.02 |
| 72 | 0.03 |
| 73 | 0.15 |
| 74 | 0.005 |
| 75 | 0.004 |
| 76 | 0.002 |
| 77 | 0.07 |
| 78 | 0.14 |
| 79 | 0.008 |
| 80 | 0.078 |
| 81 | 0.03 |
| 82 | 0.11 |
| 83 | 0.004 |
| 84 | 2.9 |
| 85 | 0.17 |
| 86 | 0.21 |
| 87 | 2.802 |
| 88 | 0.02 |
| 89 | 0.095 |
| 90 | 0.48 |
| 91 | 0.305 |
| 92 | 0.04 |
| 93 | 0.004 |
| 94 | 0.01 |
| 95 | 0.02 |
| 96 | 0.12 |
| 97 | 0.25 |
| 98 | 0.89 |
| 99 | 0.81 |
| 100 | 0.43 |
| 101 | 0.02 |
| 102 | 0.26 |
| 103 | 0.07 |
| 104 | 0.09 |
| 105 | 0.09 |
| 106 | 0.02 |
| 107 | 1.8 |
| 108 | 0.003 |
| 109 | 0.02 |
| 100 | 6.8 |
| 111 | 11.2 |
| 112 | 0.004 |
| 113 | 0.006 |
| 114 | 0.35 |
| 115 | 0.32 |
| 116 | 0.0006 |
| 117 | 1 |
| 118 | 3.2 |
| 119 | 0.01 |
| 120 | 0.08 |
| 121 | 0.0002 |
| 122 | 0.04 |
| 123 | 0.009 |
| 124 | 0.13 |
| 125 | 1.7 |
| 126 | 2.1 |
| 127 | 0.76 |
| 128 | 0.32 |
| 129 | 0.04 |
| 130 | 8.55 |
| 131 | 3.9 |
| 132 | 0.05 |
| 133 | 0.010 |
| 134 | 0.3 |
| 135 | 0.94 |
| 136 | 0.08 |
| 137 | 0.03 |
| 138 | 0.172 |
| 139 | 0.02 |
| 140 | 1.6 |
| 141 | 0.34 |
| 142 | 0.005 |
| 143 | 0.01 |
| 144 | 0.05 |
| 145 | 5.85 |
| 146 | 0.007 |
| 147 | 0.15 |
| 148 | 8.8 |
| 149 | 16.6 |
| 150 | 1.6 |
| 151 | 0.01 |
| 152 | 1.9 |
| 153 | 0.003 |
| 154 | 0.27 |
| 155 | 0.207 |
| 156 | 0.08 |
| 157 | 0.44 |
| 158 | 0.1 |
| 159 | 0.27 |
| 160 | 56% |
| 161 | 0.05 |
| 162 | 0.007 |
| 163 | 0.03 |
| 164 | 0.01 |
| 165 | 0.08 |
| 166 | 0.006 |
| 167 | 0.073 |
| 168 | 0.02 |
| 169 | 0.057 |
| 170 | 0.04 |
| 171 | 0.0045 |
| 172 | 0.032 |
| 173 | 0.43 |
| 174 | 0.15 |
| 175 | 0.188 |
| 176 | 0.07 |
| 177 | 3 |
| 178 | 0.09 |
| 179 | 0.23 |
| 180 | 0.07 |
| 181 | 0.04 |
| 182 | 0.33 |
| 183 | 0.47 |
| 184 | 1.6 |
| 185 | 0.84 |
| 186 | 0.36 |
| 187 | 0.0006 |
| 188 | 0.0295 |
| 189 | 0.17 |
| 190 | 0.21 |
| 191 | 0.1 |
| 192 | 0.22 |
| 193 | 0.14 |
| 194 | 2.3 |
| 195 | 3.3 |
| 196 | 5.7 |
| 197 | 1.2 |
| 198 | 0.0006 |
| 199 | 0.02 |
| 200 | 2 |
| 201 | 0.001 |
| 202 | 0.0193 |
| 203 | 0.51 |
| 204 | 0.004 |
| 205 | 0.04 |
| 206 | 2 |
| 207 | 0.21 |
| 208 | 0.215 |
| 209 | 52% |
| 210 | 5 |
| 211 | 0.02 |
| 212 | 58% |
| 213 | 0.08 |
| 214 | 0.07 |
| 215 | 0.09 |
| 216 | 0.25 |
| 217 | 0.21 |
| 218 | 0.37 |
| 219 | 0.34 |
| 220 | 0.44 |
| 221 | 0.41 |
| 222 | 0.68 |
| 223 | 4.1 |
| 224 | 54% |
| 225 | 1.3 |
| 226 | 2.1 |
| 227 | 0.96 |
| 228 | 2.4 |
| 229 | 1.7 |
| 230 | 2.1 |
| 231 | 4.6 |
| 232 | 4 |
| 233 | 0.66 |
| 234 | 11.2 |
| 235 | 0.03 |
| 236 | 0.02 |
| 237 | 0.215 |
| 238 | 2.4 |
| 239 | 3 |
| 240 | 4.6 |
| 241 | 58% |
| 242 | 0.23 |
| 243 | 0.09 |
| 244 | 0.26 |
| 245 | 2.17 |
| 246 | 0.07 |
| 247 | 53% |
| 248 | 1.9 |
| 249 | 0.02 |
| 250 | 2.9 |
| 251 | 0.39 |
| 252 | 5.8 |
| 253 | 42% |
| 254 | 0.12 |
| 255 | 2.4 |
| 256 | 25% |
| 258 | 0.2 |
| 259 | 0.002 |

### EXAMPLE 29

MCP-1 Induced Calcium Mobilization in THP-1 Cells

THP-1 cells were plated at a density of 8×10⁵ cells/mL (100 μL/well) into poly-D lysine coated clear bottom, black 96 well plates. The cells were loaded with 5 μM fluo-3 for 45 minutes. The fluo-3 was washed off and cells were incubated with varying concentrations of test compound for 15 minutes. The change in calcium ion concentration upon addition of 0.2 μM MCP-1 was determined using FLIPR and compared to vehicle.

Table 2 lists IC₅₀ values for inhibition of MCP-1 induced influx of calcium ions. Where an IC₅₀ value was not obtained for a particular compound, the percent inhibition is provided at a test concentration of 25 μM.

TABLE 2
Inhibition of MCP-1 Induced Calcium Ion Influx IC₅₀ (μM)

| Cpd | IC₅₀ |
|---|---|
| 6 | 0.005 |
| 9 | 0.002 |
| 13 | 0.004 |
| 14 | 1.13 |
| 65 | 0.12 |
| 87 | 0.36 |
| 88 | 0.41 |
| 89 | 0.47 |
| 91 | 0.89 |
| 96 | 0.14 |
| 97 | 0.97 |
| 98 | 1.85 |
| 99 | 1.6 |
| 100 | 0.48 |
| 101 | 0.13 |
| 102 | 0.86 |
| 103 | 0.49 |
| 104 | 1.01 |
| 105 | 0.13 |
| 106 | 0.11 |
| 108 | 0.01 |
| 109 | 0.03 |
| 112 | 0.0006 |
| 113 | 0.001 |
| 114 | 0.21 |
| 115 | 0.18 |
| 116 | 0.002 |
| 119 | 0.008 |
| 120 | 0.001 |
| 121 | 0.0001 |
| 122 | 0.0008 |
| 123 | 0.004 |
| 124 | 0.07 |
| 127 | 0.82 |
| 128 | 0.02 |
| 129 | 0.02 |
| 132 | 0.003 |
| 133 | 0.0008 |
| 134 | 0.01 |
| 135 | 7.1 |
| 136 | 0.13 |
| 137 | 0.21 |
| 138 | 1.29 |
| 139 | 0.04 |
| 141 | 6.9 |
| 142 | 0.03 |
| 143 | 0.08 |
| 144 | 1.3 |
| 146 | 0.05 |
| 147 | 0.6 |
| 153 | 0.007 |
| 154 | 4.8 |
| 155 | 0.94 |
| 156 | 50% |
| 157 | 0.32 |
| 158 | 0.14 |
| 159 | 2.1 |
| 160 | 33% |
| 161 | 0.18 |
| 162 | 0.002 |
| 163 | 0.01 |
| 164 | 0.009 |
| 165 | 0.11 |
| 166 | 0.008 |
| 167 | 0.03 |
| 168 | 0.01 |
| 169 | 0.17 |
| 170 | 0.01 |
| 171 | 0.007 |
| 172 | 0.02 |
| 173 | 21% |
| 175 | 2.30 |
| 176 | 2.61 |
| 178 | 2.35 |
| 179 | 2.06 |
| 180 | 0.12 |
| 181 | 0.16 |
| 182 | 7.87 |
| 183 | 9.25 |
| 184 | 14% |
| 185 | 4.6 |
| 186 | 6.1 |
| 187 | 0.00005 |
| 188 | 0.01 |
| 189 | 0.16 |
| 190 | 0.25 |
| 191 | 0.17 |
| 192 | 0.17 |
| 193 | 0.14 |
| 198 | 0.00002 |
| 199 | 0.004 |
| 201 | 0.0006 |
| 202 | 0.008 |
| 203 | 5 |
| 204 | 0.005 |
| 205 | 0.02 |
| 207 | 0.11 |
| 208 | 0.0008 |
| 211 | 0.005 |
| 213 | 0.09 |
| 214 | 0.18 |
| 215 | 0.02 |
| 216 | 1.8 |
| 217 | 2 |
| 218 | 1.9 |
| 219 | 52% |
| 220 | 0.96 |
| 227 | 0.87 |
| 233 | 1.8 |
| 235 | 0.02 |
| 236 | 0.03 |
| 237 | 0.07 |
| 242 | 0.04 |
| 244 | 0.08 |
| 245 | 0.4 |
| 246 | 0.02 |
| 251 | 0.56 |
| 253 | 3.9 |
| 254 | 0.03 |
| 256 | 11 |
| 258 | 2.3 |
| 259 | 88% |

### EXAMPLE 30

MCP-1 Induced Chemotaxis in THP-1 Cells

MCP-1 induced chemotaxis was run in a 24-well chemotaxis chamber. MCP-1 (0.01 μg/mL) was added to the lower chamber and 100 μL of THP-1 cells (1×10⁷ cell/mL) was added to the top chamber. Varying concentrations of test compound were added to the top and bottom chambers. Cells were allowed to chemotax for 3 hours at 37° C. and 5% CO₂. An aliquot of the cells that had migrated to the bottom chamber was taken and counted then compared to vehicle.

Table 3 lists IC₅₀ values for inhibition of MCP-1 induced chemotaxis. Where an IC₅₀ value was not obtained for a particular compound, the percent inhibition is provided at a test concentration of 25 μM.

TABLE 3
Inhibition of MCP-1 Induced Chemotaxis IC₅₀ (μM)

| Cpd | IC50 |
|---|---|
| 2 | 1.81 |
| 6 | 0.008 |
| 7 | 0.008 |
| 8 | 0.01 |
| 9 | 0.02 |
| 13 | 0.006 |
| 14 | 0.07 |
| 15 | 0.006 |
| 16 | 0.02 |
| 17 | 0.02 |
| 18 | 0.008 |
| 19 | 0.004 |
| 20 | 0.01 |
| 22 | 0.004 |
| 23 | 0.003 |
| 24 | 0.0007 |
| 25 | 0.01 |
| 26 | 0.03 |
| 27 | 0.01 |
| 28 | 0.43 |
| 29 | 0.0004 |
| 30 | 0.001 |
| 31 | 0.002 |
| 33 | 0.61 |
| 34 | 0.006 |
| 35 | 0.03 |
| 36 | 0.0004 |
| 37 | 0.38 |
| 38 | 0.004 |
| 39 | 0.0019 |
| 40 | 0.03 |
| 41 | 0.04 |
| 42 | 0.0008 |
| 46 | 0.0002 |
| 47 | 0.0002 |
| 48 | 0.04 |
| 49 | 0.004 |
| 53 | 0.0007 |
| 57 | 0.003 |
| 58 | 0.13 |
| 59 | 0.09 |
| 60 | 0.07 |
| 61 | 0.08 |
| 62 | 0.18 |
| 65 | 1.6 |
| 70 | 0.02 |
| 71 | 0.007 |
| 72 | 0.03 |
| 74 | 0.006 |
| 75 | 0.009 |
| 76 | 0.01 |
| 77 | 0.06 |
| 81 | 0.03 |
| 82 | 0.21 |
| 83 | 0.03 |
| 85 | 0.19 |
| 86 | 0.28 |
| 87 | 1 |
| 88 | 0.24 |
| 89 | 0.21 |
| 91 | 0.27 |
| 92 | 0.1 |
| 93 | 0.02 |
| 94 | 0.01 |
| 95 | 0.02 |
| 96 | 0.08 |
| 97 | 0.23 |
| 98 | 2.2 |
| 99 | 2.5 |
| 100 | 0.94 |
| 101 | 0.14 |
| 102 | 0.23 |
| 103 | 0.09 |
| 104 | 0.16 |
| 105 | 0.01 |
| 106 | 0.21 |
| 108 | 0.02 |
| 109 | 0.03 |
| 112 | 0.004 |
| 113 | 0.095 |
| 114 | 0.29 |
| 115 | 0.46 |
| 116 | 0.0004 |
| 119 | 0.01 |
| 121 | 0.012 |
| 123 | 0.005 |
| 127 | 0.75 |
| 129 | 0.08 |
| 132 | 0.07 |
| 133 | 0.04 |
| 134 | 0.09 |
| 135 | 0.77 |
| 136 | 0.14 |
| 137 | 0.08 |
| 138 | 0.217 |
| 139 | 0.05 |
| 141 | 0.76 |
| 142 | 0.06 |
| 143 | 0.08 |
| 144 | 0.5 |
| 146 | 0.053 |
| 147 | 0.04 |
| 151 | 0.03 |
| 153 | 0.009 |
| 154 | 0.16 |
| 155 | 0.13 |
| 156 | 0.12 |
| 157 | 0.46 |
| 158 | 0.1 |
| 159 | 0.86 |
| 161 | 0.09 |
| 162 | 0.02 |
| 163 | 0.15 |
| 164 | 0.04 |
| 165 | 0.025 |
| 166 | 0.03 |
| 167 | 0.03 |
| 168 | 0.04 |
| 169 | 0.055 |
| 170 | 0.009 |
| 171 | 0.006 |
| 172 | 0.03 |
| 173 | 0.13 |
| 174 | 0.45 |
| 175 | 0.3 |
| 176 | 0.09 |
| 178 | 0.18 |
| 179 | 0.14 |
| 180 | 0.09 |
| 181 | 0.07 |
| 182 | 0.35 |
| 183 | 0.4 |
| 185 | 0.34 |
| 186 | 0.96 |
| 187 | 0.002 |
| 188 | 0.02 |
| 189 | 0.72 |
| 190 | 0.2 |
| 191 | 0.15 |
| 192 | 0.35 |
| 193 | 1.3 |
| 198 | 0.0002 |
| 199 | 0.03 |
| 201 | 0.003 |
| 202 | 0.015 |
| 203 | 1.2 |
| 204 | 0.01 |
| 205 | 0.04 |
| 207 | 0.19 |
| 208 | 0.013 |
| 211 | 0.008 |
| 213 | 0.17 |
| 214 | 0.19 |
| 215 | 0.46 |
| 216 | 0.7 |
| 217 | 0.62 |
| 235 | 0.008 |
| 236 | 0.02 |
| 237 | 0.11 |
| 242 | 0.27 |
| 251 | 0.17 |
| 254 | 0.02 |
| 259 | 0.005 |

### EXAMPLE 31

Collagen-Induced Arthritis Model

In a collagen-induced arthritis model in mice, DBA1 mice were immunized with bovine type II collagen on day 0, injected (sc) with lipopolysaccharide (LPS) on day 21, and dosed (ip, bid) with a test compound at either 25, 50 or 100 mg/kg from day 20 to day 35. Body weight was monitored, and clinical disease score recorded every 2-3 days starting on day 20.

Test compound was dosed in one of two vehicles:

1) 10% Pharmasolve:20% PEG-400:70% of a 1% solution of Tween-80 in water; or,
2) 30% PEG400:20% Solutol:50% of a 0.1 N solution of NaHCO₃.

At a dose of 100 mg/kg, Compound 6 (in either vehicle) inhibited the development of arthritis (clinical disease score on day 35) by greater than 90%.

Compound 13 (Pharmasolve vehicle only) inhibited the development of arthritis (clinical disease score on day 35) by 23%, 50% and 79% at the 25, 50, and 100 mg/kg doses, respectively. Histological analyses showed that the compounds significantly inhibited infiltration of monocytes and lymphocytes into the joints, but did not significantly affect infiltration by polymorphonuclear leukocytes.

### EXAMPLE 32

Adjuvant-Induced Arthritis Model (Dosing from Day 0-14)

In the adjuvant-induced arthritis model, 7-week old male Lewis rats are injected in the right hind footpad with a mixture of heat-killed Mycobacterium Butyricum (0.5 mg) in liquid paraffin oil (50 μL). An increase in volume of the contralateral (non-injected) hind paw is a measure of arthritis severity.

Body weight and hind paw volume (as measured by mercury plethysmography volume displacement) are typically recorded on days 0, 3, 7, 10, 12, 14, and 16. Animals were dosed with test Compound 6 (ip, bid, 100 mg/kg) from days 0-14, or with a vehicle control. As a positive control for inhibition, a separate group of rats was injected with indomethacin (orally, once per day, 3 mg/kg) from days 10-14.

Animals dosed with Compound 6 demonstrated insignificant swelling of the contralateral paws and a 40% decrease in swelling in the injected paws. Indomethacin inhibited contralateral paw swelling by 72% and swelling in the adjuvant-injected paws by 38%.

### EXAMPLE 33

Adjuvant-Induced Arthritis Model (Prophylactic Dosing from Day 7-14)

Following the procedure of Example 32, animals were dosed with test Compound 13 (ip, bid, 100 mg/kg), or with vehicle alone, from days 7-14. Under these conditions, Compound 13 inhibited swelling of the contralateral paws by 94%.

### EXAMPLE 34

Adjuvant-Induced Arthritis Model (Therapeutic Dosing from Day 12-16)

Following the procedure of Example 32, animals were dosed with test Compound 6 (ip, bid, 100 mg/kg), or with vehicle alone, from days 12-16 (after the contralateral paws had already started to swell as a result of the arthritis). Again, indomethacin (orally, once per day, 3 mg/kg) was used as a positive control.

Under these conditions, Compound 6 inhibited contralateral paw swelling by 51% and decreased swelling in the injected paw by 40%. Indomethacin inhibited contralateral paw swelling by 69% and inhibited adjuvant-injected paw swelling by 40%.

### EXAMPLE 35

Mouse Model of Allergic Asthma:

An allergic asthma model in mice was used to test compounds of the invention for therapeutic effect on asthmatic response as a function of airway inflammation and hyperresponsiveness (Malaviya, et al., J. Phar. Exp. Ther., 2000, 295: 912-926). Airway hyperresponsiveness in asthmatic patients is a cardinal feature of allergic asthma and is maintained as a result of persistent airway inflammation. Eosinophils are the prominent cells involved in airway inflammation and are found in large numbers in sputum and bronchoalveolar lavage fluids.

Airway responsiveness was measured in unrestrained mice by noninvasive whole body plethysmography using a BioSystem plethysmography instrument (BUXCO, Troy, N.Y.). Each animal was individually placed in the plethysmography instrument chamber and chamber pressure was used as a measure of the difference between thoracic volume expansion or contraction and air volume removed or added to the chamber during breathing. The differential of this function with respect to time produced a pseudo flow value that was proportionate to the difference between the rate of the thoracic volume expansion and nasal air flow (Hamelmann, et al., J. Respir. Crit. Care Med., 1997, 156: 766-775).

Animals and Method:

Three treatment groups of BALB/c female mice (6-8 weeks old) were tested in the 32 day study:
Group 1: vehicle control phosphate buffered saline (PBS)-sensitized and PBS-challenged mice;
Group 2: positive control ovalbumin (OVA)-sensitized and OVA-challenged mice; and,
Group 3: OVA-sensitized and OVA-challenged mice treated with Compound 13.

The vehicle used was a mixture of 20% Solutol, 30% PEG400 and 50% 0.1N NaHCO₃.

Day 0 and 14:

Group 1 mice were sensitized by injection (ip) with PBS; and,
Group 2 mice were OVA sensitized by injection (ip) with OVA (20 μg) dissolved in PBS adsorbed on 2.25 mg alum.
   Day 28, 29 and 30:
   Challenge Phase
Group 1 mice were challenged with PBS by ultrasonic nebulization for 20 min.
A first subset of Group 2 mice was OVA-challenged by ultrasonic nebulization of OVA (5 mg/mL) for 20 min.
A second subset of Group 2 mice was also OVA-challenged by ultrasonic nebulization of OVA (5 mg/mL) for 20 min.
   Treatment Phase
Group 1 mice were treated by injection (ip) with vehicle at 30 min before and at 6 hr after the PBS challenge.
Group 2 (first subset) mice were treated by injection (ip) with vehicle at 30 min before and at 6 hr after the OVA challenge.
Group 2 (second subset) mice were treated by injection (ip) with Compound 13 (100 mg/kg) at 30 min before and at 6 hr after the OVA challenge. The second subset was then designated as treatment Group 3.
   Day 31:
Group 1 and Group 2 (first subset) mice were dosed twice with vehicle alone, the second dose for each group was administered 6 hr after the first dose; and,
Group 3 mice were dosed twice with Compound 13 (100 mg/kg), the second dose was administered 6 hr after the first dose.
   Day 32:

The three treatment groups were challenged via airway by means of methacholine inhalation and asthmatic response was measured as a function of airway hyper-responsiveness.

Baseline Phase

A baseline reading over a 5 min period for each of the mice in the three treatment groups was taken in the plethysmography instrument, then the baseline readings were averaged.

Challenge Phase

Group 1 mice were nebulized with saline at increasing doses (1-30 mg/ml) over a 2 min period.
Group 2 (first subset) and Group 3 mice were nebulized with methacholine at increasing doses (1-30 mg/ml) over a 2 min period.
   Post-Challenge Phase

A 5 min post-challenge reading for each of the mice was taken and the readings were averaged.

Reduction in airway hyperresponsiveness was calculated according to the following formula:

$$( 100 \% ) \times [ 1 - \frac{( Treated {Reading}^{Avg} - Veh . Control {Reading}^{Avg} )}{\begin{matrix}( Positive Control {Reading}^{Avg} - \\ Veh . Control {Reading}^{Avg} )\end{matrix}} ]$$

Airway inflammation was measured by eosinophil cell count in bronchoalveolar saline lavage samples (1 mL) of the mice from the three groups. The lavage fluid was centrifuged and the supernatant was removed. The cell pellet was resuspended in saline containing 0.1% BSA, then cytospin smears were made from the cell suspension and stained with Giemsa. The number of eosinophils was counted and the cell concentration adjusted to 0.1×10⁶/mL.

Airway Hyperresponsiveness Results:

Group 1 mice (661±80; n=4);
Group 2 mice (1425±128; n=7); and,
Group 3 mice (1147±49; n=4).

The result for the mice treated with Compound 13 represents an approximate average of 36% reduction in airway hyperresponsiveness compared to the non-treated mice.

Eosinophil Infiltration Results:

Group 1 mice (0±0×10⁵/mL; n=4);
Group 2 mice (0.8±0.2×10⁵/mL; n=9); and,
Group 3 mice (0.2±0.1×10⁵/ml; n=3).

The result for the mice treated with Compound 13 represents an average 75% reduction in airway inflammation compared to the non-treated mice.

### EXAMPLE 36

Inhibition of Ovalbumin-Induced Allergic Rhinitis in Mice

BALB/c mice are sensitized by i.p. injection of OVA emulsified in alum (Day 0, 5, 14, 21). Groups of mice are each challenged by intranasal injection of OVA (Day 22-35, 38). Control group mice receive an equal volume of vehicle by intranasal injection. Nasal symptoms (number of sneezes and episodes of nose rubbing by the front paws) are counted during the 5 min period following the last intranasal injection (Day 38).

Prophylactic Effect

A test compound (in PBS) is administered by intranasal injection (10 and 30 μg/nostril) to both nostrils twice daily 1 hr and 6 hrs prior to intranasal challenge (Days 22-35), once per day prior to intranasal challenge (Days 36, 37) then 1 hr and 6 hrs prior to intranasal challenge (Day 38). One or more suitable anti-allergen agents are used as a positive control.

Compared to vehicle and the positive control, a test compound inhibits nasal symptoms (sneezing/rubbing).

Therapeutic Effect

The dosing of test compound is delayed until the symptoms of rhinitis have appeared (Day 29). A test compound (in PBS) is then administered by intranasal injection (10 μg/nostril) to both nostrils four times per day prior to intranasal challenge (Days 29-38). One or more suitable anti-allergen agents are used as a positive control.

Compared to vehicle and positive control, a test compound inhibits nasal symptoms (sneezing/rubbing).

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A compound of Formula (I): 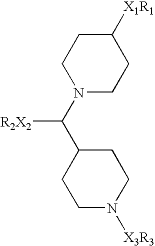
or a salt, isomer, prodrug, metabolite or polymorph thereof wherein
X₁ is absent, alkyl, carbonyl, alkylcarbamoyl or alkylcarbamoylalkyl,
R₁ is aryl or heterocyclyl, wherein heterocyclyl has an optionally present nitrogen atom and wherein the nitrogen atom is optionally oxidized, and wherein aryl and heterocyclyl are each optionally substituted with one or more of alkyl, alkoxy, cyano; halogen, hydroxy, hydroxyalkyl, nitro, amino (optionally substituted with one or more of alkyl, acyl, carbonylalkoxy, sulfonylalkyl, alkylcarboxy or alkylcarbonylalkoxy), alkylcarboxy, alkylcarbonylalkoxy, alkoxycarboxy, alkoxycarbonylalkoxy, alkylamino, alkylaminoalkyl, sulfonylamino, sulfonylaminoalkyl, alkylsulfonylamino, alkylsulfonylaminoalkyl, carboxy, acyl, carbonylalkoxy, carbamoyl or carbamoylalkyl,
X₂ is absent or alkyl,
R₂ is hydroxy, halogen, amino (optionally substituted with one or more of alkyl, formyl, acyl, sulfonylalkyl or carbonylalkoxy), cyano, nitro, alkoxy, carboxy, carbonylalkoxy, oxyacyl, oxyacylaryl, oxyacrylyl, oxyacrylylaryl (optionally substituted on aryl with one or more of alkyl, alkoxy, cyano, halogen, hydroxy, nitro, amino or aminoalkyl), oxycarbonylalkoxy, aminoacylamino, aminoacylaminoalkyl, carbamoyl, carbamoylalkyl, urea or ureaalkyl,
X₃ is carbonyl, carboxyl, acyl, acyloxy, acrylyl, carbonylalkynyl, carbonylalkoxy, carbamoyl, carbamoylalkyl, alkylcarbamoyl, thiocarbamyl or iminomethylaminocarbonyl, wherein when X₃ is carbonylalkoxy, then R₃ is optionally present, and
R₃ is cycloalkyl, aryl or heterocyclyl each optionally substituted with one or more of alkyl, alkoxy, cyano, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl, carbamoylalkyl or aryl (optionally substituted on aryl with one or more of alkyl, alkoxy, halogen, hydroxy, nitro, amino or aminoalkyl).

2. The compound of claim 1, wherein X₁ is absent, alkyl or alkylcarbamoylalkyl.

3. The compound of claim 1, wherein X₁ is alkyl or alkylcarbamoylalkyl.

4. The compound of claim 1, wherein X₁ is absent.

5. The compound of claim 1, wherein R₁ is phenyl or heterocyclyl, wherein heterocyclyl has an optionally present nitrogen atom and wherein the nitrogen atom is optionally oxidized, and wherein phenyl and heterocyclyl are each optionally substituted with one or more of alkyl, alkoxy, cyano, halogen, hydroxy, alkylhydroxy, nitro, amino (optionally substituted with one or more of alkyl, acyl, carbonylalkoxy, sulfonylalkyl, alkylcarboxy or alkylcarbonylalkoxy), alkylcarboxy, alkylcarbonylalkoxy, alkoxycarboxy, alkoxycarbonylalkoxy, alkylamino, alkylaminoalkyl, sulfonylamino, sulfonylaminoalkyl, alkylsulfonylamino, alkylsulfonylaminoalkyl, carboxy, acyl, carbonylalkoxy, carbamoyl or carbamoylalkyl.

6. The compound of claim 1, wherein R₁ is aryl or heterocyclyl, wherein heterocyclyl has an optionally present nitrogen atom and wherein the nitrogen atom is optionally oxidized, and wherein aryl and heterocyclyl are each optionally substituted with one or more of alkyl, alkoxy, cyano, halogen, hydroxy, alkylhydroxy, nitro, amino (optionally substituted with one or more of alkyl, acyl, carbonylalkoxy, sulfonylalkyl, alkylcarboxy or alkylcarbonylalkoxy), alkylcarboxy, alkylcarbonylalkoxy, alkoxycarboxy, alkoxycarbonylalkoxy, sulfonylamino, sulfonylaminoalkyl, alkylsulfonylamino, alkylsulfonylaminoalkyl, carboxy, acyl or carbonylalkoxy.

7. The compound of claim 1, wherein R₁ is phenyl or heterocyclyl, wherein heterocyclyl has an optionally present nitrogen atom and wherein the nitrogen atom is optionally oxidized, and wherein phenyl and heterocyclyl are each optionally substituted with one or more of alkyl, alkoxy, cyano, halogen, hydroxy, alkylhydroxy, nitro, amino (optionally substituted with one or more of alkyl, acyl, carbonylalkoxy, sulfonylalkyl, alkylcarboxy or alkylcarbonylalkoxy), alkylcarboxy, alkylcarbonylalkoxy, alkoxycarboxy, alkoxycarbonylalkoxy, sulfonylamino, sulfonylaminoalkyl, alkylsulfonylamino, alkylsulfonylaminoalkyl, carboxy, acyl or carbonylalkoxy.

8. The compound of claim 1, wherein R₁ is aryl or heterocyclyl, wherein heterocyclyl has an optionally present nitrogen atom and wherein the nitrogen atom is optionally oxidized, and wherein aryl and heterocyclyl are each optionally substituted with one or more of alkyl, alkoxy, halogen, hydroxy, amino (optionally substituted with one or more of alkyl, acyl, carbonylalkoxy or sulfonylalkyl), carboxy, acyl or carbonylalkoxy.

9. The compound of claim 1, wherein R₁ is phenyl or heterocyclyl, wherein heterocyclyl has an optionally present nitrogen atom and wherein the nitrogen atom is optionally oxidized, and wherein phenyl and heterocyclyl are each optionally substituted with one or more of alkyl, alkoxy, halogen, hydroxy, amino (optionally substituted with one or more of alkyl, acyl, carbonylalkoxy or sulfonylalkyl), carboxy, acyl or carbonylalkoxy.

10. The compound of claim 1, wherein X₂ is absent.

11. The compound of claim 1, wherein X₂ is alkyl.

12. The compound of claim 1, wherein R₂ is hydroxy, halogen, amino (optionally substituted with one or more of alkyl, formyl, acyl, sulfonylalkyl or carbonylalkoxy), cyano, nitro, alkoxy, carboxy, carbonylalkoxy, oxyacyl, oxyacylaryl, oxyacrylyl, oxyacrylylaryl (optionally substituted on aryl with one or more of alkyl, alkoxy, cyano, halogen, hydroxy, nitro, amino or aminoalkyl), oxycarbonylalkoxy, aminoacylamino, aminoacylaminoalkyl, carbamoyl, carbamoylalkyl, urea or ureaalkyl.

13. The compound of claim 1, wherein R₂ is hydroxy, halogen, amino (optionally substituted with one or more of alkyl, formyl, acyl, sulfonylalkyl or carbonylalkoxy), alkoxy, carboxy, carbonylalkoxy, oxyacyl, oxyacrylylaryl (optionally substituted on aryl with one or more of halogen or nitro), oxycarbonylalkoxy, aminoacylamino, aminoacylaminoalkyl, carbamoyl or ureaalkyl.

14. The compound of claim 1, wherein R₂ is hydroxy, halogen, amino (optionally substituted with one or more of alkyl, formyl, acyl, sulfonylalkyl or carbonylalkoxy), alkoxy, carboxy, carbonylalkoxy, oxyacyl, oxyacrylylphenyl (optionally substituted on phenyl with one or more of halogen or nitro), oxycarbonylalkoxy, aminoacylamino, aminoacylaminoalkyl, carbamoyl or ureaalkyl.

15. The compound of claim 1, wherein X₃ is carbonyl, acyl, acyloxy, acrylyl, carbonylalkynyl, carbonylalkoxy, carbamoyl, carbamoylalkyl, thiocarbamyl or iminomethylaminocarbonyl, wherein when X₃ is carbonylalkoxy, then R₃ is optionally present.

16. The compound of claim 1, wherein X₃ is carbonyl, acyl, acyloxy, acrylyl, carbonylalkynyl, carbonylalkoxy, carbamoyl, carbamoylalkyl, thiocarbamyl or iminomethylaminocarbonyl, wherein when X₃ is carbonylalkoxy, then R₃ is optionally present.

17. The compound of claim 1, wherein R₃ is cycloalkyl, aryl or heterocyclyl each optionally substituted with one or more of alkyl, alkoxy, cyano, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl, carbamoylalkyl or phenyl (optionally substituted on phenyl with one or more of alkyl, alkoxy, halogen, hydroxy, nitro, amino or aminoalkyl).

18. The compound of claim 1, wherein R₃ is cycloalkyl, phenyl or heterocyclyl each optionally substituted with one or more of alkyl, alkoxy, cyano, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl, carbamoylalkyl or aryl (optionally substituted on aryl with one or more of alkyl, alkoxy, halogen, hydroxy, nitro, amino or aminoalkyl).

19. The compound of claim 1, wherein R₃ is cycloalkyl, phenyl or heterocyclyl each optionally substituted with one or more of alkyl, alkoxy, cyano, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl, carbamoylalkyl or phenyl (optionally substituted on phenyl with one or more of alkyl, alkoxy, halogen, hydroxy, nitro, amino or aminoalkyl).

20. The compound of claim 1, wherein R₃ is cycloalkyl, aryl or heterocyclyl each optionally substituted with one or more of alkyl, alkoxy, halogen, alkyltrihalo, alkoxytrihalo, nitro, thioalkyl, thioalkyltrihalo, carbonylalkoxy or aryl (optionally substituted on aryl with one or more halogen).

21. The compound of claim 1, wherein R₃ is cycloalkyl, phenyl or heterocyclyl each optionally substituted with one or more of alkyl, alkoxy, halogen, alkyltrihalo, alkoxytrihalo, nitro, thioalkyl, thioalkyltrihalo, carbonylalkoxy or aryl (optionally substituted on aryl with one or more halogen).

22. The compound of claim 1, wherein R₃ is cycloalkyl optionally substituted with aryl, wherein aryl is optionally substituted with one or more of alkyl, alkoxy, halogen, hydroxy, nitro, amino or aminoalkyl.

23. The compound of claim 1, wherein R₃ is cycloalkyl optionally substituted with aryl, wherein aryl is optionally substituted with one or more of halogen.

24. The compound of claim 1, wherein R₃ is aryl optionally substituted with one or more of alkyl, alkoxy, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl or carbamoylalkyl.

25. The compound of claim 1, wherein R₃ is phenyl optionally substituted with one or more of alkyl, alkoxy, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl or carbamoylalkyl.

26. The compound of claim 1, wherein R₃ is aryl optionally substituted with one or more of alkyl, alkoxy, halogen, alkyltrihalo, alkoxytrihalo, nitro, thioalkyl, thioalkyltrihalo or carbonylalkoxy.

27. The compound of claim 1, wherein R₃ is phenyl optionally substituted with one or more of alkyl, alkoxy, halogen, alkyltrihalo, alkoxytrihalo, nitro, thioalkyl, thioalkyltrihalo or carbonylalkoxy.

28. The compound of claim 1, wherein R₃ is heterocyclyl optionally substituted with one or more of alkyl, alkoxy, halogen, hydroxy, amino or aminoalkyl.

29. The compound of claim 1, wherein R₃ is heterocyclyl optionally substituted with one or more of halogen.

30. The compound of claim 1, wherein
X₁ is absent, alkyl or alkylcarbamoylalkyl,
R₁ is aryl or heterocyclyl, wherein heterocyclyl has an optionally present nitrogen atom and wherein the nitrogen atom is optionally oxidized, and wherein aryl and heterocyclyl are each optionally substituted with one or more of alkyl, alkoxy, halogen, hydroxy, amino (optionally substituted with one or more of alkyl, acyl, carbonylalkoxy or sulfonylalkyl), carboxy, acyl or carbonylalkoxy,
X₂ is absent or alkyl,
R₂ is hydroxy, halogen, amino (optionally substituted with one or more of alkyl, formyl, acyl, sulfonylalkyl or carbonylalkoxy), alkoxy, carboxy, carbonylalkoxy, oxyacyl, oxyacrylylaryl (optionally substituted on aryl with one or more of halogen or nitro), oxycarbonylalkoxy, aminoacylamino, aminoacylaminoalkyl, carbamoyl or ureaalkyl,
X₃ is carbonyl, acyl, acyloxy, acrylyl, carbonylalkynyl, carbonylalkoxy, carbamoyl, carbamoylalkyl, thiocarbamyl or iminomethylaminocarbonyl, wherein when X₃ is carbonylalkoxy, then R₃ is optionally present, and
R₃ is cycloalkyl, aryl or heterocyclyl each optionally substituted with one or more of alkyl, alkoxy, halogen, alkyltrihalo, alkoxytrihalo, nitro, thioalkyl, thioalkyltrihalo, carbonylalkoxy or aryl (optionally substituted on aryl with one or more halogen).

31. A compound selected from the group consisting of
[4-(1H-indol-3-yl)-piperidin-1-yl]-{1-[(2E)-3-(3,4,5-trifluoro-phenyl)-acryloyl]-piperidin-4-yl}-acetic acid;
{1-[(2E)-3-(3,5-difluoro-phenyl)-acryloyl]-piperidin-4-yl}-[4-(1H-indol-3-yl)-piperidin-1-yl]-acetic acid;
{1-[(2E)-3-(3,5-difluoro-phenyl)-acryloyl]-piperidin-4-yl}-[4-(5-fluoro-1H-indol-3-yl)-piperidin-1-yl]-acetic acid;
[4-(5-fluoro-1H-indol-3-yl)-piperidin-1-yl]-{1-[(2E)-3-(3,4,5-trifluoro-phenyl)-acryloyl]-piperidin-4-yl}-acetic acid;
(S)-{[4-(1H-indol-3-yl)-piperidin-1-yl]}-{1-[(2E)-3-(3,4,5-trifluoro-phenyl)-acryloyl]-piperidin-4-yl}-acetic acid;
[4-(5-hydroxy-1H-indol-3-yl)-piperidin-1-yl]-{1-[(2E)-3-(3,4,5-trifluoro-phenyl)-acryloyl]-piperidin-4-yl}-acetic acid;
{1-[(2E)-3-(3,5-difluoro-phenyl)-acryloyl]-piperidin-4-yl}-[4-(5-hydroxy-1H-indol-3-yl)-piperidin-1-yl]-acetic acid;
{1-[(2E)-3-(3,4-dichloro-phenyl)-acryloyl]-piperidin-4-yl}-[4-(1H-indol-3-yl)-piperidin-1-yl]-acetic acid;
{1-[(2E)-3-(3,4-dichloro-phenyl)-acryloyl]-piperidin-4-yl}-[4-(5-fluoro-1H-indol-3-yl)-piperidin-1-yl]-acetic acid;
[1-(3,4-dichloro-phenylcarbamoyl)-piperidin-4-yl]-[4-(1H-indol-3-yl)-piperidin-1-yl]-acetic acid;
{1-[(2E)-3-(3,4-difluoro-phenyl)-acryloyl]-piperidin-4-yl}-[4-(1H-indol-3-yl)-piperidin-1-yl]-acetic acid;
[4-(1H-indol-3-yl-piperidin-1-yl]-{1-[(2E)-3-(4-trifluoromethyl-phenyl)-acryloyl]-piperidin-4-yl}-acetic acid;
{1-[(2E)-3-(3,5-difluoro-phenyl)-acryloyl]-piperidin-4-yl}-[4-(6-fluoro-1H-indol-3-yl)-piperidin-1-yl]-acetic acid;
[4-(6-chloro-1H-indol-3-yl)-piperidin-1-yl]-{1-[(2E)-3-(3,5-difluoro-phenyl)-acryloyl]-piperidin-4-yl }-acetic acid;
{-[(2E)-3-(3,5-difluoro-phenyl)-acryloyl]-piperidin-4-yl}-[4-(5-methoxy-1H-indol-3-yl)-piperidin-1-yl]-acetic acid;
[4-(1H-indol-3-yl)-piperidin-1-yl]-{1-[(2E)-3-phenyl-acryloyl]-piperidin-4-yl}-acetic acid;
{1-[(2E)-3-(3,5-difluoro-phenyl)-acryloyl]-piperidin-4-yl}-[4-(5-methanesulfonylamino-1H-indol-3-yl)-piperidin-1-yl]-acetic acid;
[4-(5-methoxy-1H-indol-3-yl)-piperidin-1-yl]-{1-[(2E)-3-(3,4,5-trifluoro-phenyl)-acryloyl]-piperidin-4-yl}-acetic acid;
[4-(6-chloro-1H-indol-3-yl)-piperidin-1-yl]-{1-[(2E)-3-(3,4,5-trifluoro-phenyl)-acryloyl]-piperidin-4-yl}-acetic acid;
{-[(2E)-3-(4-chloro-phenyl)-acryloyl]-piperidin-4-yl}-[4-(1H-indol-3-yl)-piperidin-1-yl]-acetic acid;
[4-(1H-indol-3-yl)-piperidin-1-yl]-{1-[(2E)-3-(3-trifluoromethyl-phenyl)-acryloyl]-piperidin-4-yl}-acetic acid;
{1-[(2E)-3-(3-bromo-4-fluoro-phenyl)-acryloyl]-piperidin-4-yl}-[4-(1H-indol-3-yl)-piperidin-1-yl]-acetic acid;
{1-[(2E)-3-(3,5-difluoro-phenyl)-acryloyl]-piperidin-4-yl}-[4-(6-methoxy-1H-indol-3-yl)-piperidin-1-yl]-acetic acid;
{1-[(2E)-3-(3,4-dichloro-phenyl)-acryloyl]-piperidin-4-yl}-[4-(6-fluoro-1H-indol-3-yl)-piperidin-1-yl]-acetic acid;
[1-(3,4-difluoro-phenylcarbamoyl)-piperidin-4-yl]-[4-(1H-indol-3-yl)-piperidin-1-yl]-acetic acid;
{1-[(2E)-3-(3,5-difluoro-phenyl)-acryloyl]-piperidin-4-yl}-[4-(4-methoxy-1H-indol-3-yl)-piperidin-1-yl]-acetic acid;
{1-[(2E)-3-(3,5-difluoro-phenyl)-acryloyl]-piperidin-4-yl)-[4-(7-methoxy-1H-indol-3-yl}-piperidin-1-yl]-acetic acid;
[1-(3,5-dichloro-phenylthiocarbamoyl)-piperidin-4-yl]-[4-(1H-indol-3-yl)-piperidin-1-yl]-acetic acid;
[4-(6-chloro-1H-indol-3-yl)-piperidin-1-yl]-{1-[(2E)-3-(3,4-dichloro-phenyl)-acryloyl]-piperidin-4-yl}-acetic acid;
{1-[(2E)-3-(3,4-dichloro-phenyl)-acryloyl]-piperidin-4-yl}-[4-(5-methoxy-1H-indol-3-yl)-piperidin-1-yl]-acetic acid;
[1-(3-chloro-4-fluoro-phenylcarbamoyl)-piperidin-4-yl]-[4-(1H-indol-3-yl)-piperidin-1-yl]-acetic acid;
[1-(3-chloro-4-methyl-phenylcarbamoyl)-piperidin-4-yl]-[4-(1H-indol-3-yl)-piperidin-1-yl]-acetic acid;
{-[(3,4-dichloro-benzoylamino)-imino-methyl]-piperidin-4-yl}-[4-(1H-indol-3-yl)-piperidin-1-yl]-acetic acid;
{1-[imino-(3,4,5-trifluoro-benzoylamino)-methyl]-piperidin-4-yl}-[4-(1H-indol-3-yl)-piperidin-1-yl]-acetic acid;
[4-(5-methanesulfonylamino-1H-indol-3-yl)-piperidin-1-yl]-{1-[(2E)-3-(3,4,5-trifluoro-phenyl)-acryloyl]-piperidin-4-yl}-acetic acid;
[1-(4-chloro-3-trifluoromethyl-phenylcarbamoyl)-piperidin-4-yl]-[4-(1H-indol-3-yl)-piperidin-1-yl]-acetic acid;
[4-(1H-indol-3-yl)-piperidin-1-yl]-{1-[(2E)-3-(4-nitro-phenyl)-acryloyl]-piperidin-4-yl}-acetic acid;
{1-[(2E)-3-(4-bromo-phenyl)-acryloyl]-piperidin-4-yl}-[4-(1H-indol-3-yl)-piperidin-1-yl]-acetic acid;
{1-[(2E)-3-(3-fluoro-phenyl)-acryloyl]-piperidin-4-yl}-[4-(1H-indol-3-yl)-piperidin-1-yl]-acetic acid;
[1-(3,4-dichloro-phenylthiocarbamoyl)-piperidin-4-yl]-[4-(1H-indol-3-yl)-piperidin-1-yl]-acetic acid;
[4-(1H-indol-3-yl)-piperidin-1-yl]-{1-[(2E)-3-m-tolyl-acryloyl]-piperidin-4-yl}-acetic acid;
{-[(2E)-3-(3-bromo-phenyl)-acryloyl]-piperidin-4-yl}-[4-(1H-indol-3-yl)-piperidin-1-yl]-acetic acid;
[4-(1H-indol-3-yl)-piperidin-1-yl]-{1-[(2E)-3-(3-methoxy-phenyl)-acryloyl]-piperidin-4-yl}-acetic acid;
{1-[(2E)-3-(3-fluoro-4-methyl-phenyl)-acryloyl]-piperidin-4-yl}-[4-(1H-indol-3-yl)-piperidin-1-yl]-acetic acid;
{1-[(2E)-3-(3-fluoro-4-trifluoromethyl-phenyl)-acryloyl]-piperidin-4-yl}-[4-(1H-indol-3-yl)-piperidin-1-yl]-acetic acid;
{1-[(2E)-3-(3-chloro-4-fluoro-phenyl)-acryloyl]-piperidin-4-yl}-[4-(1H-indol-3-yl)-piperidin-1-yl]-acetic acid;
{1-[(2E)-3-(4-fluoro-phenyl)-acryloyl]-piperidin-4-yl}-[4-(1H-indol-3-yl)-piperidin-1-yl]-acetic acid;
[4-(1H-indol-3-yl)-piperidin-1-yl]-[1-(3-trifluoromethyl-phenylthiocarbamoyl)-piperidin-4-yl]-acetic acid;
[4-(1H-indol-3-yl)-piperidin-1-yl]-[1-(4-trifluoromethyl-phenylthiocarbamoyl)-piperidin-4-yl]-acetic acid;
[4-(1H-pyrrol-3-yl)-piperidin-1-yl]-{1-[(2E)-3-(3,4,5-trifluoro-phenyl)-acryloyl]-piperidin-4-yl}-acetic acid;
[4-(6-methanesulfonylamino-1H-indol-3-yl)-piperidin-1-yl]-{1-[(2E)-3-(3,4,5-trifluoro-phenyl)-acryloyl]-piperidin-4-yl)}-acetic acid;
[1-(4-chloro-phenylcarbamoyl)-piperidin-4-yl]-[4-(1H-indol-3-yl)-piperidin-1-yl]-acetic acid;
[4-(1H-indol-3-yl)-piperidin-1-yl]-{1-[(2E)-3-(3-nitro-phenyl)-acryloyl]-piperidin-4-yl}-acetic acid;
{1-[(2E)-3-(3-chloro-phenyl)-acryloyl]-piperidin-4-yl}-[4-(1H-indol-3-yl)-piperidin-1-yl]-acetic acid;
[1-(3,4-dichloro-phenylcarbamoyl)-piperidin-4-yl]-[4-(5-methoxy-1H-indol-3-yl)-piperidin-1-yl]-acetic acid;
[1-(3,4-dichloro-phenylcarbamoyl)-piperidin-4-yl]-[4-(6-methoxy-1H-indol-3-yl)-piperidin-1-yl]-acetic acid;
[4-(1H-indol-3-yl)-piperidin-1-yl]-[1-(4-trifluoromethyl-phenylcarbamoyl)-piperidin-4-yl]-acetic acid;
[1-(4-bromo-3-methyl-phenylcarbamoyl)-piperidin-4-yl]-[4-(1H-indol-3-yl)-piperidin-1-yl]-acetic acid;
[4-(1H-indol-3-yl)-piperidin-1-yl]-[1-(4-methyl-3-trifluoromethyl-phenylcarbamoyl)-piperidin-4-yl]-acetic acid;
[4-(7-methoxy-1H-indol-3-yl)-piperidin-1-yl]-{1-[(2E)-3-(3,4,5-trifluoro-phenyl)-acryloyl]-piperidin-4-yl}-acetic acid;
[1-(3,4-dichloro-phenylcarbamoyl)-piperidin-4-yl]-[4-(5-methanesulfonylamino-1H-indol-3-yl)-piperidin-1-yl]-acetic acid;
(2E)-1-(4-{2-hydroxy-1-[4-(1H-indol-3-yl)-piperidin-1-yl]-ethyl}-piperidin-1-yl)-3-(3,4,5-trifluoro-phenyl)-propenone;
(2E)-3-(3,4-difluoro-phenyl)-1-(4-{2-hydroxy-1-[4-(1H-indol-3-yl)-piperidin-1-yl]-ethyl}-piperidin-1-yl)-propenone;
(2E)-3-(3,5-difluoro-phenyl)-1-(4-{2-hydroxy-1-[4-(1H-indol-3-yl)-piperidin-1-yl]-ethyl}-piperidin-1-yl)-propenone;
(2E)-1-(4-{2-hydroxy-1-[4-(1H-indol-3-yl)-piperidin-1-yl]-ethyl}-piperidin-1-yl)-3-(3-trifluoromethyl-phenyl)-propenone;
(2E)-3-(3,4-dichloro-phenyl)-1-(4-{2-hydroxy-1-[4-(1H-indol-3-yl)-piperidin-1-yl]-ethyl}-piperidin-1-yl)-propenone;
4-{2-hydroxy-1-[4-(1H-indol-3-yl)-piperidin-1-yl]-ethyl}-piperidine-1-carbothioic acid (3,4-dichloro-phenyl)-amide;
4-{2-hydroxy-1-[4-(1H-indol-3-yl)-piperidin-1-yl]-ethyl}-piperidine-1-carboxylic acid (3,4-dichloro-phenyl)-amide;
4-{2-hydroxy-1-[4-(1H-indol-3-yl)-piperidin-1-yl]-ethyl}-piperidine-1-carboxylic acid (3,5-difluoro-phenyl)-amide;
(2E)-1-(4-{2-hydroxy-1-[4-(6-methoxy-1H-indol-3-yl)-piperidin-1-yl]-ethyl}-piperidin-1-yl)-3-(3,4,5-trifluoro-phenyl)-propenone;
(2E)-1-(4-{2-hydroxy-1-[4-(7-methoxy-1H-indol-3-yl)-piperidin-1-yl]-ethyl}-piperidin-1-yl)-3-(3,4,5-trifluoro-phenyl)-propenone;
[1-(3,5-bis-trifluoromethyl-phenylcarbamoyl)-piperidin-4-yl]-[4-(1H-indol-3-yl)-piperidin-1-yl]-acetic acid;
(2E)-3-(3,5-difluoro-phenyl)-1-(4-{2-hydroxy-1-[4-(4-methoxy-phenyl)-piperidin-1-yl]-ethyl}-piperidin-1-yl)-propenone;
(2E)-3-(3,4-difluoro-phenyl)-1-(4-{2-hydroxy-1-[4-(4-methoxy-phenyl)-piperidin-1-yl]-ethyl}-piperidin-1-yl)-propenone;
[4-(1H-indol-3-yl)-piperidin-1-yl]-[1-(4-trifluoromethylsulfanyl-phenylcarbamoyl)-piperidin-4-yl]-acetic acid;
[4-(1H-indol-3-yl)-piperidin-1-yl]-[1-(4-trifluoromethoxy-phenylcarbamoyl)-piperidin-4-yl]-acetic acid;
[4-(1H-indol-3-yl)-piperidin-1-yl]-[1-(3-methylsulfanyl-phenylcarbamoyl)-piperidin-4-yl]-acetic acid;
3-[1-(carboxy-{1-[(2E)-3-(3,4,5-trifluoro-phenyl)-acryloyl]-piperidin-4-yl}-methyl)-piperidin-4-yl]-1H-indole-5-carboxylic acid methyl ester;
[4-(1H-pyrrolo[2,3-b]pyridin-3-yl)-piperidin-1-yl]-{1-[(2E)-3-(3,4,5-trifluoro-phenyl)-acryloyl]-piperidin-4-yl}-acetic acid;
(2E)-1-(4-{2-hydroxy-1-[4-(1H-pyrrolo[2,3-b]pyridin-3-yl)-piperidin-1-yl]-ethyl}-piperidin-1-yl)-3-(3,4,5-trifluoro-phenyl)-propenone;
(2E)-1-(4-{2-hydroxy-1-[4-(4-methoxy-phenyl)-piperidin-1-yl]-ethyl}-piperidin-1-yl)-3-(3,4,5-trifluoro-phenyl)-propenone;
(2E)-3-(3,4-dichloro-phenyl)-1-(4-{2-hydroxy-1-[4-(5-methoxy-1H-indol-3-yl)-piperidin-1-yl]-ethyl}-piperidin-1-yl)-propenone;
(2E)-1-(4-{2-hydroxy-1-[4-(5-methoxy-1H-indol-3-yl)-piperidin-1-yl]-ethyl}-piperidin-1-yl)-3-(3,4,5-trifluoro-phenyl)-propenone;
(2E)-1-(4-{1-[4-(5-fluoro-1H-indol-3-yl)-piperidin-1-yl]-2-hydroxy-ethyl}-piperidin-1-yl)-3-(3,4,5-trifluoro-phenyl)-propenone;
(2E)-3-(3,5-difluoro-phenyl)-1-(4-{1-[4-(5-fluoro-1H-indol-3-yl)-piperidin-1-yl]-2-hydroxy-ethyl}-piperidin-1-yl)-propenone;
(2E)-3-(3,5-difluoro-phenyl)-1-(4-{(1S)-2-hydroxy-1-[4-(4-methoxy-phenyl)-piperidin-1-yl]-ethyl}-piperidin-1-yl)-propenone;
(2E)-3-(3,5-difluoro-phenyl)-1-(4-{(1R)-2-hydroxy-1-[4-(4-methoxy-phenyl)-piperidin-1-yl]-ethyl}-piperidin-1-yl)-propenone;
(2E)-1-(4-{(1S)-2-hydroxy-1-[4-(1H-indol-3-yl)-piperidin-1-yl]-ethyl}-piperidin-1-yl)-3-(3,4,5-trifluoro-phenyl)-propenone;
(2E)-1-(4-{(1R)-2-hydroxy-1-[4-(1H-indol-3-yl)-piperidin-1-yl]-ethyl}-piperidin-1-yl)-3-(3,4,5-trifluoro-phenyl)-propenone;
N-{3-[1-(1-{1-[(2E)-3-(3,4-dichloro-phenyl)-acryloyl]-piperidin-4-yl}-2-hydroxy-ethyl)-piperidin-4-yl]-1H-indol-5-yl}-methanesulfonamide;
N-{3-[1-(2-hydroxy-1-{1-[(2E)-3-(3,4,5-trifluoro-phenyl)-acryloyl]-piperidin-4-yl}-ethyl)-piperidin-4-yl]-1H-indol-5-yl}-methanesulfonamide;
(2E)-3-(3,5-difluoro-phenyl)-1-(4-{2-hydroxy-1-[4-(1H-pyrrolo[2,3-b]pyridin-3-yl)-piperidin-1-yl]-ethyl}-piperidin-1-yl)-propenone;
(2E)-3-(3,5-difluoro-phenyl)-1-(4-{2-hydroxy-1-[4-(7-oxy-1H-pyrrolo[2,3-b]pyridin-3-yl)-piperidin-1-yl]-ethyl}-piperidin-1-yl)-propenone;
(2E)-3-(3,4-dichloro-phenyl)-1-(4-{2-hydroxy-1-[4-(1H-pyrrolo[2,3-b]pyridin-3-yl)-piperidin-1-yl]-ethyl}-piperidin-1-yl)-propenone;
[4-(6-fluoro-1H-indol-3-yl)-piperidin-1-yl]-{1-[(2E)-3-(3,4,5-trifluoro-phenyl)-acryloyl]-piperidin-4-yl}-acetic acid;
N-(2-[4-(1H-indol-3-yl)-piperidin-1-yl]-2-{1-[(2E)-3-(3,4,5-trifluoro-phenyl)-acryloyl]-piperidin-4-yl}-ethyl)-acetamide;
(2-[4-(1H-indol-3-yl)-piperidin-1-yl]-2-{1-[(2E)-3-(3,4,5-trifluoro-phenyl)-acryloyl]-piperidin-4-yl}-ethyl)-carbamic acid methyl ester;
acetic acid 2-{4-[5-(acetyl-methanesulfonyl-amino)-1H-indol-3-yl]-piperidin-1-yl}-2-{1-[(2E)-3-(3,5-difluoro-phenyl)-acryloyl]-piperidin-4-yl}-ethyl ester; and
(2E)-3-(3,5-difluoro-phenyl)-1-(4-{2-hydroxy-1-[4-(5-hydroxy-1H-indol-3-yl)-piperidin-1-yl]-ethyl}-piperidin-1-yl)-propenone.

32. A composition comprising an effective amount of the compound of claim 1 and a pharmaceutically acceptable carrier.

33. The composition of claim 32 selected from a topically applied composition, an intranasally applied composition or an ocularly applied composition.

34. A process for preparing the composition of claim 33 comprising the step of admixing the compound of claim 1 and a pharmaceutically acceptable carrier.

35. A method for preventing, treating or ameliorating a CCR2 mediated inflammatory syndrome, disorder or disease in a subject in need thereof comprising administering to the subject an effective amount of the compound of claim 1 or composition or medicament thereof.

36. The method of claim 35, wherein the effective amount is from about 0.1 ng/kg/day to about 300 mg/kg/day.

37. The method of claim 35, wherein the syndrome, disorder or disease is associated with elevated MCP-1 expression or MCP-1 overexpression, or is an inflammatory condition that accompanies syndromes, disorders or diseases associated with elevated MCP-1 expression or MCP-1 overexpression.

38. The method of claim 35, wherein the syndrome, disorder or disease is selected from ophthalmic disorders, uveitis, atherosclerosis, rheumatoid arthritis, psoriasis, psoriatic arthritis, atopic dermatitis, multiple sclerosis, Crohn's Disease, ulcerative colitis, nephritis, organ allograft rejection, fibroid lung, renal insufficiency, diabetes and diabetic complications, diabetic nephropathy, diabetic retinopathy, diabetic retinitis, diabetic microangiopathy, tuberculosis, chronic obstructive pulmonary disease, sarcoidosis, invasive staphyloccocia, inflammation after cataract surgery, allergic rhinitis, allergic conjunctivitis, chronic urticaria, asthma, allergic asthma, periodontal diseases, periodonitis, gingivitis, gum disease, diastolic cardiomyopathies, cardiac infarction, myocarditis, chronic heart failure, angiostenosis, restenosis, reperfusion disorders, glomerulonephritis, solid tumors and cancers, chronic lymphocytic leukemia, chronic myelocytic leukemia, multiple myeloma, malignant myeloma, Hodgkin's disease, or carcinomas of the bladder, breast, cervix, colon, lung, prostate, or stomach.

39. The method of claim 35, wherein the method further comprises preventing, treating or ameliorating CCR2 mediated ophthalmic disorders, rheumatoid arthritis, psoriasis, psoriatic arthritis, atopic dermatitis, chronic obstructive pulmonary disease, allergic rhinitis, asthma, allergic asthma, periodontal diseases in a subject in need thereof comprising administering to the subject an effective amount of the compound of claim 1 or composition or medicament thereof.

40. The method of claim 39, wherein the ophthalmic disorder is selected from uveitis or allergic conjunctivitis and the periodontal disease is selected from periodonitis, gingivitis or gum disease.

41. The method of claim 40, wherein uveitis is selected from acute, recurring or chronic uveitis.

42. The method of claim 40, wherein uveitis is selected from anterior uveitis, intermediate uveitis, posterior uveitis or panuveitis.

43. The method of claim 35, wherein the method further comprises preventing, treating or ameliorating CCR2 mediated acute uveitis, recurring uveitis, chronic uveitis, allergic conjunctivitis, rheumatoid arthritis, psoriasis, psoriatic arthritis, atopic dermatitis, chronic obstructive pulmonary disease, allergic rhinitis, asthma, allergic asthma, periodonitis, gingivitis or gum disease in a subject in need thereof comprising administering to the subject an effective amount of the compound of claim 1 or composition or medicament thereof.

44. The method of claim 35, wherein the method further comprises preventing, treating or ameliorating a CCR2 mediated inflammatory syndrome, disorder or disease in a subject in need thereof comprising administering to the subject an effective amount of the compound of claim 1 or composition or medicament thereof in a combination therapy with one or more anti-inflammatory agents, anti-infective agents or immunosuppressive agents.
